SEQUENCE LISTING (1008 sequences)

SEQ ID NO: 1 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 2 - protein, 26 aa, synthetic construct
  REGION 1..26: note = Recombinant collagen fragment
AAGARGNDGARGSDGQPGPPGPPGTA

SEQ ID NO: 3 - protein, 35 aa, synthetic construct
  REGION 1..35: note = Recombinant collagen fragment
AAGARGNDGARGSDGQPGPPGPPGTAGFPGSPGAK

SEQ ID NO: 4 - protein, 41 aa, synthetic construct
  REGION 1..41: note = Recombinant collagen fragment
AAGARGNDGARGSDGQPGPPGPPGTAGFPGSPGAKGEVGPA

SEQ ID NO: 5 - protein, 21 aa, synthetic construct
  REGION 1..21: note = Recombinant collagen fragment
AAGEPGRDGVPGGPGMRGMPG

SEQ ID NO: 6 - protein, 27 aa, synthetic construct
  REGION 1..27: note = Recombinant collagen fragment
AAGEPGRDGVPGGPGMRGMPGSPGGPG

SEQ ID NO: 7 - protein, 17 aa, synthetic construct
  REGION 1..17: note = Recombinant collagen fragment
AAGERGAPGFRGPAGPN

SEQ ID NO: 8 - protein, 13 aa, synthetic construct
  REGION 1..13: note = Recombinant collagen fragment
AGAQGPPGPPGIN

SEQ ID NO: 9 - protein, 34 aa, synthetic construct
  REGION 1..34: note = Recombinant collagen fragment
AGAQGPPGPPGINGSPGGKGEMGPAGIPGAPGLM

SEQ ID NO: 10 - protein, 25 aa, synthetic construct
  REGION 1..25: note = Recombinant collagen fragment
AGARGNDGARGSDGQPGPPGPPGTA

SEQ ID NO: 11 - protein, 40 aa, synthetic construct
  REGION 1..40: note = Recombinant collagen fragment
AGARGNDGARGSDGQPGPPGPPGTAGFPGSPGAKGEVGPA

SEQ ID NO: 12 - protein, 26 aa, synthetic construct
  REGION 1..26: note = Recombinant collagen fragment
AGEPGRDGVPGGPGMRGMPGSPGGPG

SEQ ID NO: 13 - protein, 16 aa, synthetic construct
  REGION 1..16: note = Recombinant collagen fragment
AGERGAPGFRGPAGPN

SEQ ID NO: 14 - protein, 10 aa, synthetic construct
  REGION 1..10: note = Recombinant collagen fragment
AGIPGAPGLM

SEQ ID NO: 15 - protein, 28 aa, synthetic construct
  REGION 1..28: note = Recombinant collagen fragment
AGIPGAPGLMGARGPPGPAGANGAPGLR

SEQ ID NO: 16 - protein, 12 aa, synthetic construct
  REGION 1..12: note = Recombinant collagen fragment
AGIPGFPGMKGH

SEQ ID NO: 17 - protein, 14 aa, synthetic construct
  REGION 1..14: note = Recombinant collagen fragment
AGIPGFPGMKGHRG

SEQ ID NO: 18 - protein, 15 aa, synthetic construct
  REGION 1..15: note = Recombinant collagen fragment
AGIPGFPGMKGHRGF

SEQ ID NO: 19 - protein, 19 aa, synthetic construct
  REGION 1..19: note = Recombinant collagen fragment
AGIPGFPGMKGHRGFDGRN

SEQ ID NO: 20 - protein, 31 aa, synthetic construct
  REGION 1..31: note = Recombinant collagen fragment
AGIPGFPGMKGHRGFDGRNGEKGETGAPGLK

SEQ ID NO: 21 - protein, 34 aa, synthetic construct
  REGION 1..34: note = Recombinant collagen fragment
AGIPGFPGMKGHRGFDGRNGEKGETGAPGLKGEN

SEQ ID NO: 22 - protein, 25 aa, synthetic construct
  REGION 1..25: note = Recombinant collagen fragment
AGKDGESGRPGRPGERGLPGPPGIK

SEQ ID NO: 23 - protein, 28 aa, synthetic construct
  REGION 1..28: note = Recombinant collagen fragment
AGKDGESGRPGRPGERGLPGPPGIKGPA

SEQ ID NO: 24 - protein, 13 aa, synthetic construct
  REGION 1..13: note = Recombinant collagen fragment
AGPPGPPGPPGTS

SEQ ID NO: 25 - protein, 15 aa, synthetic construct
  REGION 1..15: note = Recombinant collagen fragment
AGPPGPPGPPGTSGH

SEQ ID NO: 26 - protein, 16 aa, synthetic construct
  REGION 1..16: note = Recombinant collagen fragment
AGPSGPPGPPGAIGPS

SEQ ID NO: 27 - protein, 43 aa, synthetic construct
  REGION 1..43: note = Recombinant collagen fragment
AGPSGPPGPPGAIGPSGPAGKDGESGRPGRPGERGLPGPPGIK

SEQ ID NO: 28 - protein, 55 aa, synthetic construct
  REGION 1..55: note = Recombinant collagen fragment
AGSPGSNGAPGQRGEPGPQGHAGAQGPPGPPGINGSPGGKGEMGPAGIPGAPGLM

SEQ ID NO: 29 - protein, 19 aa, synthetic construct
  REGION 1..19: note = Recombinant collagen fragment
AGYPGPAGPPGPPGPPGTS

SEQ ID NO: 30 - protein, 55 aa, synthetic construct
  REGION 1..55: note = Recombinant collagen fragment
AGYPGPAGPPGPPGPPGTSGHPGSPGSPGYQGPPGEPGQAGPSGPPGPPGAIGPS

SEQ ID NO: 31 - protein, 32 aa, synthetic construct
  REGION 1..32: note = Recombinant collagen fragment
AIGPSGPAGKDGESGRPGRPGERGLPGPPGIK

SEQ ID NO: 32 - protein, 27 aa, synthetic construct
  REGION 1..27: note = Recombinant collagen fragment
AKGEVGPAGSPGSNGAPGQRGEPGPQG

SEQ ID NO: 33 - protein, 41 aa, synthetic construct
  REGION 1..41: note = Recombinant collagen fragment
AKGEVGPAGSPGSNGAPGQRGEPGPQGHAGAQGPPGPPGIN

SEQ ID NO: 34 - protein, 26 aa, synthetic construct
  REGION 1..26: note = Recombinant collagen fragment
ANGAPGLRGGAGEPGKNGAKGEPGPR

SEQ ID NO: 35 - protein, 23 aa, synthetic construct
  REGION 1..23: note = Recombinant collagen fragment
ANGLPGAAGERGAPGFRGPAGPN

SEQ ID NO: 36 - protein, 17 aa, synthetic construct
  REGION 1..17: note = Recombinant collagen fragment
APGERGRPGLPGAAGAR

SEQ ID NO: 37 - protein, 38 aa, synthetic construct
  REGION 1..38: note = Recombinant collagen fragment
APGERGRPGLPGAAGARGNDGARGSDGQPGPPGPPGTA

SEQ ID NO: 38 - protein, 14 aa, synthetic construct
  REGION 1..14: note = Recombinant collagen fragment
APGLKGENGLPGEN

SEQ ID NO: 39 - protein, 23 aa, synthetic construct
  REGION 1..23: note = Recombinant collagen fragment
APGLKGENGLPGENGAPGPMGPR

SEQ ID NO: 40 - protein, 14 aa, synthetic construct
  REGION 1..14: note = Recombinant collagen fragment
APGLMGARGPPGPA

SEQ ID NO: 41 - protein, 17 aa, synthetic construct
  REGION 1..17: note = Recombinant collagen fragment
APGLMGARGPPGPAGAN

SEQ ID NO: 42 - protein, 23 aa, synthetic construct
  REGION 1..23: note = Recombinant collagen fragment
APGLMGARGPPGPAGANGAPGLR

SEQ ID NO: 43 - protein, 26 aa, synthetic construct
  REGION 1..26: note = Recombinant collagen fragment
APGQRGEPGPQGHAGAQGPPGPPGIN

SEQ ID NO: 44 - protein, 11 aa, synthetic construct
  REGION 1..11: note = Recombinant collagen fragment
AQGPPGPPGIN

SEQ ID NO: 45 - protein, 15 aa, synthetic construct
  REGION 1..15: note = Recombinant collagen fragment
AQGPPGPPGINGSPG

SEQ ID NO: 46 - protein, 17 aa, synthetic construct
  REGION 1..17: note = Recombinant collagen fragment
AQGPPGPPGINGSPGGK

SEQ ID NO: 47 - protein, 23 aa, synthetic construct
  REGION 1..23: note = Recombinant collagen fragment
AQGPPGPPGINGSPGGKGEMGPA

SEQ ID NO: 48 - protein, 35 aa, synthetic construct
  REGION 1..35: note = Recombinant collagen fragment
AQGPPGPPGINGSPGGKGEMGPAGIPGAPGLMGAR

SEQ ID NO: 49 - protein, 23 aa, synthetic construct
  REGION 1..23: note = Recombinant collagen fragment
ARGNDGARGSDGQPGPPGPPGTA

SEQ ID NO: 50 - protein, 25 aa, synthetic construct
  REGION 1..25: note = Recombinant collagen fragment
ARGNDGARGSDGQPGPPGPPGTAGF

SEQ ID NO: 51 - protein, 38 aa, synthetic construct
  REGION 1..38: note = Recombinant collagen fragment
ARGNDGARGSDGQPGPPGPPGTAGFPGSPGAKGEVGPA

SEQ ID NO: 52 - protein, 44 aa, synthetic construct
  REGION 1..44: note = Recombinant collagen fragment
ARGNDGARGSDGQPGPPGPPGTAGFPGSPGAKGEVGPAGSPGSN

SEQ ID NO: 53 - protein, 17 aa, synthetic construct
  REGION 1..17: note = Recombinant collagen fragment
ARGPPGPAGANGAPGLR

SEQ ID NO: 54 - protein, 26 aa, synthetic construct
  REGION 1..26: note = Recombinant collagen fragment
ARGPPGPAGANGAPGLRGGAGEPGKN

SEQ ID NO: 55 - protein, 17 aa, synthetic construct
  REGION 1..17: note = Recombinant collagen fragment
ARGSDGQPGPPGPPGTA

SEQ ID NO: 56 - protein, 26 aa, synthetic construct
  REGION 1..26: note = Recombinant collagen fragment
ARGSDGQPGPPGPPGTAGFPGSPGAK

SEQ ID NO: 57 - protein, 32 aa, synthetic construct
  REGION 1..32: note = Recombinant collagen fragment
ARGSDGQPGPPGPPGTAGFPGSPGAKGEVGPA

SEQ ID NO: 58 - protein, 23 aa, synthetic construct
  REGION 1..23: note = Recombinant collagen fragment
DAGAPGAPGGKGDAGAPGERGPP

SEQ ID NO: 59 - protein, 23 aa, synthetic construct
  REGION 1..23: note = Recombinant collagen fragment
DGAPGKNGERGGPGGPGPQGPPG

SEQ ID NO: 60 - protein, 25 aa, synthetic construct
  REGION 1..25: note = Recombinant collagen fragment
DGAPGKNGERGGPGGPGPQGPPGKN

SEQ ID NO: 61 - protein, 27 aa, synthetic construct
  REGION 1..27: note = Recombinant collagen fragment
DGAPGKNGERGGPGGPGPQGPPGKNGE

SEQ ID NO: 62 - protein, 29 aa, synthetic construct
  REGION 1..29: note = Recombinant collagen fragment
DGAPGKNGERGGPGGPGPQGPPGKNGETG

SEQ ID NO: 63 - protein, 30 aa, synthetic construct
  REGION 1..30: note = Recombinant collagen fragment
DGAPGKNGERGGPGGPGPQGPPGKNGETGP

SEQ ID NO: 64 - protein, 31 aa, synthetic construct
  REGION 1..31: note = Recombinant collagen fragment
DGAPGKNGERGGPGGPGPQGPPGKNGETGPQ

SEQ ID NO: 65 - protein, 32 aa, synthetic construct
  REGION 1..32: note = Recombinant collagen fragment
DGAPGKNGERGGPGGPGPQGPPGKNGETGPQG

SEQ ID NO: 66 - protein, 35 aa, synthetic construct
  REGION 1..35: note = Recombinant collagen fragment
DGAPGKNGERGGPGGPGPQGPPGKNGETGPQGPPG

SEQ ID NO: 67 - protein, 36 aa, synthetic construct
  REGION 1..36: note = Recombinant collagen fragment
DGAPGKNGERGGPGGPGPQGPPGKNGETGPQGPPGP

SEQ ID NO: 68 - protein, 38 aa, synthetic construct
  REGION 1..38: note = Recombinant collagen fragment
DGAPGKNGERGGPGGPGPQGPPGKNGETGPQGPPGPTG

SEQ ID NO: 69 - protein, 40 aa, synthetic construct
  REGION 1..40: note = Recombinant collagen fragment
DGAPGKNGERGGPGGPGPQGPPGKNGETGPQGPPGPTGPG

SEQ ID NO: 70 - protein, 41 aa, synthetic construct
  REGION 1..41: note = Recombinant collagen fragment
DGAPGKNGERGGPGGPGPQGPPGKNGETGPQGPPGPTGPGG

SEQ ID NO: 71 - protein, 17 aa, synthetic construct
  REGION 1..17: note = Recombinant collagen fragment
DGARGSDGQPGPPGPPG

SEQ ID NO: 72 - protein, 19 aa, synthetic construct
  REGION 1..19: note = Recombinant collagen fragment
DGARGSDGQPGPPGPPGTA

SEQ ID NO: 73 - protein, 20 aa, synthetic construct
  REGION 1..20: note = Recombinant collagen fragment
DGARGSDGQPGPPGPPGTAG

SEQ ID NO: 74 - protein, 21 aa, synthetic construct
  REGION 1..21: note = Recombinant collagen fragment
DGARGSDGQPGPPGPPGTAGF

SEQ ID NO: 75 - protein, 23 aa, synthetic construct
  REGION 1..23: note = Recombinant collagen fragment
DGARGSDGQPGPPGPPGTAGFPG

SEQ ID NO: 76 - protein, 28 aa, synthetic construct
  REGION 1..28: note = Recombinant collagen fragment
DGARGSDGQPGPPGPPGTAGFPGSPGAK

SEQ ID NO: 77 - protein, 29 aa, synthetic construct
  REGION 1..29: note = Recombinant collagen fragment
DGARGSDGQPGPPGPPGTAGFPGSPGAKG

SEQ ID NO: 78 - protein, 34 aa, synthetic construct
  REGION 1..34: note = Recombinant collagen fragment
DGARGSDGQPGPPGPPGTAGFPGSPGAKGEVGPA

SEQ ID NO: 79 - protein, 35 aa, synthetic construct
  REGION 1..35: note = Recombinant collagen fragment
DGARGSDGQPGPPGPPGTAGFPGSPGAKGEVGPAG

SEQ ID NO: 80 - protein, 38 aa, synthetic construct
  REGION 1..38: note = Recombinant collagen fragment
DGARGSDGQPGPPGPPGTAGFPGSPGAKGEVGPAGSPG

SEQ ID NO: 81 - protein, 40 aa, synthetic construct
  REGION 1..40: note = Recombinant collagen fragment
DGARGSDGQPGPPGPPGTAGFPGSPGAKGEVGPAGSPGSN

SEQ ID NO: 82 - protein, 52 aa, synthetic construct
  REGION 1..52: note = Recombinant collagen fragment
DGARGSDGQPGPPGPPGTAGFPGSPGAKGEVGPAGSPGSNGAPGQRGEPGPQ

SEQ ID NO: 83 - protein, 22 aa, synthetic construct
  REGION 1..22: note = Recombinant collagen fragment
DGESGRPGRPGERGLPGPPGIK

SEQ ID NO: 84 - protein, 25 aa, synthetic construct
  REGION 1..25: note = Recombinant collagen fragment
DGESGRPGRPGERGLPGPPGIKGPA

SEQ ID NO: 85 - protein, 31 aa, synthetic construct
  REGION 1..31: note = Recombinant collagen fragment
DGKPGPPGSQGESGRPGPPGPSGPRGQPGVM

SEQ ID NO: 86 - protein, 13 aa, synthetic construct
  REGION 1..13: note = Recombinant collagen fragment
DGQPGPPGPPGTA

SEQ ID NO: 87 - protein, 28 aa, synthetic construct
  REGION 1..28: note = Recombinant collagen fragment
DGQPGPPGPPGTAGFPGSPGAKGEVGPA

SEQ ID NO: 88 - protein, 16 aa, synthetic construct
  REGION 1..16: note = Recombinant collagen fragment
DGRNGEKGETGAPGLK

SEQ ID NO: 89 - protein, 25 aa, synthetic construct
  REGION 1..25: note = Recombinant collagen fragment
DGRNGEKGETGAPGLKGENGLPGEN

SEQ ID NO: 90 - protein, 34 aa, synthetic construct
  REGION 1..34: note = Recombinant collagen fragment
DGRNGEKGETGAPGLKGENGLPGENGAPGPMGPR

SEQ ID NO: 91 - protein, 49 aa, synthetic construct
  REGION 1..49: note = Recombinant collagen fragment
DGRNGEKGETGAPGLKGENGLPGENGAPGPMGPRGAPGERGRPGLPGAA

SEQ ID NO: 92 - protein, 52 aa, synthetic construct
  REGION 1..52: note = Recombinant collagen fragment
DGRNGEKGETGAPGLKGENGLPGENGAPGPMGPRGAPGERGRPGLPGAAGAR

SEQ ID NO: 93 - protein, 29 aa, synthetic construct
  REGION 1..29: note = Recombinant collagen fragment
EKGETGAPGLKGENGLPGENGAPGPMGPR

SEQ ID NO: 94 - protein, 17 aa, synthetic construct
  REGION 1..17: note = Recombinant collagen fragment
EMGPAGIPGAPGLMGAR

SEQ ID NO: 95 - protein, 17 aa, synthetic construct
  REGION 1..17: note = Recombinant collagen fragment
ENGLPGENGAPGPMGPR

SEQ ID NO: 96 - protein, 35 aa, synthetic construct
  REGION 1..35: note = Recombinant collagen fragment
ENGLPGENGAPGPMGPRGAPGERGRPGLPGAAGAR

SEQ ID NO: 97 - protein, 20 aa, synthetic construct
  REGION 1..20: note = Recombinant collagen fragment
EPGQAGPSGPPGPPGAIGPS

SEQ ID NO: 98 - protein, 14 aa, synthetic construct
  REGION 1..14: note = Recombinant collagen fragment
ERGLPGPPGIKGPA

SEQ ID NO: 99 - protein, 20 aa, synthetic construct
  REGION 1..20: note = Recombinant collagen fragment
ESGRPGPPGPSGPRGQPGVM

SEQ ID NO: 100 - protein, 28 aa, synthetic construct
  REGION 1..28: note = Recombinant collagen fragment
ESGRPGPPGPSGPRGQPGVMGFPGPKGN

SEQ ID NO: 101 - protein, 20 aa, synthetic construct
  REGION 1..20: note = Recombinant collagen fragment
ESGRPGRPGERGLPGPPGIK

SEQ ID NO: 102 - protein, 17 aa, synthetic construct
  REGION 1..17: note = Recombinant collagen fragment
ETGAPGLKGENGLPGEN

SEQ ID NO: 103 - protein, 26 aa, synthetic construct
  REGION 1..26: note = Recombinant collagen fragment
ETGAPGLKGENGLPGENGAPGPMGPR

SEQ ID NO: 104 - protein, 16 aa, synthetic construct
  REGION 1..16: note = Recombinant collagen fragment
ETGPQGPPGPTGPGGD

SEQ ID NO: 105 - protein, 23 aa, synthetic construct
  REGION 1..23: note = Recombinant collagen fragment
EVGPAGSPGSNGAPGQRGEPGPQ

SEQ ID NO: 106 - protein, 25 aa, synthetic construct
  REGION 1..25: note = Recombinant collagen fragment
EVGPAGSPGSNGAPGQRGEPGPQGH

SEQ ID NO: 107 - protein, 27 aa, synthetic construct
  REGION 1..27: note = Recombinant collagen fragment
EVGPAGSPGSNGAPGQRGEPGPQGHAG

SEQ ID NO: 108 - protein, 29 aa, synthetic construct
  REGION 1..29: note = Recombinant collagen fragment
EVGPAGSPGSNGAPGQRGEPGPQGHAGAQ

SEQ ID NO: 109 - protein, 38 aa, synthetic construct
  REGION 1..38: note = Recombinant collagen fragment
EVGPAGSPGSNGAPGQRGEPGPQGHAGAQGPPGPPGIN

SEQ ID NO: 110 - protein, 44 aa, synthetic construct
  REGION 1..44: note = Recombinant collagen fragment
EVGPAGSPGSNGAPGQRGEPGPQGHAGAQGPPGPPGINGSPGGK

SEQ ID NO: 111 - protein, 20 aa, synthetic construct
  REGION 1..20: note = Recombinant collagen fragment
FDGRNGEKGETGAPGLKGEN

SEQ ID NO: 112 - protein, 26 aa, synthetic construct
  REGION 1..26: note = Recombinant collagen fragment
FDGRNGEKGETGAPGLKGENGLPGEN

SEQ ID NO: 113 - protein, 35 aa, synthetic construct
  REGION 1..35: note = Recombinant collagen fragment
FDGRNGEKGETGAPGLKGENGLPGENGAPGPMGPR

SEQ ID NO: 114 - protein, 14 aa, synthetic construct
  REGION 1..14: note = Recombinant collagen fragment
FPGSPGAKGEVGPA

SEQ ID NO: 115 - protein, 20 aa, synthetic construct
  REGION 1..20: note = Recombinant collagen fragment
FPGSPGAKGEVGPAGSPGSN

SEQ ID NO: 116 - protein, 24 aa, synthetic construct
  REGION 1..24: note = Recombinant collagen fragment
GAAGARGNDGARGSDGQPGPPGPP

SEQ ID NO: 117 - protein, 27 aa, synthetic construct
  REGION 1..27: note = Recombinant collagen fragment
GAAGARGNDGARGSDGQPGPPGPPGTA

SEQ ID NO: 118 - protein, 36 aa, synthetic construct
  REGION 1..36: note = Recombinant collagen fragment
GAAGARGNDGARGSDGQPGPPGPPGTAGFPGSPGAK

SEQ ID NO: 119 - protein, 42 aa, synthetic construct
  REGION 1..42: note = Recombinant collagen fragment
GAAGARGNDGARGSDGQPGPPGPPGTAGFPGSPGAKGEVGPA

SEQ ID NO: 120 - protein, 22 aa, synthetic construct
  REGION 1..22: note = Recombinant collagen fragment
GAAGEPGRDGVPGGPGMRGMPG

SEQ ID NO: 121 - protein, 28 aa, synthetic construct
  REGION 1..28: note = Recombinant collagen fragment
GAAGEPGRDGVPGGPGMRGMPGSPGGPG

SEQ ID NO: 122 - protein, 18 aa, synthetic construct
  REGION 1..18: note = Recombinant collagen fragment
GAAGERGAPGFRGPAGPN

SEQ ID NO: 123 - protein, 24 aa, synthetic construct
  REGION 1..24: note = Recombinant collagen fragment
GAIGPSGPAGKDGESGRPGRPGER

SEQ ID NO: 124 - protein, 30 aa, synthetic construct
  REGION 1..30: note = Recombinant collagen fragment
GAIGPSGPAGKDGESGRPGRPGERGLPGPP

SEQ ID NO: 125 - protein, 33 aa, synthetic construct
  REGION 1..33: note = Recombinant collagen fragment
GAIGPSGPAGKDGESGRPGRPGERGLPGPPGIK

SEQ ID NO: 126 - protein, 22 aa, synthetic construct
  REGION 1..22: note = Recombinant collagen fragment
GAKGEPGPRGERGEAGIPGVPG

SEQ ID NO: 127 - protein, 24 aa, synthetic construct
  REGION 1..24: note = Recombinant collagen fragment
GAKGEPGPRGERGEAGIPGVPGAK

SEQ ID NO: 128 - protein, 25 aa, synthetic construct
  REGION 1..25: note = Recombinant collagen fragment
GAKGEPGPRGERGEAGIPGVPGAKG

SEQ ID NO: 129 - protein, 29 aa, synthetic construct
  REGION 1..29: note = Recombinant collagen fragment
GAKGEPGPRGERGEAGIPGVPGAKGEDGK

SEQ ID NO: 130 - protein, 31 aa, synthetic construct
  REGION 1..31: note = Recombinant collagen fragment
GAKGEPGPRGERGEAGIPGVPGAKGEDGKDG

SEQ ID NO: 131 - protein, 32 aa, synthetic construct
  REGION 1..32: note = Recombinant collagen fragment
GAKGEPGPRGERGEAGIPGVPGAKGEDGKDGS

SEQ ID NO: 132 - protein, 39 aa, synthetic construct
  REGION 1..39: note = Recombinant collagen fragment
GAKGEPGPRGERGEAGIPGVPGAKGEDGKDGSPGEPGAN

SEQ ID NO: 133 - protein, 27 aa, synthetic construct
  REGION 1..27: note = Recombinant collagen fragment
GAKGEVGPAGSPGSNGAPGQRGEPGPQ

SEQ ID NO: 134 - protein, 28 aa, synthetic construct
  REGION 1..28: note = Recombinant collagen fragment
GAKGEVGPAGSPGSNGAPGQRGEPGPQG

SEQ ID NO: 135 - protein, 29 aa, synthetic construct
  REGION 1..29: note = Recombinant collagen fragment
GAKGEVGPAGSPGSNGAPGQRGEPGPQGH

SEQ ID NO: 136 - protein, 33 aa, synthetic construct
  REGION 1..33: note = Recombinant collagen fragment
GAKGEVGPAGSPGSNGAPGQRGEPGPQGHAGAQ

SEQ ID NO: 137 - protein, 42 aa, synthetic construct
  REGION 1..42: note = Recombinant collagen fragment
GAKGEVGPAGSPGSNGAPGQRGEPGPQGHAGAQGPPGPPGIN

SEQ ID NO: 138 - protein, 9 aa, synthetic construct
  REGION 1..9: note = Recombinant collagen fragment
GANGAPGLR

SEQ ID NO: 139 - protein, 18 aa, synthetic construct
  REGION 1..18: note = Recombinant collagen fragment
GANGAPGLRGGAGEPGKN

SEQ ID NO: 140 - protein, 21 aa, synthetic construct
  REGION 1..21: note = Recombinant collagen fragment
GANGAPGLRGGAGEPGKNGAK

SEQ ID NO: 141 - protein, 27 aa, synthetic construct
  REGION 1..27: note = Recombinant collagen fragment
GANGAPGLRGGAGEPGKNGAKGEPGPR

SEQ ID NO: 142 - protein, 28 aa, synthetic construct
  REGION 1..28: note = Recombinant collagen fragment
GANGAPGLRGGAGEPGKNGAKGEPGPRG

SEQ ID NO: 143 - protein, 30 aa, synthetic construct
  REGION 1..30: note = Recombinant collagen fragment
GANGAPGLRGGAGEPGKNGAKGEPGPRGER

SEQ ID NO: 144 - protein, 31 aa, synthetic construct
  REGION 1..31: note = Recombinant collagen fragment
GANGAPGLRGGAGEPGKNGAKGEPGPRGERG

SEQ ID NO: 145 - protein, 34 aa, synthetic construct
  REGION 1..34: note = Recombinant collagen fragment
GANGAPGLRGGAGEPGKNGAKGEPGPRGERGEAG

SEQ ID NO: 146 - protein, 15 aa, synthetic construct
  REGION 1..15: note = Recombinant collagen fragment
GAPGERGRPGLPGAA

SEQ ID NO: 147 - protein, 16 aa, synthetic construct
  REGION 1..16: note = Recombinant collagen fragment
GAPGERGRPGLPGAAG

SEQ ID NO: 148 - protein, 17 aa, synthetic construct
  REGION 1..17: note = Recombinant collagen fragment
GAPGERGRPGLPGAAGA

SEQ ID NO: 149 - protein, 18 aa, synthetic construct
  REGION 1..18: note = Recombinant collagen fragment
GAPGERGRPGLPGAAGAR

SEQ ID NO: 150 - protein, 20 aa, synthetic construct
  REGION 1..20: note = Recombinant collagen fragment
GAPGERGRPGLPGAAGARGN

SEQ ID NO: 151 - protein, 21 aa, synthetic construct
  REGION 1..21: note = Recombinant collagen fragment
GAPGERGRPGLPGAAGARGND

SEQ ID NO: 152 - protein, 22 aa, synthetic construct
  REGION 1..22: note = Recombinant collagen fragment
GAPGERGRPGLPGAAGARGNDG

SEQ ID NO: 153 - protein, 23 aa, synthetic construct
  REGION 1..23: note = Recombinant collagen fragment
GAPGERGRPGLPGAAGARGNDGA

SEQ ID NO: 154 - protein, 24 aa, synthetic construct
  REGION 1..24: note = Recombinant collagen fragment
GAPGERGRPGLPGAAGARGNDGAR

SEQ ID NO: 155 - protein, 25 aa, synthetic construct
  REGION 1..25: note = Recombinant collagen fragment
GAPGERGRPGLPGAAGARGNDGARG

SEQ ID NO: 156 - protein, 26 aa, synthetic construct
  REGION 1..26: note = Recombinant collagen fragment
GAPGERGRPGLPGAAGARGNDGARGS

SEQ ID NO: 157 - protein, 27 aa, synthetic construct
  REGION 1..27: note = Recombinant collagen fragment
GAPGERGRPGLPGAAGARGNDGARGSD

SEQ ID NO: 158 - protein, 28 aa, synthetic construct
  REGION 1..28: note = Recombinant collagen fragment
GAPGERGRPGLPGAAGARGNDGARGSDG

SEQ ID NO: 159 - protein, 29 aa, synthetic construct
  REGION 1..29: note = Recombinant collagen fragment
GAPGERGRPGLPGAAGARGNDGARGSDGQ

SEQ ID NO: 160 - protein, 31 aa, synthetic construct
  REGION 1..31: note = Recombinant collagen fragment
GAPGERGRPGLPGAAGARGNDGARGSDGQPG

SEQ ID NO: 161 - protein, 32 aa, synthetic construct
  REGION 1..32: note = Recombinant collagen fragment
GAPGERGRPGLPGAAGARGNDGARGSDGQPGP

SEQ ID NO: 162 - protein, 34 aa, synthetic construct
  REGION 1..34: note = Recombinant collagen fragment
GAPGERGRPGLPGAAGARGNDGARGSDGQPGPPG

SEQ ID NO: 163 - protein, 35 aa, synthetic construct
  REGION 1..35: note = Recombinant collagen fragment
GAPGERGRPGLPGAAGARGNDGARGSDGQPGPPGP

SEQ ID NO: 164 - protein, 37 aa, synthetic construct
  REGION 1..37: note = Recombinant collagen fragment
GAPGERGRPGLPGAAGARGNDGARGSDGQPGPPGPPG

SEQ ID NO: 165 - protein, 39 aa, synthetic construct
  REGION 1..39: note = Recombinant collagen fragment
GAPGERGRPGLPGAAGARGNDGARGSDGQPGPPGPPGTA

SEQ ID NO: 166 - protein, 48 aa, synthetic construct
  REGION 1..48: note = Recombinant collagen fragment
GAPGERGRPGLPGAAGARGNDGARGSDGQPGPPGPPGTAGFPGSPGAK

SEQ ID NO: 167 - protein, 52 aa, synthetic construct
  REGION 1..52: note = Recombinant collagen fragment
GAPGERGRPGLPGAAGARGNDGARGSDGQPGPPGPPGTAGFPGSPGAKGEVG

SEQ ID NO: 168 - protein, 54 aa, synthetic construct
  REGION 1..54: note = Recombinant collagen fragment
GAPGERGRPGLPGAAGARGNDGARGSDGQPGPPGPPGTAGFPGSPGAKGEVGPA

SEQ ID NO: 169 - protein, 55 aa, synthetic construct
  REGION 1..55: note = Recombinant collagen fragment
GAPGERGRPGLPGAAGARGNDGARGSDGQPGPPGPPGTAGFPGSPGAKGEVGPAG

SEQ ID NO: 170 - protein, 24 aa, synthetic construct
  REGION 1..24: note = Recombinant collagen fragment
GAPGLKGENGLPGENGAPGPMGPR

SEQ ID NO: 171 - protein, 15 aa, synthetic construct
  REGION 1..15: note = Recombinant collagen fragment
GAPGLMGARGPPGPA

SEQ ID NO: 172 - protein, 18 aa, synthetic construct
  REGION 1..18: note = Recombinant collagen fragment
GAPGLMGARGPPGPAGAN

SEQ ID NO: 173 - protein, 21 aa, synthetic construct
  REGION 1..21: note = Recombinant collagen fragment
GAPGLMGARGPPGPAGANGAP

SEQ ID NO: 174 - protein, 24 aa, synthetic construct
  REGION 1..24: note = Recombinant collagen fragment
GAPGLMGARGPPGPAGANGAPGLR

SEQ ID NO: 175 - protein, 33 aa, synthetic construct
  REGION 1..33: note = Recombinant collagen fragment
GAPGLMGARGPPGPAGANGAPGLRGGAGEPGKN

SEQ ID NO: 176 - protein, 15 aa, synthetic construct
  REGION 1..15: note = Recombinant collagen fragment
GAPGLRGGAGEPGKN

SEQ ID NO: 177 - protein, 30 aa, synthetic construct
  REGION 1..30: note = Recombinant collagen fragment
GAPGLRGGAGEPGKNGAKGEPGPRGERGEA

SEQ ID NO: 178 - protein, 22 aa, synthetic construct
  REGION 1..22: note = Recombinant collagen fragment
GAPGPAGPRGAAGEPGRDGVPG

SEQ ID NO: 179 - protein, 31 aa, synthetic construct
  REGION 1..31: note = Recombinant collagen fragment
GAPGPAGPRGAAGEPGRDGVPGGPGMRGMPG

SEQ ID NO: 180 - protein, 21 aa, synthetic construct
  REGION 1..21: note = Recombinant collagen fragment
GAPGPMGPRGAPGERGRPGLP

SEQ ID NO: 181 - protein, 22 aa, synthetic construct
  REGION 1..22: note = Recombinant collagen fragment
GAPGPMGPRGAPGERGRPGLPG

SEQ ID NO: 182 - protein, 24 aa, synthetic construct
  REGION 1..24: note = Recombinant collagen fragment
GAPGPMGPRGAPGERGRPGLPGAA

SEQ ID NO: 183 - protein, 25 aa, synthetic construct
  REGION 1..25: note = Recombinant collagen fragment
GAPGPMGPRGAPGERGRPGLPGAAG

SEQ ID NO: 184 - protein, 26 aa, synthetic construct
  REGION 1..26: note = Recombinant collagen fragment
GAPGPMGPRGAPGERGRPGLPGAAGA

SEQ ID NO: 185 - protein, 27 aa, synthetic construct
  REGION 1..27: note = Recombinant collagen fragment
GAPGPMGPRGAPGERGRPGLPGAAGAR

SEQ ID NO: 186 - protein, 29 aa, synthetic construct
  REGION 1..29: note = Recombinant collagen fragment
GAPGPMGPRGAPGERGRPGLPGAAGARGN

SEQ ID NO: 187 - protein, 33 aa, synthetic construct
  REGION 1..33: note = Recombinant collagen fragment
GAPGPMGPRGAPGERGRPGLPGAAGARGNDGAR

SEQ ID NO: 188 - protein, 34 aa, synthetic construct
  REGION 1..34: note = Recombinant collagen fragment
GAPGPMGPRGAPGERGRPGLPGAAGARGNDGARG

SEQ ID NO: 189 - protein, 48 aa, synthetic construct
  REGION 1..48: note = Recombinant collagen fragment
GAPGPMGPRGAPGERGRPGLPGAAGARGNDGARGSDGQPGPPGPPGTA

SEQ ID NO: 190 - protein, 24 aa, synthetic construct
  REGION 1..24: note = Recombinant collagen fragment
GAPGQRGEPGPQGHAGAQGPPGPP

SEQ ID NO: 191 - protein, 25 aa, synthetic construct
  REGION 1..25: note = Recombinant collagen fragment
GAPGQRGEPGPQGHAGAQGPPGPPG

SEQ ID NO: 192 - protein, 27 aa, synthetic construct
  REGION 1..27: note = Recombinant collagen fragment
GAPGQRGEPGPQGHAGAQGPPGPPGIN

SEQ ID NO: 193 - protein, 28 aa, synthetic construct
  REGION 1..28: note = Recombinant collagen fragment
GAPGQRGEPGPQGHAGAQGPPGPPGING

SEQ ID NO: 194 - protein, 29 aa, synthetic construct
  REGION 1..29: note = Recombinant collagen fragment
GAPGQRGEPGPQGHAGAQGPPGPPGINGS

SEQ ID NO: 195 - protein, 31 aa, synthetic construct
  REGION 1..31: note = Recombinant collagen fragment
GAPGQRGEPGPQGHAGAQGPPGPPGINGSPG

SEQ ID NO: 196 - protein, 32 aa, synthetic construct
  REGION 1..32: note = Recombinant collagen fragment
GAPGQRGEPGPQGHAGAQGPPGPPGINGSPGG

SEQ ID NO: 197 - protein, 33 aa, synthetic construct
  REGION 1..33: note = Recombinant collagen fragment
GAPGQRGEPGPQGHAGAQGPPGPPGINGSPGGK

SEQ ID NO: 198 - protein, 36 aa, synthetic construct
  REGION 1..36: note = Recombinant collagen fragment
GAPGQRGEPGPQGHAGAQGPPGPPGINGSPGGKGEM

SEQ ID NO: 199 - protein, 37 aa, synthetic construct
  REGION 1..37: note = Recombinant collagen fragment
GAPGQRGEPGPQGHAGAQGPPGPPGINGSPGGKGEMG

SEQ ID NO: 200 - protein, 39 aa, synthetic construct
  REGION 1..39: note = Recombinant collagen fragment
GAPGQRGEPGPQGHAGAQGPPGPPGINGSPGGKGEMGPA

SEQ ID NO: 201 - protein, 48 aa, synthetic construct
  REGION 1..48: note = Recombinant collagen fragment
GAPGQRGEPGPQGHAGAQGPPGPPGINGSPGGKGEMGPAGIPGAPGLM

SEQ ID NO: 202 - protein, 49 aa, synthetic construct
  REGION 1..49: note = Recombinant collagen fragment
GAPGQRGEPGPQGHAGAQGPPGPPGINGSPGGKGEMGPAGIPGAPGLMG

SEQ ID NO: 203 - protein, 50 aa, synthetic construct
  REGION 1..50: note = Recombinant collagen fragment
GAPGQRGEPGPQGHAGAQGPPGPPGINGSPGGKGEMGPAGIPGAPGLMGA

SEQ ID NO: 204 - protein, 51 aa, synthetic construct
  REGION 1..51: note = Recombinant collagen fragment
GAPGQRGEPGPQGHAGAQGPPGPPGINGSPGGKGEMGPAGIPGAPGLMGAR

SEQ ID NO: 205 - protein, 12 aa, synthetic construct
  REGION 1..12: note = Recombinant collagen fragment
GAQGPPGPPGIN

SEQ ID NO: 206 - protein, 21 aa, synthetic construct
  REGION 1..21: note = Recombinant collagen fragment
GARGNDGARGSDGQPGPPGPP

SEQ ID NO: 207 - protein, 24 aa, synthetic construct
  REGION 1..24: note = Recombinant collagen fragment
GARGNDGARGSDGQPGPPGPPGTA

SEQ ID NO: 208 - protein, 28 aa, synthetic construct
  REGION 1..28: note = Recombinant collagen fragment
GARGNDGARGSDGQPGPPGPPGTAGFPG

SEQ ID NO: 209 - protein, 32 aa, synthetic construct
  REGION 1..32: note = Recombinant collagen fragment
GARGNDGARGSDGQPGPPGPPGTAGFPGSPGA

SEQ ID NO: 210 - protein, 33 aa, synthetic construct
  REGION 1..33: note = Recombinant collagen fragment
GARGNDGARGSDGQPGPPGPPGTAGFPGSPGAK

SEQ ID NO: 211 - protein, 39 aa, synthetic construct
  REGION 1..39: note = Recombinant collagen fragment
GARGNDGARGSDGQPGPPGPPGTAGFPGSPGAKGEVGPA

SEQ ID NO: 212 - protein, 40 aa, synthetic construct
  REGION 1..40: note = Recombinant collagen fragment
GARGNDGARGSDGQPGPPGPPGTAGFPGSPGAKGEVGPAG

SEQ ID NO: 213 - protein, 43 aa, synthetic construct
  REGION 1..43: note = Recombinant collagen fragment
GARGNDGARGSDGQPGPPGPPGTAGFPGSPGAKGEVGPAGSPG

SEQ ID NO: 214 - protein, 45 aa, synthetic construct
  REGION 1..45: note = Recombinant collagen fragment
GARGNDGARGSDGQPGPPGPPGTAGFPGSPGAKGEVGPAGSPGSN

SEQ ID NO: 215 - protein, 12 aa, synthetic construct
  REGION 1..12: note = Recombinant collagen fragment
GARGPPGPAGAN

SEQ ID NO: 216 - protein, 17 aa, synthetic construct
  REGION 1..17: note = Recombinant collagen fragment
GARGPPGPAGANGAPGL

SEQ ID NO: 217 - protein, 18 aa, synthetic construct
  REGION 1..18: note = Recombinant collagen fragment
GARGPPGPAGANGAPGLR

SEQ ID NO: 218 - protein, 19 aa, synthetic construct
  REGION 1..19: note = Recombinant collagen fragment
GARGPPGPAGANGAPGLRG

SEQ ID NO: 219 - protein, 20 aa, synthetic construct
  REGION 1..20: note = Recombinant collagen fragment
GARGPPGPAGANGAPGLRGG

SEQ ID NO: 220 - protein, 21 aa, synthetic construct
  REGION 1..21: note = Recombinant collagen fragment
GARGPPGPAGANGAPGLRGGA

SEQ ID NO: 221 - protein, 22 aa, synthetic construct
  REGION 1..22: note = Recombinant collagen fragment
GARGPPGPAGANGAPGLRGGAG

SEQ ID NO: 222 - protein, 23 aa, synthetic construct
  REGION 1..23: note = Recombinant collagen fragment
GARGPPGPAGANGAPGLRGGAGE

SEQ ID NO: 223 - protein, 25 aa, synthetic construct
  REGION 1..25: note = Recombinant collagen fragment
GARGPPGPAGANGAPGLRGGAGEPG

SEQ ID NO: 224 - protein, 27 aa, synthetic construct
  REGION 1..27: note = Recombinant collagen fragment
GARGPPGPAGANGAPGLRGGAGEPGKN

SEQ ID NO: 225 - protein, 28 aa, synthetic construct
  REGION 1..28: note = Recombinant collagen fragment
GARGPPGPAGANGAPGLRGGAGEPGKNG

SEQ ID NO: 226 - protein, 29 aa, synthetic construct
  REGION 1..29: note = Recombinant collagen fragment
GARGPPGPAGANGAPGLRGGAGEPGKNGA

SEQ ID NO: 227 - protein, 30 aa, synthetic construct
  REGION 1..30: note = Recombinant collagen fragment
GARGPPGPAGANGAPGLRGGAGEPGKNGAK

SEQ ID NO: 228 - protein, 32 aa, synthetic construct
  REGION 1..32: note = Recombinant collagen fragment
GARGPPGPAGANGAPGLRGGAGEPGKNGAKGE

SEQ ID NO: 229 - protein, 34 aa, synthetic construct
  REGION 1..34: note = Recombinant collagen fragment
GARGPPGPAGANGAPGLRGGAGEPGKNGAKGEPG

SEQ ID NO: 230 - protein, 36 aa, synthetic construct
  REGION 1..36: note = Recombinant collagen fragment
GARGPPGPAGANGAPGLRGGAGEPGKNGAKGEPGPR

SEQ ID NO: 231 - protein, 39 aa, synthetic construct
  REGION 1..39: note = Recombinant collagen fragment
GARGPPGPAGANGAPGLRGGAGEPGKNGAKGEPGPRGER

SEQ ID NO: 232 - protein, 18 aa, synthetic construct
  REGION 1..18: note = Recombinant collagen fragment
GARGSDGQPGPPGPPGTA

SEQ ID NO: 233 - protein, 27 aa, synthetic construct
  REGION 1..27: note = Recombinant collagen fragment
GARGSDGQPGPPGPPGTAGFPGSPGAK

SEQ ID NO: 234 - protein, 33 aa, synthetic construct
  REGION 1..33: note = Recombinant collagen fragment
GARGSDGQPGPPGPPGTAGFPGSPGAKGEVGPA

SEQ ID NO: 235 - protein, 27 aa, synthetic construct
  REGION 1..27: note = Recombinant collagen fragment
GDKGDTGPPGPQGLQGLPGTGGPPGEN

SEQ ID NO: 236 - protein, 12 aa, synthetic construct
  REGION 1..12: note = Recombinant collagen fragment
GEKGETGAPGLK

SEQ ID NO: 237 - protein, 21 aa, synthetic construct
  REGION 1..21: note = Recombinant collagen fragment
GEKGETGAPGLKGENGLPGEN

SEQ ID NO: 238 - protein, 27 aa, synthetic construct
  REGION 1..27: note = Recombinant collagen fragment
GEKGETGAPGLKGENGLPGENGAPGPM

SEQ ID NO: 239 - protein, 30 aa, synthetic construct
  REGION 1..30: note = Recombinant collagen fragment
GEKGETGAPGLKGENGLPGENGAPGPMGPR

SEQ ID NO: 240 - protein, 45 aa, synthetic construct
  REGION 1..45: note = Recombinant collagen fragment
GEKGETGAPGLKGENGLPGENGAPGPMGPRGAPGERGRPGLPGAA

SEQ ID NO: 241 - protein, 48 aa, synthetic construct
  REGION 1..48: note = Recombinant collagen fragment
GEKGETGAPGLKGENGLPGENGAPGPMGPRGAPGERGRPGLPGAAGAR

SEQ ID NO: 242 - protein, 15 aa, synthetic construct
  REGION 1..15: note = Recombinant collagen fragment
GEMGPAGIPGAPGLM

SEQ ID NO: 243 - protein, 18 aa, synthetic construct
  REGION 1..18: note = Recombinant collagen fragment
GEMGPAGIPGAPGLMGAR

SEQ ID NO: 244 - protein, 24 aa, synthetic construct
  REGION 1..24: note = Recombinant collagen fragment
GEMGPAGIPGAPGLMGARGPPGPA

SEQ ID NO: 245 - protein, 27 aa, synthetic construct
  REGION 1..27: note = Recombinant collagen fragment
GEMGPAGIPGAPGLMGARGPPGPAGAN

SEQ ID NO: 246 - protein, 33 aa, synthetic construct
  REGION 1..33: note = Recombinant collagen fragment
GEMGPAGIPGAPGLMGARGPPGPAGANGAPGLR

SEQ ID NO: 247 - protein, 42 aa, synthetic construct
  REGION 1..42: note = Recombinant collagen fragment
GEMGPAGIPGAPGLMGARGPPGPAGANGAPGLRGGAGEPGKN

SEQ ID NO: 248 - protein, 15 aa, synthetic construct
  REGION 1..15: note = Recombinant collagen fragment
GENGLPGENGAPGPM

SEQ ID NO: 249 - protein, 18 aa, synthetic construct
  REGION 1..18: note = Recombinant collagen fragment
GENGLPGENGAPGPMGPR

SEQ ID NO: 250 - protein, 19 aa, synthetic construct
  REGION 1..19: note = Recombinant collagen fragment
GENGLPGENGAPGPMGPRG

SEQ ID NO: 251 - protein, 22 aa, synthetic construct
  REGION 1..22: note = Recombinant collagen fragment
GENGLPGENGAPGPMGPRGAPG

SEQ ID NO: 252 - protein, 24 aa, synthetic construct
  REGION 1..24: note = Recombinant collagen fragment
GENGLPGENGAPGPMGPRGAPGER

SEQ ID NO: 253 - protein, 25 aa, synthetic construct
  REGION 1..25: note = Recombinant collagen fragment
GENGLPGENGAPGPMGPRGAPGERG

SEQ ID NO: 254 - protein, 26 aa, synthetic construct
  REGION 1..26: note = Recombinant collagen fragment
GENGLPGENGAPGPMGPRGAPGERGR

SEQ ID NO: 255 - protein, 33 aa, synthetic construct
  REGION 1..33: note = Recombinant collagen fragment
GENGLPGENGAPGPMGPRGAPGERGRPGLPGAA

SEQ ID NO: 256 - protein, 35 aa, synthetic construct
  REGION 1..35: note = Recombinant collagen fragment
GENGLPGENGAPGPMGPRGAPGERGRPGLPGAAGA

SEQ ID NO: 257 - protein, 36 aa, synthetic construct
  REGION 1..36: note = Recombinant collagen fragment
GENGLPGENGAPGPMGPRGAPGERGRPGLPGAAGAR

SEQ ID NO: 258 - protein, 38 aa, synthetic construct
  REGION 1..38: note = Recombinant collagen fragment
GENGLPGENGAPGPMGPRGAPGERGRPGLPGAAGARGN

SEQ ID NO: 259 - protein, 27 aa, synthetic construct
  REGION 1..27: note = Recombinant collagen fragment
GEPGANGLPGAAGERGAPGFRGPAGPN

SEQ ID NO: 260 - protein, 25 aa, synthetic construct
  REGION 1..25: note = Recombinant collagen fragment
GEPGPKGDAGAPGAPGGKGDAGAPG

SEQ ID NO: 261 - protein, 26 aa, synthetic construct
  REGION 1..26: note = Recombinant collagen fragment
GEPGPKGDAGAPGAPGGKGDAGAPGE

SEQ ID NO: 262 - protein, 21 aa, synthetic construct
  REGION 1..21: note = Recombinant collagen fragment
GEPGPQGHAGAQGPPGPPGIN

SEQ ID NO: 263 - protein, 33 aa, synthetic construct
  REGION 1..33: note = Recombinant collagen fragment
GEPGPQGHAGAQGPPGPPGINGSPGGKGEMGPA

SEQ ID NO: 264 - protein, 42 aa, synthetic construct
  REGION 1..42: note = Recombinant collagen fragment
GEPGPQGHAGAQGPPGPPGINGSPGGKGEMGPAGIPGAPGLM

SEQ ID NO: 265 - protein, 28 aa, synthetic construct
  REGION 1..28: note = Recombinant collagen fragment
GEPGPRGERGEAGIPGVPGAKGEDGKDG

SEQ ID NO: 266 - protein, 21 aa, synthetic construct
  REGION 1..21: note = Recombinant collagen fragment
GEPGQAGPSGPPGPPGAIGPS

SEQ ID NO: 267 - protein, 27 aa, synthetic construct
  REGION 1..27: note = Recombinant collagen fragment
GEPGQAGPSGPPGPPGAIGPSGPAGKD

SEQ ID NO: 268 - protein, 25 aa, synthetic construct
  REGION 1..25: note = Recombinant collagen fragment
GEPGRDGVPGGPGMRGMPGSPGGPG

SEQ ID NO: 269 - protein, 16 aa, synthetic construct
  REGION 1..16: note = Recombinant collagen fragment
GERGEAGIPGVPGAKG

SEQ ID NO: 270 - protein, 22 aa, synthetic construct
  REGION 1..22: note = Recombinant collagen fragment
GERGEAGIPGVPGAKGEDGKDG

SEQ ID NO: 271 - protein, 30 aa, synthetic construct
  REGION 1..30: note = Recombinant collagen fragment
GERGEAGIPGVPGAKGEDGKDGSPGEPGAN

SEQ ID NO: 272 - protein, 17 aa, synthetic construct
  REGION 1..17: note = Recombinant collagen fragment
GERGGPGGPGPQGPPGK

SEQ ID NO: 273 - protein, 18 aa, synthetic construct
  REGION 1..18: note = Recombinant collagen fragment
GERGGPGGPGPQGPPGKN

SEQ ID NO: 274 - protein, 19 aa, synthetic construct
  REGION 1..19: note = Recombinant collagen fragment
GERGGPGGPGPQGPPGKNG

SEQ ID NO: 275 - protein, 23 aa, synthetic construct
  REGION 1..23: note = Recombinant collagen fragment
GERGGPGGPGPQGPPGKNGETGP

SEQ ID NO: 276 - protein, 24 aa, synthetic construct
  REGION 1..24: note = Recombinant collagen fragment
GERGGPGGPGPQGPPGKNGETGPQ

SEQ ID NO: 277 - protein, 25 aa, synthetic construct
  REGION 1..25: note = Recombinant collagen fragment
GERGGPGGPGPQGPPGKNGETGPQG

SEQ ID NO: 278 - protein, 27 aa, synthetic construct
  REGION 1..27: note = Recombinant collagen fragment
GERGGPGGPGPQGPPGKNGETGPQGPP

SEQ ID NO: 279 - protein, 28 aa, synthetic construct
  REGION 1..28: note = Recombinant collagen fragment
GERGGPGGPGPQGPPGKNGETGPQGPPG

SEQ ID NO: 280 - protein, 29 aa, synthetic construct
  REGION 1..29: note = Recombinant collagen fragment
GERGGPGGPGPQGPPGKNGETGPQGPPGP

SEQ ID NO: 281 - protein, 30 aa, synthetic construct
  REGION 1..30: note = Recombinant collagen fragment
GERGGPGGPGPQGPPGKNGETGPQGPPGPT

SEQ ID NO: 282 - protein, 31 aa, synthetic construct
  REGION 1..31: note = Recombinant collagen fragment
GERGGPGGPGPQGPPGKNGETGPQGPPGPTG

SEQ ID NO: 283 - protein, 33 aa, synthetic construct
  REGION 1..33: note = Recombinant collagen fragment
GERGGPGGPGPQGPPGKNGETGPQGPPGPTGPG

SEQ ID NO: 284 - protein, 34 aa, synthetic construct
  REGION 1..34: note = Recombinant collagen fragment
GERGGPGGPGPQGPPGKNGETGPQGPPGPTGPGG

SEQ ID NO: 285 - protein, 35 aa, synthetic construct
  REGION 1..35: note = Recombinant collagen fragment
GERGGPGGPGPQGPPGKNGETGPQGPPGPTGPGGD

SEQ ID NO: 286 - protein, 36 aa, synthetic construct
  REGION 1..36: note = Recombinant collagen fragment
GERGGPGGPGPQGPPGKNGETGPQGPPGPTGPGGDK

SEQ ID NO: 287 - protein, 37 aa, synthetic construct
  REGION 1..37: note = Recombinant collagen fragment
GERGGPGGPGPQGPPGKNGETGPQGPPGPTGPGGDKG

SEQ ID NO: 288 - protein, 38 aa, synthetic construct
  REGION 1..38: note = Recombinant collagen fragment
GERGGPGGPGPQGPPGKNGETGPQGPPGPTGPGGDKGD

SEQ ID NO: 289 - protein, 40 aa, synthetic construct
  REGION 1..40: note = Recombinant collagen fragment
GERGGPGGPGPQGPPGKNGETGPQGPPGPTGPGGDKGDTG

SEQ ID NO: 290 - protein, 43 aa, synthetic construct
  REGION 1..43: note = Recombinant collagen fragment
GERGGPGGPGPQGPPGKNGETGPQGPPGPTGPGGDKGDTGPPG

SEQ ID NO: 291 - protein, 45 aa, synthetic construct
  REGION 1..45: note = Recombinant collagen fragment
GERGGPGGPGPQGPPGKNGETGPQGPPGPTGPGGDKGDTGPPGPQ

SEQ ID NO: 292 - protein, 46 aa, synthetic construct
  REGION 1..46: note = Recombinant collagen fragment
GERGGPGGPGPQGPPGKNGETGPQGPPGPTGPGGDKGDTGPPGPQG

SEQ ID NO: 293 - protein, 48 aa, synthetic construct
  REGION 1..48: note = Recombinant collagen fragment
GERGGPGGPGPQGPPGKNGETGPQGPPGPTGPGGDKGDTGPPGPQGLQ

SEQ ID NO: 294 - protein, 49 aa, synthetic construct
  REGION 1..49: note = Recombinant collagen fragment
GERGGPGGPGPQGPPGKNGETGPQGPPGPTGPGGDKGDTGPPGPQGLQG

SEQ ID NO: 295 - protein, 12 aa, synthetic construct
  REGION 1..12: note = Recombinant collagen fragment
GERGLPGPPGIK

SEQ ID NO: 296 - protein, 15 aa, synthetic construct
  REGION 1..15: note = Recombinant collagen fragment
GERGLPGPPGIKGPA

SEQ ID NO: 297 - protein, 24 aa, synthetic construct
  REGION 1..24: note = Recombinant collagen fragment
GERGLPGPPGIKGPAGIPGFPGMK

SEQ ID NO: 298 - protein, 21 aa, synthetic construct
  REGION 1..21: note = Recombinant collagen fragment
GESGRPGPPGPSGPRGQPGVM

SEQ ID NO: 299 - protein, 29 aa, synthetic construct
  REGION 1..29: note = Recombinant collagen fragment
GESGRPGPPGPSGPRGQPGVMGFPGPKGN

SEQ ID NO: 300 - protein, 21 aa, synthetic construct
  REGION 1..21: note = Recombinant collagen fragment
GESGRPGRPGERGLPGPPGIK

SEQ ID NO: 301 - protein, 22 aa, synthetic construct
  REGION 1..22: note = Recombinant collagen fragment
GESGRPGRPGERGLPGPPGIKG

SEQ ID NO: 302 - protein, 24 aa, synthetic construct
  REGION 1..24: note = Recombinant collagen fragment
GESGRPGRPGERGLPGPPGIKGPA

SEQ ID NO: 303 - protein, 33 aa, synthetic construct
  REGION 1..33: note = Recombinant collagen fragment
GESGRPGRPGERGLPGPPGIKGPAGIPGFPGMK

SEQ ID NO: 304 - protein, 18 aa, synthetic construct
  REGION 1..18: note = Recombinant collagen fragment
GETGAPGLKGENGLPGEN

SEQ ID NO: 305 - protein, 27 aa, synthetic construct
  REGION 1..27: note = Recombinant collagen fragment
GETGAPGLKGENGLPGENGAPGPMGPR

SEQ ID NO: 306 - protein, 45 aa, synthetic construct
  REGION 1..45: note = Recombinant collagen fragment
GETGAPGLKGENGLPGENGAPGPMGPRGAPGERGRPGLPGAAGAR

SEQ ID NO: 307 - protein, 47 aa, synthetic construct
  REGION 1..47: note = Recombinant collagen fragment
GETGAPGLKGENGLPGENGAPGPMGPRGAPGERGRPGLPGAAGARGN

SEQ ID NO: 308 - protein, 15 aa, synthetic construct
  REGION 1..15: note = Recombinant collagen fragment
GETGPQGPPGPTGPG

SEQ ID NO: 309 - protein, 17 aa, synthetic construct
  REGION 1..17: note = Recombinant collagen fragment
GETGPQGPPGPTGPGGD

SEQ ID NO: 310 - protein, 22 aa, synthetic construct
  REGION 1..22: note = Recombinant collagen fragment
GETGPQGPPGPTGPGGDKGDTG

SEQ ID NO: 311 - protein, 25 aa, synthetic construct
  REGION 1..25: note = Recombinant collagen fragment
GETGPQGPPGPTGPGGDKGDTGPPG

SEQ ID NO: 312 - protein, 27 aa, synthetic construct
  REGION 1..27: note = Recombinant collagen fragment
GETGPQGPPGPTGPGGDKGDTGPPGPQ

SEQ ID NO: 313 - protein, 28 aa, synthetic construct
  REGION 1..28: note = Recombinant collagen fragment
GETGPQGPPGPTGPGGDKGDTGPPGPQG

SEQ ID NO: 314 - protein, 16 aa, synthetic construct
  REGION 1..16: note = Recombinant collagen fragment
GEVGPAGSPGSNGAPG

SEQ ID NO: 315 - protein, 17 aa, synthetic construct
  REGION 1..17: note = Recombinant collagen fragment
GEVGPAGSPGSNGAPGQ

SEQ ID NO: 316 - protein, 18 aa, synthetic construct
  REGION 1..18: note = Recombinant collagen fragment
GEVGPAGSPGSNGAPGQR

SEQ ID NO: 317 - protein, 20 aa, synthetic construct
  REGION 1..20: note = Recombinant collagen fragment
GEVGPAGSPGSNGAPGQRGE

SEQ ID NO: 318 - protein, 21 aa, synthetic construct
  REGION 1..21: note = Recombinant collagen fragment
GEVGPAGSPGSNGAPGQRGEP

SEQ ID NO: 319 - protein, 22 aa, synthetic construct
  REGION 1..22: note = Recombinant collagen fragment
GEVGPAGSPGSNGAPGQRGEPG

SEQ ID NO: 320 - protein, 23 aa, synthetic construct
  REGION 1..23: note = Recombinant collagen fragment
GEVGPAGSPGSNGAPGQRGEPGP

SEQ ID NO: 321 - protein, 24 aa, synthetic construct
  REGION 1..24: note = Recombinant collagen fragment
GEVGPAGSPGSNGAPGQRGEPGPQ

SEQ ID NO: 322 - protein, 25 aa, synthetic construct
  REGION 1..25: note = Recombinant collagen fragment
GEVGPAGSPGSNGAPGQRGEPGPQG

SEQ ID NO: 323 - protein, 26 aa, synthetic construct
  REGION 1..26: note = Recombinant collagen fragment
GEVGPAGSPGSNGAPGQRGEPGPQGH

SEQ ID NO: 324 - protein, 27 aa, synthetic construct
  REGION 1..27: note = Recombinant collagen fragment
GEVGPAGSPGSNGAPGQRGEPGPQGHA

SEQ ID NO: 325 - protein, 28 aa, synthetic construct
  REGION 1..28: note = Recombinant collagen fragment
GEVGPAGSPGSNGAPGQRGEPGPQGHAG

SEQ ID NO: 326 - protein, 29 aa, synthetic construct
  REGION 1..29: note = Recombinant collagen fragment
GEVGPAGSPGSNGAPGQRGEPGPQGHAGA

SEQ ID NO: 327 - protein, 30 aa, synthetic construct
  REGION 1..30: note = Recombinant collagen fragment
GEVGPAGSPGSNGAPGQRGEPGPQGHAGAQ

SEQ ID NO: 328 - protein, 31 aa, synthetic construct
  REGION 1..31: note = Recombinant collagen fragment
GEVGPAGSPGSNGAPGQRGEPGPQGHAGAQG

SEQ ID NO: 329 - protein, 32 aa, synthetic construct
  REGION 1..32: note = Recombinant collagen fragment
GEVGPAGSPGSNGAPGQRGEPGPQGHAGAQGP

SEQ ID NO: 330 - protein, 33 aa, synthetic construct
  REGION 1..33: note = Recombinant collagen fragment
GEVGPAGSPGSNGAPGQRGEPGPQGHAGAQGPP

SEQ ID NO: 331 - protein, 34 aa, synthetic construct
  REGION 1..34: note = Recombinant collagen fragment
GEVGPAGSPGSNGAPGQRGEPGPQGHAGAQGPPG

SEQ ID NO: 332 - protein, 35 aa, synthetic construct
  REGION 1..35: note = Recombinant collagen fragment
GEVGPAGSPGSNGAPGQRGEPGPQGHAGAQGPPGP

SEQ ID NO: 333 - protein, 36 aa, synthetic construct
  REGION 1..36: note = Recombinant collagen fragment
GEVGPAGSPGSNGAPGQRGEPGPQGHAGAQGPPGPP

SEQ ID NO: 334 - protein, 37 aa, synthetic construct
  REGION 1..37: note = Recombinant collagen fragment
GEVGPAGSPGSNGAPGQRGEPGPQGHAGAQGPPGPPG

SEQ ID NO: 335 - protein, 39 aa, synthetic construct
  REGION 1..39: note = Recombinant collagen fragment
GEVGPAGSPGSNGAPGQRGEPGPQGHAGAQGPPGPPGIN

SEQ ID NO: 336 - protein, 40 aa, synthetic construct
  REGION 1..40: note = Recombinant collagen fragment
GEVGPAGSPGSNGAPGQRGEPGPQGHAGAQGPPGPPGING

SEQ ID NO: 337 - protein, 41 aa, synthetic construct
  REGION 1..41: note = Recombinant collagen fragment
GEVGPAGSPGSNGAPGQRGEPGPQGHAGAQGPPGPPGINGS

SEQ ID NO: 338 - protein, 43 aa, synthetic construct
  REGION 1..43: note = Recombinant collagen fragment
GEVGPAGSPGSNGAPGQRGEPGPQGHAGAQGPPGPPGINGSPG

SEQ ID NO: 339 - protein, 44 aa, synthetic construct
  REGION 1..44: note = Recombinant collagen fragment
GEVGPAGSPGSNGAPGQRGEPGPQGHAGAQGPPGPPGINGSPGG

SEQ ID NO: 340 - protein, 45 aa, synthetic construct
  REGION 1..45: note = Recombinant collagen fragment
GEVGPAGSPGSNGAPGQRGEPGPQGHAGAQGPPGPPGINGSPGGK

SEQ ID NO: 341 - protein, 46 aa, synthetic construct
  REGION 1..46: note = Recombinant collagen fragment
GEVGPAGSPGSNGAPGQRGEPGPQGHAGAQGPPGPPGINGSPGGKG

SEQ ID NO: 342 - protein, 48 aa, synthetic construct
  REGION 1..48: note = Recombinant collagen fragment
GEVGPAGSPGSNGAPGQRGEPGPQGHAGAQGPPGPPGINGSPGGKGEM

SEQ ID NO: 343 - protein, 49 aa, synthetic construct
  REGION 1..49: note = Recombinant collagen fragment
GEVGPAGSPGSNGAPGQRGEPGPQGHAGAQGPPGPPGINGSPGGKGEMG

SEQ ID NO: 344 - protein, 51 aa, synthetic construct
  REGION 1..51: note = Recombinant collagen fragment
GEVGPAGSPGSNGAPGQRGEPGPQGHAGAQGPPGPPGINGSPGGKGEMGPA

SEQ ID NO: 345 - protein, 52 aa, synthetic construct
  REGION 1..52: note = Recombinant collagen fragment
GEVGPAGSPGSNGAPGQRGEPGPQGHAGAQGPPGPPGINGSPGGKGEMGPAG

SEQ ID NO: 346 - protein, 18 aa, synthetic construct
  REGION 1..18: note = Recombinant collagen fragment
GFDGRNGEKGETGAPGLK

SEQ ID NO: 347 - protein, 19 aa, synthetic construct
  REGION 1..19: note = Recombinant collagen fragment
GFDGRNGEKGETGAPGLKG

SEQ ID NO: 348 - protein, 21 aa, synthetic construct
  REGION 1..21: note = Recombinant collagen fragment
GFDGRNGEKGETGAPGLKGEN

SEQ ID NO: 349 - protein, 22 aa, synthetic construct
  REGION 1..22: note = Recombinant collagen fragment
GFDGRNGEKGETGAPGLKGENG

SEQ ID NO: 350 - protein, 27 aa, synthetic construct
  REGION 1..27: note = Recombinant collagen fragment
GFDGRNGEKGETGAPGLKGENGLPGEN

SEQ ID NO: 351 - protein, 36 aa, synthetic construct
  REGION 1..36: note = Recombinant collagen fragment
GFDGRNGEKGETGAPGLKGENGLPGENGAPGPMGPR

SEQ ID NO: 352 - protein, 51 aa, synthetic construct
  REGION 1..51: note = Recombinant collagen fragment
GFDGRNGEKGETGAPGLKGENGLPGENGAPGPMGPRGAPGERGRPGLPGAA

SEQ ID NO: 353 - protein, 27 aa, synthetic construct
  REGION 1..27: note = Recombinant collagen fragment
GFPGMKGHRGFDGRNGEKGETGAPGLK

SEQ ID NO: 354 - protein, 30 aa, synthetic construct
  REGION 1..30: note = Recombinant collagen fragment
GFPGMKGHRGFDGRNGEKGETGAPGLKGEN

SEQ ID NO: 355 - protein, 15 aa, synthetic construct
  REGION 1..15: note = Recombinant collagen fragment
GFPGPKGNDGAPGKN

SEQ ID NO: 356 - protein, 18 aa, synthetic construct
  REGION 1..18: note = Recombinant collagen fragment
GFPGPKGNDGAPGKNGER

SEQ ID NO: 357 - protein, 19 aa, synthetic construct
  REGION 1..19: note = Recombinant collagen fragment
GFPGPKGNDGAPGKNGERG

SEQ ID NO: 358 - protein, 20 aa, synthetic construct
  REGION 1..20: note = Recombinant collagen fragment
GFPGPKGNDGAPGKNGERGG

SEQ ID NO: 359 - protein, 21 aa, synthetic construct
  REGION 1..21: note = Recombinant collagen fragment
GFPGPKGNDGAPGKNGERGGP

SEQ ID NO: 360 - protein, 22 aa, synthetic construct
  REGION 1..22: note = Recombinant collagen fragment
GFPGPKGNDGAPGKNGERGGPG

SEQ ID NO: 361 - protein, 23 aa, synthetic construct
  REGION 1..23: note = Recombinant collagen fragment
GFPGPKGNDGAPGKNGERGGPGG

SEQ ID NO: 362 - protein, 24 aa, synthetic construct
  REGION 1..24: note = Recombinant collagen fragment
GFPGPKGNDGAPGKNGERGGPGGP

SEQ ID NO: 363 - protein, 25 aa, synthetic construct
  REGION 1..25: note = Recombinant collagen fragment
GFPGPKGNDGAPGKNGERGGPGGPG

SEQ ID NO: 364 - protein, 26 aa, synthetic construct
  REGION 1..26: note = Recombinant collagen fragment
GFPGPKGNDGAPGKNGERGGPGGPGP

SEQ ID NO: 365 - protein, 27 aa, synthetic construct
  REGION 1..27: note = Recombinant collagen fragment
GFPGPKGNDGAPGKNGERGGPGGPGPQ

SEQ ID NO: 366 - protein, 28 aa, synthetic construct
  REGION 1..28: note = Recombinant collagen fragment
GFPGPKGNDGAPGKNGERGGPGGPGPQG

SEQ ID NO: 367 - protein, 29 aa, synthetic construct
  REGION 1..29: note = Recombinant collagen fragment
GFPGPKGNDGAPGKNGERGGPGGPGPQGP

SEQ ID NO: 368 - protein, 30 aa, synthetic construct
  REGION 1..30: note = Recombinant collagen fragment
GFPGPKGNDGAPGKNGERGGPGGPGPQGPP

SEQ ID NO: 369 - protein, 31 aa, synthetic construct
  REGION 1..31: note = Recombinant collagen fragment
GFPGPKGNDGAPGKNGERGGPGGPGPQGPPG

SEQ ID NO: 370 - protein, 32 aa, synthetic construct
  REGION 1..32: note = Recombinant collagen fragment
GFPGPKGNDGAPGKNGERGGPGGPGPQGPPGK

SEQ ID NO: 371 - protein, 33 aa, synthetic construct
  REGION 1..33: note = Recombinant collagen fragment
GFPGPKGNDGAPGKNGERGGPGGPGPQGPPGKN

SEQ ID NO: 372 - protein, 34 aa, synthetic construct
  REGION 1..34: note = Recombinant collagen fragment
GFPGPKGNDGAPGKNGERGGPGGPGPQGPPGKNG

SEQ ID NO: 373 - protein, 35 aa, synthetic construct
  REGION 1..35: note = Recombinant collagen fragment
GFPGPKGNDGAPGKNGERGGPGGPGPQGPPGKNGE

SEQ ID NO: 374 - protein, 36 aa, synthetic construct
  REGION 1..36: note = Recombinant collagen fragment
GFPGPKGNDGAPGKNGERGGPGGPGPQGPPGKNGET

SEQ ID NO: 375 - protein, 37 aa, synthetic construct
  REGION 1..37: note = Recombinant collagen fragment
GFPGPKGNDGAPGKNGERGGPGGPGPQGPPGKNGETG

SEQ ID NO: 376 - protein, 38 aa, synthetic construct
  REGION 1..38: note = Recombinant collagen fragment
GFPGPKGNDGAPGKNGERGGPGGPGPQGPPGKNGETGP

SEQ ID NO: 377 - protein, 39 aa, synthetic construct
  REGION 1..39: note = Recombinant collagen fragment
GFPGPKGNDGAPGKNGERGGPGGPGPQGPPGKNGETGPQ

SEQ ID NO: 378 - protein, 40 aa, synthetic construct
  REGION 1..40: note = Recombinant collagen fragment
GFPGPKGNDGAPGKNGERGGPGGPGPQGPPGKNGETGPQG

SEQ ID NO: 379 - protein, 43 aa, synthetic construct
  REGION 1..43: note = Recombinant collagen fragment
GFPGPKGNDGAPGKNGERGGPGGPGPQGPPGKNGETGPQGPPG

SEQ ID NO: 380 - protein, 46 aa, synthetic construct
  REGION 1..46: note = Recombinant collagen fragment
GFPGPKGNDGAPGKNGERGGPGGPGPQGPPGKNGETGPQGPPGPTG

SEQ ID NO: 381 - protein, 50 aa, synthetic construct
  REGION 1..50: note = Recombinant collagen fragment
GFPGPKGNDGAPGKNGERGGPGGPGPQGPPGKNGETGPQGPPGPTGPGGD

SEQ ID NO: 382 - protein, 51 aa, synthetic construct
  REGION 1..51: note = Recombinant collagen fragment
GFPGPKGNDGAPGKNGERGGPGGPGPQGPPGKNGETGPQGPPGPTGPGGDK

SEQ ID NO: 383 - protein, 13 aa, synthetic construct
  REGION 1..13: note = Recombinant collagen fragment
GFPGSPGAKGEVG

SEQ ID NO: 384 - protein, 14 aa, synthetic construct
  REGION 1..14: note = Recombinant collagen fragment
GFPGSPGAKGEVGP

SEQ ID NO: 385 - protein, 15 aa, synthetic construct
  REGION 1..15: note = Recombinant collagen fragment
GFPGSPGAKGEVGPA

SEQ ID NO: 386 - protein, 16 aa, synthetic construct
  REGION 1..16: note = Recombinant collagen fragment
GFPGSPGAKGEVGPAG

SEQ ID NO: 387 - protein, 17 aa, synthetic construct
  REGION 1..17: note = Recombinant collagen fragment
GFPGSPGAKGEVGPAGS

SEQ ID NO: 388 - protein, 19 aa, synthetic construct
  REGION 1..19: note = Recombinant collagen fragment
GFPGSPGAKGEVGPAGSPG

SEQ ID NO: 389 - protein, 21 aa, synthetic construct
  REGION 1..21: note = Recombinant collagen fragment
GFPGSPGAKGEVGPAGSPGSN

SEQ ID NO: 390 - protein, 22 aa, synthetic construct
  REGION 1..22: note = Recombinant collagen fragment
GFPGSPGAKGEVGPAGSPGSNG

SEQ ID NO: 391 - protein, 23 aa, synthetic construct
  REGION 1..23: note = Recombinant collagen fragment
GFPGSPGAKGEVGPAGSPGSNGA

SEQ ID NO: 392 - protein, 25 aa, synthetic construct
  REGION 1..25: note = Recombinant collagen fragment
GFPGSPGAKGEVGPAGSPGSNGAPG

SEQ ID NO: 393 - protein, 26 aa, synthetic construct
  REGION 1..26: note = Recombinant collagen fragment
GFPGSPGAKGEVGPAGSPGSNGAPGQ

SEQ ID NO: 394 - protein, 27 aa, synthetic construct
  REGION 1..27: note = Recombinant collagen fragment
GFPGSPGAKGEVGPAGSPGSNGAPGQR

SEQ ID NO: 395 - protein, 28 aa, synthetic construct
  REGION 1..28: note = Recombinant collagen fragment
GFPGSPGAKGEVGPAGSPGSNGAPGQRG

SEQ ID NO: 396 - protein, 29 aa, synthetic construct
  REGION 1..29: note = Recombinant collagen fragment
GFPGSPGAKGEVGPAGSPGSNGAPGQRGE

SEQ ID NO: 397 - protein, 30 aa, synthetic construct
  REGION 1..30: note = Recombinant collagen fragment
GFPGSPGAKGEVGPAGSPGSNGAPGQRGEP

SEQ ID NO: 398 - protein, 31 aa, synthetic construct
  REGION 1..31: note = Recombinant collagen fragment
GFPGSPGAKGEVGPAGSPGSNGAPGQRGEPG

SEQ ID NO: 399 - protein, 33 aa, synthetic construct
  REGION 1..33: note = Recombinant collagen fragment
GFPGSPGAKGEVGPAGSPGSNGAPGQRGEPGPQ

SEQ ID NO: 400 - protein, 34 aa, synthetic construct
  REGION 1..34: note = Recombinant collagen fragment
GFPGSPGAKGEVGPAGSPGSNGAPGQRGEPGPQG

SEQ ID NO: 401 - protein, 35 aa, synthetic construct
  REGION 1..35: note = Recombinant collagen fragment
GFPGSPGAKGEVGPAGSPGSNGAPGQRGEPGPQGH

SEQ ID NO: 402 - protein, 36 aa, synthetic construct
  REGION 1..36: note = Recombinant collagen fragment
GFPGSPGAKGEVGPAGSPGSNGAPGQRGEPGPQGHA

SEQ ID NO: 403 - protein, 37 aa, synthetic construct
  REGION 1..37: note = Recombinant collagen fragment
GFPGSPGAKGEVGPAGSPGSNGAPGQRGEPGPQGHAG

SEQ ID NO: 404 - protein, 38 aa, synthetic construct
  REGION 1..38: note = Recombinant collagen fragment
GFPGSPGAKGEVGPAGSPGSNGAPGQRGEPGPQGHAGA

SEQ ID NO: 405 - protein, 39 aa, synthetic construct
  REGION 1..39: note = Recombinant collagen fragment
GFPGSPGAKGEVGPAGSPGSNGAPGQRGEPGPQGHAGAQ

SEQ ID NO: 406 - protein, 40 aa, synthetic construct
  REGION 1..40: note = Recombinant collagen fragment
GFPGSPGAKGEVGPAGSPGSNGAPGQRGEPGPQGHAGAQG

SEQ ID NO: 407 - protein, 42 aa, synthetic construct
  REGION 1..42: note = Recombinant collagen fragment
GFPGSPGAKGEVGPAGSPGSNGAPGQRGEPGPQGHAGAQGPP

SEQ ID NO: 408 - protein, 43 aa, synthetic construct
  REGION 1..43: note = Recombinant collagen fragment
GFPGSPGAKGEVGPAGSPGSNGAPGQRGEPGPQGHAGAQGPPG

SEQ ID NO: 409 - protein, 46 aa, synthetic construct
  REGION 1..46: note = Recombinant collagen fragment
GFPGSPGAKGEVGPAGSPGSNGAPGQRGEPGPQGHAGAQGPPGPPG

SEQ ID NO: 410 - protein, 48 aa, synthetic construct
  REGION 1..48: note = Recombinant collagen fragment
GFPGSPGAKGEVGPAGSPGSNGAPGQRGEPGPQGHAGAQGPPGPPGIN

SEQ ID NO: 411 - protein, 49 aa, synthetic construct
  REGION 1..49: note = Recombinant collagen fragment
GFPGSPGAKGEVGPAGSPGSNGAPGQRGEPGPQGHAGAQGPPGPPGING

SEQ ID NO: 412 - protein, 52 aa, synthetic construct
  REGION 1..52: note = Recombinant collagen fragment
GFPGSPGAKGEVGPAGSPGSNGAPGQRGEPGPQGHAGAQGPPGPPGINGSPG

SEQ ID NO: 413 - protein, 54 aa, synthetic construct
  REGION 1..54: note = Recombinant collagen fragment
GFPGSPGAKGEVGPAGSPGSNGAPGQRGEPGPQGHAGAQGPPGPPGINGSPGGK

SEQ ID NO: 414 - protein, 27 aa, synthetic construct
  REGION 1..27: note = Recombinant collagen fragment
GFRGPAGPNGIPGEKGPAGERGAPGPA

SEQ ID NO: 415 - protein, 18 aa, synthetic construct
  REGION 1..18: note = Recombinant collagen fragment
GGAGEPGKNGAKGEPGPR

SEQ ID NO: 416 - protein, 27 aa, synthetic construct
  REGION 1..27: note = Recombinant collagen fragment
GGAGEPGKNGAKGEPGPRGERGEAGIP

SEQ ID NO: 417 - protein, 28 aa, synthetic construct
  REGION 1..28: note = Recombinant collagen fragment
GGAGEPGKNGAKGEPGPRGERGEAGIPG

SEQ ID NO: 418 - protein, 40 aa, synthetic construct
  REGION 1..40: note = Recombinant collagen fragment
GGAGEPGKNGAKGEPGPRGERGEAGIPGVPGAKGEDGKDG

SEQ ID NO: 419 - protein, 48 aa, synthetic construct
  REGION 1..48: note = Recombinant collagen fragment
GGAGEPGKNGAKGEPGPRGERGEAGIPGVPGAKGEDGKDGSPGEPGAN

SEQ ID NO: 420 - protein, 18 aa, synthetic construct
  REGION 1..18: note = Recombinant collagen fragment
GGKGEMGPAGIPGAPGLM

SEQ ID NO: 421 - protein, 20 aa, synthetic construct
  REGION 1..20: note = Recombinant collagen fragment
GGKGEMGPAGIPGAPGLMGA

SEQ ID NO: 422 - protein, 21 aa, synthetic construct
  REGION 1..21: note = Recombinant collagen fragment
GGKGEMGPAGIPGAPGLMGAR

SEQ ID NO: 423 - protein, 32 aa, synthetic construct
  REGION 1..32: note = Recombinant collagen fragment
GGPGGPGPQGPPGKNGETGPQGPPGPTGPGGD

SEQ ID NO: 424 - protein, 48 aa, synthetic construct
  REGION 1..48: note = Recombinant collagen fragment
GGPGMRGMPGSPGGPGSDGKPGPPGSQGESGRPGPPGPSGPRGQPGVM

SEQ ID NO: 425 - protein, 25 aa, synthetic construct
  REGION 1..25: note = Recombinant collagen fragment
GGPGSDGKPGPPGSQGESGRPGPPG

SEQ ID NO: 426 - protein, 27 aa, synthetic construct
  REGION 1..27: note = Recombinant collagen fragment
GGPGSDGKPGPPGSQGESGRPGPPGPS

SEQ ID NO: 427 - protein, 30 aa, synthetic construct
  REGION 1..30: note = Recombinant collagen fragment
GGPGSDGKPGPPGSQGESGRPGPPGPSGPR

SEQ ID NO: 428 - protein, 32 aa, synthetic construct
  REGION 1..32: note = Recombinant collagen fragment
GGPGSDGKPGPPGSQGESGRPGPPGPSGPRGQ

SEQ ID NO: 429 - protein, 36 aa, synthetic construct
  REGION 1..36: note = Recombinant collagen fragment
GGPGSDGKPGPPGSQGESGRPGPPGPSGPRGQPGVM

SEQ ID NO: 430 - protein, 15 aa, synthetic construct
  REGION 1..15: note = Recombinant collagen fragment
GHAGAQGPPGPPGIN

SEQ ID NO: 431 - protein, 16 aa, synthetic construct
  REGION 1..16: note = Recombinant collagen fragment
GHAGAQGPPGPPGING

SEQ ID NO: 432 - protein, 19 aa, synthetic construct
  REGION 1..19: note = Recombinant collagen fragment
GHAGAQGPPGPPGINGSPG

SEQ ID NO: 433 - protein, 21 aa, synthetic construct
  REGION 1..21: note = Recombinant collagen fragment
GHAGAQGPPGPPGINGSPGGK

SEQ ID NO: 434 - protein, 25 aa, synthetic construct
  REGION 1..25: note = Recombinant collagen fragment
GHAGAQGPPGPPGINGSPGGKGEMG

SEQ ID NO: 435 - protein, 27 aa, synthetic construct
  REGION 1..27: note = Recombinant collagen fragment
GHAGAQGPPGPPGINGSPGGKGEMGPA

SEQ ID NO: 436 - protein, 36 aa, synthetic construct
  REGION 1..36: note = Recombinant collagen fragment
GHAGAQGPPGPPGINGSPGGKGEMGPAGIPGAPGLM

SEQ ID NO: 437 - protein, 37 aa, synthetic construct
  REGION 1..37: note = Recombinant collagen fragment
GHAGAQGPPGPPGINGSPGGKGEMGPAGIPGAPGLMG

SEQ ID NO: 438 - protein, 38 aa, synthetic construct
  REGION 1..38: note = Recombinant collagen fragment
GHAGAQGPPGPPGINGSPGGKGEMGPAGIPGAPGLMGA

SEQ ID NO: 439 - protein, 39 aa, synthetic construct
  REGION 1..39: note = Recombinant collagen fragment
GHAGAQGPPGPPGINGSPGGKGEMGPAGIPGAPGLMGAR

SEQ ID NO: 440 - protein, 45 aa, synthetic construct
  REGION 1..45: note = Recombinant collagen fragment
GHAGAQGPPGPPGINGSPGGKGEMGPAGIPGAPGLMGARGPPGPA

SEQ ID NO: 441 - protein, 48 aa, synthetic construct
  REGION 1..48: note = Recombinant collagen fragment
GHAGAQGPPGPPGINGSPGGKGEMGPAGIPGAPGLMGARGPPGPAGAN

SEQ ID NO: 442 - protein, 54 aa, synthetic construct
  REGION 1..54: note = Recombinant collagen fragment
GHAGAQGPPGPPGINGSPGGKGEMGPAGIPGAPGLMGARGPPGPAGANGAPGLR

SEQ ID NO: 443 - protein, 19 aa, synthetic construct
  REGION 1..19: note = Recombinant collagen fragment
GHPGSPGSPGYQGPPGEPG

SEQ ID NO: 444 - protein, 21 aa, synthetic construct
  REGION 1..21: note = Recombinant collagen fragment
GHPGSPGSPGYQGPPGEPGQA

SEQ ID NO: 445 - protein, 22 aa, synthetic construct
  REGION 1..22: note = Recombinant collagen fragment
GHPGSPGSPGYQGPPGEPGQAG

SEQ ID NO: 446 - protein, 24 aa, synthetic construct
  REGION 1..24: note = Recombinant collagen fragment
GHPGSPGSPGYQGPPGEPGQAGPS

SEQ ID NO: 447 - protein, 36 aa, synthetic construct
  REGION 1..36: note = Recombinant collagen fragment
GHPGSPGSPGYQGPPGEPGQAGPSGPPGPPGAIGPS

SEQ ID NO: 448 - protein, 39 aa, synthetic construct
  REGION 1..39: note = Recombinant collagen fragment
GHPGSPGSPGYQGPPGEPGQAGPSGPPGPPGAIGPSGPA

SEQ ID NO: 449 - protein, 42 aa, synthetic construct
  REGION 1..42: note = Recombinant collagen fragment
GHPGSPGSPGYQGPPGEPGQAGPSGPPGPPGAIGPSGPAGKD

SEQ ID NO: 450 - protein, 45 aa, synthetic construct
  REGION 1..45: note = Recombinant collagen fragment
GHPGSPGSPGYQGPPGEPGQAGPSGPPGPPGAIGPSGPAGKDGES

SEQ ID NO: 451 - protein, 54 aa, synthetic construct
  REGION 1..54: note = Recombinant collagen fragment
GHPGSPGSPGYQGPPGEPGQAGPSGPPGPPGAIGPSGPAGKDGESGRPGRPGER

SEQ ID NO: 452 - protein, 21 aa, synthetic construct
  REGION 1..21: note = Recombinant collagen fragment
GHRGFDGRNGEKGETGAPGLK

SEQ ID NO: 453 - protein, 24 aa, synthetic construct
  REGION 1..24: note = Recombinant collagen fragment
GHRGFDGRNGEKGETGAPGLKGEN

SEQ ID NO: 454 - protein, 25 aa, synthetic construct
  REGION 1..25: note = Recombinant collagen fragment
GHRGFDGRNGEKGETGAPGLKGENG

SEQ ID NO: 455 - protein, 30 aa, synthetic construct
  REGION 1..30: note = Recombinant collagen fragment
GHRGFDGRNGEKGETGAPGLKGENGLPGEN

SEQ ID NO: 456 - protein, 36 aa, synthetic construct
  REGION 1..36: note = Recombinant collagen fragment
GHRGFDGRNGEKGETGAPGLKGENGLPGENGAPGPM

SEQ ID NO: 457 - protein, 39 aa, synthetic construct
  REGION 1..39: note = Recombinant collagen fragment
GHRGFDGRNGEKGETGAPGLKGENGLPGENGAPGPMGPR

SEQ ID NO: 458 - protein, 45 aa, synthetic construct
  REGION 1..45: note = Recombinant collagen fragment
GHRGFDGRNGEKGETGAPGLKGENGLPGENGAPGPMGPRGAPGER

SEQ ID NO: 459 - protein, 9 aa, synthetic construct
  REGION 1..9: note = Recombinant collagen fragment
GIKGPAGIP

SEQ ID NO: 460 - protein, 13 aa, synthetic construct
  REGION 1..13: note = Recombinant collagen fragment
GIKGPAGIPGFPG

SEQ ID NO: 461 - protein, 16 aa, synthetic construct
  REGION 1..16: note = Recombinant collagen fragment
GIKGPAGIPGFPGMKG

SEQ ID NO: 462 - protein, 9 aa, synthetic construct
  REGION 1..9: note = Recombinant collagen fragment
GIPGAPGLM

SEQ ID NO: 463 - protein, 10 aa, synthetic construct
  REGION 1..10: note = Recombinant collagen fragment
GIPGAPGLMG

SEQ ID NO: 464 - protein, 11 aa, synthetic construct
  REGION 1..11: note = Recombinant collagen fragment
GIPGAPGLMGA

SEQ ID NO: 465 - protein, 12 aa, synthetic construct
  REGION 1..12: note = Recombinant collagen fragment
GIPGAPGLMGAR

SEQ ID NO: 466 - protein, 16 aa, synthetic construct
  REGION 1..16: note = Recombinant collagen fragment
GIPGAPGLMGARGPPG

SEQ ID NO: 467 - protein, 18 aa, synthetic construct
  REGION 1..18: note = Recombinant collagen fragment
GIPGAPGLMGARGPPGPA

SEQ ID NO: 468 - protein, 19 aa, synthetic construct
  REGION 1..19: note = Recombinant collagen fragment
GIPGAPGLMGARGPPGPAG

SEQ ID NO: 469 - protein, 20 aa, synthetic construct
  REGION 1..20: note = Recombinant collagen fragment
GIPGAPGLMGARGPPGPAGA

SEQ ID NO: 470 - protein, 21 aa, synthetic construct
  REGION 1..21: note = Recombinant collagen fragment
GIPGAPGLMGARGPPGPAGAN

SEQ ID NO: 471 - protein, 27 aa, synthetic construct
  REGION 1..27: note = Recombinant collagen fragment
GIPGAPGLMGARGPPGPAGANGAPGLR

SEQ ID NO: 472 - protein, 28 aa, synthetic construct
  REGION 1..28: note = Recombinant collagen fragment
GIPGAPGLMGARGPPGPAGANGAPGLRG

SEQ ID NO: 473 - protein, 36 aa, synthetic construct
  REGION 1..36: note = Recombinant collagen fragment
GIPGAPGLMGARGPPGPAGANGAPGLRGGAGEPGKN

SEQ ID NO: 474 - protein, 18 aa, synthetic construct
  REGION 1..18: note = Recombinant collagen fragment
GIPGEKGPAGERGAPGPA

SEQ ID NO: 475 - protein, 19 aa, synthetic construct
  REGION 1..19: note = Recombinant collagen fragment
GIPGEKGPAGERGAPGPAG

SEQ ID NO: 476 - protein, 21 aa, synthetic construct
  REGION 1..21: note = Recombinant collagen fragment
GIPGEKGPAGERGAPGPAGPR

SEQ ID NO: 477 - protein, 22 aa, synthetic construct
  REGION 1..22: note = Recombinant collagen fragment
GIPGEKGPAGERGAPGPAGPRG

SEQ ID NO: 478 - protein, 23 aa, synthetic construct
  REGION 1..23: note = Recombinant collagen fragment
GIPGEKGPAGERGAPGPAGPRGA

SEQ ID NO: 479 - protein, 24 aa, synthetic construct
  REGION 1..24: note = Recombinant collagen fragment
GIPGEKGPAGERGAPGPAGPRGAA

SEQ ID NO: 480 - protein, 25 aa, synthetic construct
  REGION 1..25: note = Recombinant collagen fragment
GIPGEKGPAGERGAPGPAGPRGAAG

SEQ ID NO: 481 - protein, 28 aa, synthetic construct
  REGION 1..28: note = Recombinant collagen fragment
GIPGEKGPAGERGAPGPAGPRGAAGEPG

SEQ ID NO: 482 - protein, 29 aa, synthetic construct
  REGION 1..29: note = Recombinant collagen fragment
GIPGEKGPAGERGAPGPAGPRGAAGEPGR

SEQ ID NO: 483 - protein, 30 aa, synthetic construct
  REGION 1..30: note = Recombinant collagen fragment
GIPGEKGPAGERGAPGPAGPRGAAGEPGRD

SEQ ID NO: 484 - protein, 34 aa, synthetic construct
  REGION 1..34: note = Recombinant collagen fragment
GIPGEKGPAGERGAPGPAGPRGAAGEPGRDGVPG

SEQ ID NO: 485 - protein, 35 aa, synthetic construct
  REGION 1..35: note = Recombinant collagen fragment
GIPGEKGPAGERGAPGPAGPRGAAGEPGRDGVPGG

SEQ ID NO: 486 - protein, 37 aa, synthetic construct
  REGION 1..37: note = Recombinant collagen fragment
GIPGEKGPAGERGAPGPAGPRGAAGEPGRDGVPGGPG

SEQ ID NO: 487 - protein, 39 aa, synthetic construct
  REGION 1..39: note = Recombinant collagen fragment
GIPGEKGPAGERGAPGPAGPRGAAGEPGRDGVPGGPGMR

SEQ ID NO: 488 - protein, 11 aa, synthetic construct
  REGION 1..11: note = Recombinant collagen fragment
GIPGFPGMKGH

SEQ ID NO: 489 - protein, 12 aa, synthetic construct
  REGION 1..12: note = Recombinant collagen fragment
GIPGFPGMKGHR

SEQ ID NO: 490 - protein, 14 aa, synthetic construct
  REGION 1..14: note = Recombinant collagen fragment
GIPGFPGMKGHRGF

SEQ ID NO: 491 - protein, 15 aa, synthetic construct
  REGION 1..15: note = Recombinant collagen fragment
GIPGFPGMKGHRGFD

SEQ ID NO: 492 - protein, 17 aa, synthetic construct
  REGION 1..17: note = Recombinant collagen fragment
GIPGFPGMKGHRGFDGR

SEQ ID NO: 493 - protein, 18 aa, synthetic construct
  REGION 1..18: note = Recombinant collagen fragment
GIPGFPGMKGHRGFDGRN

SEQ ID NO: 494 - protein, 21 aa, synthetic construct
  REGION 1..21: note = Recombinant collagen fragment
GIPGFPGMKGHRGFDGRNGEK

SEQ ID NO: 495 - protein, 22 aa, synthetic construct
  REGION 1..22: note = Recombinant collagen fragment
GIPGFPGMKGHRGFDGRNGEKG

SEQ ID NO: 496 - protein, 30 aa, synthetic construct
  REGION 1..30: note = Recombinant collagen fragment
GIPGFPGMKGHRGFDGRNGEKGETGAPGLK

SEQ ID NO: 497 - protein, 33 aa, synthetic construct
  REGION 1..33: note = Recombinant collagen fragment
GIPGFPGMKGHRGFDGRNGEKGETGAPGLKGEN

SEQ ID NO: 498 - protein, 39 aa, synthetic construct
  REGION 1..39: note = Recombinant collagen fragment
GIPGFPGMKGHRGFDGRNGEKGETGAPGLKGENGLPGEN

SEQ ID NO: 499 - protein, 48 aa, synthetic construct
  REGION 1..48: note = Recombinant collagen fragment
GIPGFPGMKGHRGFDGRNGEKGETGAPGLKGENGLPGENGAPGPMGPR

SEQ ID NO: 500 - protein, 24 aa, synthetic construct
  REGION 1..24: note = Recombinant collagen fragment
GKDGESGRPGRPGERGLPGPPGIK

SEQ ID NO: 501 - protein, 27 aa, synthetic construct
  REGION 1..27: note = Recombinant collagen fragment
GKDGESGRPGRPGERGLPGPPGIKGPA

SEQ ID NO: 502 - protein, 36 aa, synthetic construct
  REGION 1..36: note = Recombinant collagen fragment
GKDGESGRPGRPGERGLPGPPGIKGPAGIPGFPGMK

SEQ ID NO: 503 - protein, 17 aa, synthetic construct
  REGION 1..17: note = Recombinant collagen fragment
GKDGSPGEPGANGLPGA

SEQ ID NO: 504 - protein, 25 aa, synthetic construct
  REGION 1..25: note = Recombinant collagen fragment
GKDGSPGEPGANGLPGAAGERGAPG

SEQ ID NO: 505 - protein, 33 aa, synthetic construct
  REGION 1..33: note = Recombinant collagen fragment
GKDGSPGEPGANGLPGAAGERGAPGFRGPAGPN

SEQ ID NO: 506 - protein, 17 aa, synthetic construct
  REGION 1..17: note = Recombinant collagen fragment
GKGEMGPAGIPGAPGLM

SEQ ID NO: 507 - protein, 18 aa, synthetic construct
  REGION 1..18: note = Recombinant collagen fragment
GKGEMGPAGIPGAPGLMG

SEQ ID NO: 508 - protein, 19 aa, synthetic construct
  REGION 1..19: note = Recombinant collagen fragment
GKGEMGPAGIPGAPGLMGA

SEQ ID NO: 509 - protein, 20 aa, synthetic construct
  REGION 1..20: note = Recombinant collagen fragment
GKGEMGPAGIPGAPGLMGAR

SEQ ID NO: 510 - protein, 26 aa, synthetic construct
  REGION 1..26: note = Recombinant collagen fragment
GKGEMGPAGIPGAPGLMGARGPPGPA

SEQ ID NO: 511 - protein, 29 aa, synthetic construct
  REGION 1..29: note = Recombinant collagen fragment
GKGEMGPAGIPGAPGLMGARGPPGPAGAN

SEQ ID NO: 512 - protein, 35 aa, synthetic construct
  REGION 1..35: note = Recombinant collagen fragment
GKGEMGPAGIPGAPGLMGARGPPGPAGANGAPGLR

SEQ ID NO: 513 - protein, 28 aa, synthetic construct
  REGION 1..28: note = Recombinant collagen fragment
GKPGEPGPKGDAGAPGAPGGKGDAGAPG

SEQ ID NO: 514 - protein, 29 aa, synthetic construct
  REGION 1..29: note = Recombinant collagen fragment
GKPGEPGPKGDAGAPGAPGGKGDAGAPGE

SEQ ID NO: 515 - protein, 30 aa, synthetic construct
  REGION 1..30: note = Recombinant collagen fragment
GKPGEPGPKGDAGAPGAPGGKGDAGAPGER

SEQ ID NO: 516 - protein, 31 aa, synthetic construct
  REGION 1..31: note = Recombinant collagen fragment
GKPGEPGPKGDAGAPGAPGGKGDAGAPGERG

SEQ ID NO: 517 - protein, 33 aa, synthetic construct
  REGION 1..33: note = Recombinant collagen fragment
GKPGEPGPKGDAGAPGAPGGKGDAGAPGERGPP

SEQ ID NO: 518 - protein, 24 aa, synthetic construct
  REGION 1..24: note = Recombinant collagen fragment
GKPGPPGSQGESGRPGPPGPSGPR

SEQ ID NO: 519 - protein, 26 aa, synthetic construct
  REGION 1..26: note = Recombinant collagen fragment
GKPGPPGSQGESGRPGPPGPSGPRGQ

SEQ ID NO: 520 - protein, 30 aa, synthetic construct
  REGION 1..30: note = Recombinant collagen fragment
GKPGPPGSQGESGRPGPPGPSGPRGQPGVM

SEQ ID NO: 521 - protein, 21 aa, synthetic construct
  REGION 1..21: note = Recombinant collagen fragment
GLAGYPGPAGPPGPPGPPGTS

SEQ ID NO: 522 - protein, 57 aa, synthetic construct
  REGION 1..57: note = Recombinant collagen fragment
GLAGYPGPAGPPGPPGPPGTSGHPGSPGSPGYQGPPGEPGQAGPSGPPGPPGAIGPS

SEQ ID NO: 523 - protein, 21 aa, synthetic construct
  REGION 1..21: note = Recombinant collagen fragment
GLKGENGLPGENGAPGPMGPR

SEQ ID NO: 524 - protein, 21 aa, synthetic construct
  REGION 1..21: note = Recombinant collagen fragment
GLMGARGPPGPAGANGAPGLR

SEQ ID NO: 525 - protein, 30 aa, synthetic construct
  REGION 1..30: note = Recombinant collagen fragment
GLPGAAGARGNDGARGSDGQPGPPGPPGTA

SEQ ID NO: 526 - protein, 45 aa, synthetic construct
  REGION 1..45: note = Recombinant collagen fragment
GLPGAAGARGNDGARGSDGQPGPPGPPGTAGFPGSPGAKGEVGPA

SEQ ID NO: 527 - protein, 14 aa, synthetic construct
  REGION 1..14: note = Recombinant collagen fragment
GLPGAAGERGAPGF

SEQ ID NO: 528 - protein, 15 aa, synthetic construct
  REGION 1..15: note = Recombinant collagen fragment
GLPGAAGERGAPGFR

SEQ ID NO: 529 - protein, 16 aa, synthetic construct
  REGION 1..16: note = Recombinant collagen fragment
GLPGAAGERGAPGFRG

SEQ ID NO: 530 - protein, 19 aa, synthetic construct
  REGION 1..19: note = Recombinant collagen fragment
GLPGAAGERGAPGFRGPAG

SEQ ID NO: 531 - protein, 21 aa, synthetic construct
  REGION 1..21: note = Recombinant collagen fragment
GLPGAAGERGAPGFRGPAGPN

SEQ ID NO: 532 - protein, 28 aa, synthetic construct
  REGION 1..28: note = Recombinant collagen fragment
GLPGAAGERGAPGFRGPAGPNGIPGEKG

SEQ ID NO: 533 - protein, 33 aa, synthetic construct
  REGION 1..33: note = Recombinant collagen fragment
GLPGAAGERGAPGFRGPAGPNGIPGEKGPAGER

SEQ ID NO: 534 - protein, 37 aa, synthetic construct
  REGION 1..37: note = Recombinant collagen fragment
GLPGAAGERGAPGFRGPAGPNGIPGEKGPAGERGAPG

SEQ ID NO: 535 - protein, 42 aa, synthetic construct
  REGION 1..42: note = Recombinant collagen fragment
GLPGAAGERGAPGFRGPAGPNGIPGEKGPAGERGAPGPAGPR

SEQ ID NO: 536 - protein, 12 aa, synthetic construct
  REGION 1..12: note = Recombinant collagen fragment
GLPGENGAPGPM

SEQ ID NO: 537 - protein, 13 aa, synthetic construct
  REGION 1..13: note = Recombinant collagen fragment
GLPGENGAPGPMG

SEQ ID NO: 538 - protein, 15 aa, synthetic construct
  REGION 1..15: note = Recombinant collagen fragment
GLPGENGAPGPMGPR

SEQ ID NO: 539 - protein, 16 aa, synthetic construct
  REGION 1..16: note = Recombinant collagen fragment
GLPGENGAPGPMGPRG

SEQ ID NO: 540 - protein, 19 aa, synthetic construct
  REGION 1..19: note = Recombinant collagen fragment
GLPGENGAPGPMGPRGAPG

SEQ ID NO: 541 - protein, 23 aa, synthetic construct
  REGION 1..23: note = Recombinant collagen fragment
GLPGENGAPGPMGPRGAPGERGR

SEQ ID NO: 542 - protein, 25 aa, synthetic construct
  REGION 1..25: note = Recombinant collagen fragment
GLPGENGAPGPMGPRGAPGERGRPG

SEQ ID NO: 543 - protein, 33 aa, synthetic construct
  REGION 1..33: note = Recombinant collagen fragment
GLPGENGAPGPMGPRGAPGERGRPGLPGAAGAR

SEQ ID NO: 544 - protein, 35 aa, synthetic construct
  REGION 1..35: note = Recombinant collagen fragment
GLPGENGAPGPMGPRGAPGERGRPGLPGAAGARGN

SEQ ID NO: 545 - protein, 40 aa, synthetic construct
  REGION 1..40: note = Recombinant collagen fragment
GLPGENGAPGPMGPRGAPGERGRPGLPGAAGARGNDGARG

SEQ ID NO: 546 - protein, 44 aa, synthetic construct
  REGION 1..44: note = Recombinant collagen fragment
GLPGENGAPGPMGPRGAPGERGRPGLPGAAGARGNDGARGSDGQ

SEQ ID NO: 547 - protein, 54 aa, synthetic construct
  REGION 1..54: note = Recombinant collagen fragment
GLPGENGAPGPMGPRGAPGERGRPGLPGAAGARGNDGARGSDGQPGPPGPPGTA

SEQ ID NO: 548 - protein, 9 aa, synthetic construct
  REGION 1..9: note = Recombinant collagen fragment
GLPGPPGIK

SEQ ID NO: 549 - protein, 10 aa, synthetic construct
  REGION 1..10: note = Recombinant collagen fragment
GLPGPPGIKG

SEQ ID NO: 550 - protein, 11 aa, synthetic construct
  REGION 1..11: note = Recombinant collagen fragment
GLPGPPGIKGP

SEQ ID NO: 551 - protein, 12 aa, synthetic construct
  REGION 1..12: note = Recombinant collagen fragment
GLPGPPGIKGPA

SEQ ID NO: 552 - protein, 13 aa, synthetic construct
  REGION 1..13: note = Recombinant collagen fragment
GLPGPPGIKGPAG

SEQ ID NO: 553 - protein, 15 aa, synthetic construct
  REGION 1..15: note = Recombinant collagen fragment
GLPGPPGIKGPAGIP

SEQ ID NO: 554 - protein, 16 aa, synthetic construct
  REGION 1..16: note = Recombinant collagen fragment
GLPGPPGIKGPAGIPG

SEQ ID NO: 555 - protein, 17 aa, synthetic construct
  REGION 1..17: note = Recombinant collagen fragment
GLPGPPGIKGPAGIPGF

SEQ ID NO: 556 - protein, 20 aa, synthetic construct
  REGION 1..20: note = Recombinant collagen fragment
GLPGPPGIKGPAGIPGFPGM

SEQ ID NO: 557 - protein, 21 aa, synthetic construct
  REGION 1..21: note = Recombinant collagen fragment
GLPGPPGIKGPAGIPGFPGMK

SEQ ID NO: 558 - protein, 22 aa, synthetic construct
  REGION 1..22: note = Recombinant collagen fragment
GLPGPPGIKGPAGIPGFPGMKG

SEQ ID NO: 559 - protein, 23 aa, synthetic construct
  REGION 1..23: note = Recombinant collagen fragment
GLPGPPGIKGPAGIPGFPGMKGH

SEQ ID NO: 560 - protein, 24 aa, synthetic construct
  REGION 1..24: note = Recombinant collagen fragment
GLPGPPGIKGPAGIPGFPGMKGHR

SEQ ID NO: 561 - protein, 26 aa, synthetic construct
  REGION 1..26: note = Recombinant collagen fragment
GLPGPPGIKGPAGIPGFPGMKGHRGF

SEQ ID NO: 562 - protein, 12 aa, synthetic construct
  REGION 1..12: note = Recombinant collagen fragment
GLPGTGGPPGEN

SEQ ID NO: 563 - protein, 30 aa, synthetic construct
  REGION 1..30: note = Recombinant collagen fragment
GLRGGAGEPGKNGAKGEPGPRGERGEAGIP

SEQ ID NO: 564 - protein, 19 aa, synthetic construct
  REGION 1..19: note = Recombinant collagen fragment
GMPGSPGGPGSDGKPGPPG

SEQ ID NO: 565 - protein, 21 aa, synthetic construct
  REGION 1..21: note = Recombinant collagen fragment
GMPGSPGGPGSDGKPGPPGSQ

SEQ ID NO: 566 - protein, 22 aa, synthetic construct
  REGION 1..22: note = Recombinant collagen fragment
GMPGSPGGPGSDGKPGPPGSQG

SEQ ID NO: 567 - protein, 36 aa, synthetic construct
  REGION 1..36: note = Recombinant collagen fragment
GMPGSPGGPGSDGKPGPPGSQGESGRPGPPGPSGPR

SEQ ID NO: 568 - protein, 42 aa, synthetic construct
  REGION 1..42: note = Recombinant collagen fragment
GMPGSPGGPGSDGKPGPPGSQGESGRPGPPGPSGPRGQPGVM

SEQ ID NO: 569 - protein, 45 aa, synthetic construct
  REGION 1..45: note = Recombinant collagen fragment
GMRGMPGSPGGPGSDGKPGPPGSQGESGRPGPPGPSGPRGQPGVM

SEQ ID NO: 570 - protein, 27 aa, synthetic construct
  REGION 1..27: note = Recombinant collagen fragment
GNDGAPGKNGERGGPGGPGPQGPPGKN

SEQ ID NO: 571 - protein, 34 aa, synthetic construct
  REGION 1..34: note = Recombinant collagen fragment
GNDGAPGKNGERGGPGGPGPQGPPGKNGETGPQG

SEQ ID NO: 572 - protein, 38 aa, synthetic construct
  REGION 1..38: note = Recombinant collagen fragment
GNDGAPGKNGERGGPGGPGPQGPPGKNGETGPQGPPGP

SEQ ID NO: 573 - protein, 19 aa, synthetic construct
  REGION 1..19: note = Recombinant collagen fragment
GNDGARGSDGQPGPPGPPG

SEQ ID NO: 574 - protein, 21 aa, synthetic construct
  REGION 1..21: note = Recombinant collagen fragment
GNDGARGSDGQPGPPGPPGTA

SEQ ID NO: 575 - protein, 22 aa, synthetic construct
  REGION 1..22: note = Recombinant collagen fragment
GNDGARGSDGQPGPPGPPGTAG

SEQ ID NO: 576 - protein, 23 aa, synthetic construct
  REGION 1..23: note = Recombinant collagen fragment
GNDGARGSDGQPGPPGPPGTAGF

SEQ ID NO: 577 - protein, 25 aa, synthetic construct
  REGION 1..25: note = Recombinant collagen fragment
GNDGARGSDGQPGPPGPPGTAGFPG

SEQ ID NO: 578 - protein, 26 aa, synthetic construct
  REGION 1..26: note = Recombinant collagen fragment
GNDGARGSDGQPGPPGPPGTAGFPGS

SEQ ID NO: 579 - protein, 28 aa, synthetic construct
  REGION 1..28: note = Recombinant collagen fragment
GNDGARGSDGQPGPPGPPGTAGFPGSPG

SEQ ID NO: 580 - protein, 30 aa, synthetic construct
  REGION 1..30: note = Recombinant collagen fragment
GNDGARGSDGQPGPPGPPGTAGFPGSPGAK

SEQ ID NO: 581 - protein, 31 aa, synthetic construct
  REGION 1..31: note = Recombinant collagen fragment
GNDGARGSDGQPGPPGPPGTAGFPGSPGAKG

SEQ ID NO: 582 - protein, 32 aa, synthetic construct
  REGION 1..32: note = Recombinant collagen fragment
GNDGARGSDGQPGPPGPPGTAGFPGSPGAKGE

SEQ ID NO: 583 - protein, 34 aa, synthetic construct
  REGION 1..34: note = Recombinant collagen fragment
GNDGARGSDGQPGPPGPPGTAGFPGSPGAKGEVG

SEQ ID NO: 584 - protein, 35 aa, synthetic construct
  REGION 1..35: note = Recombinant collagen fragment
GNDGARGSDGQPGPPGPPGTAGFPGSPGAKGEVGP

SEQ ID NO: 585 - protein, 36 aa, synthetic construct
  REGION 1..36: note = Recombinant collagen fragment
GNDGARGSDGQPGPPGPPGTAGFPGSPGAKGEVGPA

SEQ ID NO: 586 - protein, 37 aa, synthetic construct
  REGION 1..37: note = Recombinant collagen fragment
GNDGARGSDGQPGPPGPPGTAGFPGSPGAKGEVGPAG

SEQ ID NO: 587 - protein, 38 aa, synthetic construct
  REGION 1..38: note = Recombinant collagen fragment
GNDGARGSDGQPGPPGPPGTAGFPGSPGAKGEVGPAGS

SEQ ID NO: 588 - protein, 40 aa, synthetic construct
  REGION 1..40: note = Recombinant collagen fragment
GNDGARGSDGQPGPPGPPGTAGFPGSPGAKGEVGPAGSPG

SEQ ID NO: 589 - protein, 42 aa, synthetic construct
  REGION 1..42: note = Recombinant collagen fragment
GNDGARGSDGQPGPPGPPGTAGFPGSPGAKGEVGPAGSPGSN

SEQ ID NO: 590 - protein, 43 aa, synthetic construct
  REGION 1..43: note = Recombinant collagen fragment
GNDGARGSDGQPGPPGPPGTAGFPGSPGAKGEVGPAGSPGSNG

SEQ ID NO: 591 - protein, 47 aa, synthetic construct
  REGION 1..47: note = Recombinant collagen fragment
GNDGARGSDGQPGPPGPPGTAGFPGSPGAKGEVGPAGSPGSNGAPGQ

SEQ ID NO: 592 - protein, 48 aa, synthetic construct
  REGION 1..48: note = Recombinant collagen fragment
GNDGARGSDGQPGPPGPPGTAGFPGSPGAKGEVGPAGSPGSNGAPGQR

SEQ ID NO: 593 - protein, 50 aa, synthetic construct
  REGION 1..50: note = Recombinant collagen fragment
GNDGARGSDGQPGPPGPPGTAGFPGSPGAKGEVGPAGSPGSNGAPGQRGE

SEQ ID NO: 594 - protein, 54 aa, synthetic construct
  REGION 1..54: note = Recombinant collagen fragment
GNDGARGSDGQPGPPGPPGTAGFPGSPGAKGEVGPAGSPGSNGAPGQRGEPGPQ

SEQ ID NO: 595 - protein, 55 aa, synthetic construct
  REGION 1..55: note = Recombinant collagen fragment
GNDGARGSDGQPGPPGPPGTAGFPGSPGAKGEVGPAGSPGSNGAPGQRGEPGPQG

SEQ ID NO: 596 - protein, 19 aa, synthetic construct
  REGION 1..19: note = Recombinant collagen fragment
GPAGANGAPGLRGGAGEPG

SEQ ID NO: 597 - protein, 12 aa, synthetic construct
  REGION 1..12: note = Recombinant collagen fragment
GPAGIPGAPGLM

SEQ ID NO: 598 - protein, 13 aa, synthetic construct
  REGION 1..13: note = Recombinant collagen fragment
GPAGIPGAPGLMG

SEQ ID NO: 599 - protein, 14 aa, synthetic construct
  REGION 1..14: note = Recombinant collagen fragment
GPAGIPGAPGLMGA

SEQ ID NO: 600 - protein, 15 aa, synthetic construct
  REGION 1..15: note = Recombinant collagen fragment
GPAGIPGAPGLMGAR

SEQ ID NO: 601 - protein, 30 aa, synthetic construct
  REGION 1..30: note = Recombinant collagen fragment
GPAGIPGAPGLMGARGPPGPAGANGAPGLR

SEQ ID NO: 602 - protein, 9 aa, synthetic construct
  REGION 1..9: note = Recombinant collagen fragment
GPAGIPGFP

SEQ ID NO: 603 - protein, 10 aa, synthetic construct
  REGION 1..10: note = Recombinant collagen fragment
GPAGIPGFPG

SEQ ID NO: 604 - protein, 11 aa, synthetic construct
  REGION 1..11: note = Recombinant collagen fragment
GPAGIPGFPGM

SEQ ID NO: 605 - protein, 12 aa, synthetic construct
  REGION 1..12: note = Recombinant collagen fragment
GPAGIPGFPGMK

SEQ ID NO: 606 - protein, 13 aa, synthetic construct
  REGION 1..13: note = Recombinant collagen fragment
GPAGIPGFPGMKG

SEQ ID NO: 607 - protein, 14 aa, synthetic construct
  REGION 1..14: note = Recombinant collagen fragment
GPAGIPGFPGMKGH

SEQ ID NO: 608 - protein, 15 aa, synthetic construct
  REGION 1..15: note = Recombinant collagen fragment
GPAGIPGFPGMKGHR

SEQ ID NO: 609 - protein, 16 aa, synthetic construct
  REGION 1..16: note = Recombinant collagen fragment
GPAGIPGFPGMKGHRG

SEQ ID NO: 610 - protein, 17 aa, synthetic construct
  REGION 1..17: note = Recombinant collagen fragment
GPAGIPGFPGMKGHRGF

SEQ ID NO: 611 - protein, 18 aa, synthetic construct
  REGION 1..18: note = Recombinant collagen fragment
GPAGIPGFPGMKGHRGFD

SEQ ID NO: 612 - protein, 19 aa, synthetic construct
  REGION 1..19: note = Recombinant collagen fragment
GPAGIPGFPGMKGHRGFDG

SEQ ID NO: 613 - protein, 20 aa, synthetic construct
  REGION 1..20: note = Recombinant collagen fragment
GPAGIPGFPGMKGHRGFDGR

SEQ ID NO: 614 - protein, 21 aa, synthetic construct
  REGION 1..21: note = Recombinant collagen fragment
GPAGIPGFPGMKGHRGFDGRN

SEQ ID NO: 615 - protein, 22 aa, synthetic construct
  REGION 1..22: note = Recombinant collagen fragment
GPAGIPGFPGMKGHRGFDGRNG

SEQ ID NO: 616 - protein, 23 aa, synthetic construct
  REGION 1..23: note = Recombinant collagen fragment
GPAGIPGFPGMKGHRGFDGRNGE

SEQ ID NO: 617 - protein, 24 aa, synthetic construct
  REGION 1..24: note = Recombinant collagen fragment
GPAGIPGFPGMKGHRGFDGRNGEK

SEQ ID NO: 618 - protein, 25 aa, synthetic construct
  REGION 1..25: note = Recombinant collagen fragment
GPAGIPGFPGMKGHRGFDGRNGEKG

SEQ ID NO: 619 - protein, 26 aa, synthetic construct
  REGION 1..26: note = Recombinant collagen fragment
GPAGIPGFPGMKGHRGFDGRNGEKGE

SEQ ID NO: 620 - protein, 27 aa, synthetic construct
  REGION 1..27: note = Recombinant collagen fragment
GPAGIPGFPGMKGHRGFDGRNGEKGET

SEQ ID NO: 621 - protein, 28 aa, synthetic construct
  REGION 1..28: note = Recombinant collagen fragment
GPAGIPGFPGMKGHRGFDGRNGEKGETG

SEQ ID NO: 622 - protein, 29 aa, synthetic construct
  REGION 1..29: note = Recombinant collagen fragment
GPAGIPGFPGMKGHRGFDGRNGEKGETGA

SEQ ID NO: 623 - protein, 30 aa, synthetic construct
  REGION 1..30: note = Recombinant collagen fragment
GPAGIPGFPGMKGHRGFDGRNGEKGETGAP

SEQ ID NO: 624 - protein, 31 aa, synthetic construct
  REGION 1..31: note = Recombinant collagen fragment
GPAGIPGFPGMKGHRGFDGRNGEKGETGAPG

SEQ ID NO: 625 - protein, 32 aa, synthetic construct
  REGION 1..32: note = Recombinant collagen fragment
GPAGIPGFPGMKGHRGFDGRNGEKGETGAPGL

SEQ ID NO: 626 - protein, 33 aa, synthetic construct
  REGION 1..33: note = Recombinant collagen fragment
GPAGIPGFPGMKGHRGFDGRNGEKGETGAPGLK

SEQ ID NO: 627 - protein, 34 aa, synthetic construct
  REGION 1..34: note = Recombinant collagen fragment
GPAGIPGFPGMKGHRGFDGRNGEKGETGAPGLKG

SEQ ID NO: 628 - protein, 36 aa, synthetic construct
  REGION 1..36: note = Recombinant collagen fragment
GPAGIPGFPGMKGHRGFDGRNGEKGETGAPGLKGEN

SEQ ID NO: 629 - protein, 40 aa, synthetic construct
  REGION 1..40: note = Recombinant collagen fragment
GPAGIPGFPGMKGHRGFDGRNGEKGETGAPGLKGENGLPG

SEQ ID NO: 630 - protein, 42 aa, synthetic construct
  REGION 1..42: note = Recombinant collagen fragment
GPAGIPGFPGMKGHRGFDGRNGEKGETGAPGLKGENGLPGEN

SEQ ID NO: 631 - protein, 48 aa, synthetic construct
  REGION 1..48: note = Recombinant collagen fragment
GPAGIPGFPGMKGHRGFDGRNGEKGETGAPGLKGENGLPGENGAPGPM

SEQ ID NO: 632 - protein, 51 aa, synthetic construct
  REGION 1..51: note = Recombinant collagen fragment
GPAGIPGFPGMKGHRGFDGRNGEKGETGAPGLKGENGLPGENGAPGPMGPR

SEQ ID NO: 633 - protein, 24 aa, synthetic construct
  REGION 1..24: note = Recombinant collagen fragment
GPAGKDGESGRPGRPGERGLPGPP

SEQ ID NO: 634 - protein, 25 aa, synthetic construct
  REGION 1..25: note = Recombinant collagen fragment
GPAGKDGESGRPGRPGERGLPGPPG

SEQ ID NO: 635 - protein, 27 aa, synthetic construct
  REGION 1..27: note = Recombinant collagen fragment
GPAGKDGESGRPGRPGERGLPGPPGIK

SEQ ID NO: 636 - protein, 30 aa, synthetic construct
  REGION 1..30: note = Recombinant collagen fragment
GPAGKDGESGRPGRPGERGLPGPPGIKGPA

SEQ ID NO: 637 - protein, 39 aa, synthetic construct
  REGION 1..39: note = Recombinant collagen fragment
GPAGKDGESGRPGRPGERGLPGPPGIKGPAGIPGFPGMK

SEQ ID NO: 638 - protein, 27 aa, synthetic construct
  REGION 1..27: note = Recombinant collagen fragment
GPAGPNGIPGEKGPAGERGAPGPAGPR

SEQ ID NO: 639 - protein, 12 aa, synthetic construct
  REGION 1..12: note = Recombinant collagen fragment
GPAGPPGPPGPP

SEQ ID NO: 640 - protein, 24 aa, synthetic construct
  REGION 1..24: note = Recombinant collagen fragment
GPGMRGMPGSPGGPGSDGKPGPPG

SEQ ID NO: 641 - protein, 47 aa, synthetic construct
  REGION 1..47: note = Recombinant collagen fragment
GPGMRGMPGSPGGPGSDGKPGPPGSQGESGRPGPPGPSGPRGQPGVM

SEQ ID NO: 642 - protein, 24 aa, synthetic construct
  REGION 1..24: note = Recombinant collagen fragment
GPGSDGKPGPPGSQGESGRPGPPG

SEQ ID NO: 643 - protein, 27 aa, synthetic construct
  REGION 1..27: note = Recombinant collagen fragment
GPGSDGKPGPPGSQGESGRPGPPGPSG

SEQ ID NO: 644 - protein, 29 aa, synthetic construct
  REGION 1..29: note = Recombinant collagen fragment
GPGSDGKPGPPGSQGESGRPGPPGPSGPR

SEQ ID NO: 645 - protein, 35 aa, synthetic construct
  REGION 1..35: note = Recombinant collagen fragment
GPGSDGKPGPPGSQGESGRPGPPGPSGPRGQPGVM

SEQ ID NO: 646 - protein, 43 aa, synthetic construct
  REGION 1..43: note = Recombinant collagen fragment
GPGSDGKPGPPGSQGESGRPGPPGPSGPRGQPGVMGFPGPKGN

SEQ ID NO: 647 - protein, 21 aa, synthetic construct
  REGION 1..21: note = Recombinant collagen fragment
GPMGPRGAPGERGRPGLPGAA

SEQ ID NO: 648 - protein, 24 aa, synthetic construct
  REGION 1..24: note = Recombinant collagen fragment
GPMGPRGAPGERGRPGLPGAAGAR

SEQ ID NO: 649 - protein, 21 aa, synthetic construct
  REGION 1..21: note = Recombinant collagen fragment
GPNGIPGEKGPAGERGAPGPA

SEQ ID NO: 650 - protein, 27 aa, synthetic construct
  REGION 1..27: note = Recombinant collagen fragment
GPPGAIGPSGPAGKDGESGRPGRPGER

SEQ ID NO: 651 - protein, 36 aa, synthetic construct
  REGION 1..36: note = Recombinant collagen fragment
GPPGAIGPSGPAGKDGESGRPGRPGERGLPGPPGIK

SEQ ID NO: 652 - protein, 19 aa, synthetic construct
  REGION 1..19: note = Recombinant collagen fragment
GPPGEPGQAGPSGPPGPPG

SEQ ID NO: 653 - protein, 24 aa, synthetic construct
  REGION 1..24: note = Recombinant collagen fragment
GPPGEPGQAGPSGPPGPPGAIGPS

SEQ ID NO: 654 - protein, 30 aa, synthetic construct
  REGION 1..30: note = Recombinant collagen fragment
GPPGEPGQAGPSGPPGPPGAIGPSGPAGKD

SEQ ID NO: 655 - protein, 51 aa, synthetic construct
  REGION 1..51: note = Recombinant collagen fragment
GPPGEPGQAGPSGPPGPPGAIGPSGPAGKDGESGRPGRPGERGLPGPPGIK

SEQ ID NO: 656 - protein, 18 aa, synthetic construct
  REGION 1..18: note = Recombinant collagen fragment
GPPGIKGPAGIPGFPGMK

SEQ ID NO: 657 - protein, 27 aa, synthetic construct
  REGION 1..27: note = Recombinant collagen fragment
GPPGINGSPGGKGEMGPAGIPGAPGLM

SEQ ID NO: 658 - protein, 15 aa, synthetic construct
  REGION 1..15: note = Recombinant collagen fragment
GPPGPAGANGAPGLR

SEQ ID NO: 659 - protein, 16 aa, synthetic construct
  REGION 1..16: note = Recombinant collagen fragment
GPPGPAGANGAPGLRG

SEQ ID NO: 660 - protein, 17 aa, synthetic construct
  REGION 1..17: note = Recombinant collagen fragment
GPPGPAGANGAPGLRGG

SEQ ID NO: 661 - protein, 18 aa, synthetic construct
  REGION 1..18: note = Recombinant collagen fragment
GPPGPAGANGAPGLRGGA

SEQ ID NO: 662 - protein, 19 aa, synthetic construct
  REGION 1..19: note = Recombinant collagen fragment
GPPGPAGANGAPGLRGGAG

SEQ ID NO: 663 - protein, 20 aa, synthetic construct
  REGION 1..20: note = Recombinant collagen fragment
GPPGPAGANGAPGLRGGAGE

SEQ ID NO: 664 - protein, 21 aa, synthetic construct
  REGION 1..21: note = Recombinant collagen fragment
GPPGPAGANGAPGLRGGAGEP

SEQ ID NO: 665 - protein, 22 aa, synthetic construct
  REGION 1..22: note = Recombinant collagen fragment
GPPGPAGANGAPGLRGGAGEPG

SEQ ID NO: 666 - protein, 23 aa, synthetic construct
  REGION 1..23: note = Recombinant collagen fragment
GPPGPAGANGAPGLRGGAGEPGK

SEQ ID NO: 667 - protein, 24 aa, synthetic construct
  REGION 1..24: note = Recombinant collagen fragment
GPPGPAGANGAPGLRGGAGEPGKN

SEQ ID NO: 668 - protein, 25 aa, synthetic construct
  REGION 1..25: note = Recombinant collagen fragment
GPPGPAGANGAPGLRGGAGEPGKNG

SEQ ID NO: 669 - protein, 26 aa, synthetic construct
  REGION 1..26: note = Recombinant collagen fragment
GPPGPAGANGAPGLRGGAGEPGKNGA

SEQ ID NO: 670 - protein, 27 aa, synthetic construct
  REGION 1..27: note = Recombinant collagen fragment
GPPGPAGANGAPGLRGGAGEPGKNGAK

SEQ ID NO: 671 - protein, 28 aa, synthetic construct
  REGION 1..28: note = Recombinant collagen fragment
GPPGPAGANGAPGLRGGAGEPGKNGAKG

SEQ ID NO: 672 - protein, 29 aa, synthetic construct
  REGION 1..29: note = Recombinant collagen fragment
GPPGPAGANGAPGLRGGAGEPGKNGAKGE

SEQ ID NO: 673 - protein, 31 aa, synthetic construct
  REGION 1..31: note = Recombinant collagen fragment
GPPGPAGANGAPGLRGGAGEPGKNGAKGEPG

SEQ ID NO: 674 - protein, 33 aa, synthetic construct
  REGION 1..33: note = Recombinant collagen fragment
GPPGPAGANGAPGLRGGAGEPGKNGAKGEPGPR

SEQ ID NO: 675 - protein, 34 aa, synthetic construct
  REGION 1..34: note = Recombinant collagen fragment
GPPGPAGANGAPGLRGGAGEPGKNGAKGEPGPRG

SEQ ID NO: 676 - protein, 35 aa, synthetic construct
  REGION 1..35: note = Recombinant collagen fragment
GPPGPAGANGAPGLRGGAGEPGKNGAKGEPGPRGE

SEQ ID NO: 677 - protein, 36 aa, synthetic construct
  REGION 1..36: note = Recombinant collagen fragment
GPPGPAGANGAPGLRGGAGEPGKNGAKGEPGPRGER

SEQ ID NO: 678 - protein, 37 aa, synthetic construct
  REGION 1..37: note = Recombinant collagen fragment
GPPGPAGANGAPGLRGGAGEPGKNGAKGEPGPRGERG

SEQ ID NO: 679 - protein, 38 aa, synthetic construct
  REGION 1..38: note = Recombinant collagen fragment
GPPGPAGANGAPGLRGGAGEPGKNGAKGEPGPRGERGE

SEQ ID NO: 680 - protein, 39 aa, synthetic construct
  REGION 1..39: note = Recombinant collagen fragment
GPPGPAGANGAPGLRGGAGEPGKNGAKGEPGPRGERGEA

SEQ ID NO: 681 - protein, 12 aa, synthetic construct
  REGION 1..12: note = Recombinant collagen fragment
GPPGPPGAIGPS

SEQ ID NO: 682 - protein, 15 aa, synthetic construct
  REGION 1..15: note = Recombinant collagen fragment
GPPGPPGAIGPSGPA

SEQ ID NO: 683 - protein, 30 aa, synthetic construct
  REGION 1..30: note = Recombinant collagen fragment
GPPGPPGAIGPSGPAGKDGESGRPGRPGER

SEQ ID NO: 684 - protein, 39 aa, synthetic construct
  REGION 1..39: note = Recombinant collagen fragment
GPPGPPGAIGPSGPAGKDGESGRPGRPGERGLPGPPGIK

SEQ ID NO: 685 - protein, 21 aa, synthetic construct
  REGION 1..21: note = Recombinant collagen fragment
GPPGPPGINGSPGGKGEMGPA

SEQ ID NO: 686 - protein, 30 aa, synthetic construct
  REGION 1..30: note = Recombinant collagen fragment
GPPGPPGINGSPGGKGEMGPAGIPGAPGLM

SEQ ID NO: 687 - protein, 32 aa, synthetic construct
  REGION 1..32: note = Recombinant collagen fragment
GPPGPPGINGSPGGKGEMGPAGIPGAPGLMGA

SEQ ID NO: 688 - protein, 33 aa, synthetic construct
  REGION 1..33: note = Recombinant collagen fragment
GPPGPPGINGSPGGKGEMGPAGIPGAPGLMGAR

SEQ ID NO: 689 - protein, 48 aa, synthetic construct
  REGION 1..48: note = Recombinant collagen fragment
GPPGPPGINGSPGGKGEMGPAGIPGAPGLMGARGPPGPAGANGAPGLR

SEQ ID NO: 690 - protein, 12 aa, synthetic construct
  REGION 1..12: note = Recombinant collagen fragment
GPPGPPGPPGTS

SEQ ID NO: 691 - protein, 16 aa, synthetic construct
  REGION 1..16: note = Recombinant collagen fragment
GPPGPPGPPGTSGHPG

SEQ ID NO: 692 - protein, 48 aa, synthetic construct
  REGION 1..48: note = Recombinant collagen fragment
GPPGPPGPPGTSGHPGSPGSPGYQGPPGEPGQAGPSGPPGPPGAIGPS

SEQ ID NO: 693 - protein, 18 aa, synthetic construct
  REGION 1..18: note = Recombinant collagen fragment
GPPGPPGTAGFPGSPGAK

SEQ ID NO: 694 - protein, 24 aa, synthetic construct
  REGION 1..24: note = Recombinant collagen fragment
GPPGPPGTAGFPGSPGAKGEVGPA

SEQ ID NO: 695 - protein, 15 aa, synthetic construct
  REGION 1..15: note = Recombinant collagen fragment
GPPGPSGPRGQPGVM

SEQ ID NO: 696 - protein, 30 aa, synthetic construct
  REGION 1..30: note = Recombinant collagen fragment
GPPGPSGPRGQPGVMGFPGPKGNDGAPGKN

SEQ ID NO: 697 - protein, 27 aa, synthetic construct
  REGION 1..27: note = Recombinant collagen fragment
GPPGSQGESGRPGPPGPSGPRGQPGVM

SEQ ID NO: 698 - protein, 21 aa, synthetic construct
  REGION 1..21: note = Recombinant collagen fragment
GPPGTAGFPGSPGAKGEVGPA

SEQ ID NO: 699 - protein, 18 aa, synthetic construct
  REGION 1..18: note = Recombinant collagen fragment
GPQGHAGAQGPPGPPGIN

SEQ ID NO: 700 - protein, 25 aa, synthetic construct
  REGION 1..25: note = Recombinant collagen fragment
GPRGAAGEPGRDGVPGGPGMRGMPG

SEQ ID NO: 701 - protein, 18 aa, synthetic construct
  REGION 1..18: note = Recombinant collagen fragment
GPRGAPGERGRPGLPGAA

SEQ ID NO: 702 - protein, 20 aa, synthetic construct
  REGION 1..20: note = Recombinant collagen fragment
GPRGAPGERGRPGLPGAAGA

SEQ ID NO: 703 - protein, 21 aa, synthetic construct
  REGION 1..21: note = Recombinant collagen fragment
GPRGAPGERGRPGLPGAAGAR

SEQ ID NO: 704 - protein, 23 aa, synthetic construct
  REGION 1..23: note = Recombinant collagen fragment
GPRGAPGERGRPGLPGAAGARGN

SEQ ID NO: 705 - protein, 28 aa, synthetic construct
  REGION 1..28: note = Recombinant collagen fragment
GPRGAPGERGRPGLPGAAGARGNDGARG

SEQ ID NO: 706 - protein, 42 aa, synthetic construct
  REGION 1..42: note = Recombinant collagen fragment
GPRGAPGERGRPGLPGAAGARGNDGARGSDGQPGPPGPPGTA

SEQ ID NO: 707 - protein, 17 aa, synthetic construct
  REGION 1..17: note = Recombinant collagen fragment
GPRGQPGVMGFPGPKGN

SEQ ID NO: 708 - protein, 24 aa, synthetic construct
  REGION 1..24: note = Recombinant collagen fragment
GPRGQPGVMGFPGPKGNDGAPGKN

SEQ ID NO: 709 - protein, 30 aa, synthetic construct
  REGION 1..30: note = Recombinant collagen fragment
GPSGPAGKDGESGRPGRPGERGLPGPPGIK

SEQ ID NO: 710 - protein, 33 aa, synthetic construct
  REGION 1..33: note = Recombinant collagen fragment
GPSGPAGKDGESGRPGRPGERGLPGPPGIKGPA

SEQ ID NO: 711 - protein, 15 aa, synthetic construct
  REGION 1..15: note = Recombinant collagen fragment
GPSGPPGPPGAIGPS

SEQ ID NO: 712 - protein, 27 aa, synthetic construct
  REGION 1..27: note = Recombinant collagen fragment
GPSGPRGQPGVMGFPGPKGNDGAPGKN

SEQ ID NO: 713 - protein, 18 aa, synthetic construct
  REGION 1..18: note = Recombinant collagen fragment
GQAGPSGPPGPPGAIGPS

SEQ ID NO: 714 - protein, 12 aa, synthetic construct
  REGION 1..12: note = Recombinant collagen fragment
GQPGPPGPPGTA

SEQ ID NO: 715 - protein, 21 aa, synthetic construct
  REGION 1..21: note = Recombinant collagen fragment
GQPGPPGPPGTAGFPGSPGAK

SEQ ID NO: 716 - protein, 27 aa, synthetic construct
  REGION 1..27: note = Recombinant collagen fragment
GQPGPPGPPGTAGFPGSPGAKGEVGPA

SEQ ID NO: 717 - protein, 12 aa, synthetic construct
  REGION 1..12: note = Recombinant collagen fragment
GQPGVMGFPGPK

SEQ ID NO: 718 - protein, 14 aa, synthetic construct
  REGION 1..14: note = Recombinant collagen fragment
GQPGVMGFPGPKGN

SEQ ID NO: 719 - protein, 21 aa, synthetic construct
  REGION 1..21: note = Recombinant collagen fragment
GQPGVMGFPGPKGNDGAPGKN

SEQ ID NO: 720 - protein, 24 aa, synthetic construct
  REGION 1..24: note = Recombinant collagen fragment
GQRGEPGPQGHAGAQGPPGPPGIN

SEQ ID NO: 721 - protein, 22 aa, synthetic construct
  REGION 1..22: note = Recombinant collagen fragment
GRDGVPGGPGMRGMPGSPGGPG

SEQ ID NO: 722 - protein, 54 aa, synthetic construct
  REGION 1..54: note = Recombinant collagen fragment
GRDGVPGGPGMRGMPGSPGGPGSDGKPGPPGSQGESGRPGPPGPSGPRGQPGVM

SEQ ID NO: 723 - protein, 24 aa, synthetic construct
  REGION 1..24: note = Recombinant collagen fragment
GRNGEKGETGAPGLKGENGLPGEN

SEQ ID NO: 724 - protein, 33 aa, synthetic construct
  REGION 1..33: note = Recombinant collagen fragment
GRNGEKGETGAPGLKGENGLPGENGAPGPMGPR

SEQ ID NO: 725 - protein, 15 aa, synthetic construct
  REGION 1..15: note = Recombinant collagen fragment
GRPGERGLPGPPGIK

SEQ ID NO: 726 - protein, 18 aa, synthetic construct
  REGION 1..18: note = Recombinant collagen fragment
GRPGERGLPGPPGIKGPA

SEQ ID NO: 727 - protein, 12 aa, synthetic construct
  REGION 1..12: note = Recombinant collagen fragment
GRPGLPGAAGAR

SEQ ID NO: 728 - protein, 33 aa, synthetic construct
  REGION 1..33: note = Recombinant collagen fragment
GRPGLPGAAGARGNDGARGSDGQPGPPGPPGTA

SEQ ID NO: 729 - protein, 42 aa, synthetic construct
  REGION 1..42: note = Recombinant collagen fragment
GRPGLPGAAGARGNDGARGSDGQPGPPGPPGTAGFPGSPGAK

SEQ ID NO: 730 - protein, 48 aa, synthetic construct
  REGION 1..48: note = Recombinant collagen fragment
GRPGLPGAAGARGNDGARGSDGQPGPPGPPGTAGFPGSPGAKGEVGPA

SEQ ID NO: 731 - protein, 18 aa, synthetic construct
  REGION 1..18: note = Recombinant collagen fragment
GRPGPPGPSGPRGQPGVM

SEQ ID NO: 732 - protein, 18 aa, synthetic construct
  REGION 1..18: note = Recombinant collagen fragment
GRPGRPGERGLPGPPGIK

SEQ ID NO: 733 - protein, 21 aa, synthetic construct
  REGION 1..21: note = Recombinant collagen fragment
GRPGRPGERGLPGPPGIKGPA

SEQ ID NO: 734 - protein, 33 aa, synthetic construct
  REGION 1..33: note = Recombinant collagen fragment
GSDGKPGPPGSQGESGRPGPPGPSGPRGQPGVM

SEQ ID NO: 735 - protein, 15 aa, synthetic construct
  REGION 1..15: note = Recombinant collagen fragment
GSDGQPGPPGPPGTA

SEQ ID NO: 736 - protein, 24 aa, synthetic construct
  REGION 1..24: note = Recombinant collagen fragment
GSDGQPGPPGPPGTAGFPGSPGAK

SEQ ID NO: 737 - protein, 30 aa, synthetic construct
  REGION 1..30: note = Recombinant collagen fragment
GSDGQPGPPGPPGTAGFPGSPGAKGEVGPA

SEQ ID NO: 738 - protein, 30 aa, synthetic construct
  REGION 1..30: note = Recombinant collagen fragment
GSNGAPGQRGEPGPQGHAGAQGPPGPPGIN

SEQ ID NO: 739 - protein, 30 aa, synthetic construct
  REGION 1..30: note = Recombinant collagen fragment
GSPGEPGANGLPGAAGERGAPGFRGPAGPN

SEQ ID NO: 740 - protein, 19 aa, synthetic construct
  REGION 1..19: note = Recombinant collagen fragment
GSPGGKGEMGPAGIPGAPG

SEQ ID NO: 741 - protein, 21 aa, synthetic construct
  REGION 1..21: note = Recombinant collagen fragment
GSPGGKGEMGPAGIPGAPGLM

SEQ ID NO: 742 - protein, 22 aa, synthetic construct
  REGION 1..22: note = Recombinant collagen fragment
GSPGGKGEMGPAGIPGAPGLMG

SEQ ID NO: 743 - protein, 23 aa, synthetic construct
  REGION 1..23: note = Recombinant collagen fragment
GSPGGKGEMGPAGIPGAPGLMGA

SEQ ID NO: 744 - protein, 24 aa, synthetic construct
  REGION 1..24: note = Recombinant collagen fragment
GSPGGKGEMGPAGIPGAPGLMGAR

SEQ ID NO: 745 - protein, 25 aa, synthetic construct
  REGION 1..25: note = Recombinant collagen fragment
GSPGGKGEMGPAGIPGAPGLMGARG

SEQ ID NO: 746 - protein, 28 aa, synthetic construct
  REGION 1..28: note = Recombinant collagen fragment
GSPGGKGEMGPAGIPGAPGLMGARGPPG

SEQ ID NO: 747 - protein, 30 aa, synthetic construct
  REGION 1..30: note = Recombinant collagen fragment
GSPGGKGEMGPAGIPGAPGLMGARGPPGPA

SEQ ID NO: 748 - protein, 31 aa, synthetic construct
  REGION 1..31: note = Recombinant collagen fragment
GSPGGKGEMGPAGIPGAPGLMGARGPPGPAG

SEQ ID NO: 749 - protein, 33 aa, synthetic construct
  REGION 1..33: note = Recombinant collagen fragment
GSPGGKGEMGPAGIPGAPGLMGARGPPGPAGAN

SEQ ID NO: 750 - protein, 39 aa, synthetic construct
  REGION 1..39: note = Recombinant collagen fragment
GSPGGKGEMGPAGIPGAPGLMGARGPPGPAGANGAPGLR

SEQ ID NO: 751 - protein, 48 aa, synthetic construct
  REGION 1..48: note = Recombinant collagen fragment
GSPGGKGEMGPAGIPGAPGLMGARGPPGPAGANGAPGLRGGAGEPGKN

SEQ ID NO: 752 - protein, 39 aa, synthetic construct
  REGION 1..39: note = Recombinant collagen fragment
GSPGGPGSDGKPGPPGSQGESGRPGPPGPSGPRGQPGVM

SEQ ID NO: 753 - protein, 18 aa, synthetic construct
  REGION 1..18: note = Recombinant collagen fragment
GSPGSNGAPGQRGEPGPQ

SEQ ID NO: 754 - protein, 24 aa, synthetic construct
  REGION 1..24: note = Recombinant collagen fragment
GSPGSNGAPGQRGEPGPQGHAGAQ

SEQ ID NO: 755 - protein, 26 aa, synthetic construct
  REGION 1..26: note = Recombinant collagen fragment
GSPGSNGAPGQRGEPGPQGHAGAQGP

SEQ ID NO: 756 - protein, 27 aa, synthetic construct
  REGION 1..27: note = Recombinant collagen fragment
GSPGSNGAPGQRGEPGPQGHAGAQGPP

SEQ ID NO: 757 - protein, 28 aa, synthetic construct
  REGION 1..28: note = Recombinant collagen fragment
GSPGSNGAPGQRGEPGPQGHAGAQGPPG

SEQ ID NO: 758 - protein, 29 aa, synthetic construct
  REGION 1..29: note = Recombinant collagen fragment
GSPGSNGAPGQRGEPGPQGHAGAQGPPGP

SEQ ID NO: 759 - protein, 30 aa, synthetic construct
  REGION 1..30: note = Recombinant collagen fragment
GSPGSNGAPGQRGEPGPQGHAGAQGPPGPP

SEQ ID NO: 760 - protein, 31 aa, synthetic construct
  REGION 1..31: note = Recombinant collagen fragment
GSPGSNGAPGQRGEPGPQGHAGAQGPPGPPG

SEQ ID NO: 761 - protein, 33 aa, synthetic construct
  REGION 1..33: note = Recombinant collagen fragment
GSPGSNGAPGQRGEPGPQGHAGAQGPPGPPGIN

SEQ ID NO: 762 - protein, 34 aa, synthetic construct
  REGION 1..34: note = Recombinant collagen fragment
GSPGSNGAPGQRGEPGPQGHAGAQGPPGPPGING

SEQ ID NO: 763 - protein, 35 aa, synthetic construct
  REGION 1..35: note = Recombinant collagen fragment
GSPGSNGAPGQRGEPGPQGHAGAQGPPGPPGINGS

SEQ ID NO: 764 - protein, 37 aa, synthetic construct
  REGION 1..37: note = Recombinant collagen fragment
GSPGSNGAPGQRGEPGPQGHAGAQGPPGPPGINGSPG

SEQ ID NO: 765 - protein, 38 aa, synthetic construct
  REGION 1..38: note = Recombinant collagen fragment
GSPGSNGAPGQRGEPGPQGHAGAQGPPGPPGINGSPGG

SEQ ID NO: 766 - protein, 39 aa, synthetic construct
  REGION 1..39: note = Recombinant collagen fragment
GSPGSNGAPGQRGEPGPQGHAGAQGPPGPPGINGSPGGK

SEQ ID NO: 767 - protein, 40 aa, synthetic construct
  REGION 1..40: note = Recombinant collagen fragment
GSPGSNGAPGQRGEPGPQGHAGAQGPPGPPGINGSPGGKG

SEQ ID NO: 768 - protein, 42 aa, synthetic construct
  REGION 1..42: note = Recombinant collagen fragment
GSPGSNGAPGQRGEPGPQGHAGAQGPPGPPGINGSPGGKGEM

SEQ ID NO: 769 - protein, 43 aa, synthetic construct
  REGION 1..43: note = Recombinant collagen fragment
GSPGSNGAPGQRGEPGPQGHAGAQGPPGPPGINGSPGGKGEMG

SEQ ID NO: 770 - protein, 45 aa, synthetic construct
  REGION 1..45: note = Recombinant collagen fragment
GSPGSNGAPGQRGEPGPQGHAGAQGPPGPPGINGSPGGKGEMGPA

SEQ ID NO: 771 - protein, 46 aa, synthetic construct
  REGION 1..46: note = Recombinant collagen fragment
GSPGSNGAPGQRGEPGPQGHAGAQGPPGPPGINGSPGGKGEMGPAG

SEQ ID NO: 772 - protein, 54 aa, synthetic construct
  REGION 1..54: note = Recombinant collagen fragment
GSPGSNGAPGQRGEPGPQGHAGAQGPPGPPGINGSPGGKGEMGPAGIPGAPGLM

SEQ ID NO: 773 - protein, 55 aa, synthetic construct
  REGION 1..55: note = Recombinant collagen fragment
GSPGSNGAPGQRGEPGPQGHAGAQGPPGPPGINGSPGGKGEMGPAGIPGAPGLMG

SEQ ID NO: 774 - protein, 56 aa, synthetic construct
  REGION 1..56: note = Recombinant collagen fragment
GSPGSNGAPGQRGEPGPQGHAGAQGPPGPPGINGSPGGKGEMGPAGIPGAPGLMGA

SEQ ID NO: 775 - protein, 24 aa, synthetic construct
  REGION 1..24: note = Recombinant collagen fragment
GSQGESGRPGPPGPSGPRGQPGVM

SEQ ID NO: 776 - protein, 18 aa, synthetic construct
  REGION 1..18: note = Recombinant collagen fragment
GTAGFPGSPGAKGEVGPA

SEQ ID NO: 777 - protein, 39 aa, synthetic construct
  REGION 1..39: note = Recombinant collagen fragment
GTSGHPGSPGSPGYQGPPGEPGQAGPSGPPGPPGAIGPS

SEQ ID NO: 778 - protein, 30 aa, synthetic construct
  REGION 1..30: note = Recombinant collagen fragment
GVPGGPGMRGMPGSPGGPGSDGKPGPPGSQ

SEQ ID NO: 779 - protein, 51 aa, synthetic construct
  REGION 1..51: note = Recombinant collagen fragment
GVPGGPGMRGMPGSPGGPGSDGKPGPPGSQGESGRPGPPGPSGPRGQPGVM

SEQ ID NO: 780 - protein, 17 aa, synthetic construct
  REGION 1..17: note = Recombinant collagen fragment
GYPGPAGPPGPPGPPGT

SEQ ID NO: 781 - protein, 18 aa, synthetic construct
  REGION 1..18: note = Recombinant collagen fragment
GYPGPAGPPGPPGPPGTS

SEQ ID NO: 782 - protein, 20 aa, synthetic construct
  REGION 1..20: note = Recombinant collagen fragment
GYPGPAGPPGPPGPPGTSGH

SEQ ID NO: 783 - protein, 22 aa, synthetic construct
  REGION 1..22: note = Recombinant collagen fragment
GYPGPAGPPGPPGPPGTSGHPG

SEQ ID NO: 784 - protein, 54 aa, synthetic construct
  REGION 1..54: note = Recombinant collagen fragment
GYPGPAGPPGPPGPPGTSGHPGSPGSPGYQGPPGEPGQAGPSGPPGPPGAIGPS

SEQ ID NO: 785 - protein, 57 aa, synthetic construct
  REGION 1..57: note = Recombinant collagen fragment
GYPGPAGPPGPPGPPGTSGHPGSPGSPGYQGPPGEPGQAGPSGPPGPPGAIGPSGPA

SEQ ID NO: 786 - protein, 14 aa, synthetic construct
  REGION 1..14: note = Recombinant collagen fragment
HAGAQGPPGPPGIN

SEQ ID NO: 787 - protein, 18 aa, synthetic construct
  REGION 1..18: note = Recombinant collagen fragment
HAGAQGPPGPPGINGSPG

SEQ ID NO: 788 - protein, 24 aa, synthetic construct
  REGION 1..24: note = Recombinant collagen fragment
HAGAQGPPGPPGINGSPGGKGEMG

SEQ ID NO: 789 - protein, 26 aa, synthetic construct
  REGION 1..26: note = Recombinant collagen fragment
HAGAQGPPGPPGINGSPGGKGEMGPA

SEQ ID NO: 790 - protein, 38 aa, synthetic construct
  REGION 1..38: note = Recombinant collagen fragment
HAGAQGPPGPPGINGSPGGKGEMGPAGIPGAPGLMGAR

SEQ ID NO: 791 - protein, 35 aa, synthetic construct
  REGION 1..35: note = Recombinant collagen fragment
HPGSPGSPGYQGPPGEPGQAGPSGPPGPPGAIGPS

SEQ ID NO: 792 - protein, 23 aa, synthetic construct
  REGION 1..23: note = Recombinant collagen fragment
HRGFDGRNGEKGETGAPGLKGEN

SEQ ID NO: 793 - protein, 38 aa, synthetic construct
  REGION 1..38: note = Recombinant collagen fragment
HRGFDGRNGEKGETGAPGLKGENGLPGENGAPGPMGPR

SEQ ID NO: 794 - protein, 31 aa, synthetic construct
  REGION 1..31: note = Recombinant collagen fragment
IGPSGPAGKDGESGRPGRPGERGLPGPPGIK

SEQ ID NO: 795 - protein, 12 aa, synthetic construct
  REGION 1..12: note = Recombinant collagen fragment
IKGPAGIPGFPG

SEQ ID NO: 796 - protein, 14 aa, synthetic construct
  REGION 1..14: note = Recombinant collagen fragment
IKGPAGIPGFPGMK

SEQ ID NO: 797 - protein, 26 aa, synthetic construct
  REGION 1..26: note = Recombinant collagen fragment
IPGAPGLMGARGPPGPAGANGAPGLR

SEQ ID NO: 798 - protein, 17 aa, synthetic construct
  REGION 1..17: note = Recombinant collagen fragment
IPGFPGMKGHRGFDGRN

SEQ ID NO: 799 - protein, 23 aa, synthetic construct
  REGION 1..23: note = Recombinant collagen fragment
KDGESGRPGRPGERGLPGPPGIK

SEQ ID NO: 800 - protein, 26 aa, synthetic construct
  REGION 1..26: note = Recombinant collagen fragment
KDGESGRPGRPGERGLPGPPGIKGPA

SEQ ID NO: 801 - protein, 32 aa, synthetic construct
  REGION 1..32: note = Recombinant collagen fragment
KDGSPGEPGANGLPGAAGERGAPGFRGPAGPN

SEQ ID NO: 802 - protein, 19 aa, synthetic construct
  REGION 1..19: note = Recombinant collagen fragment
KGENGLPGENGAPGPMGPR

SEQ ID NO: 803 - protein, 13 aa, synthetic construct
  REGION 1..13: note = Recombinant collagen fragment
KGPAGIPGFPGMK

SEQ ID NO: 804 - protein, 23 aa, synthetic construct
  REGION 1..23: note = Recombinant collagen fragment
KPGPPGSQGESGRPGPPGPSGPR

SEQ ID NO: 805 - protein, 29 aa, synthetic construct
  REGION 1..29: note = Recombinant collagen fragment
KPGPPGSQGESGRPGPPGPSGPRGQPGVM

SEQ ID NO: 806 - protein, 20 aa, synthetic construct
  REGION 1..20: note = Recombinant collagen fragment
LKGENGLPGENGAPGPMGPR

SEQ ID NO: 807 - protein, 20 aa, synthetic construct
  REGION 1..20: note = Recombinant collagen fragment
LMGARGPPGPAGANGAPGLR

SEQ ID NO: 808 - protein, 29 aa, synthetic construct
  REGION 1..29: note = Recombinant collagen fragment
LPGAAGARGNDGARGSDGQPGPPGPPGTA

SEQ ID NO: 809 - protein, 20 aa, synthetic construct
  REGION 1..20: note = Recombinant collagen fragment
LPGAAGERGAPGFRGPAGPN

SEQ ID NO: 810 - protein, 8 aa, synthetic construct
  REGION 1..8: note = Recombinant collagen fragment
LPGPPGIK

SEQ ID NO: 811 - protein, 11 aa, synthetic construct
  REGION 1..11: note = Recombinant collagen fragment
LPGPPGIKGPA

SEQ ID NO: 812 - protein, 20 aa, synthetic construct
  REGION 1..20: note = Recombinant collagen fragment
LPGPPGIKGPAGIPGFPGMK

SEQ ID NO: 813 - protein, 19 aa, synthetic construct
  REGION 1..19: note = Recombinant collagen fragment
MGARGPPGPAGANGAPGLR

SEQ ID NO: 814 - protein, 28 aa, synthetic construct
  REGION 1..28: note = Recombinant collagen fragment
MGARGPPGPAGANGAPGLRGGAGEPGKN

SEQ ID NO: 815 - protein, 31 aa, synthetic construct
  REGION 1..31: note = Recombinant collagen fragment
MGPAGIPGAPGLMGARGPPGPAGANGAPGLR

SEQ ID NO: 816 - protein, 22 aa, synthetic construct
  REGION 1..22: note = Recombinant collagen fragment
MGPRGAPGERGRPGLPGAAGAR

SEQ ID NO: 817 - protein, 41 aa, synthetic construct
  REGION 1..41: note = Recombinant collagen fragment
MPGSPGGPGSDGKPGPPGSQGESGRPGPPGPSGPRGQPGVM

SEQ ID NO: 818 - protein, 44 aa, synthetic construct
  REGION 1..44: note = Recombinant collagen fragment
MRGMPGSPGGPGSDGKPGPPGSQGESGRPGPPGPSGPRGQPGVM

SEQ ID NO: 819 - protein, 20 aa, synthetic construct
  REGION 1..20: note = Recombinant collagen fragment
NDGARGSDGQPGPPGPPGTA

SEQ ID NO: 820 - protein, 35 aa, synthetic construct
  REGION 1..35: note = Recombinant collagen fragment
NDGARGSDGQPGPPGPPGTAGFPGSPGAKGEVGPA

SEQ ID NO: 821 - protein, 15 aa, synthetic construct
  REGION 1..15: note = Recombinant collagen fragment
NGAPGPMGPRGAPGE

SEQ ID NO: 822 - protein, 28 aa, synthetic construct
  REGION 1..28: note = Recombinant collagen fragment
NGAPGPMGPRGAPGERGRPGLPGAAGAR

SEQ ID NO: 823 - protein, 31 aa, synthetic construct
  REGION 1..31: note = Recombinant collagen fragment
NGEKGETGAPGLKGENGLPGENGAPGPMGPR

SEQ ID NO: 824 - protein, 18 aa, synthetic construct
  REGION 1..18: note = Recombinant collagen fragment
NGETGPQGPPGPTGPGGD

SEQ ID NO: 825 - protein, 16 aa, synthetic construct
  REGION 1..16: note = Recombinant collagen fragment
NGLPGENGAPGPMGPR

SEQ ID NO: 826 - protein, 22 aa, synthetic construct
  REGION 1..22: note = Recombinant collagen fragment
NGSPGGKGEMGPAGIPGAPGLM

SEQ ID NO: 827 - protein, 40 aa, synthetic construct
  REGION 1..40: note = Recombinant collagen fragment
NGSPGGKGEMGPAGIPGAPGLMGARGPPGPAGANGAPGLR

SEQ ID NO: 828 - protein, 11 aa, synthetic construct
  REGION 1..11: note = Recombinant collagen fragment
PAGIPGAPGLM

SEQ ID NO: 829 - protein, 12 aa, synthetic construct
  REGION 1..12: note = Recombinant collagen fragment
PAGIPGAPGLMG

SEQ ID NO: 830 - protein, 11 aa, synthetic construct
  REGION 1..11: note = Recombinant collagen fragment
PAGIPGFPGMK

SEQ ID NO: 831 - protein, 12 aa, synthetic construct
  REGION 1..12: note = Recombinant collagen fragment
PAGIPGFPGMKG

SEQ ID NO: 832 - protein, 14 aa, synthetic construct
  REGION 1..14: note = Recombinant collagen fragment
PAGIPGFPGMKGHR

SEQ ID NO: 833 - protein, 15 aa, synthetic construct
  REGION 1..15: note = Recombinant collagen fragment
PAGIPGFPGMKGHRG

SEQ ID NO: 834 - protein, 16 aa, synthetic construct
  REGION 1..16: note = Recombinant collagen fragment
PAGIPGFPGMKGHRGF

SEQ ID NO: 835 - protein, 18 aa, synthetic construct
  REGION 1..18: note = Recombinant collagen fragment
PAGIPGFPGMKGHRGFDG

SEQ ID NO: 836 - protein, 20 aa, synthetic construct
  REGION 1..20: note = Recombinant collagen fragment
PAGIPGFPGMKGHRGFDGRN

SEQ ID NO: 837 - protein, 26 aa, synthetic construct
  REGION 1..26: note = Recombinant collagen fragment
PAGKDGESGRPGRPGERGLPGPPGIK

SEQ ID NO: 838 - protein, 19 aa, synthetic construct
  REGION 1..19: note = Recombinant collagen fragment
PGAAGERGAPGFRGPAGPN

SEQ ID NO: 839 - protein, 16 aa, synthetic construct
  REGION 1..16: note = Recombinant collagen fragment
PGANGLPGAAGERGAP

SEQ ID NO: 840 - protein, 25 aa, synthetic construct
  REGION 1..25: note = Recombinant collagen fragment
PGAPGLMGARGPPGPAGANGAPGLR

SEQ ID NO: 841 - protein, 27 aa, synthetic construct
  REGION 1..27: note = Recombinant collagen fragment
PGEPGPKGDAGAPGAPGGKGDAGAPGE

SEQ ID NO: 842 - protein, 13 aa, synthetic construct
  REGION 1..13: note = Recombinant collagen fragment
PGERGLPGPPGIK

SEQ ID NO: 843 - protein, 16 aa, synthetic construct
  REGION 1..16: note = Recombinant collagen fragment
PGERGRPGLPGAAGAR

SEQ ID NO: 844 - protein, 9 aa, synthetic construct
  REGION 1..9: note = Recombinant collagen fragment
PGFPGMKGH

SEQ ID NO: 845 - protein, 10 aa, synthetic construct
  REGION 1..10: note = Recombinant collagen fragment
PGFPGMKGHR

SEQ ID NO: 846 - protein, 12 aa, synthetic construct
  REGION 1..12: note = Recombinant collagen fragment
PGFPGMKGHRGF

SEQ ID NO: 847 - protein, 15 aa, synthetic construct
  REGION 1..15: note = Recombinant collagen fragment
PGFPGMKGHRGFDGR

SEQ ID NO: 848 - protein, 21 aa, synthetic construct
  REGION 1..21: note = Recombinant collagen fragment
PGLMGARGPPGPAGANGAPGL

SEQ ID NO: 849 - protein, 41 aa, synthetic construct
  REGION 1..41: note = Recombinant collagen fragment
PGLPGAAGARGNDGARGSDGQPGPPGPPGTAGFPGSPGAKG

SEQ ID NO: 850 - protein, 22 aa, synthetic construct
  REGION 1..22: note = Recombinant collagen fragment
PGPAGANGAPGLRGGAGEPGKN

SEQ ID NO: 851 - protein, 14 aa, synthetic construct
  REGION 1..14: note = Recombinant collagen fragment
PGPAGPPGPPGPPG

SEQ ID NO: 852 - protein, 16 aa, synthetic construct
  REGION 1..16: note = Recombinant collagen fragment
PGPAGPPGPPGPPGTS

SEQ ID NO: 853 - protein, 17 aa, synthetic construct
  REGION 1..17: note = Recombinant collagen fragment
PGPAGPPGPPGPPGTSG

SEQ ID NO: 854 - protein, 18 aa, synthetic construct
  REGION 1..18: note = Recombinant collagen fragment
PGPAGPPGPPGPPGTSGH

SEQ ID NO: 855 - protein, 42 aa, synthetic construct
  REGION 1..42: note = Recombinant collagen fragment
PGPAGPPGPPGPPGTSGHPGSPGSPGYQGPPGEPGQAGPSGP

SEQ ID NO: 856 - protein, 57 aa, synthetic construct
  REGION 1..57: note = Recombinant collagen fragment
PGPAGPPGPPGPPGTSGHPGSPGSPGYQGPPGEPGQAGPSGPPGPPGAIGPSGPAGK

SEQ ID NO: 857 - protein, 13 aa, synthetic construct
  REGION 1..13: note = Recombinant collagen fragment
PGPKGNDGAPGKN

SEQ ID NO: 858 - protein, 31 aa, synthetic construct
  REGION 1..31: note = Recombinant collagen fragment
PGPKGNDGAPGKNGERGGPGGPGPQGPPGKN

SEQ ID NO: 859 - protein, 25 aa, synthetic construct
  REGION 1..25: note = Recombinant collagen fragment
PGPMGPRGAPGERGRPGLPGAAGAR

SEQ ID NO: 860 - protein, 10 aa, synthetic construct
  REGION 1..10: note = Recombinant collagen fragment
PGPPGAIGPS

SEQ ID NO: 861 - protein, 19 aa, synthetic construct
  REGION 1..19: note = Recombinant collagen fragment
PGPPGIKGPAGIPGFPGMK

SEQ ID NO: 862 - protein, 19 aa, synthetic construct
  REGION 1..19: note = Recombinant collagen fragment
PGPPGPPGTAGFPGSPGAK

SEQ ID NO: 863 - protein, 16 aa, synthetic construct
  REGION 1..16: note = Recombinant collagen fragment
PGPPGPSGPRGQPGVM

SEQ ID NO: 864 - protein, 16 aa, synthetic construct
  REGION 1..16: note = Recombinant collagen fragment
PGRPGERGLPGPPGIK

SEQ ID NO: 865 - protein, 34 aa, synthetic construct
  REGION 1..34: note = Recombinant collagen fragment
PGSDGKPGPPGSQGESGRPGPPGPSGPRGQPGVM

SEQ ID NO: 866 - protein, 33 aa, synthetic construct
  REGION 1..33: note = Recombinant collagen fragment
PGSNGAPGQRGEPGPQGHAGAQGPPGPPGINGS

SEQ ID NO: 867 - protein, 13 aa, synthetic construct
  REGION 1..13: note = Recombinant collagen fragment
PGSPGAKGEVGPA

SEQ ID NO: 868 - protein, 26 aa, synthetic construct
  REGION 1..26: note = Recombinant collagen fragment
PPGAIGPSGPAGKDGESGRPGRPGER

SEQ ID NO: 869 - protein, 35 aa, synthetic construct
  REGION 1..35: note = Recombinant collagen fragment
PPGAIGPSGPAGKDGESGRPGRPGERGLPGPPGIK

SEQ ID NO: 870 - protein, 23 aa, synthetic construct
  REGION 1..23: note = Recombinant collagen fragment
PPGEPGQAGPSGPPGPPGAIGPS

SEQ ID NO: 871 - protein, 29 aa, synthetic construct
  REGION 1..29: note = Recombinant collagen fragment
PPGEPGQAGPSGPPGPPGAIGPSGPAGKD

SEQ ID NO: 872 - protein, 17 aa, synthetic construct
  REGION 1..17: note = Recombinant collagen fragment
PPGIKGPAGIPGFPGMK

SEQ ID NO: 873 - protein, 26 aa, synthetic construct
  REGION 1..26: note = Recombinant collagen fragment
PPGINGSPGGKGEMGPAGIPGAPGLM

SEQ ID NO: 874 - protein, 14 aa, synthetic construct
  REGION 1..14: note = Recombinant collagen fragment
PPGPAGANGAPGLR

SEQ ID NO: 875 - protein, 11 aa, synthetic construct
  REGION 1..11: note = Recombinant collagen fragment
PPGPPGAIGPS

SEQ ID NO: 876 - protein, 38 aa, synthetic construct
  REGION 1..38: note = Recombinant collagen fragment
PPGPPGAIGPSGPAGKDGESGRPGRPGERGLPGPPGIK

SEQ ID NO: 877 - protein, 29 aa, synthetic construct
  REGION 1..29: note = Recombinant collagen fragment
PPGPPGINGSPGGKGEMGPAGIPGAPGLM

SEQ ID NO: 878 - protein, 23 aa, synthetic construct
  REGION 1..23: note = Recombinant collagen fragment
PPGPPGTAGFPGSPGAKGEVGPA

SEQ ID NO: 879 - protein, 26 aa, synthetic construct
  REGION 1..26: note = Recombinant collagen fragment
PPGSQGESGRPGPPGPSGPRGQPGVM

SEQ ID NO: 880 - protein, 20 aa, synthetic construct
  REGION 1..20: note = Recombinant collagen fragment
PPGTAGFPGSPGAKGEVGPA

SEQ ID NO: 881 - protein, 17 aa, synthetic construct
  REGION 1..17: note = Recombinant collagen fragment
PQGHAGAQGPPGPPGIN

SEQ ID NO: 882 - protein, 29 aa, synthetic construct
  REGION 1..29: note = Recombinant collagen fragment
PSGPAGKDGESGRPGRPGERGLPGPPGIK

SEQ ID NO: 883 - protein, 14 aa, synthetic construct
  REGION 1..14: note = Recombinant collagen fragment
PSGPPGPPGAIGPS

SEQ ID NO: 884 - protein, 17 aa, synthetic construct
  REGION 1..17: note = Recombinant collagen fragment
QAGPSGPPGPPGAIGPS

SEQ ID NO: 885 - protein, 31 aa, synthetic construct
  REGION 1..31: note = Recombinant collagen fragment
QGPPGEPGQAGPSGPPGPPGAIGPSGPAGKD

SEQ ID NO: 886 - protein, 23 aa, synthetic construct
  REGION 1..23: note = Recombinant collagen fragment
QRGEPGPQGHAGAQGPPGPPGIN

SEQ ID NO: 887 - protein, 22 aa, synthetic construct
  REGION 1..22: note = Recombinant collagen fragment
RGFDGRNGEKGETGAPGLKGEN

SEQ ID NO: 888 - protein, 10 aa, synthetic construct
  REGION 1..10: note = Recombinant collagen fragment
RGLPGPPGIK

SEQ ID NO: 889 - protein, 13 aa, synthetic construct
  REGION 1..13: note = Recombinant collagen fragment
RGLPGPPGIKGPA

SEQ ID NO: 890 - protein, 22 aa, synthetic construct
  REGION 1..22: note = Recombinant collagen fragment
RGLPGPPGIKGPAGIPGFPGMK

SEQ ID NO: 891 - protein, 43 aa, synthetic construct
  REGION 1..43: note = Recombinant collagen fragment
RGMPGSPGGPGSDGKPGPPGSQGESGRPGPPGPSGPRGQPGVM

SEQ ID NO: 892 - protein, 22 aa, synthetic construct
  REGION 1..22: note = Recombinant collagen fragment
RGNDGARGSDGQPGPPGPPGTA

SEQ ID NO: 893 - protein, 31 aa, synthetic construct
  REGION 1..31: note = Recombinant collagen fragment
RGNDGARGSDGQPGPPGPPGTAGFPGSPGAK

SEQ ID NO: 894 - protein, 37 aa, synthetic construct
  REGION 1..37: note = Recombinant collagen fragment
RGNDGARGSDGQPGPPGPPGTAGFPGSPGAKGEVGPA

SEQ ID NO: 895 - protein, 15 aa, synthetic construct
  REGION 1..15: note = Recombinant collagen fragment
RGPPGPAGANGAPGL

SEQ ID NO: 896 - protein, 16 aa, synthetic construct
  REGION 1..16: note = Recombinant collagen fragment
RGPPGPAGANGAPGLR

SEQ ID NO: 897 - protein, 17 aa, synthetic construct
  REGION 1..17: note = Recombinant collagen fragment
RGPPGPAGANGAPGLRG

SEQ ID NO: 898 - protein, 25 aa, synthetic construct
  REGION 1..25: note = Recombinant collagen fragment
RGPPGPAGANGAPGLRGGAGEPGKN

SEQ ID NO: 899 - protein, 28 aa, synthetic construct
  REGION 1..28: note = Recombinant collagen fragment
RGPPGPAGANGAPGLRGGAGEPGKNGAK

SEQ ID NO: 900 - protein, 25 aa, synthetic construct
  REGION 1..25: note = Recombinant collagen fragment
RGSDGQPGPPGPPGTAGFPGSPGAK

SEQ ID NO: 901 - protein, 31 aa, synthetic construct
  REGION 1..31: note = Recombinant collagen fragment
RGSDGQPGPPGPPGTAGFPGSPGAKGEVGPA

SEQ ID NO: 902 - protein, 14 aa, synthetic construct
  REGION 1..14: note = Recombinant collagen fragment
RPGERGLPGPPGIK

SEQ ID NO: 903 - protein, 17 aa, synthetic construct
  REGION 1..17: note = Recombinant collagen fragment
RPGERGLPGPPGIKGPA

SEQ ID NO: 904 - protein, 28 aa, synthetic construct
  REGION 1..28: note = Recombinant collagen fragment
RPGLPGAAGARGNDGARGSDGQPGPPGP

SEQ ID NO: 905 - protein, 32 aa, synthetic construct
  REGION 1..32: note = Recombinant collagen fragment
RPGLPGAAGARGNDGARGSDGQPGPPGPPGTA

SEQ ID NO: 906 - protein, 17 aa, synthetic construct
  REGION 1..17: note = Recombinant collagen fragment
RPGPPGPSGPRGQPGVM

SEQ ID NO: 907 - protein, 17 aa, synthetic construct
  REGION 1..17: note = Recombinant collagen fragment
RPGRPGERGLPGPPGIK

SEQ ID NO: 908 - protein, 20 aa, synthetic construct
  REGION 1..20: note = Recombinant collagen fragment
RPGRPGERGLPGPPGIKGPA

SEQ ID NO: 909 - protein, 23 aa, synthetic construct
  REGION 1..23: note = Recombinant collagen fragment
SDGKPGPPGSQGESGRPGPPGPS

SEQ ID NO: 910 - protein, 24 aa, synthetic construct
  REGION 1..24: note = Recombinant collagen fragment
SDGKPGPPGSQGESGRPGPPGPSG

SEQ ID NO: 911 - protein, 26 aa, synthetic construct
  REGION 1..26: note = Recombinant collagen fragment
SDGKPGPPGSQGESGRPGPPGPSGPR

SEQ ID NO: 912 - protein, 27 aa, synthetic construct
  REGION 1..27: note = Recombinant collagen fragment
SDGKPGPPGSQGESGRPGPPGPSGPRG

SEQ ID NO: 913 - protein, 30 aa, synthetic construct
  REGION 1..30: note = Recombinant collagen fragment
SDGKPGPPGSQGESGRPGPPGPSGPRGQPG

SEQ ID NO: 914 - protein, 32 aa, synthetic construct
  REGION 1..32: note = Recombinant collagen fragment
SDGKPGPPGSQGESGRPGPPGPSGPRGQPGVM

SEQ ID NO: 915 - protein, 40 aa, synthetic construct
  REGION 1..40: note = Recombinant collagen fragment
SDGKPGPPGSQGESGRPGPPGPSGPRGQPGVMGFPGPKGN

SEQ ID NO: 916 - protein, 14 aa, synthetic construct
  REGION 1..14: note = Recombinant collagen fragment
SDGQPGPPGPPGTA

SEQ ID NO: 917 - protein, 23 aa, synthetic construct
  REGION 1..23: note = Recombinant collagen fragment
SDGQPGPPGPPGTAGFPGSPGAK

SEQ ID NO: 918 - protein, 29 aa, synthetic construct
  REGION 1..29: note = Recombinant collagen fragment
SDGQPGPPGPPGTAGFPGSPGAKGEVGPA

SEQ ID NO: 919 - protein, 28 aa, synthetic construct
  REGION 1..28: note = Recombinant collagen fragment
SGPAGKDGESGRPGRPGERGLPGPPGIK

SEQ ID NO: 920 - protein, 13 aa, synthetic construct
  REGION 1..13: note = Recombinant collagen fragment
SGPPGPPGAIGPS

SEQ ID NO: 921 - protein, 19 aa, synthetic construct
  REGION 1..19: note = Recombinant collagen fragment
SGPPGPPGAIGPSGPAGKD

SEQ ID NO: 922 - protein, 31 aa, synthetic construct
  REGION 1..31: note = Recombinant collagen fragment
SGPPGPPGAIGPSGPAGKDGESGRPGRPGER

SEQ ID NO: 923 - protein, 40 aa, synthetic construct
  REGION 1..40: note = Recombinant collagen fragment
SGPPGPPGAIGPSGPAGKDGESGRPGRPGERGLPGPPGIK

SEQ ID NO: 924 - protein, 43 aa, synthetic construct
  REGION 1..43: note = Recombinant collagen fragment
SGPPGPPGAIGPSGPAGKDGESGRPGRPGERGLPGPPGIKGPA

SEQ ID NO: 925 - protein, 18 aa, synthetic construct
  REGION 1..18: note = Recombinant collagen fragment
SGPRGQPGVMGFPGPKGN

SEQ ID NO: 926 - protein, 19 aa, synthetic construct
  REGION 1..19: note = Recombinant collagen fragment
SGRPGPPGPSGPRGQPGVM

SEQ ID NO: 927 - protein, 27 aa, synthetic construct
  REGION 1..27: note = Recombinant collagen fragment
SGRPGPPGPSGPRGQPGVMGFPGPKGN

SEQ ID NO: 928 - protein, 16 aa, synthetic construct
  REGION 1..16: note = Recombinant collagen fragment
SGRPGRPGERGLPGPP

SEQ ID NO: 929 - protein, 17 aa, synthetic construct
  REGION 1..17: note = Recombinant collagen fragment
SGRPGRPGERGLPGPPG

SEQ ID NO: 930 - protein, 19 aa, synthetic construct
  REGION 1..19: note = Recombinant collagen fragment
SGRPGRPGERGLPGPPGIK

SEQ ID NO: 931 - protein, 22 aa, synthetic construct
  REGION 1..22: note = Recombinant collagen fragment
SGRPGRPGERGLPGPPGIKGPA

SEQ ID NO: 932 - protein, 29 aa, synthetic construct
  REGION 1..29: note = Recombinant collagen fragment
SNGAPGQRGEPGPQGHAGAQGPPGPPGIN

SEQ ID NO: 933 - protein, 35 aa, synthetic construct
  REGION 1..35: note = Recombinant collagen fragment
SNGAPGQRGEPGPQGHAGAQGPPGPPGINGSPGGK

SEQ ID NO: 934 - protein, 41 aa, synthetic construct
  REGION 1..41: note = Recombinant collagen fragment
SNGAPGQRGEPGPQGHAGAQGPPGPPGINGSPGGKGEMGPA

SEQ ID NO: 935 - protein, 21 aa, synthetic construct
  REGION 1..21: note = Recombinant collagen fragment
SPGAKGEVGPAGSPGSNGAPG

SEQ ID NO: 936 - protein, 22 aa, synthetic construct
  REGION 1..22: note = Recombinant collagen fragment
SPGAKGEVGPAGSPGSNGAPGQ

SEQ ID NO: 937 - protein, 25 aa, synthetic construct
  REGION 1..25: note = Recombinant collagen fragment
SPGAKGEVGPAGSPGSNGAPGQRGE

SEQ ID NO: 938 - protein, 27 aa, synthetic construct
  REGION 1..27: note = Recombinant collagen fragment
SPGAKGEVGPAGSPGSNGAPGQRGEPG

SEQ ID NO: 939 - protein, 29 aa, synthetic construct
  REGION 1..29: note = Recombinant collagen fragment
SPGAKGEVGPAGSPGSNGAPGQRGEPGPQ

SEQ ID NO: 940 - protein, 30 aa, synthetic construct
  REGION 1..30: note = Recombinant collagen fragment
SPGAKGEVGPAGSPGSNGAPGQRGEPGPQG

SEQ ID NO: 941 - protein, 31 aa, synthetic construct
  REGION 1..31: note = Recombinant collagen fragment
SPGAKGEVGPAGSPGSNGAPGQRGEPGPQGH

SEQ ID NO: 942 - protein, 33 aa, synthetic construct
  REGION 1..33: note = Recombinant collagen fragment
SPGAKGEVGPAGSPGSNGAPGQRGEPGPQGHAG

SEQ ID NO: 943 - protein, 44 aa, synthetic construct
  REGION 1..44: note = Recombinant collagen fragment
SPGAKGEVGPAGSPGSNGAPGQRGEPGPQGHAGAQGPPGPPGIN

SEQ ID NO: 944 - protein, 29 aa, synthetic construct
  REGION 1..29: note = Recombinant collagen fragment
SPGEPGANGLPGAAGERGAPGFRGPAGPN

SEQ ID NO: 945 - protein, 20 aa, synthetic construct
  REGION 1..20: note = Recombinant collagen fragment
SPGGKGEMGPAGIPGAPGLM

SEQ ID NO: 946 - protein, 22 aa, synthetic construct
  REGION 1..22: note = Recombinant collagen fragment
SPGGKGEMGPAGIPGAPGLMGA

SEQ ID NO: 947 - protein, 23 aa, synthetic construct
  REGION 1..23: note = Recombinant collagen fragment
SPGGKGEMGPAGIPGAPGLMGAR

SEQ ID NO: 948 - protein, 32 aa, synthetic construct
  REGION 1..32: note = Recombinant collagen fragment
SPGGKGEMGPAGIPGAPGLMGARGPPGPAGAN

SEQ ID NO: 949 - protein, 27 aa, synthetic construct
  REGION 1..27: note = Recombinant collagen fragment
SPGGPGSDGKPGPPGSQGESGRPGPPG

SEQ ID NO: 950 - protein, 28 aa, synthetic construct
  REGION 1..28: note = Recombinant collagen fragment
SPGGPGSDGKPGPPGSQGESGRPGPPGP

SEQ ID NO: 951 - protein, 29 aa, synthetic construct
  REGION 1..29: note = Recombinant collagen fragment
SPGGPGSDGKPGPPGSQGESGRPGPPGPS

SEQ ID NO: 952 - protein, 32 aa, synthetic construct
  REGION 1..32: note = Recombinant collagen fragment
SPGGPGSDGKPGPPGSQGESGRPGPPGPSGPR

SEQ ID NO: 953 - protein, 33 aa, synthetic construct
  REGION 1..33: note = Recombinant collagen fragment
SPGGPGSDGKPGPPGSQGESGRPGPPGPSGPRG

SEQ ID NO: 954 - protein, 38 aa, synthetic construct
  REGION 1..38: note = Recombinant collagen fragment
SPGGPGSDGKPGPPGSQGESGRPGPPGPSGPRGQPGVM

SEQ ID NO: 955 - protein, 46 aa, synthetic construct
  REGION 1..46: note = Recombinant collagen fragment
SPGGPGSDGKPGPPGSQGESGRPGPPGPSGPRGQPGVMGFPGPKGN

SEQ ID NO: 956 - protein, 27 aa, synthetic construct
  REGION 1..27: note = Recombinant collagen fragment
SPGSNGAPGQRGEPGPQGHAGAQGPPG

SEQ ID NO: 957 - protein, 32 aa, synthetic construct
  REGION 1..32: note = Recombinant collagen fragment
SPGSNGAPGQRGEPGPQGHAGAQGPPGPPGIN

SEQ ID NO: 958 - protein, 36 aa, synthetic construct
  REGION 1..36: note = Recombinant collagen fragment
SPGSNGAPGQRGEPGPQGHAGAQGPPGPPGINGSPG

SEQ ID NO: 959 - protein, 38 aa, synthetic construct
  REGION 1..38: note = Recombinant collagen fragment
SPGSNGAPGQRGEPGPQGHAGAQGPPGPPGINGSPGGK

SEQ ID NO: 960 - protein, 42 aa, synthetic construct
  REGION 1..42: note = Recombinant collagen fragment
SPGSNGAPGQRGEPGPQGHAGAQGPPGPPGINGSPGGKGEMG

SEQ ID NO: 961 - protein, 44 aa, synthetic construct
  REGION 1..44: note = Recombinant collagen fragment
SPGSNGAPGQRGEPGPQGHAGAQGPPGPPGINGSPGGKGEMGPA

SEQ ID NO: 962 - protein, 32 aa, synthetic construct
  REGION 1..32: note = Recombinant collagen fragment
SPGSPGYQGPPGEPGQAGPSGPPGPPGAIGPS

SEQ ID NO: 963 - protein, 29 aa, synthetic construct
  REGION 1..29: note = Recombinant collagen fragment
SPGYQGPPGEPGQAGPSGPPGPPGAIGPS

SEQ ID NO: 964 - protein, 23 aa, synthetic construct
  REGION 1..23: note = Recombinant collagen fragment
SQGESGRPGPPGPSGPRGQPGVM

SEQ ID NO: 965 - protein, 31 aa, synthetic construct
  REGION 1..31: note = Recombinant collagen fragment
SQGESGRPGPPGPSGPRGQPGVMGFPGPKGN

SEQ ID NO: 966 - protein, 17 aa, synthetic construct
  REGION 1..17: note = Recombinant collagen fragment
TAGFPGSPGAKGEVGPA

SEQ ID NO: 967 - protein, 18 aa, synthetic construct
  REGION 1..18: note = Recombinant collagen fragment
TAGFPGSPGAKGEVGPAG

SEQ ID NO: 968 - protein, 21 aa, synthetic construct
  REGION 1..21: note = Recombinant collagen fragment
TAGFPGSPGAKGEVGPAGSPG

SEQ ID NO: 969 - protein, 23 aa, synthetic construct
  REGION 1..23: note = Recombinant collagen fragment
TAGFPGSPGAKGEVGPAGSPGSN

SEQ ID NO: 970 - protein, 25 aa, synthetic construct
  REGION 1..25: note = Recombinant collagen fragment
TGAPGLKGENGLPGENGAPGPMGPR

SEQ ID NO: 971 - protein, 15 aa, synthetic construct
  REGION 1..15: note = Recombinant collagen fragment
TGPQGPPGPTGPGGD

SEQ ID NO: 972 - protein, 23 aa, synthetic construct
  REGION 1..23: note = Recombinant collagen fragment
VGPAGSPGSNGAPGQRGEPGPQG

SEQ ID NO: 973 - protein, 1062 aa, synthetic construct
  REGION 1..1062: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRERGSEGSPGHPGQPGPPGPPGAPGPCCGGVGAAAIAGIGGEKAGGFAPYYG

SEQ ID NO: 974 - protein, 546 aa, synthetic construct; identical to SEQ ID NO: 1
  REGION 1..546: note = Recombinant collagen fragment

SEQ ID NO: 975 - protein, 546 aa, synthetic construct
  REGION 1..546: note = Recombinant collagen fragment
DVKSGVAVGGLAGYPGPAGAPGPPGPPGASGHPGSPGAPGYQGPPGAPGQAGPSGAPGPPGAIGASGPAGKDGASGRPGRPGERGLPGPPGAKGPAGIPGAPGMKGHRGFDGRNGEKGATGAPGLKGANGLPGENGAPGPMGPRGAPGERGRPGAPGAAGARGADGARGSDGAPGPPGPPGAAGFPGSPGAKGEVGPAGAPGSNGAPGARGEPGPQGAAGAQGPPGAPGINGSPGAKGEMGPAGAPGAPGLMGARGPPGPAGANGAPGLRGAAGEPGKNGAKGEPGPRGARGEAGIPGAPGAKGEDGADGSPGEPGANGLPGAAGARGAPGFRGAAGPNGIPGAKGPAGERGAPGPAGPRGAAGEPGRDGAPGGPGMRGAPGSPGGPGADGKPGPPGAQGESGRPGAPGPSGPRGAPGVMGFPGAKGNDGAPGANGERGGPGAPGPQGPPGANGETGPQGAPGPTGPGGAKGDTGPPGAQGLQGLPGAGGPPGENGAPGEPGPKGAAGAPGAPGAKGDAGAPGERGPPAIAGIGGEKAGGFAPYYG

SEQ ID NO: 976 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGEPGLMGPRGLPGSPGGPGPAGKEGPVGLPGIDGRPGPIGPAGARGEPGVMGFPGPKGNDGDPGKNGDKGHAGLAGARGAPGPDGETGAQGPPGPQGVQGGKGEQGPAGPPGFQGLPGTGGPAGEVGKPGEQGLHGEFGLPGPAGPRGERGAPGERGPPAIAGIGGEKAGGFAPYYG

SEQ ID NO: 977 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKRGESGRPGRPGERGLPGPPGIKGPAGIPGFPGMKGHRGFRGRNGEKGETGAPGLKGENGLPGENGAPGPMGPRGAPGERGRPGLPGAAGARGNRGARGSDGQPGPPGPPGTAGFPGSPGAKGRVGPAGSPGSNGAPGQRGEPGPQGHAGAQGPPGPPGINGSPGGKGEMGPAGIPGAPGLMGARGPPGPAGANGAPGLRGGAGRPGKNGAKGEPGPRGERGRAGIPGVPGAKGERGKDGSPGEPGANGLPGAAGERGAPGFRGPAGPNGIPGEKGPAGERGAPGPAGPRGAAGEPGRRGVPGGPGMRGMPGSPGGPGSRGKPGPPGSQGESGRPGPPGPSGPRGQPGVMGFPGPKGNDGAPGKNGERGGPGGPGPQGPPGKNGETGPQGPPGPTGPGGRKGDTGPPGPQGLQGLPGTGGPPGENGKPGRPGPKGDAGAPGAPGGKGRAGAPGERGPPAIAGIGGEKAGGFAPYYG

SEQ ID NO: 978 - protein, 546 aa, synthetic construct
  REGION 1..546: note = Recombinant collagen fragment
DVKSGVAVGGLAGYPGPAGPPGPPGPPGASGHPGSPGSPGYQGPPGEPGQAGPAGPPGPPGAIGPSGPAGKDGESGRPGRPGERGLPGPPGIKGPAGIPGFPGMKGHRGFDGRNGEKGEAGAPGLKGENGLPGENGAPGPMGPRGAPGERGRPGLPGAAGARGNDGARGSDGQPGPPGPPGAAGFPGAPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGAQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 979 - protein, 546 aa, synthetic construct
  REGION 1..546: note = Recombinant collagen fragment
DVKSGVAVGGLAGYPGPAGPPGPPGPPGNSGHPGSPGSPGYQGPPGEPGQAGPNGPPGPPGAIGPSGPAGKDGESGRPGRPGERGLPGPPGIKGPAGIPGFPGMKGHRGFDGRNGEKGENGAPGLKGENGLPGENGAPGPMGPRGAPGERGRPGLPGAAGARGNDGARGSDGQPGPPGPPGNAGFPGNPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGNQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 980 - protein, 546 aa, synthetic construct; identical to SEQ ID NO: 975
  REGION 1..546: note = Recombinant collagen fragment

SEQ ID NO: 981 - protein, 549 aa, synthetic construct
  REGION 1..549: note = Recombinant collagen fragment
DSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 982 - protein, 318 aa, synthetic construct
  REGION 1..318: note = human Col1A1
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPAIAGIGGEKAGGFAPYYG

SEQ ID NO: 983 - protein, 318 aa, synthetic construct; identical to SEQ ID NO: 982
  REGION 1..318: note = human Col1A2

SEQ ID NO: 984 - protein, 438 aa, synthetic construct
  REGION 1..438: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPAIAGIGGEKAGGFAPYYG

SEQ ID NO: 985 - protein, 546 aa, synthetic construct
  REGION 1..438: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGHRGFPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 986 - protein, 556 aa, synthetic construct
  REGION 1..556: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGAAGLPGPPGIKGPAGIPGFPGMKGHRGFDGRNGEKGETGAPGLKGENGLPGAAGAPGPMGPRGAPGAAGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGPLVLRQLLVLVEKRPAVSLHIMVNQEDVRMPFA

SEQ ID NO: 987 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGKPGRPGERGLPGPPGIKGPAGIPGFPGMKGHRGFDGKNGEKGETGAPGLKGENGLPGENGAPGPMGPKGAPGERGKPGLPGAAGAKGNDGAKGSDGQPGPPGPPGTAGFPGSPGAKGEVGPAGSPGSNGAPGQKGEPGPQGHAGAQGPPGPPGINGSPGGKGEMGPAGIPGAPGLMGAKGPPGPAGANGAPGLKGGAGEPGKNGAKGEPGPKGEKGEAGIPGVPGAKGEDGKDGSPGEPGANGLPGAAGEKGAPGFKGPAGPNGIPGEKGPAGEKGAPGPAGPKGAAGEPGKDGVPGGPGMKGMPGSPGGPGSDGKPGPPGSQGESGKPGPPGPSGPKGQPGVMGFPGPKGNDGAPGKNGEKGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 988 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGAVGAKGEAGPQGPRGSEGPQGVRGEPGPPGPAGAAGPAGPPGADGQPGAKGAMGAPGIAGAPGFPGARGPPGPQGAGGPPGPKGGAGEPGAPGAKGDTGAKGERGPVGVQGPPGPAGEEGKRGARGEPGPTGLPGPPGERGGPGFRGFPGADGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 989 - protein, 546 aa, synthetic construct
  REGION 1..546: note = Recombinant collagen fragment
DVKSGVAVGGIAGYPGPAGPPGPPGPPGTSGHPGSPGSPGYQGPPGEPGQAGPSGPPGPPGALGPSGPAGKDGESGRPGRPGERGVPGPPGVKGPAGLPGFPGMKGHRGFDGRNGEKGETGAPGLKGENGLPGENGAPGPMGPRGAPGERGRPGLPGAAGARGNDGARGSDGQPGPPGPPGTAGFPGSPGAKGELGPAGSPGSNGAPGQRGEPGPQGHAGAQGPPGPPGLNGSPGGKGEMGPAGVPGAPGIMGARGPPGPAGANGAPGIRGGAGEPGKNGAKGEPGPRGERGEAGLPGLPGAKGEDGKDGSPGEPGANGVPGAAGERGAPGFRGPAGPNGLPGEKGPAGERGAPGPAGPRGAAGEPGRDGIPGGPGMRGMPGSPGGPGSDGKPGPPGSQGESGRPGPPGPSGPRGQPGLMGFPGPKGNDGAPGKNGERGGPGGPGPQGPPGKNGETGPQGPPGPTGPGGDKGDTGPPGPQGVQGLPGTGGPPGENGKPGEPGPKGDAGAPGAPGGKGDAGAPGERGPPAIAGIGGEKAGGFAPYYG

SEQ ID NO: 990 - protein, 546 aa, synthetic construct
  REGION 1..546: note = Recombinant collagen fragment
DVKSGVAVGGLAGYPGPAGPPGPPGPPGAAGHPGAPGAPGYQGPPGEPGQAGPAGPPGPPGAIGPAGPAGKDGEAGRPGRPGERGLPGPPGIKGPAGIPGFPGMKGHRGFDGRDGEKGEAGAPGLKGEDGLPGENGAPGPMGPRGAPGERGRPGLPGAAGARGDDGARGADGQPGPPGPPGAAGFPGAPGAKGEVGPAGAPGADGAPGQRGEPGPQGHAGAQGPPGPPGIDGAPGGKGEMGPAGIPGAPGLMGARGPPGPAGADGAPGLRGGAGEPGKDGAKGEPGPRGERGEAGIPGVPGAKGEDGKDGAPGEPGADGLPGAAGERGAPGFRGPAGPDGIPGEKGPAGERGAPGPAGPRGAAGEPGRDGVPGGPGMRGMPGAPGGPGADGKPGPPGAQGEAGRPGPPGPAGPRGQPGVMGFPGPKGDDGAPGKDGERGGPGGPGPQGPPGKDGEAGPQGPPGPAGPGGDKGDAGPPGPQGLQGLPGAGGPPGEDGKPGEPGPKGDAGAPGAPGGKGDAGAPGERGPPAIAGIGGEKAGGFAPYYG

SEQ ID NO: 991 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIEPGRPEGEKGGGRGGAPGAPGRGAGNGEGARPPPLMGGAGGNKAGPLAAPIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 992 - protein, 546 aa, synthetic construct; identical to SEQ ID NO: 991
  REGION 1..546: note = Recombinant collagen fragment

SEQ ID NO: 993 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKRGESGRPGRPGERGLPGPPGIKGPAGIPGFPGMKGHRGFRGRNGEKGRTGAPGLKGENGLPGENGAPGPMGPRGAPGERGRPGLPGAAGARGNRGARGSRGQPGPPGPPGTAGFPGSPGAKGRVGPAGSPGSNGAPGQRGRPGPQGHAGAQGPPGPPGINGSPGGKGRMGPAGIPGAPGLMGARGPPGPAGANGAPGLRGGAGRPGKNGAKGRPGPRGERGRAGIPGVPGAKGRRGKRGSPGEPGANGLPGAAGERGAPGFRGPAGPNGIPGEKGPAGERGAPGPAGPRGAAGRPGRRGVPGGPGMRGMPGSPGGPGSRGKPGPPGSQGESGRPGPPGPSGPRGQPGVMGFPGPKGNRGAPGKNGRRGGPGGPGPQGPPGKNGETGPQGPPGPTGPGGRKGRTGPPGPQGLQGLPGTGGPPGENGKPGRPGPKGDAGAPGAPGGKGRAGAPGERGPPAIAGIGGEKAGGFAPYYG

SEQ ID NO: 994 - protein, 556 aa, synthetic construct
  REGION 1..556: note = Recombinant collagen fragment
DVKSGVAVGGMGGYPGPAGPPGPPGPPGVSGHPGAPGAPGYQGPPGEPGQAGPAGPPGPPGAMGPAGPAGKDGESGRPGRPGERGFPGPPGIKGPAGMPGFPGMKGHRGFDGRNGEKGDTGAPGLKGENGLPGENGAPGPMGPRGAPGERGRPGLPGAAGARGNDGARGSDGQPGPPGPPGTAGFPGSPGAKGEVGPAGAPGPNGAPGQRGEPGLQGQAGAPGPPGPPGINGSPGGKGEMGPAGIPGAPGLIGARGPPGPPGANGVPGQRGAAGEPGKNGAKGDPGARGERGEAGIPGIAGPKGEDGKDGSPGEPGANGLPGAPGERGPPGFRGAPGANGIPGEKGPPGERGGPGPAGPRGVAGEPGRDGLPGGPGLRGIPGSPGGPGSDGKPGPPGSQGESGRPGPPAHLVQGVSLVSWVSPVQRVMTEHRVKMVNVVAQVVQDCPVQQEKTVRQVLKDLQDLPVLQEIREMQDRQDLLDCKVCLVQEALPEKMVSLESQDQKAMSEPQVFPEVRETPVLRVSVVLRQLLVLVEKRPAVSLHIMVNQEDVRMPFA

SEQ ID NO: 995 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGVAGPKGPAGERGAPGPAGPKGAAGEAGRPGEAGLPGAKGMPGSPGGPGPDGKPGPPGPAGQDGRPGPPGPPGARGQAGVMGFPGPKGAAGEPGKAGERGVPGPPGAVGPAGKDGEAGAQGPPGPAGPAGERGEQGPAGPQGFQGLPGPAGPPGEAGKPGEQGVPGDLGAPGAPGARGERGAPGERGPPAIAGIGGEKAGGFAPYYG

SEQ ID NO: 996 - protein, 531 aa, synthetic construct
  REGION 1..531: note = Recombinant collagen fragment
DSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPP

SEQ ID NO: 997 - protein, 556 aa, synthetic construct
  REGION 1..556: note = Recombinant collagen fragment
DVKSGVAVGKTTKSGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPKTTKSAIAGIGGEKAGGFAPYYG

SEQ ID NO: 998 - protein, 556 aa, synthetic construct; identical to SEQ ID NO: 997
  REGION 1..556: note = Recombinant collagen fragment

SEQ ID NO: 999 - protein, 556 aa, synthetic construct
  REGION 1..556: note = Recombinant collagen fragment
DVKSGVAVGKTTKSGLAGYPGPAGPPGPPGPPGTSGHPGSPGSPGYQGPPGEPGQAGPSGPPGPPGAIGPSGPAGKDGESGRPGRPGAAGLPGPPGIKGPAGIPGFPGMKGHRGFDGRNGEKGETGAPGLKGENGLPGAAGAPGPMGPRGAPGAAGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGAAGPPKTTKSAIAGIGGEKAGGFAPYYG

SEQ ID NO: 1000 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEEGKRGARGEAGSAGPPGPPGLRGAPGFRGLPGADGRAGVMGPPGERGAPGPAGVRGAAGDAGRPGEPGLMGPRGLPGSPGGPGPAGKEGPVGLPGIDGRPGPIGPAGARGEPGVMGFPGPKGNDGDPGKNGDKGHAGLAGARGAPGPDGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 1001 - protein, 546 aa, synthetic construct
  REGION 1..546: note = Recombinant collagen fragment
DVKSGVAVGGMGGYPGPAGPPGPPGPPGVSGHPGAPGAPGYQGPPGEPGQAGPAGPPGPPGAMGPAGPAGKDGESGRPGRPGERGFPGPPGIKGPAGMPGFPGMKGHRGFDGRNGEKGDTGAPGLKGENGLPGENGAPGPMGPRGAPGERGRPGLPGAAGARGNDGARGSDGQPGPPGPPGTAGFPGSPGAKGEVGPAGAPGPNGAPGQRGEPGLQGQAGAPGPPGPPGINGSPGGKGEMGPAGIPGAPGLIGARGPPGPPGANGVPGQRGAAGEPGKNGAKGDPGARGERGEAGIPGIAGPKGEDGKDGSPGEPGANGLPGAPGERGPPGFRGAPGANGIPGEKGPPGERGGPGPAGPRGVAGEPGRDGLPGGPGLRGIPGSPGGPGSDGKPGPPGSQGESGRPGPPGSPGPRGQPGVMGFPGPKGNDGAPGKNGERGGPGGPGLPGPAGKNGETGPQGPPGPTGPAGDKGDAGPPGPPGLQGLPGTGGPPGENGKPGEPGPKGDVGAPGIPGGKGDAGAPGERGPPAIAGIGGEKAGGFAPYYG

SEQ ID NO: 1002 - protein, 546 aa, synthetic construct
  REGION 1..546: note = Recombinant collagen fragment
DVKSGVAVGGLAGYPGPAGPPGPPGPPGTSGHPGSPGSPGYQGPPGEPGQAGPAGPPGPIGNVGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 1003 - protein, 1466 aa, synthetic construct
  REGION 1..1466: note = human Col3A1
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 1004 - protein, 1463 aa, synthetic construct
  REGION 1..1463: note = bovine Col1A1
MFSFVDLRLLLLLAATALLTHGQEEGQEEGQEEDIPPVTCVQNGLRYHDRDVWKPVPCQICVCDNGNVLCDDVICDELKDCPNAKVPTDECCPVCPEGQESPTDQETTGVEGPKGDTGPRGPRGPAGPPGRDGIPGQPGLPGPPGPPGPPGPPGLGGNFAPQLSYGYDEKSTGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEGGPQGPRGSEGPQGVRGEPGPPGPAGAAGPAGNPGADGQPGAKGANGAPGIAGAPGFPGARGPSGPQGPSGPPGPKGNSGEPGAPGSKGDTGAKGEPGPTGIQGPPGPAGEEGKRGARGEPGPAGLPGPPGERGGPGSRGFPGADGVAGPKGPAGERGAPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGAPGKDGVRGLTGPIGPPGPAGAPGDKGEAGPSGPAGPTGARGAPGDRGEPGPPGPAGFAGPPGADGQPGAKGEPGDAGAKGDAGPPGPAGPAGPPGPIGNVGAPGPKGARGSAGPPGATGFPGAAGRVGPPGPSGNAGPPGPPGPAGKEGSKGPRGETGPAGRPGEVGPPGPPGPAGEKGAPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPIGPVGARGPAGPQGPRGDKGETGEQGDRGIKGHRGFSGLQGPPGPPGSPGEQGPSGASGPAGPRGPPGSAGSPGKDGLNGLPGPIGPPGPRGRTGDAGPAGPPGPPGPPGPPGPPSGGYDLSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKEKRHVWYGESMTGGFQFEYGGQGSDPADVAIQLTFLRLMSTEASQNITYHCKNSVAYMDQQTGNLKKALLLQGSNEIEIRAEGNSRFTYSVTYDGCTSHTGAWGKTVIEYKTTKTSRLPIIDVAPLDVGAPDQEFGFDVGPACFL

SEQ ID NO: 1005 - protein, 1364 aa, synthetic construct
  REGION 1..1364: note = bovine Col1A2
MLSFVDTRTLLLLAVTSCLATCQSLQEATARKGPSGDRGPRGERGPPGPPGRDGDDGIPGPPGPPGPPGPPGLGGNFAAQFDAKGGGPGPMGLMGPRGPPGASGAPGPQGFQGPPGEPGEPGQTGPAGARGPPGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGELGPVGNPGPAGPAGPRGEVGLPGLSGPVGPPGNPGANGLPGAKGAAGLPGVAGAPGLPGPRGIPGPVGAAGATGARGLVGEPGPAGSKGESGNKGEPGAVGQPGPPGPSGEEGKRGSTGEIGPAGPPGPPGLRGNPGSRGLPGADGRAGVMGPAGSRGATGPAGVRGPNGDSGRPGEPGLMGPRGFPGSPGNIGPAGKEGPVGLPGIDGRPGPIGPAGARGEPGNIGFPGPKGPSGDPGKAGEKGHAGLAGARGAPGPDGNNGAQGPPGLQGVQGGKGEQGPAGPPGFQGLPGPAGTAGEAGKPGERGIPGEFGLPGPAGARGERGPPGESGAAGPTGPIGSRGPSGPPGPDGNKGEPGVVGAPGTAGPSGPSGLPGERGAAGIPGGKGEKGETGLRGDIGSPGRDGARGAPGAIGAPGPAGANGDRGEAGPAGPAGPAGPRGSPGERGEVGPAGPNGFAGPAGAAGQPGAKGERGTKGPKGENGPVGPTGPVGAAGPSGPNGPPGPAGSRGDGGPPGATGFPGAAGRTGPPGPSGISGPPGPPGPAGKEGLRGPRGDQGPVGRSGETGASGPPGFVGEKGPSGEPGTAGPPGTPGPQGLLGAPGFLGLPGSRGERGLPGVAGSVGEPGPLGIAGPPGARGPPGNVGNPGVNGAPGEAGRDGNPGNDGPPGRDGQPGHKGERGYPGNAGPVGAAGAPGPQGPVGPVGKHGNRGEPGPAGAVGPAGAVGPRGPSGPQGIRGDKGEPGDKGPRGLPGLKGHNGLQGLPGLAGHHGDQGAPGAVGPAGPRGPAGPSGPAGKDGRIGQPGAVGPAGIRGSQGSQGPAGPPGPPGPPGPPGPSGGGYEFGFDGDFYRADQPRSPTSLRPKDYEVDATLKSLNNQIETLLTPEGSRKNPARTCRDLRLSHPEWSSGYYWIDPNQGCTMDAIKVYCDFSTGETCIRAQPEDIPVKNWYRNSKAKKHVWVGETINGGTQFEYNVEGVTTKEMATQLAFMRLLANHASQNITYHCKNSIAYMDEETGNLKKAVILQGSNDVELVAEGNSRFTYTVLVDGCSKKTNEWQKTIIEYKTNKPSRLPILDIAPLDIGGADQEIRLNIGPVCFK

SEQ ID NO: 1006 - protein, 1466 aa, synthetic construct
  REGION 1..1466: note = bovine Col3A1
MMSFVQKGTWLLFALLHPTVILAQQEAVDGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGPPGSPGSPGSPGPPGICESCPTGGQNYSPQYEAYDVKSGVAGGGIAGYPGPAGPPGPPGPPGTSGHPGAPGAPGYQGPPGEPGQAGPAGPPGPPGAIGPSGPAGKDGESGRPGRPGERGFPGPPGMKGPAGMPGFPGMKGHRGFDGRNGEKGETGAPGLKGENGVPGENGAPGPMGPRGAPGERGRPGLPGAAGARGNDGARGSDGQPGPPGPPGTAGFPGSPGAKGEVGPAGSPGSSGAPGQRGEPGPQGHAGAPGPPGPPGSNGSPGGKGEMGPAGIPGAPGLIGARGPPGPPGTNGVPGQRGAAGEPGKNGAKGDPGPRGERGEAGSPGIAGPKGEDGKDGSPGEPGANGLPGAAGERGVPGFRGPAGANGLPGEKGPPGDRGGPGPAGPRGVAGEPGRDGLPGGPGLRGIPGSPGGPGSDGKPGPPGSQGETGRPGPPGSPGPRGQPGVMGFPGPKGNDGAPGKNGERGGPGGPGPQGPAGKNGETGPQGPPGPTGPSGDKGDTGPPGPQGLQGLPGTSGPPGENGKPGEPGPKGEAGAPGIPGGKGDSGAPGERGPPGAGGPPGPRGGAGPPGPEGGKGAAGPPGPPGSAGTPGLQGMPGERGGPGGPGPKGDKGEPGSSGVDGAPGKDGPRGPTGPIGPPGPAGQPGDKGESGAPGVPGIAGPRGGPGERGEQGPPGPAGFPGAPGQNGEPGAKGERGAPGEKGEGGPPGAAGPAGGSGPAGPPGPQGVKGERGSPGGPGAAGFPGGRGPPGPPGSNGNPGPPGSSGAPGKDGPPGPPGSNGAPGSPGISGPKGDSGPPGERGAPGPQGPPGAPGPLGIAGLTGARGLAGPPGMPGARGSPGPQGIKGENGKPGPSGQNGERGPPGPQGLPGLAGTAGEPGRDGNPGSDGLPGRDGAPGAKGDRGENGSPGAPGAPGHPGPPGPVGPAGKSGDRGETGPAGPSGAPGPAGSRGPPGPQGPRGDKGETGERGAMGIKGHRGFPGNPGAPGSPGPAGHQGAVGSPGPAGPRGPVGPSGPPGKDGASGHPGPIGPPGPRGNRGERGSEGSPGHPGQPGPPGPPGAPGPCCGAGGVAAIAGVGAEKAGGFAPYYGDEPIDFKINTDEIMTSLKSVNGQIESLISPDGSRKNPARNCRDLKFCHPELQSGEYWVDPNQGCKLDAIKVYCNMETGETCISASPLTIPQKNWWTDSGAEKKHVWFGESMEGGFQFSYGNPELPEDVLDVQLAFLRLLSSRASQNITYHCKNSIAYMDHASGNVKKALKLMGSNEGEFKAEGNSKFTYTVLEDGCTKHTGEWGKTVFQYQTRKAVRLPIVDIAPYDIGGPDQEFGADIGPVCFL

SEQ ID NO: 1007 - protein, 546 aa, synthetic construct
  REGION 1..546: note = Recombinant Collagen fragment
DVKSGVAVGGLAGYPGPAGPPGPPGPPGTSGHKGSPGSPGYQGPPGEPGQAGPAGPPGPIGNVGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPRPPGPPGTAGFPGSPGAKGEVGPAGSPGSNGAPGQRGEPGPQGHKGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 1008 - protein, 547 aa, synthetic construct
  REGION 1..547: note = Collagent recombinant fragment
DSYDVKSGVAVGGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYG

(19) United States
(12) Patent Application Publication
(10) Pub. No.: US 2025/0381317 A1
(43) Pub. Date: Dec. 18, 2025

(54) COLLAGEN COMPOSITIONS AND METHODS OF USE THEREOF

(71) Applicant: MODERN MEADOW, INC., Nutley, NJ (US)

(72) Inventors: Lixin DAI, Nutley, NJ (US); Monique DE LEEUW, Nutley, NJ (US); Zhiyuan QIAN, Nutley, NJ (US); Dale L. HANDLIN, JR., Nutley, NJ (US); David WILLIAMSON, Nutley, NJ (US); Shawn HUXEL, Nutley, NJ (US); Isabelle HANSENNE-CERVANTES, Nutley, NJ (US); Paul BONVALLET, Nutley, NJ (US)

(21) Appl. No.: 18/868,290

(22) PCT Filed: May 31, 2023
(86) PCT No.: PCT/US2023/067726

§ 371 Date: Nov. 22, 2024

Related U.S. Application Data
(60) Provisional application No. 63/347,750, filed on Jun. 1, 2022.

(51) Int. Cl. A61L 27/26; A61L 27/54

(52) U.S. Cl. CPC A61L 27/26; A61L 27/54; A61L 2300/236; A61L 2300/252; A61L 2300/412; A61L 2430/34

(57) ABSTRACT

The present disclosure relates to compositions comprising collagen and hyaluronic acid, wherein the collagen is fully miscible with the hyaluronic acid. The present disclosure also relates to a composition comprising collagen, hyaluronic acid, and additional polymers, wherein the collagen is fully miscible with the hyaluronic acid and polymers. Methods of using the compositions described herein for medical and cosmetic treatments are also provided.

The content of the electronically submitted sequence listing XML (Name: 4431_090PC01_SequenceListing_ST26.xml; Size: 1,309,312 bytes; and Date of Creation: May 30, 2023) filed with the application is incorporated herein by reference in its entirety.

### BACKGROUND

Collagen is one of the most important proteins in the human body, and is present in connective tissue such as cartilage, bones, tendons, ligaments, and skin, and it is the major protein in the extra-cellular matrix of human cells. “Recombinant collagen” refers to the family of at least 28 distinct naturally occurring collagen Types prepared using recombinant techniques.

There are many known uses for collagen. In the cosmetics and skincare industry, for example, skincare compositions that include collagen can be used to combat the effects of aging and environmental stress on the appearance, elasticity, and thickness of skin. For example, ageing and environmental factors can lead to dermatological conditions including, but not limited to fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, and rhinophyma.

Separately, dermal hyaluronic acid (HA) fillers are popular for soft tissue augmentation. Additional age-related conditions that can benefit from collagen and hyaluronic acid treatment include, but are not limited to, arthritis, osteoarthritis, ocular issues, cell adhesions, and similar conditions. Although there are numerous skincare products and dermal fillers on the market to improve skin appearance, many consumers are hesitant to use chemically synthesized products they perceive as being environmentally unfriendly or otherwise unsafe. A miscible product comprising HA and collagen that would retain the benefits of both components is highly desirable, but natural collagen is not compatible with HA—each tends to separate during storage and use. Like HA, there is a need in the field for miscible collagen blends of polymers used in the delivery of therapeutics.

### BRIEF SUMMARY

In some embodiments, the present disclosure provides a composition comprising collagen and hyaluronic acid, wherein the collagen is fully miscible with the hyaluronic acid. In some embodiments, the collagen is a recombinantly produced collagen. In some embodiments, the collagen is a recombinantly produced collagen fragment.

In some embodiments, the composition has an isoelectric point (pI) of from about 4.0 to about 12.0. In some embodiments, the composition has a pI of from about 4.0 to about 5.0. In some embodiments, the composition has a pI of from about 6.0 to about 7. In some embodiments, the composition has a pI of from about 9.0 to about 10.0. In some embodiments, the composition has a pI of from about 9.0 to about 9.7.

In some embodiments, the molecular weight of the collagen is from about 5 kDa to about 150 kDa.

In some embodiments, the hyaluronic acid has a molecular weight of from about 3 kDa to about 10,000 kDa. In some embodiments, the hyaluronic acid has a molecular weight of from about 20 kDa to about 2,000 kDa. In some embodiments, the hyaluronic acid has a molecular weight of from about 1,000 kDa to at least about 5,000 kDa. In some embodiments, the hyaluronic acid has a molecular weight of from about 2,000 kDa to at least about 3,000 kDa. In some embodiments, the hyaluronic acid has a molecular weight of about 1,000 kDa. In some embodiments, the hyaluronic acid has a molecular weight of about 50 kDa.

In some embodiments, the collagen comprises no hydroxyproline (Hyp) residues. In some embodiments, the collagen comprises from about 1% to about 20% Hyp residues, based on the total number of prolines in the collagen. In some embodiments, the collagen comprises no hydroxyl groups. In some embodiments, the collagen comprises from about 1 to about 120 hydroxyl groups. In some embodiments, the collagen comprises no amine groups. In some embodiments, the collagen comprises from about 1 to about 100 amine groups. In some embodiments, the collagen comprises no carboxylic acid groups. In some embodiments, the collagen comprises from about 1 to about 80 carboxylic acid groups.

In some embodiments, the hyaluronic acid is crosslinked. In some embodiments, the hyaluronic acid is not crosslinked. In some embodiments, the hyaluronic acid is a biphasic crosslinked hyaluronic acid. In some embodiments, the hyaluronic acid is a monophasic crosslinked hyaluronic acid.

In some embodiments, the hyaluronic acid solution has a pH in a range of from about 5.0 to about 12. In some embodiments, the hyaluronic acid has a pH in a range of from about 9.0 to about 12. In some embodiments, the hyaluronic acid has a pH in a range of from about 5.2 to about 7.2.

In some embodiments, the (w/w) ratio of hyaluronic acid to collagen is in a range of from about 1:9 to 9:1.

In some embodiments, the collagen is crosslinked. In some embodiments, the collagen is not crosslinked.

In some embodiments, the hyaluronic acid is crosslinked by a chemical crosslinking agent, ultraviolet (UV) radiation, microwave radiation, or heat. In some embodiments, the collagen is crosslinked by a chemical crosslinking agent, ultraviolet (UV) radiation, microwave radiation, or heat.

In some embodiments, the composition has a viscosity of from about 1 cps to about 100,000 cps. In some embodiments, the composition has a viscosity of from about 10,000 cps to about 80,000 cps. In some embodiments, the composition has a viscosity of from about 50,000 cps to about 70,000 cps.

In some embodiments, the composition is a solution. In some embodiments, the composition is a gel. In some embodiments, the composition is a foam.

In some embodiments, the present disclosure provides a film comprising the composition disclosed herein.

In some embodiments, the present disclosure provides a fiber or plurality of fibers comprising the composition disclosed herein.

In some embodiments, the present disclosure provides a coating prepared from the composition disclosed herein.

In some embodiments, the present disclosure provides a powder prepared from the composition disclosed herein.

In some embodiments, the present disclosure provides a slurry prepared from the composition disclosed herein.

In some embodiments, the present disclosure provides a particle or plurality of particles prepared from the composition disclosed herein.

In some embodiments, the collagen is unhydroxylated. In some embodiments, the recombinant collagen fragment is unhydroxylated.

In some embodiments, the collagen is hydroxylated. In some embodiments, the recombinant collagen fragment is hydroxylated. In some embodiments, the recombinant collagen fragment is from about 1% to about 20% hydroxylated. In some embodiments, the recombinant collagen fragment is about 12% hydroxylated. In some embodiments, the recombinant collagen fragment is 5% hydroxylated.

In some embodiments, the composition further comprises a pharmaceutically active agent. In some embodiments, the composition further comprises a cosmetically active agent.

In some embodiments, the collagen or the recombinant collagen fragment has a sequence identity of at least about 85% to the amino acid sequence set forth in SEQ ID NO: 1. In some embodiments, the collagen or the recombinant collagen fragment has a sequence identity of at least about 85% to the amino acid sequence set forth in in any one of SEQ ID NOs: 2-1002.

In some embodiments, the collagen or the recombinant collagen is fermented or chemically synthesized.

In some embodiments, the present disclosure provides a method of increasing collagen production in cells, comprising administering or applying an effective amount of composition disclosed herein.

In some embodiments, the present disclosure provides a method of medical or cosmetic treatment comprising administering or applying an effective amount of the composition disclosed herein.

In some embodiments, the treatment comprises filling wrinkles, increasing viscoelasticity, or increasing volume in the face or body. In some embodiments, the treatment comprises forming a cosmetic implant in a patient. In some embodiments, the treatment comprises tissue augmentation.

In some embodiments, the composition is administered to the face, eyes, nose, nasolabial folds, neck, décolletage, arms, abdomen, blood vessels, bladder, urethra, nerves, cartilage, bones, breasts, vocal cords, vocal folds, tympanic membranes, joints, spinal discs, buttocks, hips, calfs, thighs, flanks, or hands of the subject.

In some embodiments, the treatment comprises treating an inflammatory skin disease or mucous membrane disease. In some embodiments, the treatment comprises treating lipoatrophy.

In some embodiments, the present disclosure provides a method of treating a wound in a human subject in need thereof, the method comprising applying the composition of any one of claims 1-59 to the wound on the subject. In some embodiments, the wound is a chronic wound, a surgical wound, a superficial wound, an abrasion, or wherein the wound is the result of thermal damage.

In some embodiments, the present disclosure provides a skincare product comprising the composition disclosed herein, for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, sun care, color cosmetics, hair care, fragrances, oral care, or any combination thereof.

In some embodiments, the present disclosure provides a dermal filler comprising the composition disclosed herein.

In some embodiments, the dermal filler is a heat stable, sterile, soft tissue filler. In some embodiments, the dermal filler is heat stable up to at least about 125° C. In some embodiments, the dermal filler is heat stable at temperatures ranging up to at least about 135° C. In some embodiments, the dermal filler is terminally sterilized. In some embodiments, the dermal filler is injectable.

In some embodiments, the dermal filler further comprises carboxymethyl cellulose. In some embodiments, the dermal filler further comprises calcium hydroxyapatite. In some embodiments, the dermal filler further comprises polylactic acid, aliphatic polyesters, polyorthoesters, polyoxaesters, polyanhydrides, polycarbonates, polyurethanes, polyamides, polyester amides, hydrolyzable amines, polyalkylene oxides, poloxamers, functionalized isoflavonoids, amino acids, solid homopolymers of poly(e-caprolactone), solid homopolymers of poly(p-dioxanone), solid homopolymers of poly(trimethylene carbonate), solid copolymers of a plurality of e-caprolactone repeating units and third lactone repeating units, solid copolymers of a plurality of trimethylene carbonate repeating units and second lactone repeating unit; wherein the third lactone repeating units are selected from the group consisting of glycolide repeating units, lactide repeating units, trimethylene carbonate repeating units, p-dioxanone repeating units, 1,4-dioxepan-2-one repeating units, 1-5-dioxepan-2-one repeating units, or any combination thereof.

In some embodiments, the dermal filler further comprises a pharmaceutically active agent. In some embodiments, the pharmaceutically active agent comprises an anesthetic agent.

In some embodiments, the present disclosure provides a use of the compositions, the skincare product, or the dermal fillers disclosed herein, for medical or cosmetic treatment of a subject in need thereof.

In some embodiments, the present disclosure provides a method of preparing a composition comprising collagen and hyaluronic acid, wherein the collagen is fully miscible with the hyaluronic acid, the method comprising: dissolving hyaluronic acid in an NaOH solution; adding collagen; and adjusting the pH by adding HCl.

In some embodiments, the present disclosure provides a method of preparing a composition comprising collagen and hyaluronic acid, wherein the collagen is fully miscible with the hyaluronic acid, the method comprising: dissolving hyaluronic acid in an NaOH solution; adjusting the pH by adding HCl; and adding collagen.

In some embodiments, the present disclosure provides a method of preparing a composition comprising collagen and hyaluronic acid, wherein the collagen is fully miscible with the hyaluronic acid, the method comprising: dissolving hyaluronic acid in water to form a hyaluronic acid solution; dissolving collagen in water to form a collagen solution; and mixing the hyaluronic acid and collagen solutions.

In some embodiments, the method further comprises: optionally crosslinking the hyaluronic acid and the collagen in the solution by adding a coupling agent.

In some embodiments, the method further comprises optionally crosslinking the hyaluronic acid and the collagen in the solution by adding a coupling agent before adjusting the pH with HCl and adding the collagen.

In some embodiments, the method, further comprises: adding sodium chloride salt to cause a gel to form; and allowing the gel to set. In some embodiments, the method further comprises sterilizing the gel.

In some embodiments, the solution comprises NaCl.

In some embodiments, the method further comprises adding calcium hydroxyapatite, carboxymethyl cellulose, polylactic acid, polylactic acid, aliphatic polyesters, polyorthoesters, polyoxaesters, polyanhydrides, polycarbonates, polyurethanes, polyamides, polyester amides, hydrolyzable amines, polyalkylene oxides, poloxamers, functionalized isoflavonoids, amino acids, solid homopolymers of poly(e-caprolactone), solid homopolymers of poly(p-dioxanone), solid homopolymers of poly(trimethylene carbonate), solid copolymers of a plurality of e-caprolactone repeating units and third lactone repeating units, solid copolymers of a plurality of trimethylene carbonate repeating units and second lactone repeating unit; wherein the third lactone repeating units are selected from the group consisting of glycolide repeating units, lactide repeating units, trimethylene carbonate repeating units, p-dioxanone repeating units, 1,4-dioxepan-2-one repeating units, 1-5-dioxepan-2-one repeating units, and or combinations thereof. or any combination thereof.

In some embodiments, the method further comprises adding an anesthetic agent. In some embodiments, the method further comprises dispensing the gel into a syringe under aseptic conditions.

In some embodiments, the present disclosure provides a composition comprising collagen and polyvinylpyrrolidone, wherein the collagen is fully miscible with the polyvinylpyrrolidone. In some embodiments, the polyvinylpyrrolidone has a molecular weight of from about 10 kDa to about 400 kDa. In some embodiments, the polyvinylpyrrolidone has a molecular weight of about 50 kDa.

In some embodiments, the collagen is fully miscible with the polyacrylamide. In some embodiments, the polyacrylamide has a molecular weight of from about 5,000 kDa to about 6,000 kDa. In some embodiments, the polyacrylamide has a molecular weight of from about 30 kDa to about 50 kDa.

In some embodiments, the present disclosure provides a composition comprising collagen and poly(ethylene oxide), wherein the collagen is fully miscible with the poly(ethylene oxide). In some embodiments, the collagen is fully miscible with the poly(2-oxazoline)s. In some embodiments, the collagen is fully miscible with the polyethylenimine.

In some embodiments, the present disclosure provides a composition comprising collagen and sodium carboxymethylcellulose, wherein the collagen is fully miscible with the carboxymethylcellulose. In some embodiments, the carboxymethylcellulose has a molecular weight of about 250 kDa and a degree of substitution of from about 1.00 to about 1.25. In some embodiments, the carboxymethylcellulose has a molecular weight of about 250 kDa and a degree of substitution of of from about 0.70 to about 0.90. In some embodiments, the carboxymethylcellulose has a molecular weight of about 250 kDa and a degree of substitution of about 0.79. In some embodiments, the carboxymethylcellulose has a degree of substitution of about 0.87.

In some embodiments, the carboxymethylcellulose has a high viscosity.

In some embodiments, the present disclosure provides a composition comprising collagen and chondroitin sulfate, wherein the collagen is fully miscible with the chondroitin sulfate. In some embodiments, the chondroitin sulfate has a molecular weight of about 50 kDa.

In some embodiments, the present disclosure provides a composition comprising collagen and an acetylated hyaluronate. In some embodiments, the collagen is fully miscible with the acetylated hyaluronate.

In some embodiments, the present disclosure provides a composition comprising collagen and zinc hyaluronate. In some embodiments, the collagen has a sequence identity of at least about 85%, at least about 87%, at least about 90%, at least about 91%, at least about 92%, at least about 93%, at least about 94%, at least about 95%, at least about 96%, at least about 97%, at least about 98%, at least about 99%, or 100% identical to the amino acid sequence set forth in SEQ ID NO: 1. In some embodiments, the collagen has a sequence identity of at least about 98% to the amino acid sequence set forth in SEQ ID NO: 1.

In some embodiments, the present disclosure provides a composition comprising a recombinant collagen fragment having an amino acid sequence identity of at least about 85% to SEQ ID NO: 1, and/or one or more collagen fragment sequence variants having an amino acid sequence identity of at least about 85% to the amino acid sequence set forth in in any one of SEQ ID NOs: 975-1002, and hyaluronic acid, wherein the recombinant collagen fragments are fully miscible with the hyaluronic acid. In some embodiments, the hyaluronic acid is a biphasic crosslinked hyaluronic acid comprising crosslinked HA and uncrosslinked HA.

In some embodiments, the present disclosure provides a composition comprising a recombinant collagen fragment having an amino acid sequence identity of at least about 85% to SEQ ID NO: 1, and/or one or more collagen fragment sequence variants having an amino acid sequence identity of at least about 85% to the amino acid sequence set forth in in any one of SEQ ID NOs: 975-1002, and polyvinylpyrrolidone, and polyvinylpyrrolidone, wherein the recombinant collagen fragments are fully miscible with the polyvinylpyrrolidone.

In some embodiments, the present disclosure provides a composition comprising one or more recombinant collagen fragments having an amino acid sequence identity of at least about 85% to SEQ ID NO: 1, and/or one or more collagen fragment sequence variants having an amino acid sequence identity of at least about 85% to the amino acid sequence set forth in in any one of SEQ ID NOs: 975-1002, and sodium carboxymethylcellulose, wherein the recombinant collagen fragments are fully miscible with the sodium carboxymethylcellulose.

In some embodiments, the present disclosure provides a film comprising a recombinantly produced collagen fragment and hyaluronic acid. In some embodiments, the film is visually transparent. In some embodiments, the film is hazy. In some embodiments, the film shows phase separation.

In some embodiments, the present disclosure provides a gel comprising a recombinantly produced collagen fragment and hyaluronic acid.

In some embodiments, the present disclosure provides a fiber or plurality of fibers comprising a recombinantly produced collagen fragment and hyaluronic acid.

In some embodiments, the present disclosure provides a coating comprising a recombinantly produced collagen fragment and hyaluronic acid.

In some embodiments, the present disclosure provides a slurry comprising a recombinantly produced collagen fragment and hyaluronic acid.

In some embodiments, the present disclosure provides a particle or particles comprising a recombinantly produced collagen fragment and hyaluronic acid.

In some embodiments, the present disclosure provides a foam comprising a recombinantly produced collagen fragment and hyaluronic acid.

In some embodiments, the present disclosure provides a composition comprising collagen and alginic acid sodium salt, wherein the collagen is fully miscible with the alginic acid sodium salt. In some embodiments, the alginic acid sodium salt has a viscosity of about 4-12 cps for 1% aqueous solution at 25° C.

In some embodiments, the present disclosure provides a composition comprising collagen and alginic acid sodium salt, wherein the collagen is fully miscible with the alginic acid sodium salt. In some embodiments, the alginic acid sodium salt has a viscosity of equal to or greater than 2,000 cps for 2% aqueous solution at 25° C.

### BRIEF DESCRIPTION OF THE FIGURES

FIGS. 1A-1C are a series of photographs showing combinations of uncrosslinked HA with collagens. FIG. 1A shows that native animal-derived collagen is not miscible with 50 kDa uncrosslinked HA. FIG. 1B shows that a recombinant collagen fragment is miscible with 50 kDa uncrosslinked HA. FIG. 1C shows that a recombinant collagen fragment is miscible with 1,000 kDa uncrosslinked HA.

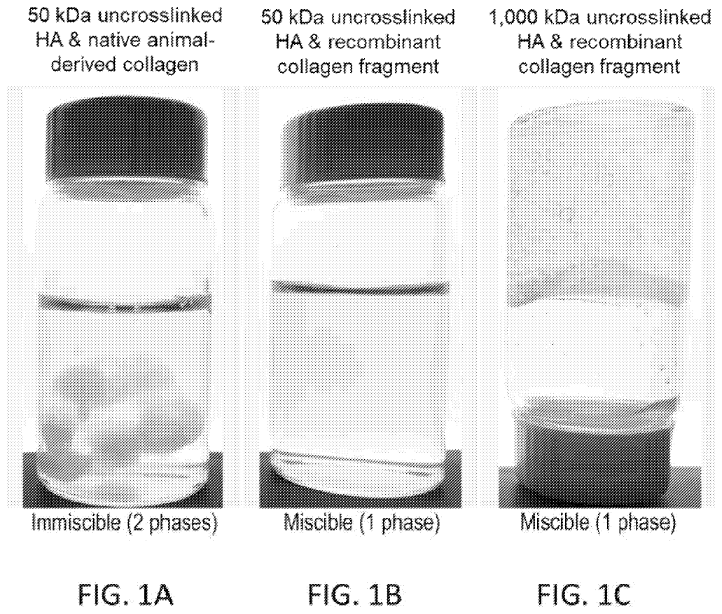

FIGS. 2A-2C are a series of photographs showing that fluorescently labelled recombinant collagen fragment forms a homogeneous miscible blend with both mono-phasic and bi-phasic crosslinked HA. FIG. 2A shows that 1,000 kDa uncrosslinked HA is miscible with a labeled recombinant collagen. FIG. 2B shows that a mono-phasic uncrosslinked HA is miscible with a recombinant collagen. FIG. 2C shows that a bi-phasic uncrosslinked HA is miscible with a recombinant collagen.

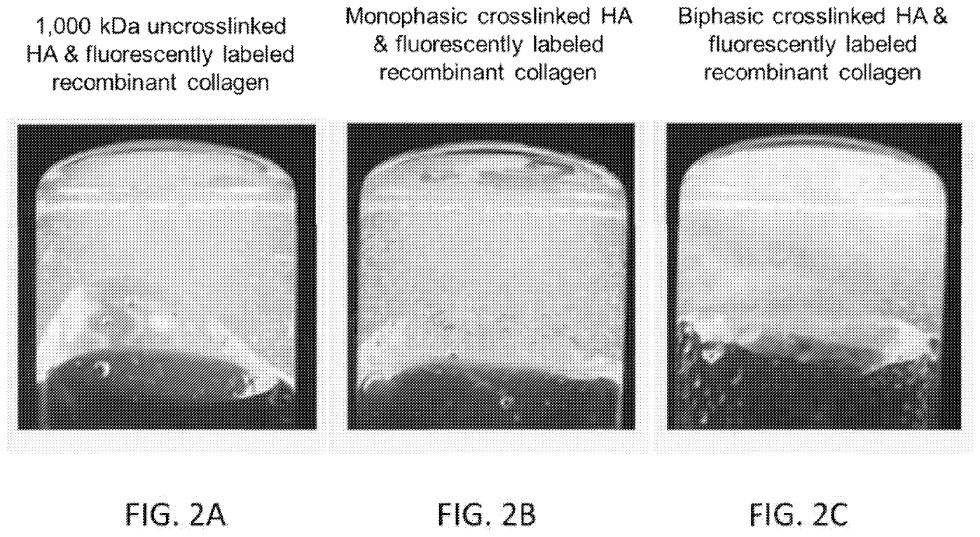

FIGS. 3A-3C are a series of photographs showing that certain percentages of hydroxylation on a recombinant collagen fragment reduces its miscibility with uncrosslinked HA. FIG. 3A shows that 50 kDa uncrosslinked HA is miscible with a 0% hydroxylated recombinant collagen fragment. FIG. 3B shows small scale phase separation in a 50 kDa uncrosslinked HA and 22% hydroxylated recombinant collagen fragment mixture that does not separate into two distinct phases. FIG. 3C shows that 50 kDa uncrosslinked HA is not miscible with a 44% hydroxylated recombinant collagen fragment.

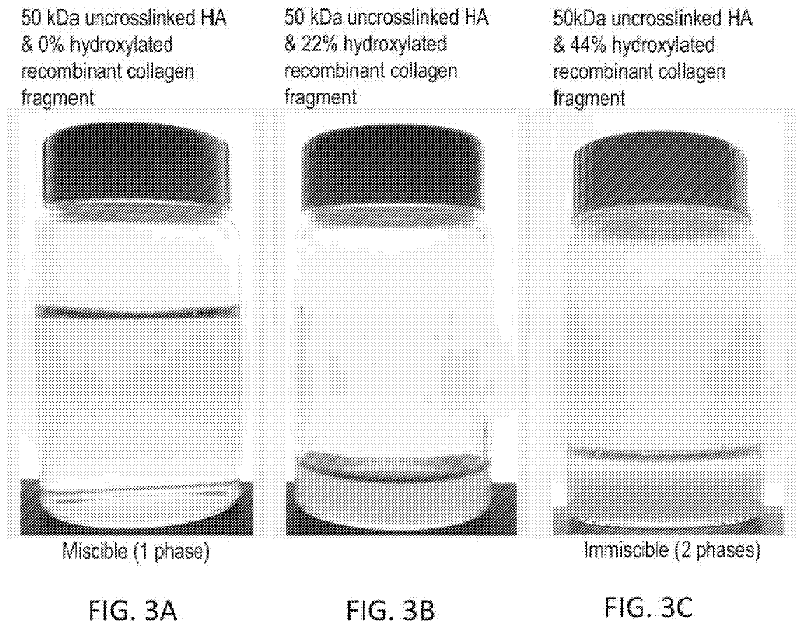

FIG. 4A is a standard curve used to convert UV absorbance to concentration of protein by showing the relationship between absorbance (Y-axis) as a function of protein concentration (X-axis).

FIG. 4B shows concentration of protein released from a PBS solution of protein compared to a formulation of HA and protein in PBS (Y-axis) over time in days (X-axis). The measurements in FIGS. 4A-B were taken outside the 300 kDa molecular weight cutoff (MWCO) dialysis membrane.

FIG. 5A is a standard curve used to convert absorbance to concentration of protein by showing the relationship between absorbance (Y-axis) as a function of protein concentration (X-axis).

FIG. 5B shows concentration of protein released (Y-axis) from the PBS compared to the protein released from the HA Formulation over time in days (X-axis). The measurements for FIGS. 5A-5B were taken from outside the 300 kDa MWCO dialysis membrane as in FIGS. 4A and 4B.

FIG. 6A is a standard curve used to convert absorbance to concentration of protein by showing the relationship between absorbance (Y-axis) as a function of protein concentration (X-axis).

FIG. 6B shows protein concentrations plotted against timepoints to give a trend of the amount of protein remaining inside the PBS Solution and HA Formulation after dialysis. In this experiment, data was taken from inside the starting solutions as opposed to FIGS. 4-5 which were taken from outside the 300 kDa MWCO dialysis membrane.

FIG. 7A is a standard curve used to convert absorbance to concentration of protein by showing the relationship between absorbance (Y-axis) as a function of protein concentration (X-axis).

FIG. 7B shows concentration of protein released (Y-axis) from the PBS solution compared to the protein released from the HA Formulation over time in days (X-axis).

### DETAILED DESCRIPTION

### Definitions

The indefinite articles “a” and “an” to describe an element or component means that one or at least one of these elements or components is present. Although these articles are conventionally employed to signify that the modified noun is a singular noun, as used herein the articles “a” and “an” also include the plural, unless otherwise stated in specific instances. Similarly, the definite article “the,” as used herein, also signifies that the modified noun can be singular or plural, again unless otherwise stated in specific instances.

As used herein, the term “about” used with numerical values means “within 10% of the stated value,” unless expressly noted otherwise. For example, “about 5% by weight” means from 4.5% by weight to 5.5% by weight.

The term “modified,” as applied to the collagens and collagen fragments refers to collagen fragments comprising an amino acid sequence that is at least 40%, 45%, 50%, 55%, 60%, 65%, 70%, 80%, 90%, 91%, 92%, 93%, 94% 95%, 96%, 97%, 98%, or 99% identical or similar to the amino acid sequence of a biologically active molecule. In some embodiments, the modified collagen fragment comprises an amino acid sequence that is at least 40%, 45%, 50%, 55%, 60%, 65%, 75%, 80%, 85%, 86%, 87%, 88%, 89%, 90%, 91%, 92%, 93%, 94%, 95%, 96%, 97%, 98%, or 99% identical to the amino acid sequence of a native or previously engineered sequence. The modified sequence can comprise additions, deletions, substitutions, or a combination thereof to the amino acid sequence of a native or previously engineered molecule. For example, a modified collagen fragment can incorporate or delete 1, 2, 3, 4, 5, 6, 7, 8, 9, 10 or more amino acid residues compared to a native collagen sequence. Such selections can be made to modify the looseness or tightness of a recombinant collagen. The degree of hydroxylation of collagen correlates with the looseness or tightness of the collagen triple helix. A modified collagen fragment can also include chemical modifications to a polypeptide, such as crosslinks between cysteine residues, or hydroxylated or glycosylated residues. As used herein, the terms “variant” and “sequence variant” refer to a polypeptide sequence that is about 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% identical to the amino acid sequence set forth in SEQ ID NO: 1.

The terms “pharmaceutically acceptable” and “cosmetically acceptable,” as applied to carriers, excipients, or stabilizers that can be used in the compositions described herein, refer to carriers, excipients, or stabilizers that are nontoxic to recipients at the dosages and concentrations employed.

As used herein, the term “tissue repair” refers to the restoration of tissue architecture and function after an injury in the context of the healing of damaged tissue. Tissue regeneration refers to a type of healing in which new growth restores portions of damaged tissue to a normal state.

As used herein, “hyaluronic acid” and “HA” refer to acidic polysaccharides with different molecular weights made up of residues of D-glucuronic and N-acetyl-D-glucosamine acids, and their corresponding salts, including but not limited to sodium hyaluronate. Compositions described herein as comprising HA should be understood to comprise a combination of hyaluronic acid and sodium hyaluronate unless otherwise specified. Hyaluronic acid occurs naturally in cell surfaces, in the basic extracellular substances of the connective tissue of vertebrates, in the synovial fluid of the joints, in the endobulbar fluid of the eye, and in human umbilical cord tissue. “Hyaluronic acid” can mean a whole series of polysaccharides with alternating residues of D-glucuronic and N-acetyl-D-glucosamine acids with varying molecular weights or degraded fractions of the same. Hyaluronic acid also refers to an unsulphated glycosaminoglycan composed of repeating disaccharide units of N-acetylglucosamine (GlcNAc) and glucuronic acid (GlcUA) linked together by alternating beta-1,4 and beta-1,3 glycosidic bonds. Hyaluronic acid is also known as hyaluronan, hyaluronate, or HA.

As used herein, the terms “sodium carboxymethylcellulose,” “carboxymethylcellulose” and “CMC” can be used interchangeably, and refer to a polymer with different ranges of molecular weights as a derivative of cellulose with carboxymethyl groups bound to some of the monomers that make up the cellulose backbone. Compositions described herein as comprising carboxymethylcellulose should be understood to comprise a combination of carboxymethylcellulose and sodium carboxymethylcellulose unless otherwise specified.

As used herein, the term “polyvinylpyrrolidone” refers to a polymer with different ranges of molecular weights made from the monomer N-vinylpyrrolidone.

Polyvinylpyrrolidone (PVP) is also known as polyvidone and povidone.

As used herein, the term “polyacrylamide” refers to a polymer with different ranges of molecular weights made from the monomer acrylamide. Polyacrylamide (PAM) is also known as poly(2-propenamide).

As used herein, the term “poly(ethylene oxide)” refers to a polymer with different ranges of molecular weights made from the monomer ethylene oxide. Poly(ethylene oxide) (PEO) is also known as poly(ethylene glycol) (PEG) or poly(oxyethylene).

As used herein, the term “poly(2-oxazoline)” refers to a polymer with different ranges of molecular weights made from the monomer 2-oxazoline and its derivatives such as 2-methyl-2-oxazoline and 2-ethyl-2-oxazoline.

As used herein, the term “polyethylenimine” refers to a polymer with different ranges of molecular weights made from the monomer aziridine. Polyethylenimine (PEI) is also known as polyaziridine.

As used herein, the term “miscible” means that one substance is soluble with the other such that a molecularly homogeneous mixture is formed when the two components are mixed in water or in a buffer system. An exemplary buffer system is phosphate buffered saline (PBS). For example, collagen described as miscible with HA means that when a solution of collagen dissolved in water, or a buffer system is mixed with a solution of HA dissolved in water (for example a solution of from 1% to 10% HA), the aqueous mixture of the collagen solution and the HA solution will form a homogenous mixture at room temperature. The miscibility of collagen with hyaluronic acid in a composition can depend on the ratio of hyaluronic acid to collagen, the molecular weights of the collagen and the hyaluronic acid used in the composition, and the presence of other additional components in the composition, such as salts, that affect the compatibility of the collagen and hyaluronic acid. Temperature can also affect the segregation of the hyaluronic acid and the collagen in the composition. Collagen described as immiscible with hyaluronic acid means that the mixture of the collagen solution and the HA solution will not form a stable, transparent, homogenous mixture at room temperature. Instead, the solution can show turbidity or phase separation. Solid films prepared from fully miscible solutions of HA and collagen can remain fully miscible as a solid film as demonstrated by being transparent and homogenous or may show small scale phase separation indicated by haziness. Transparency can be readily determined by a person of ordinary skill in the art and can be measured, for example, using a haze meter.

A collagen that is “fully miscible” can apply to the ability of a collagen to form a homogeneous aqueous mixture with any substance or polymer (for example, polyvinylpyrrolidone (PVP), polyacrylamide (PAM), poly(ethylene oxide) (PEO), poly(2-oxazoline)s, polyethyleninime (PEI), carboxymethylcellulose, chondroitin sulfate, acetylated hyaluronate and/or zinc hyaluronate), and is not limited to HA.

“Viscosity” refers to a measurement of the resistance to flow of a liquid at a given temperature. Viscosity can be determined using a variety of methods and instruments known in the art, including but not limited to measurement by rheometer and viscometer. Viscosity can also represent the stiffness of a liquid at a given temperature.

The composition disclosed herein can have a viscosity at 25° C. of at least about 1 centipoise (cps), at least about 10 cps, at least about 100 cps, at least about 1000 cps, at least about 10,000 cps, at least about 20,000 cps, at least about 50,000 cps at least about 100,000 cps or more, at a shear rate of 0.1/second. In some embodiments, a viscous carrier in the composition of the present disclosure can have a viscosity at 25° C. of about 1 cps, about 10 cps, about 100 cps, about 1000 cps, about 10,000 cps, about 20,000 cps, about 50,000 cps, about 100,000 cps or more, at a shear rate of 0.1/second.

As used herein, the term “isoelectric point” or “pI” refers to the pH value where the overall net charge of a macromolecule such as a protein is zero. In proteins there can be many charged groups, and at the isoelectric point, the sum of all these charges is zero, i.e., the number of negative charges balances the number of positive charges. At a pH above the isoelectric point the overall net charge of the protein will be negative, whereas at pH values below the isoelectric point, the overall net charge of the protein will be positive. The isoelectric point of molecules can be determined by using isoelectric focusing, a well-known technique for separating different molecules by differences in their isoelectric point. The isoelectric point of proteins can also be determined by electrophoresis. Isoelectric point can also be calculated (see e.g., Levene and Simms, “Calculation of isoelectric point” J. Biol. Chem., 1923, pp. 801-813).

The composition described herein can have an isoelectric point in a range from about 4.0 to about 12.0, from about 4.5 to about 11.5, from about 5.0 to about 11.0, from about 5.5 to about 10.5, from about 6.0 to about 10.0, from about 6.5 to about 9.5, from about 7.0 to 9.0, from about 7.5 to 8.5 or about 8.0. In certain embodiments, the composition described herein has an isoelectric point of at least about 4.0, about 4.5, about 5.0, about 5.5, about 6.0, at least about 6.5, at least about 7.0, at least about 7.5, at least about 8.0, at least about 8.5, at least about 9, at least about 9.5, at least about 10, at least about 10.5, at least about 11, at least about 11.5, or at least about 12. In some embodiments, the composition described herein has an isoelectric point of about 4.0, about 4.5, about 5.0, about 5.5, about 6.0, about 6.5, about 7.0, about 7.5, about 8.0, about 8.5, about 9, about 9.5, about 10, about 10.5, about 11, about 11.5, or about 12.

The term “crosslinked” as used herein describes a polymer with at least one covalent bond that is not found in the repeating units of the polymer or found between repeating units of the polymer. The crosslinking bonds occur between individual strands or molecules of the polymer; however, intramolecular reactions can occur within a polymer chain to form macrocyclic structures. The latter are not considered crosslinks in this disclosure. The crosslinks are formed between any two functional groups of the polymer (e.g., at the ends, on the side chains, etc.). In certain embodiments, the crosslinks are formed between terminal units of the polymers. In certain embodiments, the crosslinks are formed between sidechain units of the polymers. Any type of covalent bond can form the crosslink (e.g., carbon-carbon, carbon-oxygen, carbon-nitrogen, oxygen-nitrogen, sulfur-sulfur, oxygen-phosphorus, etc.). The resulting crosslinked material can be branched, linear, dendritic, etc. In certain embodiments, the crosslinks form a 3-D network of crosslinks. The crosslinks can be formed by any chemical crosslinking agent in a chemical reaction that results in the covalent bonds. The crosslinks can be formed applying ultraviolet (UV) radiation or microwave radiation to the polymer. The crosslinks can be formed by free radical initiated reactions, for example, with a photoinitiator or thermal initiator, or by applying heat.

Typical chemical crosslinking agents are generally bi- or polyfunctional crosslinking agents of various types and can, for example, be selected from DVS (divinyl sulfone) in an alkaline medium (see U.S. Pat. No. 4,582,865), bi- or polyfunctional epoxy compounds (see U.S. Pat. No. 4,716,154), carbodiimides, or formaldehyde (see GB 2 151 244). Other suitable crosslinking agent include epoxides or derivatives thereof, in particular 1,4-butanediol diglycidyl ether (BDDE), diepoxyoctane or 1,2-bis(2,3-epoxypropyl)-2,3-ethylene. Known crosslinking agents also include divinylsulfone, glycerol diglycidyl ether, and 1, 4-butanediyl diglycidyl ether.

In certain embodiments, the composition according to the present disclosure can react to form a crosslinked macromolecular matrix. Because the crosslinking reaction occurs in an aqueous solution, a crosslinked macromolecular matrix can be dispersed in an aqueous liquid in hydrogel form as it is formed by a crosslinking reaction. The term “hydrogel” as used herein refers to a three-dimensional network comprising hydrophilic polymers that contains a large amount of water. A hydrogel can, for example contain 30%, 40%, 50%, 60%, 70%, 80%, 90%, 95%, or an even greater amount of water on a w/w basis. A “hydrogel precursor” is a polymer that is at least partly soluble in an aqueous medium and is capable of becoming crosslinked to form a hydrogel.

In some embodiments, the composition of the present disclosure can exhibit a physiologically acceptable osmolarity. As used herein, the term “osmolarity” refers to the concentration of osmotically active solutes in solution. As used herein, the term “a physiologically acceptable osmolarity” refers to an osmolarity in accord with, or characteristic of, the normal functioning of a living organism. As such, administration of a hydrogel disclosed herein exhibits an osmolarity that has substantially no long term or permanent detrimental effect when administered to a mammal. Osmolarity is expressed in terms of osmoles of osmotically active solute per liter of solvent (Osmol/L or Osm/L).

As used herein, “solubility” refers to the amount of a substance that dissolves in a given volume of solvent at a specified temperature and pH, e.g., to form a saturated solution. Solubility can be determined, for example, using the shake-flask solubility method (ASTM: E 1148-02, Standard Test Method for Measurements of Aqueous Solubility, Book of Standards Volume 11.05). Solubility can be determined at any pH. Solubility can be tested at any temperature between 20° C. and 40° C. For example, solubility can be determined at approximately pH 7.0-7.4 and approximately 37° C.

### A. Collagen

The term “collagen” refers to any one of the known collagen Types, including collagen Types I through XXVIII, as well as to any other collagens, whether natural, synthetic, semi-synthetic, or recombinant. The term collagen includes collagen, collagen fragments, collagen-like proteins, triple helical collagen, alpha chains, monomers, gelatin, trimers, and combinations thereof. It includes all of the collagens, modified collagens and collagen-like proteins described herein. In particular embodiments, the term collagen refers to a collagen of any known type, that 1) is soluble in HA; and 2) induces collagen Type I production, collagen Type III production, ECM production, or a combination thereof. The term also encompasses procollagens and collagen-like proteins or collagenous proteins comprising the motif (Gly-X-Y) n where n is an integer. It encompasses molecules of collagen and collagen-like proteins, trimers of collagen molecules, fibrils of collagen, and fibers of collagen fibrils. It also refers to chemically, enzymatically or recombinantly modified collagens or collagen-like molecules that can be fibrillated as well as fragments of collagen, collagen-like molecules and collagenous molecules capable of assembling into a nanofiber. Recombinant collagen molecules whether native or engineered will generally comprise a repeated-(Gly-X-Y) n-sequence. As used herein, collagen is a generic term for a family of at least 28 distinct collagen Types. Various distinct collagen Types have been identified in a range of species, including bovine, ovine, porcine, galliform, marine, plant, and human collagens.

Collagen has a distinctive amino acid composition; nearly one-third of its amino acid residues are glycine; another 15% to 30% are proline or 4-hydroxyproline (Hyp) residues. Hydroxylysine is also characteristic of collagen. These hydroxylated amino acid residues, which are formed as the result of posttranslational modification, confer stability on collagen through intramolecular hydrogen bonds involving bridging water molecules. The collagen content in a tissue can be determined by measurement of its hydroxyproline content. Hydroxyproline content can be measured by methods known in the art, for example by liquid chromatography-mass spectrometry (LC-MS). Collagen contains many repeated iterations of the sequence Gly-X-Y, where X is often proline and Y is often 4-hydroxyproline.

Collagen variants containing such amino acid residue substitutions can be produced by a variety of known methods, including genetic engineering techniques and in vitro peptide synthesis. Collagen variants can also be produced by varying the amount of hydroxylysines and/or hydroxyprolines present in a given molecule, by the varied expression of lysine hydroxylases, and/or proline hydroxylases, wherein the hydroxylase genes (recombinant or otherwise) are expressed in a host cell for the expression of recombinant collagen, or derivatives thereof. Hydroxyproline content can be measured by methods known in the art, for example by liquid chromatography-mass spectrometry (LC-MS).

Collagen is the most abundant protein in vertebrates. Vertebrate collagen is a family of proteins produced by several cell types. Within this protein family, the collagen Types are distinguishable by their chemical compositions, different morphological and pathological features, distributions within tissues, and their functions. Although many types of collagens have been described, five major types have been recognized. Mammal skin is typically collagen Type I, although other types of collagen can be used in forming leather, including collagen Type III. The term “collagen” encompasses unprocessed (e.g., procollagens) as well as post-translationally modified and proteolyzed collagens having a triple helical structure.

Any type of collagen, including collagen Types I through XXVIII are suitable for use in the compositions described herein.

### B. Recombinant Collagen and Recombinant Collagen Fragments

As used herein, the term “recombinant collagen” refers to the family of at least 28 distinct naturally occurring collagen Types including, but not limited to collagen Types I through XXVIII, prepared using recombinant techniques.

In some embodiments, the recombinant collagen described herein is a recombinant collagen fragment. A recombinant collagen fragment can be a fragment of the full amino acid sequence of a native collagen molecule capable of forming tropocollagen (trimeric collagen) or the fragment can be a fragment of a modified collagen molecule or truncated collagen molecule having an amino acid sequence at least 70, 80, 90, 95, 96, 97, 98, or 99% identical or similar to a native collagen amino acid sequence (or to a fibril forming region thereof or to a segment substantially comprising [Gly-X-Y]n). In particular embodiments, the term recombinant collagen refers to a recombinant collagen of any known type, that (1) is soluble in HA; and (2) induces collagen Type I and/or collagen Type III production. Production of collagen Type I and/or collagen Type III can be determined by assays known in the art, including those described in PCT/US2022/027016, the entirety of which is incorporated herein by reference.

Exemplary collagen sequences from which fragments can be derived include amino acid sequences of Col1A1, Col1A2, and Col3A1, such as those described by Accession Nos. P02461.4 (SEQ ID NO: 1003; human Col3A1) (www.ncbi.nlm.nih.gov/protein/124056490), NP_001029211.1 (SEQ ID NO: 1004; bovine Col1A1) (www.ncbi.nlm.nih.gov/protein/77404252), NP_776945.1 (SEQ ID NO: 1005 bovine Col1A2) (www.ncbi.nlm.nih.gov/protein/27806257) and NP_001070299.1 (SEQ ID NO: 1006; bovine Col3A1) (www.ncbi.nlm.nih.gov/protein/116003881), which are incorporated herein by reference.

A gene encoding a collagen can be truncated or otherwise modified to add or remove sequences, for example to encode the collagen fragments disclosed herein. In addition, gene modifications can be made to customize the size of a polynucleotide or vector, to target the expressed protein to the endoplasmic reticulum or other cellular or extracellular compartment, or to control the length of an encoded protein. Modifications can be made to polynucleotides encoding collagen fragments. For example, a polynucleotide coding sequence for a collagen or collagen fragment can be modified to encode a protein that is at least 70, 80, 85, 86, 87, 88, 89, 90, 91, 92, 93, 94, 95, 96, 97, 98, or 100% identical or similar to a known amino acid sequence. Such modifications can include codon-modifying or codon-optimizing a polynucleotide encoding a collagen fragment.

In some embodiments, the collagen fragment disclosed herein can have a molecular weight from about 1 kDa to about 150 kDa. In some embodiments, the collagen fragment can have a molecular weight of about 1 kDa, 2 kDa, 3 kDa, 4 kDa, 5 kDa, 6 kDa, 7, kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, 33 kDa, 34 kDa, 35 kDa, 36 kDa, 37 kDa, 38 kDa, 39 kDa, 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, about 55 kDa, about 56 kDa, about 57 kDa, about 58 kDa, about 59 kDa, about 60 kDa, about 70 kDa, about 80 kDa, about 90 kDa, about 100 kDa, about 110 kDa, about 120 kDa, about 130 kDa, about 140 kDa, or about 150 kDa. In a particular embodiment, the collagen fragment can have a molecular weight of about 1 kDa. In a particular embodiment, the collagen fragment can have a molecular weight of about 10 kDa. In a particular embodiment, the collagen fragment can have a molecular weight of about 20 kDa. In a particular embodiment, the collagen fragment can have a molecular weight of about 30 kDa. In a particular embodiment, the collagen fragment can have a molecular weight of about 40 kDa. In a particular embodiment, the collagen fragment can have a molecular weight of about 50 kDa. In a particular embodiment, the collagen fragment can have a molecular weight of about 60 kDa. In a particular embodiment, the collagen fragment can have a molecular weight of about 100 kDa. In a particular embodiment, the collagen fragment can have a molecular weight of about 140 kDa.

In some embodiments, the collagen fragment described herein can have an amino acid chain length from about 350 amino acids to about 600 amino acids and can overlap with the amino acid sequence of SEQ ID NO: 1. In some embodiments, the overlapping collagen fragment described herein can have a length of about about 150 amino acids, about 200 amino acids, about 250 amino acids, about 300 amino acids, 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids.

In some embodiments, the collagen fragment described herein can have an amino acid sequence according to SEQ ID NO: 1. In some embodiments, the collagen fragment can have at least about 40%, at least about 45%, at least about 50%, at least about 55%, at least about 60%, at least about 65%, at least about 70%, at least about 75%, at least about 80%, about 85%, at least about 86%, at least about 87%, at least about 88%, at least about 89%, at least about 90%, at least about 91%, at least about 9%, at least about 93%, at least about 94%, at least about 95%, at least about 96%, at least about 97%, at least about 98%, at least about 99%, or 100% sequence identity, or similarity, to SEQ ID NO: 1.

The amino acid sequence of SEQ ID NO: 1 is:

DVKSGVAVGGLAGYPGPAGPPGPPGPPGTSGHPGSPGSPGYQGPPG
EPGQAGPSGPPGPPGAIGPSGPAGKDGESGRPGRPGERGLPGPPGI
KGPAGIPGFPGMKGHRGFDGRNGEKGETGAPGLKGENGLPGENGAP
GPMGPRGAPGERGRPGLPGAAGARGNDGARGSDGQPGPPGPPGTAG
FPGSPGAKGEVGPAGSPGSNGAPGQRGEPGPQGHAGAQGPPGPPGI
NGSPGGKGEMGPAGIPGAPGLMGARGPPGPAGANGAPGLRGGAGEP
GKNGAKGEPGPRGERGEAGIPGVPGAKGEDGKDGSPGEPGANGLPG
AAGERGAPGFRGPAGPNGIPGEKGPAGERGAPGPAGPRGAAGEPGR
DGVPGGPGMRGMPGSPGGPGSDGKPGPPGSQGESGRPGPPGPSGPR
GQPGVMGFPGPKGNDGAPGKNGERGGPGGPGPQGPPGKNGETGPQG
PPGPTGPGGDKGDTGPPGPQGLQGLPGTGGPPGENGKPGEPGPKGD
AGAPGAPGGKGDAGAPGERGPPAIAGIGGEKAGGFAPYYG.

In some embodiments, the collagen fragment described herein can have an amino acid chain length from about 350 amino acids to about 600 amino acids and can have at least about 40%, at least about 45%, at least about 50%, at least about 55%, at least about 60%, at least about 65%, at least about 70%, at least about 75%, at least about 80%, about 85%, at least about 86%, at least about 87%, at least about 88%, at least about 89%, at least about 90%, at least about 91%, at least about 9%, at least about 93%, at least about 94%, at least about 95%, at least about 96%, at least about 97%, at least about 98%, at least about 99%, or 100% sequence identity, or similarity to, SEQ ID NO: 1. In some embodiments, the collagen fragment described herein can have a length of about, 150 amino acids, about 200 amino acids, about 250 amino acids, about 300 amino acids, about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids.

In some embodiments, the recombinant collagen can comprise a hydrolysis product of a collagen fragment, wherein the hydrolysis product can have a sequence that is a portion of SEQ ID NO: 1. In some embodiments, the hydrolysis product can have a sequence identity of at least about 85% to the sequence according to one of SEQ ID NOs: 2-973. In some embodiments, the collagen fragment can be a collagen fragment sequence variant having the amino acid sequence according to any one of SEQ ID NOs: 975-1002.

In some embodiments, the lysine, proline, or lysine and proline residues present in the collagen or collagen fragment are hydroxylated. In some embodiments, the lysine, proline, or lysine and proline residues present in the collagen fragment are not hydroxylated. In some embodiments, about 1%, about 2%, about 3%, about 4%, about 5%, about 10%, about 11%, about 12%, about 13%, about 14%, about 15%, about 16%, about 17%, about 18%, about 19%, or about 20%, (or any intermediate value or subrange) of the lysine, proline, or lysine and proline residues in the recombinant collagen fragment can be hydroxylated.

In certain embodiments, the proline residues in the recombinant collagen fragment can be from about 1% to about 20% hydroxylated.

In some embodiments, the recombinant collagen fragment can be about 12% hydroxylated. In some embodiments, the recombinant collagen fragment can be about 5% hydroxylated.

In some embodiments, the collagen fragment can be a collagen fragment, hydrolysis product, or sequence variant having the amino acid sequence set forth in Table 1.

TABLE 1

| SEQ ID NO. | SEQUENCE |
|---|---|
| 1 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGSPGAKGEVGPAGSPGSNGAPGQRGE |
| | PGPQGHAGAQGPPGPPGINGSPGGKGEMGPAGIPGAPGLMGARGPPGPAGANG |
| | APGLRGGAGEPGKNGAKGEPGPRGERGEAGIPGVPGAKGEDGKDGSPGEPGAN |
| | GLPGAAGERGAPGFRGPAGPNGIPGEKGPAGERGAPGPAGPRGAAGEPGRDGV |
| | PGGPGMRGMPGSPGGPGSDGKPGPPGSQGESGRPGPPGPSGPRGQPGVMGFPG |
| | PKGNDGAPGKNGERGGPGGPGPQGPPGKNGETGPQGPPGPTGPGGDKGDTGPP |
| | GPQGLQGLPGTGGPPGENGKPGEPGPKGDAGAPGAPGGKGDAGAPGERGPPAI |
| | AGIGGEKAGGFAPYYG |
| 2 | AAGARGNDGARGSDGQPGPPGPPGTA |
| 3 | AAGARGNDGARGSDGQPGPPGPPGTAGFPGSPGAK |
| 4 | AAGARGNDGARGSDGQPGPPGPPGTAGFPGSPGAKGEVGPA |
| 5 | AAGEPGRDGVPGGPGMRGMPG |
| 6 | AAGEPGRDGVPGGPGMRGMPGSPGGPG |
| 7 | AAGERGAPGFRGPAGPN |
| 8 | AGAQGPPGPPGIN |
| 9 | AGAQGPPGPPGINGSPGGKGEMGPAGIPGAPGLM |
| 10 | AGARGNDGARGSDGQPGPPGPPGTA |
| 11 | AGARGNDGARGSDGQPGPPGPPGTAGFPGSPGAKGEVGPA |
| 12 | AGEPGRDGVPGGPGMRGMPGSPGGPG |
| 13 | AGERGAPGFRGPAGPN |
| 14 | AGIPGAPGLM |
| 15 | AGIPGAPGLMGARGPPGPAGANGAPGLR |
| 16 | AGIPGFPGMKGH |
| 17 | AGIPGFPGMKGHRG |
| 18 | AGIPGFPGMKGHRGF |
| 19 | AGIPGFPGMKGHRGFDGRN |
| 20 | AGIPGFPGMKGHRGFDGRNGEKGETGAPGLK |
| 21 | AGIPGFPGMKGHRGFDGRNGEKGETGAPGLKGEN |
| 22 | AGKDGESGRPGRPGERGLPGPPGIK |
| 23 | AGKDGESGRPGRPGERGLPGPPGIKGPA |
| 24 | AGPPGPPGPPGTS |
| 25 | AGPPGPPGPPGTSGH |
| 26 | AGPSGPPGPPGAIGPS |
| 27 | AGPSGPPGPPGAIGPSGPAGKDGESGRPGRPGERGLPGPPGIK |
| 28 | AGSPGSNGAPGQRGEPGPQGHAGAQGPPGPPGINGSPGGKGEMGPAGIPGAPG |
| | LM |
| 29 | AGYPGPAGPPGPPGPPGTS |
| 30 | AGYPGPAGPPGPPGPPGTSGHPGSPGSPGYQGPPGEPGQAGPSGPPGPPGAIG |
| | PS |
| 31 | AIGPSGPAGKDGESGRPGRPGERGLPGPPGIK |
| 32 | AKGEVGPAGSPGSNGAPGQRGEPGPQG |
| 33 | AKGEVGPAGSPGSNGAPGQRGEPGPQGHAGAQGPPGPPGIN |
| 34 | ANGAPGLRGGAGEPGKNGAKGEPGPR |
| 35 | ANGLPGAAGERGAPGFRGPAGPN |
| 36 | APGERGRPGLPGAAGAR |
| 37 | APGERGRPGLPGAAGARGNDGARGSDGQPGPPGPPGTA |
| 38 | APGLKGENGLPGEN |
| 39 | APGLKGENGLPGENGAPGPMGPR |
| 40 | APGLMGARGPPGPA |
| 41 | APGLMGARGPPGPAGAN |
| 42 | APGLMGARGPPGPAGANGAPGLR |
| 43 | APGQRGEPGPQGHAGAQGPPGPPGIN |
| 44 | AQGPPGPPGIN |
| 45 | AQGPPGPPGINGSPG |
| 46 | AQGPPGPPGINGSPGGK |
| 47 | AQGPPGPPGINGSPGGKGEMGPA |
| 48 | AQGPPGPPGINGSPGGKGEMGPAGIPGAPGLMGAR |
| 49 | ARGNDGARGSDGQPGPPGPPGTA |
| 50 | ARGNDGARGSDGQPGPPGPPGTAGF |
| 51 | ARGNDGARGSDGQPGPPGPPGTAGFPGSPGAKGEVGPA |
| 52 | ARGNDGARGSDGQPGPPGPPGTAGFPGSPGAKGEVGPAGSPGSN |
| 53 | ARGPPGPAGANGAPGLR |
| 54 | ARGPPGPAGANGAPGLRGGAGEPGKN |
| 55 | ARGSDGQPGPPGPPGTA |
| 56 | ARGSDGQPGPPGPPGTAGFPGSPGAK |
| 57 | ARGSDGQPGPPGPPGTAGFPGSPGAKGEVGPA |
| 58 | DAGAPGAPGGKGDAGAPGERGPP |
| 59 | DGAPGKNGERGGPGGPGPQGPPG |
| 60 | DGAPGKNGERGGPGGPGPQGPPGKN |
| 61 | DGAPGKNGERGGPGGPGPQGPPGKNGE |
| 62 | DGAPGKNGERGGPGGPGPQGPPGKNGETG |
| 63 | DGAPGKNGERGGPGGPGPQGPPGKNGETGP |
| 64 | DGAPGKNGERGGPGGPGPQGPPGKNGETGPQ |
| 65 | DGAPGKNGERGGPGGPGPQGPPGKNGETGPQG |
| 66 | DGAPGKNGERGGPGGPGPQGPPGKNGETGPQGPPG |
| 67 | DGAPGKNGERGGPGGPGPQGPPGKNGETGPQGPPGP |
| 68 | DGAPGKNGERGGPGGPGPQGPPGKNGETGPQGPPGPTG |
| 69 | DGAPGKNGERGGPGGPGPQGPPGKNGETGPQGPPGPTGPG |
| 70 | DGAPGKNGERGGPGGPGPQGPPGKNGETGPQGPPGPTGPGG |
| 71 | DGARGSDGQPGPPGPPG |
| 72 | DGARGSDGQPGPPGPPGTA |
| 73 | DGARGSDGQPGPPGPPGTAG |
| 74 | DGARGSDGQPGPPGPPGTAGF |
| 75 | DGARGSDGQPGPPGPPGTAGFPG |
| 76 | DGARGSDGQPGPPGPPGTAGFPGSPGAK |
| 77 | DGARGSDGQPGPPGPPGTAGFPGSPGAKG |
| 78 | DGARGSDGQPGPPGPPGTAGFPGSPGAKGEVGPA |
| 79 | DGARGSDGQPGPPGPPGTAGFPGSPGAKGEVGPAG |
| 80 | DGARGSDGQPGPPGPPGTAGFPGSPGAKGEVGPAGSPG |
| 81 | DGARGSDGQPGPPGPPGTAGFPGSPGAKGEVGPAGSPGSN |
| 82 | DGARGSDGQPGPPGPPGTAGFPGSPGAKGEVGPAGSPGSNGAPGQRGEPGPQ |
| 83 | DGESGRPGRPGERGLPGPPGIK |
| 84 | DGESGRPGRPGERGLPGPPGIKGPA |
| 85 | DGKPGPPGSQGESGRPGPPGPSGPRGQPGVM |
| 86 | DGQPGPPGPPGTA |
| 87 | DGQPGPPGPPGTAGFPGSPGAKGEVGPA |
| 88 | DGRNGEKGETGAPGLK |
| 89 | DGRNGEKGETGAPGLKGENGLPGEN |
| 90 | DGRNGEKGETGAPGLKGENGLPGENGAPGPMGPR |
| 91 | DGRNGEKGETGAPGLKGENGLPGENGAPGPMGPRGAPGERGRPGLPGAA |
| 92 | DGRNGEKGETGAPGLKGENGLPGENGAPGPMGPRGAPGERGRPGLPGAAGAR |
| 93 | EKGETGAPGLKGENGLPGENGAPGPMGPR |
| 94 | EMGPAGIPGAPGLMGAR |
| 95 | ENGLPGENGAPGPMGPR |
| 96 | ENGLPGENGAPGPMGPRGAPGERGRPGLPGAAGAR |
| 97 | EPGQAGPSGPPGPPGAIGPS |
| 98 | ERGLPGPPGIKGPA |
| 99 | ESGRPGPPGPSGPRGQPGVM |
| 100 | ESGRPGPPGPSGPRGQPGVMGFPGPKGN |
| 101 | ESGRPGRPGERGLPGPPGIK |
| 102 | ETGAPGLKGENGLPGEN |
| 103 | ETGAPGLKGENGLPGENGAPGPMGPR |
| 104 | ETGPQGPPGPTGPGGD |
| 105 | EVGPAGSPGSNGAPGQRGEPGPQ |
| 106 | EVGPAGSPGSNGAPGQRGEPGPQGH |
| 107 | EVGPAGSPGSNGAPGQRGEPGPQGHAG |
| 108 | EVGPAGSPGSNGAPGQRGEPGPQGHAGAQ |
| 109 | EVGPAGSPGSNGAPGQRGEPGPQGHAGAQGPPGPPGIN |
| 110 | EVGPAGSPGSNGAPGQRGEPGPQGHAGAQGPPGPPGINGSPGGK |
| 111 | FDGRNGEKGETGAPGLKGEN |
| 112 | FDGRNGEKGETGAPGLKGENGLPGEN |
| 113 | FDGRNGEKGETGAPGLKGENGLPGENGAPGPMGPR |
| 114 | FPGSPGAKGEVGPA |
| 115 | FPGSPGAKGEVGPAGSPGSN |
| 116 | GAAGARGNDGARGSDGQPGPPGPP |
| 117 | GAAGARGNDGARGSDGQPGPPGPPGTA |
| 118 | GAAGARGNDGARGSDGQPGPPGPPGTAGFPGSPGAK |
| 119 | GAAGARGNDGARGSDGQPGPPGPPGTAGFPGSPGAKGEVGPA |
| 120 | GAAGEPGRDGVPGGPGMRGMPG |
| 121 | GAAGEPGRDGVPGGPGMRGMPGSPGGPG |
| 122 | GAAGERGAPGFRGPAGPN |
| 123 | GAIGPSGPAGKDGESGRPGRPGER |
| 124 | GAIGPSGPAGKDGESGRPGRPGERGLPGPP |
| 125 | GAIGPSGPAGKDGESGRPGRPGERGLPGPPGIK |
| 126 | GAKGEPGPRGERGEAGIPGVPG |
| 127 | GAKGEPGPRGERGEAGIPGVPGAK |
| 128 | GAKGEPGPRGERGEAGIPGVPGAKG |
| 129 | GAKGEPGPRGERGEAGIPGVPGAKGEDGK |
| 130 | GAKGEPGPRGERGEAGIPGVPGAKGEDGKDG |
| 131 | GAKGEPGPRGERGEAGIPGVPGAKGEDGKDGS |
| 132 | GAKGEPGPRGERGEAGIPGVPGAKGEDGKDGSPGEPGAN |
| 133 | GAKGEVGPAGSPGSNGAPGQRGEPGPQ |
| 134 | GAKGEVGPAGSPGSNGAPGQRGEPGPQG |
| 135 | GAKGEVGPAGSPGSNGAPGQRGEPGPQGH |
| 136 | GAKGEVGPAGSPGSNGAPGQRGEPGPQGHAGAQ |
| 137 | GAKGEVGPAGSPGSNGAPGQRGEPGPQGHAGAQGPPGPPGIN |
| 138 | GANGAPGLR |
| 139 | GANGAPGLRGGAGEPGKN |
| 140 | GANGAPGLRGGAGEPGKNGAK |
| 141 | GANGAPGLRGGAGEPGKNGAKGEPGPR |
| 142 | GANGAPGLRGGAGEPGKNGAKGEPGPRG |
| 143 | GANGAPGLRGGAGEPGKNGAKGEPGPRGER |
| 144 | GANGAPGLRGGAGEPGKNGAKGEPGPRGERG |
| 145 | GANGAPGLRGGAGEPGKNGAKGEPGPRGERGEAG |
| 146 | GAPGERGRPGLPGAA |
| 147 | GAPGERGRPGLPGAAG |
| 148 | GAPGERGRPGLPGAAGA |
| 149 | GAPGERGRPGLPGAAGAR |
| 150 | GAPGERGRPGLPGAAGARGN |
| 151 | GAPGERGRPGLPGAAGARGND |
| 152 | GAPGERGRPGLPGAAGARGNDG |
| 153 | GAPGERGRPGLPGAAGARGNDGA |
| 154 | GAPGERGRPGLPGAAGARGNDGAR |
| 155 | GAPGERGRPGLPGAAGARGNDGARG |
| 156 | GAPGERGRPGLPGAAGARGNDGARGS |
| 157 | GAPGERGRPGLPGAAGARGNDGARGSD |
| 158 | GAPGERGRPGLPGAAGARGNDGARGSDG |
| 159 | GAPGERGRPGLPGAAGARGNDGARGSDGQ |
| 160 | GAPGERGRPGLPGAAGARGNDGARGSDGQPG |
| 161 | GAPGERGRPGLPGAAGARGNDGARGSDGQPGP |
| 162 | GAPGERGRPGLPGAAGARGNDGARGSDGQPGPPG |
| 163 | GAPGERGRPGLPGAAGARGNDGARGSDGQPGPPGP |
| 164 | GAPGERGRPGLPGAAGARGNDGARGSDGQPGPPGPPG |
| 165 | GAPGERGRPGLPGAAGARGNDGARGSDGQPGPPGPPGTA |
| 166 | GAPGERGRPGLPGAAGARGNDGARGSDGQPGPPGPPGTAGFPGSPGAK |
| 167 | GAPGERGRPGLPGAAGARGNDGARGSDGQPGPPGPPGTAGFPGSPGAKGEVG |
| 168 | GAPGERGRPGLPGAAGARGNDGARGSDGQPGPPGPPGTAGFPGSPGAKGEVGP |
| | A |
| 169 | GAPGERGRPGLPGAAGARGNDGARGSDGQPGPPGPPGTAGFPGSPGAKGEVGP |
| | AG |
| 170 | GAPGLKGENGLPGENGAPGPMGPR |
| 171 | GAPGLMGARGPPGPA |
| 172 | GAPGLMGARGPPGPAGAN |
| 173 | GAPGLMGARGPPGPAGANGAP |
| 174 | GAPGLMGARGPPGPAGANGAPGLR |
| 175 | GAPGLMGARGPPGPAGANGAPGLRGGAGEPGKN |
| 176 | GAPGLRGGAGEPGKN |
| 177 | GAPGLRGGAGEPGKNGAKGEPGPRGERGEA |
| 178 | GAPGPAGPRGAAGEPGRDGVPG |
| 179 | GAPGPAGPRGAAGEPGRDGVPGGPGMRGMPG |
| 180 | GAPGPMGPRGAPGERGRPGLP |
| 181 | GAPGPMGPRGAPGERGRPGLPG |
| 182 | GAPGPMGPRGAPGERGRPGLPGAA |
| 183 | GAPGPMGPRGAPGERGRPGLPGAAG |
| 184 | GAPGPMGPRGAPGERGRPGLPGAAGA |
| 185 | GAPGPMGPRGAPGERGRPGLPGAAGAR |
| 186 | GAPGPMGPRGAPGERGRPGLPGAAGARGN |
| 187 | GAPGPMGPRGAPGERGRPGLPGAAGARGNDGAR |
| 188 | GAPGPMGPRGAPGERGRPGLPGAAGARGNDGARG |
| 189 | GAPGPMGPRGAPGERGRPGLPGAAGARGNDGARGSDGQPGPPGPPGTA |
| 190 | GAPGQRGEPGPQGHAGAQGPPGPP |
| 191 | GAPGQRGEPGPQGHAGAQGPPGPPG |
| 192 | GAPGQRGEPGPQGHAGAQGPPGPPGIN |
| 193 | GAPGQRGEPGPQGHAGAQGPPGPPGING |
| 194 | GAPGQRGEPGPQGHAGAQGPPGPPGINGS |
| 195 | GAPGQRGEPGPQGHAGAQGPPGPPGINGSPG |
| 196 | GAPGQRGEPGPQGHAGAQGPPGPPGINGSPGG |
| 197 | GAPGQRGEPGPQGHAGAQGPPGPPGINGSPGGK |
| 198 | GAPGQRGEPGPQGHAGAQGPPGPPGINGSPGGKGEM |
| 199 | GAPGQRGEPGPQGHAGAQGPPGPPGINGSPGGKGEMG |
| 200 | GAPGQRGEPGPQGHAGAQGPPGPPGINGSPGGKGEMGPA |
| 201 | GAPGQRGEPGPQGHAGAQGPPGPPGINGSPGGKGEMGPAGIPGAPGLM |
| 202 | GAPGQRGEPGPQGHAGAQGPPGPPGINGSPGGKGEMGPAGIPGAPGLMG |
| 203 | GAPGQRGEPGPQGHAGAQGPPGPPGINGSPGGKGEMGPAGIPGAPGLMGA |
| 204 | GAPGQRGEPGPQGHAGAQGPPGPPGINGSPGGKGEMGPAGIPGAPGLMGAR |
| 205 | GAQGPPGPPGIN |
| 206 | GARGNDGARGSDGQPGPPGPP |
| 207 | GARGNDGARGSDGQPGPPGPPGTA |
| 208 | GARGNDGARGSDGQPGPPGPPGTAGFPG |
| 209 | GARGNDGARGSDGQPGPPGPPGTAGFPGSPGA |
| 210 | GARGNDGARGSDGQPGPPGPPGTAGFPGSPGAK |
| 211 | GARGNDGARGSDGQPGPPGPPGTAGFPGSPGAKGEVGPA |
| 212 | GARGNDGARGSDGQPGPPGPPGTAGFPGSPGAKGEVGPAG |
| 213 | GARGNDGARGSDGQPGPPGPPGTAGFPGSPGAKGEVGPAGSPG |
| 214 | GARGNDGARGSDGQPGPPGPPGTAGFPGSPGAKGEVGPAGSPGSN |
| 215 | GARGPPGPAGAN |
| 216 | GARGPPGPAGANGAPGL |
| 217 | GARGPPGPAGANGAPGLR |
| 218 | GARGPPGPAGANGAPGLRG |
| 219 | GARGPPGPAGANGAPGLRGG |
| 220 | GARGPPGPAGANGAPGLRGGA |
| 221 | GARGPPGPAGANGAPGLRGGAG |
| 222 | GARGPPGPAGANGAPGLRGGAGE |
| 223 | GARGPPGPAGANGAPGLRGGAGEPG |
| 224 | GARGPPGPAGANGAPGLRGGAGEPGKN |
| 225 | GARGPPGPAGANGAPGLRGGAGEPGKNG |
| 226 | GARGPPGPAGANGAPGLRGGAGEPGKNGA |
| 227 | GARGPPGPAGANGAPGLRGGAGEPGKNGAK |
| 228 | GARGPPGPAGANGAPGLRGGAGEPGKNGAKGE |
| 229 | GARGPPGPAGANGAPGLRGGAGEPGKNGAKGEPG |
| 230 | GARGPPGPAGANGAPGLRGGAGEPGKNGAKGEPGPR |
| 231 | GARGPPGPAGANGAPGLRGGAGEPGKNGAKGEPGPRGER |
| 232 | GARGSDGQPGPPGPPGTA |
| 233 | GARGSDGQPGPPGPPGTAGFPGSPGAK |
| 234 | GARGSDGQPGPPGPPGTAGFPGSPGAKGEVGPA |
| 235 | GDKGDTGPPGPQGLQGLPGTGGPPGEN |
| 236 | GEKGETGAPGLK |
| 237 | GEKGETGAPGLKGENGLPGEN |
| 238 | GEKGETGAPGLKGENGLPGENGAPGPM |
| 239 | GEKGETGAPGLKGENGLPGENGAPGPMGPR |
| 240 | GEKGETGAPGLKGENGLPGENGAPGPMGPRGAPGERGRPGLPGAA |
| 241 | GEKGETGAPGLKGENGLPGENGAPGPMGPRGAPGERGRPGLPGAAGAR |
| 242 | GEMGPAGIPGAPGLM |
| 243 | GEMGPAGIPGAPGLMGAR |
| 244 | GEMGPAGIPGAPGLMGARGPPGPA |
| 245 | GEMGPAGIPGAPGLMGARGPPGPAGAN |
| 246 | GEMGPAGIPGAPGLMGARGPPGPAGANGAPGLR |
| 247 | GEMGPAGIPGAPGLMGARGPPGPAGANGAPGLRGGAGEPGKN |
| 248 | GENGLPGENGAPGPM |
| 249 | GENGLPGENGAPGPMGPR |
| 250 | GENGLPGENGAPGPMGPRG |
| 251 | GENGLPGENGAPGPMGPRGAPG |
| 252 | GENGLPGENGAPGPMGPRGAPGER |
| 253 | GENGLPGENGAPGPMGPRGAPGERG |
| 254 | GENGLPGENGAPGPMGPRGAPGERGR |
| 255 | GENGLPGENGAPGPMGPRGAPGERGRPGLPGAA |
| 256 | GENGLPGENGAPGPMGPRGAPGERGRPGLPGAAGA |
| 257 | GENGLPGENGAPGPMGPRGAPGERGRPGLPGAAGAR |
| 258 | GENGLPGENGAPGPMGPRGAPGERGRPGLPGAAGARGN |
| 259 | GEPGANGLPGAAGERGAPGFRGPAGPN |
| 260 | GEPGPKGDAGAPGAPGGKGDAGAPG |
| 261 | GEPGPKGDAGAPGAPGGKGDAGAPGE |
| 262 | GEPGPQGHAGAQGPPGPPGIN |
| 263 | GEPGPQGHAGAQGPPGPPGINGSPGGKGEMGPA |
| 264 | GEPGPQGHAGAQGPPGPPGINGSPGGKGEMGPAGIPGAPGLM |
| 265 | GEPGPRGERGEAGIPGVPGAKGEDGKDG |
| 266 | GEPGQAGPSGPPGPPGAIGPS |
| 267 | GEPGQAGPSGPPGPPGAIGPSGPAGKD |
| 268 | GEPGRDGVPGGPGMRGMPGSPGGPG |
| 269 | GERGEAGIPGVPGAKG |
| 270 | GERGEAGIPGVPGAKGEDGKDG |
| 271 | GERGEAGIPGVPGAKGEDGKDGSPGEPGAN |
| 272 | GERGGPGGPGPQGPPGK |
| 273 | GERGGPGGPGPQGPPGKN |
| 274 | GERGGPGGPGPQGPPGKNG |
| 275 | GERGGPGGPGPQGPPGKNGETGP |
| 276 | GERGGPGGPGPQGPPGKNGETGPQ |
| 277 | GERGGPGGPGPQGPPGKNGETGPQG |
| 278 | GERGGPGGPGPQGPPGKNGETGPQGPP |
| 279 | GERGGPGGPGPQGPPGKNGETGPQGPPG |
| 280 | GERGGPGGPGPQGPPGKNGETGPQGPPGP |
| 281 | GERGGPGGPGPQGPPGKNGETGPQGPPGPT |
| 282 | GERGGPGGPGPQGPPGKNGETGPQGPPGPTG |
| 283 | GERGGPGGPGPQGPPGKNGETGPQGPPGPTGPG |
| 284 | GERGGPGGPGPQGPPGKNGETGPQGPPGPTGPGG |
| 285 | GERGGPGGPGPQGPPGKNGETGPQGPPGPTGPGGD |
| 286 | GERGGPGGPGPQGPPGKNGETGPQGPPGPTGPGGDK |
| 287 | GERGGPGGPGPQGPPGKNGETGPQGPPGPTGPGGDKG |
| 288 | GERGGPGGPGPQGPPGKNGETGPQGPPGPTGPGGDKGD |
| 289 | GERGGPGGPGPQGPPGKNGETGPQGPPGPTGPGGDKGDTG |
| 290 | GERGGPGGPGPQGPPGKNGETGPQGPPGPTGPGGDKGDTGPPG |
| 291 | GERGGPGGPGPQGPPGKNGETGPQGPPGPTGPGGDKGDTGPPGPQ |
| 292 | GERGGPGGPGPQGPPGKNGETGPQGPPGPTGPGGDKGDTGPPGPQG |
| 293 | GERGGPGGPGPQGPPGKNGETGPQGPPGPTGPGGDKGDTGPPGPQGLQ |
| 294 | GERGGPGGPGPQGPPGKNGETGPQGPPGPTGPGGDKGDTGPPGPQGLQG |
| 295 | GERGLPGPPGIK |
| 296 | GERGLPGPPGIKGPA |
| 297 | GERGLPGPPGIKGPAGIPGFPGMK |
| 298 | GESGRPGPPGPSGPRGQPGVM |
| 299 | GESGRPGPPGPSGPRGQPGVMGFPGPKGN |
| 300 | GESGRPGRPGERGLPGPPGIK |
| 301 | GESGRPGRPGERGLPGPPGIKG |
| 302 | GESGRPGRPGERGLPGPPGIKGPA |
| 303 | GESGRPGRPGERGLPGPPGIKGPAGIPGFPGMK |
| 304 | GETGAPGLKGENGLPGEN |
| 305 | GETGAPGLKGENGLPGENGAPGPMGPR |
| 306 | GETGAPGLKGENGLPGENGAPGPMGPRGAPGERGRPGLPGAAGAR |
| 307 | GETGAPGLKGENGLPGENGAPGPMGPRGAPGERGRPGLPGAAGARGN |
| 308 | GETGPQGPPGPTGPG |
| 309 | GETGPQGPPGPTGPGGD |
| 310 | GETGPQGPPGPTGPGGDKGDTG |
| 311 | GETGPQGPPGPTGPGGDKGDTGPPG |
| 312 | GETGPQGPPGPTGPGGDKGDTGPPGPQ |
| 313 | GETGPQGPPGPTGPGGDKGDTGPPGPQG |
| 314 | GEVGPAGSPGSNGAPG |
| 315 | GEVGPAGSPGSNGAPGQ |
| 316 | GEVGPAGSPGSNGAPGQR |
| 317 | GEVGPAGSPGSNGAPGQRGE |
| 318 | GEVGPAGSPGSNGAPGQRGEP |
| 319 | GEVGPAGSPGSNGAPGQRGEPG |
| 320 | GEVGPAGSPGSNGAPGQRGEPGP |
| 321 | GEVGPAGSPGSNGAPGQRGEPGPQ |
| 322 | GEVGPAGSPGSNGAPGQRGEPGPQG |
| 323 | GEVGPAGSPGSNGAPGQRGEPGPQGH |
| 324 | GEVGPAGSPGSNGAPGQRGEPGPQGHA |
| 325 | GEVGPAGSPGSNGAPGQRGEPGPQGHAG |
| 326 | GEVGPAGSPGSNGAPGQRGEPGPQGHAGA |
| 327 | GEVGPAGSPGSNGAPGQRGEPGPQGHAGAQ |
| 328 | GEVGPAGSPGSNGAPGQRGEPGPQGHAGAQG |
| 329 | GEVGPAGSPGSNGAPGQRGEPGPQGHAGAQGP |
| 330 | GEVGPAGSPGSNGAPGQRGEPGPQGHAGAQGPP |
| 331 | GEVGPAGSPGSNGAPGQRGEPGPQGHAGAQGPPG |
| 332 | GEVGPAGSPGSNGAPGQRGEPGPQGHAGAQGPPGP |
| 333 | GEVGPAGSPGSNGAPGQRGEPGPQGHAGAQGPPGPP |
| 334 | GEVGPAGSPGSNGAPGQRGEPGPQGHAGAQGPPGPPG |
| 335 | GEVGPAGSPGSNGAPGQRGEPGPQGHAGAQGPPGPPGIN |
| 336 | GEVGPAGSPGSNGAPGQRGEPGPQGHAGAQGPPGPPGING |
| 337 | GEVGPAGSPGSNGAPGQRGEPGPQGHAGAQGPPGPPGINGS |
| 338 | GEVGPAGSPGSNGAPGQRGEPGPQGHAGAQGPPGPPGINGSPG |
| 339 | GEVGPAGSPGSNGAPGQRGEPGPQGHAGAQGPPGPPGINGSPGG |
| 340 | GEVGPAGSPGSNGAPGQRGEPGPQGHAGAQGPPGPPGINGSPGGK |
| 341 | GEVGPAGSPGSNGAPGQRGEPGPQGHAGAQGPPGPPGINGSPGGKG |
| 342 | GEVGPAGSPGSNGAPGQRGEPGPQGHAGAQGPPGPPGINGSPGGKGEM |
| 343 | GEVGPAGSPGSNGAPGQRGEPGPQGHAGAQGPPGPPGINGSPGGKGEMG |
| 344 | GEVGPAGSPGSNGAPGQRGEPGPQGHAGAQGPPGPPGINGSPGGKGEMGPA |
| 345 | GEVGPAGSPGSNGAPGQRGEPGPQGHAGAQGPPGPPGINGSPGGKGEMGPAG |
| 346 | GFDGRNGEKGETGAPGLK |
| 347 | GFDGRNGEKGETGAPGLKG |
| 348 | GFDGRNGEKGETGAPGLKGEN |
| 349 | GFDGRNGEKGETGAPGLKGENG |
| 350 | GFDGRNGEKGETGAPGLKGENGLPGEN |
| 351 | GFDGRNGEKGETGAPGLKGENGLPGENGAPGPMGPR |
| 352 | GFDGRNGEKGETGAPGLKGENGLPGENGAPGPMGPRGAPGERGRPGLPGAA |
| 353 | GFPGMKGHRGFDGRNGEKGETGAPGLK |
| 354 | GFPGMKGHRGFDGRNGEKGETGAPGLKGEN |
| 355 | GFPGPKGNDGAPGKN |
| 356 | GFPGPKGNDGAPGKNGER |
| 357 | GFPGPKGNDGAPGKNGERG |
| 358 | GFPGPKGNDGAPGKNGERGG |
| 359 | GFPGPKGNDGAPGKNGERGGP |
| 360 | GFPGPKGNDGAPGKNGERGGPG |
| 361 | GFPGPKGNDGAPGKNGERGGPGG |
| 362 | GFPGPKGNDGAPGKNGERGGPGGP |
| 363 | GFPGPKGNDGAPGKNGERGGPGGPG |
| 364 | GFPGPKGNDGAPGKNGERGGPGGPGP |
| 365 | GFPGPKGNDGAPGKNGERGGPGGPGPQ |
| 366 | GFPGPKGNDGAPGKNGERGGPGGPGPQG |
| 367 | GFPGPKGNDGAPGKNGERGGPGGPGPQGP |
| 368 | GFPGPKGNDGAPGKNGERGGPGGPGPQGPP |
| 369 | GFPGPKGNDGAPGKNGERGGPGGPGPQGPPG |
| 370 | GFPGPKGNDGAPGKNGERGGPGGPGPQGPPGK |
| 371 | GFPGPKGNDGAPGKNGERGGPGGPGPQGPPGKN |
| 372 | GFPGPKGNDGAPGKNGERGGPGGPGPQGPPGKNG |
| 373 | GFPGPKGNDGAPGKNGERGGPGGPGPQGPPGKNGE |
| 374 | GFPGPKGNDGAPGKNGERGGPGGPGPQGPPGKNGET |
| 375 | GFPGPKGNDGAPGKNGERGGPGGPGPQGPPGKNGETG |
| 376 | GFPGPKGNDGAPGKNGERGGPGGPGPQGPPGKNGETGP |
| 377 | GFPGPKGNDGAPGKNGERGGPGGPGPQGPPGKNGETGPQ |
| 378 | GFPGPKGNDGAPGKNGERGGPGGPGPQGPPGKNGETGPQG |
| 379 | GFPGPKGNDGAPGKNGERGGPGGPGPQGPPGKNGETGPQGPPG |
| 380 | GFPGPKGNDGAPGKNGERGGPGGPGPQGPPGKNGETGPQGPPGPTG |
| 381 | GFPGPKGNDGAPGKNGERGGPGGPGPQGPPGKNGETGPQGPPGPTGPGGD |
| 382 | GFPGPKGNDGAPGKNGERGGPGGPGPQGPPGKNGETGPQGPPGPTGPGGDK |
| 383 | GFPGSPGAKGEVG |
| 384 | GFPGSPGAKGEVGP |
| 385 | GFPGSPGAKGEVGPA |
| 386 | GFPGSPGAKGEVGPAG |
| 387 | GFPGSPGAKGEVGPAGS |
| 388 | GFPGSPGAKGEVGPAGSPG |
| 389 | GFPGSPGAKGEVGPAGSPGSN |
| 390 | GFPGSPGAKGEVGPAGSPGSNG |
| 391 | GFPGSPGAKGEVGPAGSPGSNGA |
| 392 | GFPGSPGAKGEVGPAGSPGSNGAPG |
| 393 | GFPGSPGAKGEVGPAGSPGSNGAPGQ |
| 394 | GFPGSPGAKGEVGPAGSPGSNGAPGQR |
| 395 | GFPGSPGAKGEVGPAGSPGSNGAPGQRG |
| 396 | GFPGSPGAKGEVGPAGSPGSNGAPGQRGE |
| 397 | GFPGSPGAKGEVGPAGSPGSNGAPGQRGEP |
| 398 | GFPGSPGAKGEVGPAGSPGSNGAPGQRGEPG |
| 399 | GFPGSPGAKGEVGPAGSPGSNGAPGQRGEPGPQ |
| 400 | GFPGSPGAKGEVGPAGSPGSNGAPGQRGEPGPQG |
| 401 | GFPGSPGAKGEVGPAGSPGSNGAPGQRGEPGPQGH |
| 402 | GFPGSPGAKGEVGPAGSPGSNGAPGQRGEPGPQGHA |
| 403 | GFPGSPGAKGEVGPAGSPGSNGAPGQRGEPGPQGHAG |
| 404 | GFPGSPGAKGEVGPAGSPGSNGAPGQRGEPGPQGHAGA |
| 405 | GFPGSPGAKGEVGPAGSPGSNGAPGQRGEPGPQGHAGAQ |
| 406 | GFPGSPGAKGEVGPAGSPGSNGAPGQRGEPGPQGHAGAQG |
| 407 | GFPGSPGAKGEVGPAGSPGSNGAPGQRGEPGPQGHAGAQGPP |
| 408 | GFPGSPGAKGEVGPAGSPGSNGAPGQRGEPGPQGHAGAQGPPG |
| 409 | GFPGSPGAKGEVGPAGSPGSNGAPGQRGEPGPQGHAGAQGPPGPPG |
| 410 | GFPGSPGAKGEVGPAGSPGSNGAPGQRGEPGPQGHAGAQGPPGPPGIN |
| 411 | GFPGSPGAKGEVGPAGSPGSNGAPGQRGEPGPQGHAGAQGPPGPPGING |
| 412 | GFPGSPGAKGEVGPAGSPGSNGAPGQRGEPGPQGHAGAQGPPGPPGINGSPG |
| 413 | GFPGSPGAKGEVGPAGSPGSNGAPGQRGEPGPQGHAGAQGPPGPPGINGSPGG |
| | K |
| 414 | GFRGPAGPNGIPGEKGPAGERGAPGPA |
| 415 | GGAGEPGKNGAKGEPGPR |
| 416 | GGAGEPGKNGAKGEPGPRGERGEAGIP |
| 417 | GGAGEPGKNGAKGEPGPRGERGEAGIPG |
| 418 | GGAGEPGKNGAKGEPGPRGERGEAGIPGVPGAKGEDGKDG |
| 419 | GGAGEPGKNGAKGEPGPRGERGEAGIPGVPGAKGEDGKDGSPGEPGAN |
| 420 | GGKGEMGPAGIPGAPGLM |
| 421 | GGKGEMGPAGIPGAPGLMGA |
| 422 | GGKGEMGPAGIPGAPGLMGAR |
| 423 | GGPGGPGPQGPPGKNGETGPQGPPGPTGPGGD |
| 424 | GGPGMRGMPGSPGGPGSDGKPGPPGSQGESGRPGPPGPSGPRGQPGVM |
| 425 | GGPGSDGKPGPPGSQGESGRPGPPG |
| 426 | GGPGSDGKPGPPGSQGESGRPGPPGPS |
| 427 | GGPGSDGKPGPPGSQGESGRPGPPGPSGPR |
| 428 | GGPGSDGKPGPPGSQGESGRPGPPGPSGPRGQ |
| 429 | GGPGSDGKPGPPGSQGESGRPGPPGPSGPRGQPGVM |
| 430 | GHAGAQGPPGPPGIN |
| 431 | GHAGAQGPPGPPGING |
| 432 | GHAGAQGPPGPPGINGSPG |
| 433 | GHAGAQGPPGPPGINGSPGGK |
| 434 | GHAGAQGPPGPPGINGSPGGKGEMG |
| 435 | GHAGAQGPPGPPGINGSPGGKGEMGPA |
| 436 | GHAGAQGPPGPPGINGSPGGKGEMGPAGIPGAPGLM |
| 437 | GHAGAQGPPGPPGINGSPGGKGEMGPAGIPGAPGLMG |
| 438 | GHAGAQGPPGPPGINGSPGGKGEMGPAGIPGAPGLMGA |
| 439 | GHAGAQGPPGPPGINGSPGGKGEMGPAGIPGAPGLMGAR |
| 440 | GHAGAQGPPGPPGINGSPGGKGEMGPAGIPGAPGLMGARGPPGPA |
| 441 | GHAGAQGPPGPPGINGSPGGKGEMGPAGIPGAPGLMGARGPPGPAGAN |
| 442 | GHAGAQGPPGPPGINGSPGGKGEMGPAGIPGAPGLMGARGPPGPAGANGAPGL |
| | R |
| 443 | GHPGSPGSPGYQGPPGEPG |
| 444 | GHPGSPGSPGYQGPPGEPGQA |
| 445 | GHPGSPGSPGYQGPPGEPGQAG |
| 446 | GHPGSPGSPGYQGPPGEPGQAGPS |
| 447 | GHPGSPGSPGYQGPPGEPGQAGPSGPPGPPGAIGPS |
| 448 | GHPGSPGSPGYQGPPGEPGQAGPSGPPGPPGAIGPSGPA |
| 449 | GHPGSPGSPGYQGPPGEPGQAGPSGPPGPPGAIGPSGPAGKD |
| 450 | GHPGSPGSPGYQGPPGEPGQAGPSGPPGPPGAIGPSGPAGKDGES |
| 451 | GHPGSPGSPGYQGPPGEPGQAGPSGPPGPPGAIGPSGPAGKDGESGRPGRPGE |
| | R |
| 452 | GHRGFDGRNGEKGETGAPGLK |
| 453 | GHRGFDGRNGEKGETGAPGLKGEN |
| 454 | GHRGFDGRNGEKGETGAPGLKGENG |
| 455 | GHRGFDGRNGEKGETGAPGLKGENGLPGEN |
| 456 | GHRGFDGRNGEKGETGAPGLKGENGLPGENGAPGPM |
| 457 | GHRGFDGRNGEKGETGAPGLKGENGLPGENGAPGPMGPR |
| 458 | GHRGFDGRNGEKGETGAPGLKGENGLPGENGAPGPMGPRGAPGER |
| 459 | GIKGPAGIP |
| 460 | GIKGPAGIPGFPG |
| 461 | GIKGPAGIPGFPGMKG |
| 462 | GIPGAPGLM |
| 463 | GIPGAPGLMG |
| 464 | GIPGAPGLMGA |
| 465 | GIPGAPGLMGAR |
| 466 | GIPGAPGLMGARGPPG |
| 467 | GIPGAPGLMGARGPPGPA |
| 468 | GIPGAPGLMGARGPPGPAG |
| 469 | GIPGAPGLMGARGPPGPAGA |
| 470 | GIPGAPGLMGARGPPGPAGAN |
| 471 | GIPGAPGLMGARGPPGPAGANGAPGLR |
| 472 | GIPGAPGLMGARGPPGPAGANGAPGLRG |
| 473 | GIPGAPGLMGARGPPGPAGANGAPGLRGGAGEPGKN |
| 474 | GIPGEKGPAGERGAPGPA |
| 475 | GIPGEKGPAGERGAPGPAG |
| 476 | GIPGEKGPAGERGAPGPAGPR |
| 477 | GIPGEKGPAGERGAPGPAGPRG |
| 478 | GIPGEKGPAGERGAPGPAGPRGA |
| 479 | GIPGEKGPAGERGAPGPAGPRGAA |
| 480 | GIPGEKGPAGERGAPGPAGPRGAAG |
| 481 | GIPGEKGPAGERGAPGPAGPRGAAGEPG |
| 482 | GIPGEKGPAGERGAPGPAGPRGAAGEPGR |
| 483 | GIPGEKGPAGERGAPGPAGPRGAAGEPGRD |
| 484 | GIPGEKGPAGERGAPGPAGPRGAAGEPGRDGVPG |
| 485 | GIPGEKGPAGERGAPGPAGPRGAAGEPGRDGVPGG |
| 486 | GIPGEKGPAGERGAPGPAGPRGAAGEPGRDGVPGGPG |
| 487 | GIPGEKGPAGERGAPGPAGPRGAAGEPGRDGVPGGPGMR |
| 488 | GIPGFPGMKGH |
| 489 | GIPGFPGMKGHR |
| 490 | GIPGFPGMKGHRGF |
| 491 | GIPGFPGMKGHRGFD |
| 492 | GIPGFPGMKGHRGFDGR |
| 493 | GIPGFPGMKGHRGFDGRN |
| 494 | GIPGFPGMKGHRGFDGRNGEK |
| 495 | GIPGFPGMKGHRGFDGRNGEKG |
| 496 | GIPGFPGMKGHRGFDGRNGEKGETGAPGLK |
| 497 | GIPGFPGMKGHRGFDGRNGEKGETGAPGLKGEN |
| 498 | GIPGFPGMKGHRGFDGRNGEKGETGAPGLKGENGLPGEN |
| 499 | GIPGFPGMKGHRGFDGRNGEKGETGAPGLKGENGLPGENGAPGPMGPR |
| 500 | GKDGESGRPGRPGERGLPGPPGIK |
| 501 | GKDGESGRPGRPGERGLPGPPGIKGPA |
| 502 | GKDGESGRPGRPGERGLPGPPGIKGPAGIPGFPGMK |
| 503 | GKDGSPGEPGANGLPGA |
| 504 | GKDGSPGEPGANGLPGAAGERGAPG |
| 505 | GKDGSPGEPGANGLPGAAGERGAPGFRGPAGPN |
| 506 | GKGEMGPAGIPGAPGLM |
| 507 | GKGEMGPAGIPGAPGLMG |
| 508 | GKGEMGPAGIPGAPGLMGA |
| 509 | GKGEMGPAGIPGAPGLMGAR |
| 510 | GKGEMGPAGIPGAPGLMGARGPPGPA |
| 511 | GKGEMGPAGIPGAPGLMGARGPPGPAGAN |
| 512 | GKGEMGPAGIPGAPGLMGARGPPGPAGANGAPGLR |
| 513 | GKPGEPGPKGDAGAPGAPGGKGDAGAPG |
| 514 | GKPGEPGPKGDAGAPGAPGGKGDAGAPGE |
| 515 | GKPGEPGPKGDAGAPGAPGGKGDAGAPGER |
| 516 | GKPGEPGPKGDAGAPGAPGGKGDAGAPGERG |
| 517 | GKPGEPGPKGDAGAPGAPGGKGDAGAPGERGPP |
| 518 | GKPGPPGSQGESGRPGPPGPSGPR |
| 519 | GKPGPPGSQGESGRPGPPGPSGPRGQ |
| 520 | GKPGPPGSQGESGRPGPPGPSGPRGQPGVM |
| 521 | GLAGYPGPAGPPGPPGPPGTS |
| 522 | GLAGYPGPAGPPGPPGPPGTSGHPGSPGSPGYQGPPGEPGQAGPSGPPGPPGA |
| | IGPS |
| 523 | GLKGENGLPGENGAPGPMGPR |
| 524 | GLMGARGPPGPAGANGAPGLR |
| 525 | GLPGAAGARGNDGARGSDGQPGPPGPPGTA |
| 526 | GLPGAAGARGNDGARGSDGQPGPPGPPGTAGFPGSPGAKGEVGPA |
| 527 | GLPGAAGERGAPGF |
| 528 | GLPGAAGERGAPGFR |
| 529 | GLPGAAGERGAPGFRG |
| 530 | GLPGAAGERGAPGFRGPAG |
| 531 | GLPGAAGERGAPGFRGPAGPN |
| 532 | GLPGAAGERGAPGFRGPAGPNGIPGEKG |
| 533 | GLPGAAGERGAPGFRGPAGPNGIPGEKGPAGER |
| 534 | GLPGAAGERGAPGFRGPAGPNGIPGEKGPAGERGAPG |
| 535 | GLPGAAGERGAPGFRGPAGPNGIPGEKGPAGERGAPGPAGPR |
| 536 | GLPGENGAPGPM |
| 537 | GLPGENGAPGPMG |
| 538 | GLPGENGAPGPMGPR |
| 539 | GLPGENGAPGPMGPRG |
| 540 | GLPGENGAPGPMGPRGAPG |
| 541 | GLPGENGAPGPMGPRGAPGERGR |
| 542 | GLPGENGAPGPMGPRGAPGERGRPG |
| 543 | GLPGENGAPGPMGPRGAPGERGRPGLPGAAGAR |
| 544 | GLPGENGAPGPMGPRGAPGERGRPGLPGAAGARGN |
| 545 | GLPGENGAPGPMGPRGAPGERGRPGLPGAAGARGNDGARG |
| 546 | GLPGENGAPGPMGPRGAPGERGRPGLPGAAGARGNDGARGSDGQ |
| 547 | GLPGENGAPGPMGPRGAPGERGRPGLPGAAGARGNDGARGSDGQPGPPGPPGT |
| | A |
| 548 | GLPGPPGIK |
| 549 | GLPGPPGIKG |
| 550 | GLPGPPGIKGP |
| 551 | GLPGPPGIKGPA |
| 552 | GLPGPPGIKGPAG |
| 553 | GLPGPPGIKGPAGIP |
| 554 | GLPGPPGIKGPAGIPG |
| 555 | GLPGPPGIKGPAGIPGF |
| 556 | GLPGPPGIKGPAGIPGFPGM |
| 557 | GLPGPPGIKGPAGIPGFPGMK |
| 558 | GLPGPPGIKGPAGIPGFPGMKG |
| 559 | GLPGPPGIKGPAGIPGFPGMKGH |
| 560 | GLPGPPGIKGPAGIPGFPGMKGHR |
| 561 | GLPGPPGIKGPAGIPGFPGMKGHRGF |
| 562 | GLPGTGGPPGEN |
| 563 | GLRGGAGEPGKNGAKGEPGPRGERGEAGIP |
| 564 | GMPGSPGGPGSDGKPGPPG |
| 565 | GMPGSPGGPGSDGKPGPPGSQ |
| 566 | GMPGSPGGPGSDGKPGPPGSQG |
| 567 | GMPGSPGGPGSDGKPGPPGSQGESGRPGPPGPSGPR |
| 568 | GMPGSPGGPGSDGKPGPPGSQGESGRPGPPGPSGPRGQPGVM |
| 569 | GMRGMPGSPGGPGSDGKPGPPGSQGESGRPGPPGPSGPRGQPGVM |
| 570 | GNDGAPGKNGERGGPGGPGPQGPPGKN |
| 571 | GNDGAPGKNGERGGPGGPGPQGPPGKNGETGPQG |
| 572 | GNDGAPGKNGERGGPGGPGPQGPPGKNGETGPQGPPGP |
| 573 | GNDGARGSDGQPGPPGPPG |
| 574 | GNDGARGSDGQPGPPGPPGTA |
| 575 | GNDGARGSDGQPGPPGPPGTAG |
| 576 | GNDGARGSDGQPGPPGPPGTAGF |
| 577 | GNDGARGSDGQPGPPGPPGTAGFPG |
| 578 | GNDGARGSDGQPGPPGPPGTAGFPGS |
| 579 | GNDGARGSDGQPGPPGPPGTAGFPGSPG |
| 580 | GNDGARGSDGQPGPPGPPGTAGFPGSPGAK |
| 581 | GNDGARGSDGQPGPPGPPGTAGFPGSPGAKG |
| 582 | GNDGARGSDGQPGPPGPPGTAGFPGSPGAKGE |
| 583 | GNDGARGSDGQPGPPGPPGTAGFPGSPGAKGEVG |
| 584 | GNDGARGSDGQPGPPGPPGTAGFPGSPGAKGEVGP |
| 585 | GNDGARGSDGQPGPPGPPGTAGFPGSPGAKGEVGPA |
| 586 | GNDGARGSDGQPGPPGPPGTAGFPGSPGAKGEVGPAG |
| 587 | GNDGARGSDGQPGPPGPPGTAGFPGSPGAKGEVGPAGS |
| 588 | GNDGARGSDGQPGPPGPPGTAGFPGSPGAKGEVGPAGSPG |
| 589 | GNDGARGSDGQPGPPGPPGTAGFPGSPGAKGEVGPAGSPGSN |
| 590 | GNDGARGSDGQPGPPGPPGTAGFPGSPGAKGEVGPAGSPGSNG |
| 591 | GNDGARGSDGQPGPPGPPGTAGFPGSPGAKGEVGPAGSPGSNGAPGQ |
| 592 | GNDGARGSDGQPGPPGPPGTAGFPGSPGAKGEVGPAGSPGSNGAPGQR |
| 593 | GNDGARGSDGQPGPPGPPGTAGFPGSPGAKGEVGPAGSPGSNGAPGQRGE |
| 594 | GNDGARGSDGQPGPPGPPGTAGFPGSPGAKGEVGPAGSPGSNGAPGQRGEPGP |
| | Q |
| 595 | GNDGARGSDGQPGPPGPPGTAGFPGSPGAKGEVGPAGSPGSNGAPGQRGEPGP |
| | QG |
| 596 | GPAGANGAPGLRGGAGEPG |
| 597 | GPAGIPGAPGLM |
| 598 | GPAGIPGAPGLMG |
| 599 | GPAGIPGAPGLMGA |
| 600 | GPAGIPGAPGLMGAR |
| 601 | GPAGIPGAPGLMGARGPPGPAGANGAPGLR |
| 602 | GPAGIPGFP |
| 603 | GPAGIPGFPG |
| 604 | GPAGIPGFPGM |
| 605 | GPAGIPGFPGMK |
| 606 | GPAGIPGFPGMKG |
| 607 | GPAGIPGFPGMKGH |
| 608 | GPAGIPGFPGMKGHR |
| 609 | GPAGIPGFPGMKGHRG |
| 610 | GPAGIPGFPGMKGHRGF |
| 611 | GPAGIPGFPGMKGHRGFD |
| 612 | GPAGIPGFPGMKGHRGFDG |
| 613 | GPAGIPGFPGMKGHRGFDGR |
| 614 | GPAGIPGFPGMKGHRGFDGRN |
| 615 | GPAGIPGFPGMKGHRGFDGRNG |
| 616 | GPAGIPGFPGMKGHRGFDGRNGE |
| 617 | GPAGIPGFPGMKGHRGFDGRNGEK |
| 618 | GPAGIPGFPGMKGHRGFDGRNGEKG |
| 619 | GPAGIPGFPGMKGHRGFDGRNGEKGE |
| 620 | GPAGIPGFPGMKGHRGFDGRNGEKGET |
| 621 | GPAGIPGFPGMKGHRGFDGRNGEKGETG |
| 622 | GPAGIPGFPGMKGHRGFDGRNGEKGETGA |
| 623 | GPAGIPGFPGMKGHRGFDGRNGEKGETGAP |
| 624 | GPAGIPGFPGMKGHRGFDGRNGEKGETGAPG |
| 625 | GPAGIPGFPGMKGHRGFDGRNGEKGETGAPGL |
| 626 | GPAGIPGFPGMKGHRGFDGRNGEKGETGAPGLK |
| 627 | GPAGIPGFPGMKGHRGFDGRNGEKGETGAPGLKG |
| 628 | GPAGIPGFPGMKGHRGFDGRNGEKGETGAPGLKGEN |
| 629 | GPAGIPGFPGMKGHRGFDGRNGEKGETGAPGLKGENGLPG |
| 630 | GPAGIPGFPGMKGHRGFDGRNGEKGETGAPGLKGENGLPGEN |
| 631 | GPAGIPGFPGMKGHRGFDGRNGEKGETGAPGLKGENGLPGENGAPGPM |
| 632 | GPAGIPGFPGMKGHRGFDGRNGEKGETGAPGLKGENGLPGENGAPGPMGPR |
| 633 | GPAGKDGESGRPGRPGERGLPGPP |
| 634 | GPAGKDGESGRPGRPGERGLPGPPG |
| 635 | GPAGKDGESGRPGRPGERGLPGPPGIK |
| 636 | GPAGKDGESGRPGRPGERGLPGPPGIKGPA |
| 637 | GPAGKDGESGRPGRPGERGLPGPPGIKGPAGIPGFPGMK |
| 638 | GPAGPNGIPGEKGPAGERGAPGPAGPR |
| 639 | GPAGPPGPPGPP |
| 640 | GPGMRGMPGSPGGPGSDGKPGPPG |
| 641 | GPGMRGMPGSPGGPGSDGKPGPPGSQGESGRPGPPGPSGPRGQPGVM |
| 642 | GPGSDGKPGPPGSQGESGRPGPPG |
| 643 | GPGSDGKPGPPGSQGESGRPGPPGPSG |
| 644 | GPGSDGKPGPPGSQGESGRPGPPGPSGPR |
| 645 | GPGSDGKPGPPGSQGESGRPGPPGPSGPRGQPGVM |
| 646 | GPGSDGKPGPPGSQGESGRPGPPGPSGPRGQPGVMGFPGPKGN |
| 647 | GPMGPRGAPGERGRPGLPGAA |
| 648 | GPMGPRGAPGERGRPGLPGAAGAR |
| 649 | GPNGIPGEKGPAGERGAPGPA |
| 650 | GPPGAIGPSGPAGKDGESGRPGRPGER |
| 651 | GPPGAIGPSGPAGKDGESGRPGRPGERGLPGPPGIK |
| 652 | GPPGEPGQAGPSGPPGPPG |
| 653 | GPPGEPGQAGPSGPPGPPGAIGPS |
| 654 | GPPGEPGQAGPSGPPGPPGAIGPSGPAGKD |
| 655 | GPPGEPGQAGPSGPPGPPGAIGPSGPAGKDGESGRPGRPGERGLPGPPGIK |
| 656 | GPPGIKGPAGIPGFPGMK |
| 657 | GPPGINGSPGGKGEMGPAGIPGAPGLM |
| 658 | GPPGPAGANGAPGLR |
| 659 | GPPGPAGANGAPGLRG |
| 660 | GPPGPAGANGAPGLRGG |
| 661 | GPPGPAGANGAPGLRGGA |
| 662 | GPPGPAGANGAPGLRGGAG |
| 663 | GPPGPAGANGAPGLRGGAGE |
| 664 | GPPGPAGANGAPGLRGGAGEP |
| 665 | GPPGPAGANGAPGLRGGAGEPG |
| 666 | GPPGPAGANGAPGLRGGAGEPGK |
| 667 | GPPGPAGANGAPGLRGGAGEPGKN |
| 668 | GPPGPAGANGAPGLRGGAGEPGKNG |
| 669 | GPPGPAGANGAPGLRGGAGEPGKNGA |
| 670 | GPPGPAGANGAPGLRGGAGEPGKNGAK |
| 671 | GPPGPAGANGAPGLRGGAGEPGKNGAKG |
| 672 | GPPGPAGANGAPGLRGGAGEPGKNGAKGE |
| 673 | GPPGPAGANGAPGLRGGAGEPGKNGAKGEPG |
| 674 | GPPGPAGANGAPGLRGGAGEPGKNGAKGEPGPR |
| 675 | GPPGPAGANGAPGLRGGAGEPGKNGAKGEPGPRG |
| 676 | GPPGPAGANGAPGLRGGAGEPGKNGAKGEPGPRGE |
| 677 | GPPGPAGANGAPGLRGGAGEPGKNGAKGEPGPRGER |
| 678 | GPPGPAGANGAPGLRGGAGEPGKNGAKGEPGPRGERG |
| 679 | GPPGPAGANGAPGLRGGAGEPGKNGAKGEPGPRGERGE |
| 680 | GPPGPAGANGAPGLRGGAGEPGKNGAKGEPGPRGERGEA |
| 681 | GPPGPPGAIGPS |
| 682 | GPPGPPGAIGPSGPA |
| 683 | GPPGPPGAIGPSGPAGKDGESGRPGRPGER |
| 684 | GPPGPPGAIGPSGPAGKDGESGRPGRPGERGLPGPPGIK |
| 685 | GPPGPPGINGSPGGKGEMGPA |
| 686 | GPPGPPGINGSPGGKGEMGPAGIPGAPGLM |
| 687 | GPPGPPGINGSPGGKGEMGPAGIPGAPGLMGA |
| 688 | GPPGPPGINGSPGGKGEMGPAGIPGAPGLMGAR |
| 689 | GPPGPPGINGSPGGKGEMGPAGIPGAPGLMGARGPPGPAGANGAPGLR |
| 690 | GPPGPPGPPGTS |
| 691 | GPPGPPGPPGTSGHPG |
| 692 | GPPGPPGPPGTSGHPGSPGSPGYQGPPGEPGQAGPSGPPGPPGAIGPS |
| 693 | GPPGPPGTAGFPGSPGAK |
| 694 | GPPGPPGTAGFPGSPGAKGEVGPA |
| 695 | GPPGPSGPRGQPGVM |
| 696 | GPPGPSGPRGQPGVMGFPGPKGNDGAPGKN |
| 697 | GPPGSQGESGRPGPPGPSGPRGQPGVM |
| 698 | GPPGTAGFPGSPGAKGEVGPA |
| 699 | GPQGHAGAQGPPGPPGIN |
| 700 | GPRGAAGEPGRDGVPGGPGMRGMPG |
| 701 | GPRGAPGERGRPGLPGAA |
| 702 | GPRGAPGERGRPGLPGAAGA |
| 703 | GPRGAPGERGRPGLPGAAGAR |
| 704 | GPRGAPGERGRPGLPGAAGARGN |
| 705 | GPRGAPGERGRPGLPGAAGARGNDGARG |
| 706 | GPRGAPGERGRPGLPGAAGARGNDGARGSDGQPGPPGPPGTA |
| 707 | GPRGQPGVMGFPGPKGN |
| 708 | GPRGQPGVMGFPGPKGNDGAPGKN |
| 709 | GPSGPAGKDGESGRPGRPGERGLPGPPGIK |
| 710 | GPSGPAGKDGESGRPGRPGERGLPGPPGIKGPA |
| 711 | GPSGPPGPPGAIGPS |
| 712 | GPSGPRGQPGVMGFPGPKGNDGAPGKN |
| 713 | GQAGPSGPPGPPGAIGPS |
| 714 | GQPGPPGPPGTA |
| 715 | GQPGPPGPPGTAGFPGSPGAK |
| 716 | GQPGPPGPPGTAGFPGSPGAKGEVGPA |
| 717 | GQPGVMGFPGPK |
| 718 | GQPGVMGFPGPKGN |
| 719 | GQPGVMGFPGPKGNDGAPGKN |
| 720 | GQRGEPGPQGHAGAQGPPGPPGIN |
| 721 | GRDGVPGGPGMRGMPGSPGGPG |
| 722 | GRDGVPGGPGMRGMPGSPGGPGSDGKPGPPGSQGESGRPGPPGPSGPRGQPGV |
| | M |
| 723 | GRNGEKGETGAPGLKGENGLPGEN |
| 724 | GRNGEKGETGAPGLKGENGLPGENGAPGPMGPR |
| 725 | GRPGERGLPGPPGIK |
| 726 | GRPGERGLPGPPGIKGPA |
| 727 | GRPGLPGAAGAR |
| 728 | GRPGLPGAAGARGNDGARGSDGQPGPPGPPGTA |
| 729 | GRPGLPGAAGARGNDGARGSDGQPGPPGPPGTAGFPGSPGAK |
| 730 | GRPGLPGAAGARGNDGARGSDGQPGPPGPPGTAGFPGSPGAKGEVGPA |
| 731 | GRPGPPGPSGPRGQPGVM |
| 732 | GRPGRPGERGLPGPPGIK |
| 733 | GRPGRPGERGLPGPPGIKGPA |
| 734 | GSDGKPGPPGSQGESGRPGPPGPSGPRGQPGVM |
| 735 | GSDGQPGPPGPPGTA |
| 736 | GSDGQPGPPGPPGTAGFPGSPGAK |
| 737 | GSDGQPGPPGPPGTAGFPGSPGAKGEVGPA |
| 738 | GSNGAPGQRGEPGPQGHAGAQGPPGPPGIN |
| 739 | GSPGEPGANGLPGAAGERGAPGFRGPAGPN |
| 740 | GSPGGKGEMGPAGIPGAPG |
| 741 | GSPGGKGEMGPAGIPGAPGLM |
| 742 | GSPGGKGEMGPAGIPGAPGLMG |
| 743 | GSPGGKGEMGPAGIPGAPGLMGA |
| 744 | GSPGGKGEMGPAGIPGAPGLMGAR |
| 745 | GSPGGKGEMGPAGIPGAPGLMGARG |
| 746 | GSPGGKGEMGPAGIPGAPGLMGARGPPG |
| 747 | GSPGGKGEMGPAGIPGAPGLMGARGPPGPA |
| 748 | GSPGGKGEMGPAGIPGAPGLMGARGPPGPAG |
| 749 | GSPGGKGEMGPAGIPGAPGLMGARGPPGPAGAN |
| 750 | GSPGGKGEMGPAGIPGAPGLMGARGPPGPAGANGAPGLR |
| 751 | GSPGGKGEMGPAGIPGAPGLMGARGPPGPAGANGAPGLRGGAGEPGKN |
| 752 | GSPGGPGSDGKPGPPGSQGESGRPGPPGPSGPRGQPGVM |
| 753 | GSPGSNGAPGQRGEPGPQ |
| 754 | GSPGSNGAPGQRGEPGPQGHAGAQ |
| 755 | GSPGSNGAPGQRGEPGPQGHAGAQGP |
| 756 | GSPGSNGAPGQRGEPGPQGHAGAQGPP |
| 757 | GSPGSNGAPGQRGEPGPQGHAGAQGPPG |
| 758 | GSPGSNGAPGQRGEPGPQGHAGAQGPPGP |
| 759 | GSPGSNGAPGQRGEPGPQGHAGAQGPPGPP |
| 760 | GSPGSNGAPGQRGEPGPQGHAGAQGPPGPPG |
| 761 | GSPGSNGAPGQRGEPGPQGHAGAQGPPGPPGIN |
| 762 | GSPGSNGAPGQRGEPGPQGHAGAQGPPGPPGING |
| 763 | GSPGSNGAPGQRGEPGPQGHAGAQGPPGPPGINGS |
| 764 | GSPGSNGAPGQRGEPGPQGHAGAQGPPGPPGINGSPG |
| 765 | GSPGSNGAPGQRGEPGPQGHAGAQGPPGPPGINGSPGG |
| 766 | GSPGSNGAPGQRGEPGPQGHAGAQGPPGPPGINGSPGGK |
| 767 | GSPGSNGAPGQRGEPGPQGHAGAQGPPGPPGINGSPGGKG |
| 768 | GSPGSNGAPGQRGEPGPQGHAGAQGPPGPPGINGSPGGKGEM |
| 769 | GSPGSNGAPGQRGEPGPQGHAGAQGPPGPPGINGSPGGKGEMG |
| 770 | GSPGSNGAPGQRGEPGPQGHAGAQGPPGPPGINGSPGGKGEMGPA |
| 771 | GSPGSNGAPGQRGEPGPQGHAGAQGPPGPPGINGSPGGKGEMGPAG |
| 772 | GSPGSNGAPGQRGEPGPQGHAGAQGPPGPPGINGSPGGKGEMGPAGIPGAPGL |
| | M |
| 773 | GSPGSNGAPGQRGEPGPQGHAGAQGPPGPPGINGSPGGKGEMGPAGIPGAPGL |
| | MG |
| 774 | GSPGSNGAPGQRGEPGPQGHAGAQGPPGPPGINGSPGGKGEMGPAGIPGAPGL |
| | MGA |
| 775 | GSQGESGRPGPPGPSGPRGQPGVM |
| 776 | GTAGFPGSPGAKGEVGPA |
| 777 | GTSGHPGSPGSPGYQGPPGEPGQAGPSGPPGPPGAIGPS |
| 778 | GVPGGPGMRGMPGSPGGPGSDGKPGPPGSQ |
| 779 | GVPGGPGMRGMPGSPGGPGSDGKPGPPGSQGESGRPGPPGPSGPRGQPGVM |
| 780 | GYPGPAGPPGPPGPPGT |
| 781 | GYPGPAGPPGPPGPPGTS |
| 782 | GYPGPAGPPGPPGPPGTSGH |
| 783 | GYPGPAGPPGPPGPPGTSGHPG |
| 784 | GYPGPAGPPGPPGPPGTSGHPGSPGSPGYQGPPGEPGQAGPSGPPGPPGAIGP |
| | S |
| 785 | GYPGPAGPPGPPGPPGTSGHPGSPGSPGYQGPPGEPGQAGPSGPPGPPGAIGP |
| | SGPA |
| 786 | HAGAQGPPGPPGIN |
| 787 | HAGAQGPPGPPGINGSPG |
| 788 | HAGAQGPPGPPGINGSPGGKGEMG |
| 789 | HAGAQGPPGPPGINGSPGGKGEMGPA |
| 790 | HAGAQGPPGPPGINGSPGGKGEMGPAGIPGAPGLMGAR |
| 791 | HPGSPGSPGYQGPPGEPGQAGPSGPPGPPGAIGPS |
| 792 | HRGFDGRNGEKGETGAPGLKGEN |
| 793 | HRGFDGRNGEKGETGAPGLKGENGLPGENGAPGPMGPR |
| 794 | IGPSGPAGKDGESGRPGRPGERGLPGPPGIK |
| 795 | IKGPAGIPGFPG |
| 796 | IKGPAGIPGFPGMK |
| 797 | IPGAPGLMGARGPPGPAGANGAPGLR |
| 798 | IPGFPGMKGHRGFDGRN |
| 799 | KDGESGRPGRPGERGLPGPPGIK |
| 800 | KDGESGRPGRPGERGLPGPPGIKGPA |
| 801 | KDGSPGEPGANGLPGAAGERGAPGFRGPAGPN |
| 802 | KGENGLPGENGAPGPMGPR |
| 803 | KGPAGIPGFPGMK |
| 804 | KPGPPGSQGESGRPGPPGPSGPR |
| 805 | KPGPPGSQGESGRPGPPGPSGPRGQPGVM |
| 806 | LKGENGLPGENGAPGPMGPR |
| 807 | LMGARGPPGPAGANGAPGLR |
| 808 | LPGAAGARGNDGARGSDGQPGPPGPPGTA |
| 809 | LPGAAGERGAPGFRGPAGPN |
| 810 | LPGPPGIK |
| 811 | LPGPPGIKGPA |
| 812 | LPGPPGIKGPAGIPGFPGMK |
| 813 | MGARGPPGPAGANGAPGLR |
| 814 | MGARGPPGPAGANGAPGLRGGAGEPGKN |
| 815 | MGPAGIPGAPGLMGARGPPGPAGANGAPGLR |
| 816 | MGPRGAPGERGRPGLPGAAGAR |
| 817 | MPGSPGGPGSDGKPGPPGSQGESGRPGPPGPSGPRGQPGVM |
| 818 | MRGMPGSPGGPGSDGKPGPPGSQGESGRPGPPGPSGPRGQPGVM |
| 819 | NDGARGSDGQPGPPGPPGTA |
| 820 | NDGARGSDGQPGPPGPPGTAGFPGSPGAKGEVGPA |
| 821 | NGAPGPMGPRGAPGE |
| 822 | NGAPGPMGPRGAPGERGRPGLPGAAGAR |
| 823 | NGEKGETGAPGLKGENGLPGENGAPGPMGPR |
| 824 | NGETGPQGPPGPTGPGGD |
| 825 | NGLPGENGAPGPMGPR |
| 826 | NGSPGGKGEMGPAGIPGAPGLM |
| 827 | NGSPGGKGEMGPAGIPGAPGLMGARGPPGPAGANGAPGLR |
| 828 | PAGIPGAPGLM |
| 829 | PAGIPGAPGLMG |
| 830 | PAGIPGFPGMK |
| 831 | PAGIPGFPGMKG |
| 832 | PAGIPGFPGMKGHR |
| 833 | PAGIPGFPGMKGHRG |
| 834 | PAGIPGFPGMKGHRGF |
| 835 | PAGIPGFPGMKGHRGFDG |
| 836 | PAGIPGFPGMKGHRGFDGRN |
| 837 | PAGKDGESGRPGRPGERGLPGPPGIK |
| 838 | PGAAGERGAPGFRGPAGPN |
| 839 | PGANGLPGAAGERGAP |
| 840 | PGAPGLMGARGPPGPAGANGAPGLR |
| 841 | PGEPGPKGDAGAPGAPGGKGDAGAPGE |
| 842 | PGERGLPGPPGIK |
| 843 | PGERGRPGLPGAAGAR |
| 844 | PGFPGMKGH |
| 845 | PGFPGMKGHR |
| 846 | PGFPGMKGHRGF |
| 847 | PGFPGMKGHRGFDGR |
| 848 | PGLMGARGPPGPAGANGAPGL |
| 849 | PGLPGAAGARGNDGARGSDGQPGPPGPPGTAGFPGSPGAKG |
| 850 | PGPAGANGAPGLRGGAGEPGKN |
| 851 | PGPAGPPGPPGPPG |
| 852 | PGPAGPPGPPGPPGTS |
| 853 | PGPAGPPGPPGPPGTSG |
| 854 | PGPAGPPGPPGPPGTSGH |
| 855 | PGPAGPPGPPGPPGTSGHPGSPGSPGYQGPPGEPGQAGPSGP |
| 856 | PGPAGPPGPPGPPGTSGHPGSPGSPGYQGPPGEPGQAGPSGPPGPPGAIGPSG |
| | PAGK |
| 857 | PGPKGNDGAPGKN |
| 858 | PGPKGNDGAPGKNGERGGPGGPGPQGPPGKN |
| 859 | PGPMGPRGAPGERGRPGLPGAAGAR |
| 860 | PGPPGAIGPS |
| 861 | PGPPGIKGPAGIPGFPGMK |
| 862 | PGPPGPPGTAGFPGSPGAK |
| 863 | PGPPGPSGPRGQPGVM |
| 864 | PGRPGERGLPGPPGIK |
| 865 | PGSDGKPGPPGSQGESGRPGPPGPSGPRGQPGVM |
| 866 | PGSNGAPGQRGEPGPQGHAGAQGPPGPPGINGS |
| 867 | PGSPGAKGEVGPA |
| 868 | PPGAIGPSGPAGKDGESGRPGRPGER |
| 869 | PPGAIGPSGPAGKDGESGRPGRPGERGLPGPPGIK |
| 870 | PPGEPGQAGPSGPPGPPGAIGPS |
| 871 | PPGEPGQAGPSGPPGPPGAIGPSGPAGKD |
| 872 | PPGIKGPAGIPGFPGMK |
| 873 | PPGINGSPGGKGEMGPAGIPGAPGLM |
| 874 | PPGPAGANGAPGLR |
| 875 | PPGPPGAIGPS |
| 876 | PPGPPGAIGPSGPAGKDGESGRPGRPGERGLPGPPGIK |
| 877 | PPGPPGINGSPGGKGEMGPAGIPGAPGLM |
| 878 | PPGPPGTAGFPGSPGAKGEVGPA |
| 879 | PPGSQGESGRPGPPGPSGPRGQPGVM |
| 880 | PPGTAGFPGSPGAKGEVGPA |
| 881 | PQGHAGAQGPPGPPGIN |
| 882 | PSGPAGKDGESGRPGRPGERGLPGPPGIK |
| 883 | PSGPPGPPGAIGPS |
| 884 | QAGPSGPPGPPGAIGPS |
| 885 | QGPPGEPGQAGPSGPPGPPGAIGPSGPAGKD |
| 886 | QRGEPGPQGHAGAQGPPGPPGIN |
| 887 | RGFDGRNGEKGETGAPGLKGEN |
| 888 | RGLPGPPGIK |
| 889 | RGLPGPPGIKGPA |
| 890 | RGLPGPPGIKGPAGIPGFPGMK |
| 891 | RGMPGSPGGPGSDGKPGPPGSQGESGRPGPPGPSGPRGQPGVM |
| 892 | RGNDGARGSDGQPGPPGPPGTA |
| 893 | RGNDGARGSDGQPGPPGPPGTAGFPGSPGAK |
| 894 | RGNDGARGSDGQPGPPGPPGTAGFPGSPGAKGEVGPA |
| 895 | RGPPGPAGANGAPGL |
| 896 | RGPPGPAGANGAPGLR |
| 897 | RGPPGPAGANGAPGLRG |
| 898 | RGPPGPAGANGAPGLRGGAGEPGKN |
| 899 | RGPPGPAGANGAPGLRGGAGEPGKNGAK |
| 900 | RGSDGQPGPPGPPGTAGFPGSPGAK |
| 901 | RGSDGQPGPPGPPGTAGFPGSPGAKGEVGPA |
| 902 | RPGERGLPGPPGIK |
| 903 | RPGERGLPGPPGIKGPA |
| 904 | RPGLPGAAGARGNDGARGSDGQPGPPGP |
| 905 | RPGLPGAAGARGNDGARGSDGQPGPPGPPGTA |
| 906 | RPGPPGPSGPRGQPGVM |
| 907 | RPGRPGERGLPGPPGIK |
| 908 | RPGRPGERGLPGPPGIKGPA |
| 909 | SDGKPGPPGSQGESGRPGPPGPS |
| 910 | SDGKPGPPGSQGESGRPGPPGPSG |
| 911 | SDGKPGPPGSQGESGRPGPPGPSGPR |
| 912 | SDGKPGPPGSQGESGRPGPPGPSGPRG |
| 913 | SDGKPGPPGSQGESGRPGPPGPSGPRGQPG |
| 914 | SDGKPGPPGSQGESGRPGPPGPSGPRGQPGVM |
| 915 | SDGKPGPPGSQGESGRPGPPGPSGPRGQPGVMGFPGPKGN |
| 916 | SDGQPGPPGPPGTA |
| 917 | SDGQPGPPGPPGTAGFPGSPGAK |
| 918 | SDGQPGPPGPPGTAGFPGSPGAKGEVGPA |
| 919 | SGPAGKDGESGRPGRPGERGLPGPPGIK |
| 920 | SGPPGPPGAIGPS |
| 921 | SGPPGPPGAIGPSGPAGKD |
| 922 | SGPPGPPGAIGPSGPAGKDGESGRPGRPGER |
| 923 | SGPPGPPGAIGPSGPAGKDGESGRPGRPGERGLPGPPGIK |
| 924 | SGPPGPPGAIGPSGPAGKDGESGRPGRPGERGLPGPPGIKGPA |
| 925 | SGPRGQPGVMGFPGPKGN |
| 926 | SGRPGPPGPSGPRGQPGVM |
| 927 | SGRPGPPGPSGPRGQPGVMGFPGPKGN |
| 928 | SGRPGRPGERGLPGPP |
| 929 | SGRPGRPGERGLPGPPG |
| 930 | SGRPGRPGERGLPGPPGIK |
| 931 | SGRPGRPGERGLPGPPGIKGPA |
| 932 | SNGAPGQRGEPGPQGHAGAQGPPGPPGIN |
| 933 | SNGAPGQRGEPGPQGHAGAQGPPGPPGINGSPGGK |
| 934 | SNGAPGQRGEPGPQGHAGAQGPPGPPGINGSPGGKGEMGPA |
| 935 | SPGAKGEVGPAGSPGSNGAPG |
| 936 | SPGAKGEVGPAGSPGSNGAPGQ |
| 937 | SPGAKGEVGPAGSPGSNGAPGQRGE |
| 938 | SPGAKGEVGPAGSPGSNGAPGQRGEPG |
| 939 | SPGAKGEVGPAGSPGSNGAPGQRGEPGPQ |
| 940 | SPGAKGEVGPAGSPGSNGAPGQRGEPGPQG |
| 941 | SPGAKGEVGPAGSPGSNGAPGQRGEPGPQGH |
| 942 | SPGAKGEVGPAGSPGSNGAPGQRGEPGPQGHAG |
| 943 | SPGAKGEVGPAGSPGSNGAPGQRGEPGPQGHAGAQGPPGPPGIN |
| 944 | SPGEPGANGLPGAAGERGAPGFRGPAGPN |
| 945 | SPGGKGEMGPAGIPGAPGLM |
| 946 | SPGGKGEMGPAGIPGAPGLMGA |
| 947 | SPGGKGEMGPAGIPGAPGLMGAR |
| 948 | SPGGKGEMGPAGIPGAPGLMGARGPPGPAGAN |
| 949 | SPGGPGSDGKPGPPGSQGESGRPGPPG |
| 950 | SPGGPGSDGKPGPPGSQGESGRPGPPGP |
| 951 | SPGGPGSDGKPGPPGSQGESGRPGPPGPS |
| 952 | SPGGPGSDGKPGPPGSQGESGRPGPPGPSGPR |
| 953 | SPGGPGSDGKPGPPGSQGESGRPGPPGPSGPRG |
| 954 | SPGGPGSDGKPGPPGSQGESGRPGPPGPSGPRGQPGVM |
| 955 | SPGGPGSDGKPGPPGSQGESGRPGPPGPSGPRGQPGVMGFPGPKGN |
| 956 | SPGSNGAPGQRGEPGPQGHAGAQGPPG |
| 957 | SPGSNGAPGQRGEPGPQGHAGAQGPPGPPGIN |
| 958 | SPGSNGAPGQRGEPGPQGHAGAQGPPGPPGINGSPG |
| 959 | SPGSNGAPGQRGEPGPQGHAGAQGPPGPPGINGSPGGK |
| 960 | SPGSNGAPGQRGEPGPQGHAGAQGPPGPPGINGSPGGKGEMG |
| 961 | SPGSNGAPGQRGEPGPQGHAGAQGPPGPPGINGSPGGKGEMGPA |
| 962 | SPGSPGYQGPPGEPGQAGPSGPPGPPGAIGPS |
| 963 | SPGYQGPPGEPGQAGPSGPPGPPGAIGPS |
| 964 | SQGESGRPGPPGPSGPRGQPGVM |
| 965 | SQGESGRPGPPGPSGPRGQPGVMGFPGPKGN |
| 966 | TAGFPGSPGAKGEVGPA |
| 967 | TAGFPGSPGAKGEVGPAG |
| 968 | TAGFPGSPGAKGEVGPAGSPG |
| 969 | TAGFPGSPGAKGEVGPAGSPGSN |
| 970 | TGAPGLKGENGLPGENGAPGPMGPR |
| 971 | TGPQGPPGPTGPGGD |
| 972 | VGPAGSPGSNGAPGQRGEPGPQG |
| 973 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGSPGAKGEVGPAGSPGSNGAPGQRGE |
| | PGPQGHAGAQGPPGPPGINGSPGGKGEMGPAGIPGAPGLMGARGPPGPAGANG |
| | APGLRGGAGEPGKNGAKGEPGPRGERGEAGIPGVPGAKGEDGKDGSPGEPGAN |
| | GLPGAAGERGAPGFRGPAGPNGIPGEKGPAGERGAPGPAGPRGAAGEPGRDGV |
| | PGGPGMRGMPGSPGGPGSDGKPGPPGSQGESGRPGPPGPSGPRGQPGVMGFPG |
| | PKGNDGAPGKNGERGGPGGPGPQGPPGKNGETGPQGPPGPTGPGGDKGDTGPP |
| | GPQGLQGLPGTGGPPGENGKPGEPGPKGDAGAPGAPGGKGDAGAPGERGPPGL |
| | AGAPGLRGGAGPPGPEGGKGAAGPPGPPGAAGTPGLQGMPGERGGLGSPGPKG |
| | DKGEPGGPGADGVPGKDGPRGPTGPIGPPGPAGQPGDKGEGGAPGLPGIAGPR |
| | GSPGERGETGPPGPAGFPGAPGQNGEPGGKGERGAPGEKGEGGPPGVAGPPGG |
| | SGPAGPPGPQGVKGERGSPGGPGAAGFPGARGLPGPPGSNGNPGPPGPSGSPG |
| | KDGPPGPAGNTGAPGSPGVSGPKGDAGQPGEKGSPGAQGPPGAPGPLGIAGIT |
| | GARGLAGPPGMPGPRGSPGPQGVKGESGKPGANGLSGERGPPGPQGLPGLAGT |
| | AGEPGRDGNPGSDGLPGRDGSPGGKGDRGENGSPGAPGAPGHPGPPGPVGPAG |
| | KSGDRGESGPAGPAGAPGPAGSRGAPGPQGPRGDKGETGERGAAGIKGHRGFP |
| | GNPGAPGSPGPAGQQGAIGSPGPAGPRGPVGPSGPPGKDGTSGHPGPIGPPGP |
| | RGNRERGSEGSPGHPGQPGPPGPPGAPGPCCGGVGAAAIAGIGGEKAGGFAPY |
| | YG |
| 974 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGSPGAKGEVGPAGSPGSNGAPGQRGE |
| | PGPQGHAGAQGPPGPPGINGSPGGKGEMGPAGIPGAPGLMGARGPPGPAGANG |
| | APGLRGGAGEPGKNGAKGEPGPRGERGEAGIPGVPGAKGEDGKDGSPGEPGAN |
| | GLPGAAGERGAPGFRGPAGPNGIPGEKGPAGERGAPGPAGPRGAAGEPGRDGV |
| | PGGPGMRGMPGSPGGPGSDGKPGPPGSQGESGRPGPPGPSGPRGQPGVMGFPG |
| | PKGNDGAPGKNGERGGPGGPGPQGPPGKNGETGPQGPPGPTGPGGDKGDTGPP |
| | GPQGLQGLPGTGGPPGENGKPGEPGPKGDAGAPGAPGGKGDAGAPGERGPPAI |
| | AGIGGEKAGGFAPYYG |
| 975 | DVKSGVAVGGLAGYPGPAGAPGPPGPPGASGHPGSPGAPGYQGPPGAPGQAGP |
| | SGAPGPPGAIGASGPAGKDGASGRPGRPGERGLPGPPGAKGPAGIPGAPGMKG |
| | HRGFDGRNGEKGATGAPGLKGANGLPGENGAPGPMGPRGAPGERGRPGAPGAA |
| | GARGADGARGSDGAPGPPGPPGAAGFPGSPGAKGEVGPAGAPGSNGAPGARGE |
| | PGPQGAAGAQGPPGAPGINGSPGAKGEMGPAGAPGAPGLMGARGPPGPAGANG |
| | APGLRGAAGEPGKNGAKGEPGPRGARGEAGIPGAPGAKGEDGADGSPGEPGAN |
| | GLPGAAGARGAPGFRGAAGPNGIPGAKGPAGERGAPGPAGPRGAAGEPGRDGA |
| | PGGPGMRGAPGSPGGPGADGKPGPPGAQGESGRPGAPGPSGPRGAPGVMGFPG |
| | AKGNDGAPGANGERGGPGAPGPQGPPGANGETGPQGAPGPTGPGGAKGDTGPP |
| | GAQGLQGLPGAGGPPGENGAPGEPGPKGAAGAPGAPGAKGDAGAPGERGPPAI |
| | AGIGGEKAGGFAPYYG |
| 976 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGSPGAKGEVGPAGSPGSNGAPGQRGE |
| | PGPQGHAGAQGPPGPPGINGSPGGKGEMGPAGIPGAPGLMGARGPPGPAGANG |
| | APGLRGGAGEPGKNGAKGEPGPRGERGEAGIPGVPGAKGEDGKDGSPGEPGAN |
| | GLPGAAGERGAPGFRGPAGPNGIPGEKGPAGERGAPGPAGPRGAAGEPGRDGE |
| | PGLMGPRGLPGSPGGPGPAGKEGPVGLPGIDGRPGPIGPAGARGEPGVMGFPG |
| | PKGNDGDPGKNGDKGHAGLAGARGAPGPDGETGAQGPPGPQGVQGGKGEQGPA |
| | GPPGFQGLPGTGGPAGEVGKPGEQGLHGEFGLPGPAGPRGERGAPGERGPPAI |
| | AGIGGEKAGGFAPYYG |
| 977 | DVKSGVAVGGLAGYPGPAGPPGPPGPPGTSGHPGSPGSPGYQGPPGEPGQAGP |
| | SGPPGPPGAIGPSGPAGKRGESGRPGRPGERGLPGPPGIKGPAGIPGFPGMKG |
| | HRGFRGRNGEKGETGAPGLKGENGLPGENGAPGPMGPRGAPGERGRPGLPGAA |
| | GARGNRGARGSDGQPGPPGPPGTAGFPGSPGAKGRVGPAGSPGSNGAPGQRGE |
| | PGPQGHAGAQGPPGPPGINGSPGGKGEMGPAGIPGAPGLMGARGPPGPAGANG |
| | APGLRGGAGRPGKNGAKGEPGPRGERGRAGIPGVPGAKGERGKDGSPGEPGAN |
| | GLPGAAGERGAPGFRGPAGPNGIPGEKGPAGERGAPGPAGPRGAAGEPGRRGV |
| | PGGPGMRGMPGSPGGPGSRGKPGPPGSQGESGRPGPPGPSGPRGQPGVMGFPG |
| | PKGNDGAPGKNGERGGPGGPGPQGPPGKNGETGPQGPPGPTGPGGRKGDTGPP |
| | GPQGLQGLPGTGGPPGENGKPGRPGPKGDAGAPGAPGGKGRAGAPGERGPPAI |
| | AGIGGEKAGGFAPYYG |
| 978 | DVKSGVAVGGLAGYPGPAGPPGPPGPPGASGHPGSPGSPGYQGPPGEPGQAG |
| | PAGPPGPPGAIGPSGPAGKDGESGRPGRPGERGLPGPPGIKGPAGIPGFPGMK |
| | GHRGFDGRNGEKGEAGAPGLKGENGLPGENGAPGPMGPRGAPGERGRPGLPGA |
| | AGARGNDGARGSDGQPGPPGPPGAAGFPGAPGAKGEVGPAGSPGSNGAPGQRG |
| | EPGPQGHAGAQGPPGPPGINGSPGGKGEMGPAGIPGAPGLMGARGPPGPAGAN |
| | GAPGLRGGAGEPGKNGAKGEPGPRGERGEAGIPGVPGAKGEDGKDGSPGEPGA |
| | NGLPGAAGERGAPGFRGPAGPNGIPGEKGPAGERGAPGPAGPRGAAGEPGRDG |
| | VPGGPGMRGMPGSPGGPGSDGKPGPPGAQGESGRPGPPGPSGPRGQPGVMGFP |
| | GPKGNDGAPGKNGERGGPGGPGPQGPPGKNGETGPQGPPGPTGPGGDKGDTGP |
| | PGPQGLQGLPGTGGPPGENGKPGEPGPKGDAGAPGAPGGKGDAGAPGERGPPA |
| | IAGIGGEKAGGFAPYYG |
| 979 | DVKSGVAVGGLAGYPGPAGPPGPPGPPGNSGHPGSPGSPGYQGPPGEPGQAGP |
| | NGPPGPPGAIGPSGPAGKDGESGRPGRPGERGLPGPPGIKGPAGIPGFPGMKG |
| | HRGFDGRNGEKGENGAPGLKGENGLPGENGAPGPMGPRGAPGERGRPGLPGAA |
| | GARGNDGARGSDGQPGPPGPPGNAGFPGNPGAKGEVGPAGSPGSNGAPGQRGE |
| | PGPQGHAGAQGPPGPPGINGSPGGKGEMGPAGIPGAPGLMGARGPPGPAGANG |
| | APGLRGGAGEPGKNGAKGEPGPRGERGEAGIPGVPGAKGEDGKDGSPGEPGAN |
| | GLPGAAGERGAPGFRGPAGPNGIPGEKGPAGERGAPGPAGPRGAAGEPGRDGV |
| | PGGPGMRGMPGSPGGPGSDGKPGPPGNQGESGRPGPPGPSGPRGQPGVMGFPG |
| | PKGNDGAPGKNGERGGPGGPGPQGPPGKNGETGPQGPPGPTGPGGDKGDTGPP |
| | GPQGLQGLPGTGGPPGENGKPGEPGPKGDAGAPGAPGGKGDAGAPGERGPPAI |
| | AGIGGEKAGGFAPYYG |
| 980 | DVKSGVAVGGLAGYPGPAGAPGPPGPPGASGHPGSPGAPGYQGPPGAPGQAGP |
| | SGAPGPPGAIGASGPAGKDGASGRPGRPGERGLPGPPGAKGPAGIPGAPGMKG |
| | HRGFDGRNGEKGATGAPGLKGANGLPGENGAPGPMGPRGAPGERGRPGAPGAA |
| | GARGADGARGSDGAPGPPGPPGAAGFPGSPGAKGEVGPAGAPGSNGAPGARGE |
| | PGPQGAAGAQGPPGAPGINGSPGAKGEMGPAGAPGAPGLMGARGPPGPAGANG |
| | APGLRGAAGEPGKNGAKGEPGPRGARGEAGIPGAPGAKGEDGADGSPGEPGAN |
| | GLPGAAGARGAPGFRGAAGPNGIPGAKGPAGERGAPGPAGPRGAAGEPGRDGA |
| | PGGPGMRGAPGSPGGPGADGKPGPPGAQGESGRPGAPGPSGPRGAPGVMGFPG |
| | AKGNDGAPGANGERGGPGAPGPQGPPGANGETGPQGAPGPTGPGGAKGDTGPP |
| | GAQGLQGLPGAGGPPGENGAPGEPGPKGAAGAPGAPGAKGDAGAPGERGPPAI |
| | AGIGGEKAGGFAPYYG |
| 981 | DSYDVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFPG |
| | MKGHRGFDGRNGEKGETGAPGLKGENGLPGENGAPGPMGPRGAPGERGRPGLP |
| | GAAGARGNDGARGSDGQPGPPGPPGTAGFPGSPGAKGEVGPAGSPGSNGAPGQ |
| | RGEPGPQGHAGAQGPPGPPGINGSPGGKGEMGPAGIPGAPGLMGARGPPGPAG |
| | ANGAPGLRGGAGEPGKNGAKGEPGPRGERGEAGIPGVPGAKGEDGKDGSPGEP |
| | GANGLPGAAGERGAPGFRGPAGPNGIPGEKGPAGERGAPGPAGPRGAAGEPGR |
| | DGVPGGPGMRGMPGSPGGPGSDGKPGPPGSQGESGRPGPPGPSGPRGQPGVMG |
| | FPGPKGNDGAPGKNGERGGPGGPGPQGPPGKNGETGPQGPPGPTGPGGDKGDT |
| | GPPGPQGLQGLPGTGGPPGENGKPGEPGPKGDAGAPGAPGGKGDAGAPGERGP |
| | PAIAGIGGEKAGGFAPYYG |
| 982 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGAIGPSGPAGKDGESGRPGRPGERGLPGPPGI |
| | KGPAGIPGFPGMKGHRGFDGRNGEKGETGAPGLKGENGLPGENGAPGPMGPRG |
| | APGERGRPGLPGAAGARGNDGARGSDGQPGPPGPPAIAGIGGEKAGGFAPYYG |
| 983 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGAIGPSGPAGKDGESGRPGRPGERGLPGPPGI |
| | KGPAGIPGFPGMKGHRGFDGRNGEKGETGAPGLKGENGLPGENGAPGPMGPRG |
| | APGERGRPGLPGAAGARGNDGARGSDGQPGPPGPPAIAGIGGEKAGGFAPYYG |
| 984 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGAIGPSGPAGKDGESGRPGRPGERGLPGPPGI |
| | KGPAGIPGFPGMKGHRGFDGRNGEKGETGAPGLKGENGLPGENGAPGPMGPRG |
| | APGERGRPGLPGAAGARGNDGARGSDGQPGPPGPPGAIGPSGPAGKDGESGRP |
| | GRPGERGLPGPPGIKGPAGIPGFPGMKGHRGFDGRNGEKGETGAPGLKGENGL |
| | PGENGAPGPMGPRGAPGERGRPGLPGAAGARGNDGARGSDGQPGPPGPPAIAG |
| | IGGEKAGGFAPYYG |
| 985 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGSPGAKGEVGPAGSPGSNGAPGQRGE |
| | PGPQGHAGAQGPPGPPGINGSPGGKGEMGPAGIPGAPGLMGARGPPGPAGANG |
| | APGLRGGAGEPGKNGAKGEPGPRGERGEAGIPGVPGAKGEDGKDGSPGEPGAN |
| | GLPGAAGERGAPGFRGPAGPNGIPGEKGPAGERGAPGPAGPRGAAGEPGRDGV |
| | PGGPGMRGMPGSPGGPGSDGKPGPPGSQGESGRPGPPGPSGPRGQPGVMGFPG |
| | PKGHRGFPGKNGERGGPGGPGPQGPPGKNGETGPQGPPGPTGPGGDKGDTGPP |
| | GPQGLQGLPGTGGPPGENGKPGEPGPKGDAGAPGAPGGKGDAGAPGERGPPAI |
| | AGIGGEKAGGFAPYYG |
| 986 | DVKSGVAVGGLAGYPGPAGPPGPPGPPGTSGHPGSPGSPGYQGPPGEPGQAGP |
| | SGPPGPPGAIGPSGPAGKDGESGRPGRPGAAGLPGPPGIKGPAGIPGFPGMKG |
| | HRGFDGRNGEKGETGAPGLKGENGLPGAAGAPGPMGPRGAPGAAGRPGLPGAA |
| | GARGNDGARGSDGQPGPPGPPGTAGFPGSPGAKGEVGPAGSPGSNGAPGQRGE |
| | PGPQGHAGAQGPPGPPGINGSPGGKGEMGPAGIPGAPGLMGARGPPGPAGANG |
| | APGLRGGAGEPGKNGAKGEPGPRGERGEAGIPGVPGAKGEDGKDGSPGEPGAN |
| | GLPGAAGERGAPGFRGPAGPNGIPGEKGPAGERGAPGPAGPRGAAGEPGRDGV |
| | PGGPGMRGMPGSPGGPGSDGKPGPPGSQGESGRPGPPGPSGPRGQPGVMGFPG |
| | PKGNDGAPGKNGERGGPGGPGPQGPPGKNGETGPQGPPGPTGPGGDKGDTGPP |
| | GPQGLQGLPGTGGPPGENGKPGEPGPKGDAGAPGAPGGKGDAGAPGPLVLRQL |
| | LVLVEKRPAVSLHIMVNQEDVRMPFA |
| 987 | DVKSGVAVGGLAGYPGPAGPPGPPGPPGTSGHPGSPGSPGYQGPPGEPGQAGP |
| | SGPPGPPGAIGPSGPAGKDGESGKPGRPGERGLPGPPGIKGPAGIPGFPGMKG |
| | HRGFDGKNGEKGETGAPGLKGENGLPGENGAPGPMGPKGAPGERGKPGLPGAA |
| | GAKGNDGAKGSDGQPGPPGPPGTAGFPGSPGAKGEVGPAGSPGSNGAPGQKGE |
| | PGPQGHAGAQGPPGPPGINGSPGGKGEMGPAGIPGAPGLMGAKGPPGPAGANG |
| | APGLKGGAGEPGKNGAKGEPGPKGEKGEAGIPGVPGAKGEDGKDGSPGEPGAN |
| | GLPGAAGEKGAPGFKGPAGPNGIPGEKGPAGEKGAPGPAGPKGAAGEPGKDGV |
| | PGGPGMKGMPGSPGGPGSDGKPGPPGSQGESGKPGPPGPSGPKGQPGVMGFPG |
| | PKGNDGAPGKNGEKGGPGGPGPQGPPGKNGETGPQGPPGPTGPGGDKGDTGPP |
| | GPQGLQGLPGTGGPPGENGKPGEPGPKGDAGAPGAPGGKGDAGAPGERGPPAI |
| | AGIGGEKAGGFAPYYG |
| 988 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGAVGAKGEAGPQGPRGSEGPQGVRGE |
| | PGPPGPAGAAGPAGPPGADGQPGAKGAMGAPGIAGAPGFPGARGPPGPQGAGG |
| | PPGPKGGAGEPGAPGAKGDTGAKGERGPVGVQGPPGPAGEEGKRGARGEPGPT |
| | GLPGPPGERGGPGFRGFPGADGIPGEKGPAGERGAPGPAGPRGAAGEPGRDGV |
| | PGGPGMRGMPGSPGGPGSDGKPGPPGSQGESGRPGPPGPSGPRGQPGVMGFPG |
| | PKGNDGAPGKNGERGGPGGPGPQGPPGKNGETGPQGPPGPTGPGGDKGDTGPP |
| | GPQGLQGLPGTGGPPGENGKPGEPGPKGDAGAPGAPGGKGDAGAPGERGPPAI |
| | AGIGGEKAGGFAPYYG |
| 989 | DVKSGVAVGGIAGYPGPAGPPGPPGPPGTSGHPGSPGSPGYQGPPGEPGQAGP |
| | SGPPGPPGALGPSGPAGKDGESGRPGRPGERGVPGPPGVKGPAGLPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGSPGAKGELGPAGSPGSNGAPGQRGE |
| | PGPQGHAGAQGPPGPPGLNGSPGGKGEMGPAGVPGAPGIMGARGPPGPAGANG |
| | APGIRGGAGEPGKNGAKGEPGPRGERGEAGLPGLPGAKGEDGKDGSPGEPGAN |
| | GVPGAAGERGAPGFRGPAGPNGLPGEKGPAGERGAPGPAGPRGAAGEPGRDGI |
| | PGGPGMRGMPGSPGGPGSDGKPGPPGSQGESGRPGPPGPSGPRGQPGLMGFPG |
| | PKGNDGAPGKNGERGGPGGPGPQGPPGKNGETGPQGPPGPTGPGGDKGDTGPP |
| | GPQGVQGLPGTGGPPGENGKPGEPGPKGDAGAPGAPGGKGDAGAPGERGPPAI |
| | AGIGGEKAGGFAPYYG |
| 990 | DVKSGVAVGGLAGYPGPAGPPGPPGPPGAAGHPGAPGAPGYQGPPGEPGQAGP |
| | AGPPGPPGAIGPAGPAGKDGEAGRPGRPGERGLPGPPGIKGPAGIPGFPGMKG |
| | HRGFDGRDGEKGEAGAPGLKGEDGLPGENGAPGPMGPRGAPGERGRPGLPGAA |
| | GARGDDGARGADGQPGPPGPPGAAGFPGAPGAKGEVGPAGAPGADGAPGQRGE |
| | PGPQGHAGAQGPPGPPGIDGAPGGKGEMGPAGIPGAPGLMGARGPPGPAGADG |
| | APGLRGGAGEPGKDGAKGEPGPRGERGEAGIPGVPGAKGEDGKDGAPGEPGAD |
| | GLPGAAGERGAPGFRGPAGPDGIPGEKGPAGERGAPGPAGPRGAAGEPGRDGV |
| | PGGPGMRGMPGAPGGPGADGKPGPPGAQGEAGRPGPPGPAGPRGQPGVMGFPG |
| | PKGDDGAPGKDGERGGPGGPGPQGPPGKDGEAGPQGPPGPAGPGGDKGDAGPP |
| | GPQGLQGLPGAGGPPGEDGKPGEPGPKGDAGAPGAPGGKGDAGAPGERGPPAI |
| | AGIGGEKAGGFAPYYG |
| 991 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGSPGAKGEVGPAGSPGSNGAPGQRGE |
| | PGPQGHAGAQGPPGPPGINGSPGGKGEMGPAGIEPGRPEGEKGGGRGGAPGAP |
| | GRGAGNGEGARPPPLMGGAGGNKAGPLAAPIPGVPGAKGEDGKDGSPGEPGAN |
| | GLPGAAGERGAPGFRGPAGPNGIPGEKGPAGERGAPGPAGPRGAAGEPGRDGV |
| | PGGPGMRGMPGSPGGPGSDGKPGPPGSQGESGRPGPPGPSGPRGQPGVMGFPG |
| | PKGNDGAPGKNGERGGPGGPGPQGPPGKNGETGPQGPPGPTGPGGDKGDTGPP |
| | GPQGLQGLPGTGGPPGENGKPGEPGPKGDAGAPGAPGGKGDAGAPGERGPPAI |
| | AGIGGEKAGGFAPYYG |
| 992 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGSPGAKGEVGPAGSPGSNGAPGQRGE |
| | PGPQGHAGAQGPPGPPGINGSPGGKGEMGPAGIEPGRPEGEKGGGRGGAPGAP |
| | GRGAGNGEGARPPPLMGGAGGNKAGPLAAPIPGVPGAKGEDGKDGSPGEPGAN |
| | GLPGAAGERGAPGFRGPAGPNGIPGEKGPAGERGAPGPAGPRGAAGEPGRDGV |
| | PGGPGMRGMPGSPGGPGSDGKPGPPGSQGESGRPGPPGPSGPRGQPGVMGFPG |
| | PKGNDGAPGKNGERGGPGGPGPQGPPGKNGETGPQGPPGPTGPGGDKGDTGPP |
| | GPQGLQGLPGTGGPPGENGKPGEPGPKGDAGAPGAPGGKGDAGAPGERGPPAI |
| | AGIGGEKAGGFAPYYG |
| 993 | DVKSGVAVGGLAGYPGPAGPPGPPGPPGTSGHPGSPGSPGYQGPPGEPGQAGP |
| | SGPPGPPGAIGPSGPAGKRGESGRPGRPGERGLPGPPGIKGPAGIPGFPGMKG |
| | HRGFRGRNGEKGRTGAPGLKGENGLPGENGAPGPMGPRGAPGERGRPGLPGAA |
| | GARGNRGARGSRGQPGPPGPPGTAGFPGSPGAKGRVGPAGSPGSNGAPGQRGR |
| | PGPQGHAGAQGPPGPPGINGSPGGKGRMGPAGIPGAPGLMGARGPPGPAGANG |
| | APGLRGGAGRPGKNGAKGRPGPRGERGRAGIPGVPGAKGRRGKRGSPGEPGAN |
| | GLPGAAGERGAPGFRGPAGPNGIPGEKGPAGERGAPGPAGPRGAAGRPGRRGV |
| | PGGPGMRGMPGSPGGPGSRGKPGPPGSQGESGRPGPPGPSGPRGQPGVMGFPG |
| | PKGNRGAPGKNGRRGGPGGPGPQGPPGKNGETGPQGPPGPTGPGGRKGRTGPP |
| | GPQGLQGLPGTGGPPGENGKPGRPGPKGDAGAPGAPGGKGRAGAPGERGPPAI |
| | AGIGGEKAGGFAPYYG |
| 994 | DVKSGVAVGGMGGYPGPAGPPGPPGPPGVSGHPGAPGAPGYQGPPGEPGQAGP |
| | AGPPGPPGAMGPAGPAGKDGESGRPGRPGERGFPGPPGIKGPAGMPGFPGMKG |
| | HRGFDGRNGEKGDTGAPGLKGENGLPGENGAPGPMGPRGAPGERGRPGLPGAA |
| | GARGNDGARGSDGQPGPPGPPGTAGFPGSPGAKGEVGPAGAPGPNGAPGQRGE |
| | PGLQGQAGAPGPPGPPGINGSPGGKGEMGPAGIPGAPGLIGARGPPGPPGANG |
| | VPGQRGAAGEPGKNGAKGDPGARGERGEAGIPGIAGPKGEDGKDGSPGEPGAN |
| | GLPGAPGERGPPGFRGAPGANGIPGEKGPPGERGGPGPAGPRGVAGEPGRDGL |
| | PGGPGLRGIPGSPGGPGSDGKPGPPGSQGESGRPGPPAHLVQGVSLVSWVSPV |
| | QRVMTEHRVKMVNVVAQVVQDCPVQQEKTVRQVLKDLQDLPVLQEIREMQDRQ |
| | DLLDCKVCLVQEALPEKMVSLESQDQKAMSEPQVFPEVRETPVLRVSVVLRQL |
| | LVLVEKRPAVSLHIMVNQEDVRMPFA |
| 995 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGSPGAKGEVGPAGSPGSNGAPGQRGE |
| | PGPQGHAGAQGPPGPPGINGSPGGKGEMGPAGIPGAPGLMGARGPPGPAGANG |
| | APGLRGGAGEPGKNGAKGEPGPRGERGEAGIPGVPGAKGEDGKDGSPGEPGAN |
| | GLPGAAGERGAPGFRGPAGPNGVAGPKGPAGERGAPGPAGPKGAAGEAGRPGE |
| | AGLPGAKGMPGSPGGPGPDGKPGPPGPAGQDGRPGPPGPPGARGQAGVMGFPG |
| | PKGAAGEPGKAGERGVPGPPGAVGPAGKDGEAGAQGPPGPAGPAGERGEQGPA |
| | GPQGFQGLPGPAGPPGEAGKPGEQGVPGDLGAPGAPGARGERGAPGERGPPAI |
| | AGIGGEKAGGFAPYYG |
| 996 | DSYDVKSGVAVGGLAGYPGPAGPPGPPGPPGTSGHPGSPGSPGYQGPPGEPGQ |
| | AGPSGPPGPPGAIGPSGPAGKDGESGRPGRPGERGLPGPPGIKGPAGIPGFPG |
| | MKGHRGFDGRNGEKGETGAPGLKGENGLPGENGAPGPMGPRGAPGERGRPGLP |
| | GAAGARGNDGARGSDGQPGPPGPPGTAGFPGSPGAKGEVGPAGSPGSNGAPGQ |
| | RGEPGPQGHAGAQGPPGPPGINGSPGGKGEMGPAGIPGAPGLMGARGPPGPAG |
| | ANGAPGLRGGAGEPGKNGAKGEPGPRGERGEAGIPGVPGAKGEDGKDGSPGEP |
| | GANGLPGAAGERGAPGFRGPAGPNGIPGEKGPAGERGAPGPAGPRGAAGEPGR |
| | DGVPGGPGMRGMPGSPGGPGSDGKPGPPGSQGESGRPGPPGPSGPRGQPGVMG |
| | FPGPKGNDGAPGKNGERGGPGGPGPQGPPGKNGETGPQGPPGPTGPGGDKGDT |
| | GPPGPQGLQGLPGTGGPPGENGKPGEPGPKGDAGAPGAPGGKGDAGAPGERGP |
| | P |
| 997 | DVKSGVAVGKTTKSGLAGYPGPAGPPGPPGPPGTSGHPGSPGSPGYQGPPGEP |
| | GQAGPSGPPGPPGAIGPSGPAGKDGESGRPGRPGERGLPGPPGIKGPAGIPGF |
| | PGMKGHRGFDGRNGEKGETGAPGLKGENGLPGENGAPGPMGPRGAPGERGRPG |
| | LPGAAGARGNDGARGSDGQPGPPGPPGTAGFPGSPGAKGEVGPAGSPGSNGAP |
| | GQRGEPGPQGHAGAQGPPGPPGINGSPGGKGEMGPAGIPGAPGLMGARGPPGP |
| | AGANGAPGLRGGAGEPGKNGAKGEPGPRGERGEAGIPGVPGAKGEDGKDGSPG |
| | EPGANGLPGAAGERGAPGFRGPAGPNGIPGEKGPAGERGAPGPAGPRGAAGEP |
| | GRDGVPGGPGMRGMPGSPGGPGSDGKPGPPGSQGESGRPGPPGPSGPRGQPGV |
| | MGFPGPKGNDGAPGKNGERGGPGGPGPQGPPGKNGETGPQGPPGPTGPGGDKG |
| | DTGPPGPQGLQGLPGTGGPPGENGKPGEPGPKGDAGAPGAPGGKGDAGAPGER |
| | GPPKTTKSAIAGIGGEKAGGFAPYYG |
| 998 | DVKSGVAVGKTTKSGLAGYPGPAGPPGPPGPPGTSGHPGSPGSPGYQGPPGEP |
| | GQAGPSGPPGPPGAIGPSGPAGKDGESGRPGRPGERGLPGPPGIKGPAGIPGF |
| | PGMKGHRGFDGRNGEKGETGAPGLKGENGLPGENGAPGPMGPRGAPGERGRPG |
| | LPGAAGARGNDGARGSDGQPGPPGPPGTAGFPGSPGAKGEVGPAGSPGSNGAP |
| | GQRGEPGPQGHAGAQGPPGPPGINGSPGGKGEMGPAGIPGAPGLMGARGPPGP |
| | AGANGAPGLRGGAGEPGKNGAKGEPGPRGERGEAGIPGVPGAKGEDGKDGSPG |
| | EPGANGLPGAAGERGAPGFRGPAGPNGIPGEKGPAGERGAPGPAGPRGAAGEP |
| | GRDGVPGGPGMRGMPGSPGGPGSDGKPGPPGSQGESGRPGPPGPSGPRGQPGV |
| | MGFPGPKGNDGAPGKNGERGGPGGPGPQGPPGKNGETGPQGPPGPTGPGGDKG |
| | DTGPPGPQGLQGLPGTGGPPGENGKPGEPGPKGDAGAPGAPGGKGDAGAPGER |
| | GPPKTTKSAIAGIGGEKAGGFAPYYG |
| 999 | DVKSGVAVGKTTKSGLAGYPGPAGPPGPPGPPGTSGHPGSPGSPGYQGPPGEP |
| | GQAGPSGPPGPPGAIGPSGPAGKDGESGRPGRPGAAGLPGPPGIKGPAGIPGF |
| | PGMKGHRGFDGRNGEKGETGAPGLKGENGLPGAAGAPGPMGPRGAPGAAGRPG |
| | LPGAAGARGNDGARGSDGQPGPPGPPGTAGFPGSPGAKGEVGPAGSPGSNGAP |
| | GQRGEPGPQGHAGAQGPPGPPGINGSPGGKGEMGPAGIPGAPGLMGARGPPGP |
| | AGANGAPGLRGGAGEPGKNGAKGEPGPRGERGEAGIPGVPGAKGEDGKDGSPG |
| | EPGANGLPGAAGERGAPGFRGPAGPNGIPGEKGPAGERGAPGPAGPRGAAGEP |
| | GRDGVPGGPGMRGMPGSPGGPGSDGKPGPPGSQGESGRPGPPGPSGPRGQPGV |
| | MGFPGPKGNDGAPGKNGERGGPGGPGPQGPPGKNGETGPQGPPGPTGPGGDKG |
| | DTGPPGPQGLQGLPGTGGPPGENGKPGEPGPKGDAGAPGAPGGKGDAGAPGAA |
| | GPPKTTKSAIAGIGGEKAGGFAPYYG |
| 1000 | DVKSGVAVGGLAGYPGPAGPPGPPGPPGTSGHPGSPGSPGYQGPPGEPGQAGP |
| | SGPPGPPGAIGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGSPGAKGEVGPAGSPGSNGAPGQRGE |
| | PGPQGHAGAQGPPGPPGINGSPGGKGEMGPAGIPGAPGLMGARGPPGPAGANG |
| | APGLRGGAGEPGKNGAKGEPGPRGERGEAGIPGVPGAKGEEGKRGARGEAGSA |
| | GPPGPPGLRGAPGFRGLPGADGRAGVMGPPGERGAPGPAGVRGAAGDAGRPGE |
| | PGLMGPRGLPGSPGGPGPAGKEGPVGLPGIDGRPGPIGPAGARGEPGVMGFPG |
| | PKGNDGDPGKNGDKGHAGLAGARGAPGPDGETGPQGPPGPTGPGGDKGDTGPP |
| | GPQGLQGLPGTGGPPGENGKPGEPGPKGDAGAPGAPGGKGDAGAPGERGPPAI |
| | AGIGGEKAGGFAPYYG |
| 1001 | DVKSGVAVGGMGGYPGPAGPPGPPGPPGVSGHPGAPGAPGYQGPPGEPGQAGP |
| | AGPPGPPGAMGPAGPAGKDGESGRPGRPGERGFPGPPGIKGPAGMPGFPGMKG |
| | HRGFDGRNGEKGDTGAPGLKGENGLPGENGAPGPMGPRGAPGERGRPGLPGAA |
| | GARGNDGARGSDGQPGPPGPPGTAGFPGSPGAKGEVGPAGAPGPNGAPGQRGE |
| | PGLQGQAGAPGPPGPPGINGSPGGKGEMGPAGIPGAPGLIGARGPPGPPGANG |
| | VPGQRGAAGEPGKNGAKGDPGARGERGEAGIPGIAGPKGEDGKDGSPGEPGAN |
| | GLPGAPGERGPPGFRGAPGANGIPGEKGPPGERGGPGPAGPRGVAGEPGRDGL |
| | PGGPGLRGIPGSPGGPGSDGKPGPPGSQGESGRPGPPGSPGPRGQPGVMGFPG |
| | PKGNDGAPGKNGERGGPGGPGLPGPAGKNGETGPQGPPGPTGPAGDKGDAGPP |
| | GPPGLQGLPGTGGPPGENGKPGEPGPKGDVGAPGIPGGKGDAGAPGERGPPAI |
| | AGIGGEKAGGFAPYYG |
| 1002 | DVKSGVAVGGLAGYPGPAGPPGPPGPPGTSGHPGSPGSPGYQGPPGEPGQAGP |
| | AGPPGPIGNVGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPGPPGPPGTAGFPGSPGAKGEVGPAGSPGSNGAPGQRGE |
| | PGPQGHAGAQGPPGPPGINGSPGGKGEMGPAGIPGAPGLMGARGPPGPAGANG |
| | APGLRGGAGEPGKNGAKGEPGPRGERGEAGIPGVPGAKGEDGKDGSPGEPGAN |
| | GLPGAAGERGAPGFRGPAGPNGIPGEKGPAGERGAPGPAGPRGAAGEPGRDGV |
| | PGGPGMRGMPGSPGGPGSDGKPGPPGSQGESGRPGPPGPSGPRGQPGVMGFPG |
| | PKGNDGAPGKNGERGGPGGPGPQGPPGKNGETGPQGPPGPTGPGGDKGDTGPP |
| | GPQGLQGLPGTGGPPGENGKPGEPGPKGDAGAPGAPGGKGDAGAPGERGPPAI |
| | AGIGGEKAGGFAPYYG |
| 1007 | DVKSGVAVGGLAGYPGPAGPPGPPGPPGTSGHKGSPGSPGYQGPPGEPGQAGP |
| | AGPPGPIGNVGPSGPAGKDGESGRPGRPGERGLPGPPGIKGPAGIPGFPGMKG |
| | HRGFDGRNGEKGETGAPGLKGENGLPGENGAPGPMGPRGAPGERGRPGLPGAA |
| | GARGNDGARGSDGQPRPPGPPGTAGFPGSPGAKGEVGPAGSPGSNGAPGQRGE |
| | PGPQGHKGAQGPPGPPGINGSPGGKGEMGPAGIPGAPGLMGARGPPGPAGANG |
| | APGLRGGAGEPGKNGAKGEPGPRGERGEAGIPGVPGAKGEDGKDGSPGEPGAN |
| | GLPGAAGERGAPGFRGPAGPNGIPGEKGPAGERGAPGPAGPRGAAGEPGRDGV |
| | PGGPGMRGMPGSPGGPGSDGKPGPPGSQGESGRPGPPGPSGPRGQPGVMGFPG |
| | PKGNDGAPGKNGERGGPGGPGPQGPPGKNGETGPQGPPGPTGPGGDKGDTGPP |
| | GPQGLQGLPGTGGPPGENGKPGEPGPKGDAGAPGAPGGKGDAGAPGERGPPAI |
| | AGIGGEKAGGFAPYYG |
| 1008 | DSYDVKSGVAVGGLAGAPGLRGGAGPPGPEGGKGAAGPPGPPGAAGTPGLQGM |
| | PGERGGLGSPGPKGDKGEPGGPGADGVPGKDGPRGPTGPIGPPGPAGQPGDKG |
| | EGGAPGLPGIAGPRGSPGERGETGPPGPAGFPGAPGQNGEPGGKGERGAPGEK |
| | GEGGPPGVAGPPGGSGPAGPPGPQGVKGERGSPGGPGAAGFPGARGLPGPPGS |
| | NGNPGPPGPSGSPGKDGPPGPAGNTGAPGSPGVSGPKGDAGQPGEKGSPGAQG |
| | PPGAPGPLGIAGITGARGLAGPPGMPGPRGSPGPQGVKGESGKPGANGLSGER |
| | GPPGPQGLPGLAGTAGEPGRDGNPGSDGLPGRDGSPGGKGDRGENGSPGAPGA |
| | PGHPGPPGPVGPAGKSGDRGESGPAGPAGAPGPAGSRGAPGPQGPRGDKGETG |
| | ERGAAGIKGHRGFPGNPGAPGSPGPAGQQGAIGSPGPAGPRGPVGPSGPPGKD |
| | GTSGHPGPIGPPGPRGNRGERGSEGSPGHPGQPGPPGPPGAPGPCCGGVGAAA |
| | IAGIGGEKAGGFAPYYG |

### C. Hyaluronic Acid

Hyaluronic acid (HA) is a natural, linear carbohydrate polymer belonging to the class of non-sulfated glycosaminoglycans. It is composed of beta-1,3-N-acetyl glucosamine and beta-1,4-glucuronic acid repeating disaccharide units with a molecular weight (MW) up to 6 MDa. HA is present in hyaline cartilage, synovial joint fluid, and skin tissue, both dermis and epidermis. HA can be extracted from natural tissues including the connective tissue of vertebrates or prepared by microbiological methods.

Numerous roles of HA in the body have been identified. It plays an important role in the biological organism, as a mechanical support for the cells of many tissues, such as the skin, tendons, muscles and cartilage. HA is involved in key biological processes, such as the moistening of tissues, and lubrication. It is also suspected of having a role in numerous physiological functions, such as adhesion, development, cell motility, cancer, angiogenesis, and wound healing. Due to the unique physical and biological properties of HA including viscoelasticity, biocompatibility, biodegradability, HA is employed in a wide range of current and developing applications within cosmetics, aesthetics, skincare, opthalmology, rheumatology, drug delivery, wound healing, and tissue engineering. The use of HA in some of these applications is limited by the fact that HA is soluble in water at room temperature, i.e. about 20° C., it is rapidly degraded by hyaluronidase in the body, and it is difficult to process into biomaterials. Crosslinking of HA has therefore been introduced to improve the physical and mechanical properties of HA and its in vivo residence time. Crosslinking of HA can be described as monophasic or biphasic. A monophasic structure is when there are cross-links formed between HA chains, which makes a uniform gel-like structure. A biphasic structure is when the HA is made to be gel-like as well as having free HA particles within the mixture which are not part of the crossed structure. This type of structure is not as smooth as monophasic but historically gives more volume.

U.S. Pat. No. 4,582,865, which is incorporated by reference in its entirety, describes methods for preparing crosslinked gels of HA, alone, or mixed with other hydrophilic polymers, using divinyl sulfone (DVS) as the crosslinking agent. The preparation of a crosslinked HA or salt thereof using a polyfunctional epoxy compound is disclosed in EP 0 161 887 B1, which is also incorporated by reference in its entirety. Other bi- or poly-functional reagents that have been employed to crosslink HA through covalent linkages include formaldehyde (see U.S. Pat. No. 4,713,448, which is incorporated by reference in its entirety), polyaziridine (see WO 03/089476, which is incorporated by reference in its entirety), and L-aminoacids or L-aminoesters (see WO 2004/067575, which is incorporated by reference in its entirety). Carbodiimides have also been reported for the crosslinking of HA (see U.S. Pat. Nos. 5,017,229 and 6,013,679, each of which is incorporated by reference in its entirety). Total or partial crosslinked esters of HA with an aliphatic alcohol, and salts of such partial esters with inorganic or organic bases, are disclosed in U.S. Pat. No. 4,957,744, also incorporated by reference in its entirety. Crosslinking of HA chains with 1-ethyl-3-(3-dimethylaminopropyl) carbodiimide hydrochloride (“EDAC”) and adipic acid dihydrazide in a water/acetone mixture is disclosed in U.S. 2006/0040892 (incorporated by reference in its entirety). WO 2006/56204, also incorporated by reference in its entirety, also discloses methods for preparing crosslinked gels of HA using divinyl sulfone (DVS) as the crosslinking agent.

HA is commercially available from various suppliers, such as Pure Health Botanicals (Saint Charles, IL) and DSM (Kaiseraugst, Switzerland).

### D. Compositions Comprising Collagen Fragments and Sequence Variants Thereof

The present disclosure provides a composition comprising collagen and hyaluronic acid, wherein the collagen is fully miscible with the hyaluronic acid. In some embodiments, the collagen is fully miscible with hyaluronates, for example, acetylated hyaluronate. In some embodiments, the collagen is fully miscible with acetylated hyaluronate. Other hyaluronates include zinc hyaluronate.

In some embodiments, the collagen is fully miscible with other polymers used for therapeutic applications. In some embodiments, the collagen can be fully miscible with the polymer in both aqueous solution and as a solid film or article. Contemplated articles include, but are not limited to: scaffolds and wound dressings. Such polymers include, but are not limited to, polyvinylpyrrolidone (PVP), polyacrylamide (PAM), poly(ethylene oxide) (PEO), poly(2-oxazoline)s, polyethyleninime (PEI), sodium carboxymethylcellulose, and chondroitin sulfate.

In some embodiments, the collagen is fully miscible with polyvinylpyrrolidone both in aqueous solution and as a solid film or article.

In some embodiments, the collagen is fully miscible with polyacrylamide both in aqueous solution and as a solid film or article.

In some embodiments, the collagen is fully miscible with poly(ethylene oxideglycol) In some embodiments, the collagen is fully miscible with poly(2-oxazoline)s.

In some embodiments, the collagen is fully miscible with polyethyleninime.

In some embodiments, the collagen is fully miscible with carboxymethylcellulose.

In some embodiments, the collagen is fully miscible with chondroitin sulfate.

In some embodiments, the collagen is fully miscible with acetylated hyaluronate.

In some embodiments, the collagen can be mixed with zinc hyaluronate. In some embodiments, the collagen is fully miscible with zinc hyaluronate.

In some embodiments, the collagen is fully miscible with an alginic acid sodium salt. In some embodiments, the alginic acid sodium salt has a viscosity of about 4-12 cps for a 1% aqueous solution at 25° C. In some embodiments, the alginic acid sodium salt has a viscosity of equal to or greater than about 2,000 cps for a 2% aqueous solution at 25° C.

In some embodiments, the collagen is a recombinantly produced collagen fragment. In some embodiments, the collagen within the composition can be physically and/or chemically modified. Chemical modifications and physical modifications can include but are not limited to hydroxylation, crosslinking, charge modification, linking agents, and modifications known in the art.

In some embodiments of the compositions disclosed herein, the compositions can further comprise one or more polymers or copolymers including, but not limited to, aliphatic polyesters, polyorthoesters, polyoxyesters, polyanhydrides, polycarbonates, polyurethanes, polyamides, polyester amides, hydrolyzable amines, polyalkylene oxides, poloxamers, functionalized isoflavonoids, amino acids, and combinations thereof.

In some embodiments of the compositions disclosed herein, the compositions can further comprise one or more polymers or copolymers selected from the group consisting of poly(hydroxyethyl methacrylate), poly(sulfobetaine), poly(dimethylsiloxane), poly(caprolactone), poly(p-dioxanone), poly(trimethylene carbonate), poly(glycolide), poly(L-lactide), poly(D,L-lactide), poly(L-lactide-co-glycolide), poly(D,L-lactide-co-glycolide), poly(L-lactide-co-caprolactone), poly(L-lactide-co-D,L-lactide), poly(caprolactone-co-trimethylene carbonate), poly(lactide-co-trimethylene carbonate), poly(caprolactone-co-p-dioxanone), poly(trimethylene carbonate-co-p-dioxanone), poly(caprolactone-co-lactide), poly(lactide-co-1,5-dioxepan-2-one), and poly(1,5-dioxepan-2-one-co-p-dioxanone), poly(lactide-co-1,4-dioxepan-2-one), poly(1,4-dioxepan-2-one-co-p-dioxanone), poly(glycolide-co-caprolactone-co-dioxanone), poly(lactide-co-caprolactone-co-dioxanone), poly(dioxanone-co-D,L-lactide), poly(dioxanone-co-glycolide), poly(dioxanone-co-caprolactone), poly(dioxanone-co-L-lactide), poly(dioxanone-co-D,L-lactide-co-caprolactone), poly(dioxanone-co-glycolide-co-caprolactone), poly(dioxanone-co-L-lactide-co-D,L-lactide), poly(dioxanone-co-L-lactide-co-glycolide), poly(dioxanone-co-L-lactide-do-caprolactone), poly(dioxanone-co-D,L-lactide-co-glycolide), poly(dioxanone-co-D,L-lactide-co-caprolactone), poly(dioxanone-co-glycolide-co-caprolactone, poly(dioxanone-co-L-lactide-co-glycolide-co-caprolactone), poly(dioxanone-co-D,L-lactide-co-glycolide-co-caprolactine), and combinations thereof.

In some embodiments, the polymers can comprise PEG copolymers such as PEG/poly(L-lactide), PEG/poly(D,L-lactide), PEG/poly(caprolactone), PEG/poly(L-lactide-co-glycolide), PEG/poly(sulfobetaine methacrylate), PEG diacrylates, and PEG dimethacrylates.

In some embodiments of the compositions disclosed herein, the compositions can further comprise biodegradable glasses or ceramics, such as calcium phosphates, and other biocompatible metal oxides (i.e., CaO) or a combination of biodegradable ceramics, glasses, polymers and copolymers.

In some embodiments of the composition disclosed herein, the collagen described herein has an isoelectric point (pI) of from about 4.5 to about 12, such as from about from about 5.0 to about 11.5, from about 5.5 to about 11.0, from about 6.0 to about 10.5, from about 6.5 to about 10.0, from about 7.0 to about 9.5, from about 7.5 to about 9.0, from about 8.0 to about 8.5, from 8.5 to about 11.5, about 9 to about 11, about 9 to about 10.5, about 9 to about 10, about 9 to about 9.5, and in some embodiments about 9.2, and in some embodiments about 9.66. In some embodiments of the composition disclosed herein, the composition has an isoelectric point (pI) of from about 1 to about 12.0, from about 4.0 to about 5.0, from about 5.0 to about 6.0, from about 6.0 to about 7.0, from about 7.0 to about 8.0, from about 8.0 to about 9.0, from about 9.0 to about 10.0, or from about 9.0 to about 9.5, or from about 9.5 to about 10.0.

In some embodiments, the HA can have a molecular weight that is compatible with biological tissue. In some embodiments, the collagen can have a molecular weight between 5 kDa and 150 kDa, 10 kDa and 120 kDa or from about 30 kDa to about 60 kDa.

In some embodiments, the molecular weight of HA in the composition described herein can range from about 10 kDa to about 10,000 kDa, about 25 kDa to about 5,000 kDa or from about 50 kDa to about 3,000 kDa. In some embodiments, the composition disclosed herein comprises hyaluronic acid or salt thereof with an average molecular weight in the range of from about 300 kDa to about 3,000 kDa, from about 400 kDa to about 2,500 kDa, from about 500 kDa to about 2,000 kDa, and from about 500 kDa to about 1,000 kDa.

In some embodiments, the molecular weight of PVP in the composition described herein can range from about 2.0 kDa to about 6,000 kDa, about 2.5 kDa to about 2,500 kDa, about 10 kDa to about 2,000 kDa, about 40 kDa to about 1,500 kDa, about 100 kDa to about 1,000 kDa.

In some embodiments, the molecular weight of PAM in the composition described herein can range from about 10 kDa to about 7,000 kDa and can be purchased from various sources, such as Sigma-Aldrich.

In some embodiments, the molecular weight of PEO in the composition described herein can range from about 5 kDa to about 300 kDa, about 50 kDa to about 200 kDa.

In some embodiments, the molecular weight of Poly(2-ethyl-2-oxazoline) in the composition described herein can range from about 10 kDa to about 1,000 kDa, about 50 kDa to about 500 kDa.

In some embodiments, the molecular weight of PEI in the composition described herein can range from about 2.5 kDa to about 2,500 kDa.

In some embodiments, the molecular weight of carboxymethylcellulose in the composition described herein can range from about 10 kDa to about 300 kDa. In some embodiments, the degree of substitution of the carboxymethylcellulose in the composition can range from about 1.00 to about 1.25. In some embodiments, the carboxymethylcellulose can have a molecular weight of about 250 kDa and a degree of substitution from about 0.70 to about 0.90. In some embodiments, the carboxymethylcellulose can have a molecular weight of about 250 kDa and a degree of substitution of about 0.79. In some embodiments, the carboxymethylcellulose can have a degree of substitution of about 0.87.

In some embodiments, the carboxymethylcellulose in the composition described herein can have a high viscosity. In some embodiments, the carboxymethylcellulose in the composition described herein can have a viscosity from about 100 to about 5,000 cps for a 1% aqueous solution. In some embodiments, the carboxymethylcellulose in the composition described herein can have a viscosity from about 1,000 to about 3,000 cps for a 1% aqueous solution. In some embodiments, the carboxymethylcellulose in the composition described herein can have a viscosity of about 2,700 cps for 1% aqueous solution.

In some embodiments, the molecular weight of the chondroitin sulfate in the composition can range from about 20 kDa to about 80 kDa. In some embodiments, the molecular weight of the chondroitin sulfate in the composition can be about 50 kDa.

In particular embodiments of the composition disclosed herein, the hyaluronic acid can have a molecular weight of from about 3 kDa to about 10,000 kDa. In some embodiments, the hyaluronic acid can have a molecular weight of from about 1,000 kDa to about 5,000 kDa. In some embodiments, the hyaluronic acid can have a molecular weight of from about 2,000 kDa to about 3,000 kDa.

In some embodiments of the composition disclosed herein, the hyaluronic acid can be crosslinked. In some embodiments, the hyaluronic acid is not crosslinked. In some embodiments of the composition disclosed herein, the collagen can be crosslinked. In some embodiments, the collagen is not crosslinked.

In some embodiments of the composition disclosed herein, the hyaluronic acid can be a biphasic crosslinked hyaluronic acid. In some embodiments, the hyaluronic acid can be a monophasic crosslinked hyaluronic acid.

In some embodiments, the hyaluronic acid in solution can have a pH in a range of from about 5.0 to about 12.

In some embodiments, the hyaluronic acid solution can have a pH in a range of from about 5.2 to about 7.2. In some embodiments the hyaluronic acid can have a pH of about 5.0, about 5.2, about 5.5, about 6.0, about 6.5, about 7.0, about 7.2, about 7.5, about 8.0, about 8.5, about 9.0, about 9.5, about 10.0, about 10.5, about 11, about 11.5, or about 12, or any measure therebetween.

In some embodiments of the composition disclosed herein, the hyaluronic acid and collagen can be present in a ratio (w/w) ranging from about 1:9 to 9:1. In some embodiments, the ratio (w/w) of hyaluronic acid to collagen can be about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, or about 9:1. In some embodiments the ratio (w/w) of hyaluronic acid to collagen can be 1:9. In some embodiments the ratio (w/w) of hyaluronic acid to collagen can be 1:3. In some embodiments the ratio (w/w) of hyaluronic acid to collagen can be 2:3. In some embodiments the ratio (w/w) of hyaluronic acid to collagen can be 1:1. In some embodiments the ratio (w/w) of hyaluronic acid to collagen can be 3:2. In some embodiments the ratio (w/w) of hyaluronic acid to collagen can be 3:1. In some embodiments the ratio (w/w) of hyaluronic acid to collagen can be 9:1.

In some embodiments, the collagen in the composition comprising collagen and HA can be one or more recombinant collagen fragments disclosed herein. In some embodiments, the one or more collagen fragments can be crosslinked to the HA. In some embodiments, the one or more collagen fragments can be covalently or noncovalently crosslinked to HA. In some embodiments, crosslinks can be formed by chemical reactions that result in the formation of covalent crosslinks and that are initiated by chemical agents, heat, pressure, pH, or irradiation. In some embodiments, non-covalent crosslinks can be formed using multivalent metal ions, such as, for example, iron, copper, zinc, calcium, magnesium, barium, and other chelating metal ions. Covalent linking can be accomplished using one or more suitable crosslinking agents, such as glutaraldehyde, a carbodiimide, a bi-functional amino acid (such as lysine), a protamine, or albumin. It is also possible to produce crosslinking by means of an amide, ester, or ether bond. Linking agents that have been employed to crosslink HA through covalent linkages are disclosed and discussed elsewhere herein.

In some embodiments, crosslinks can be formed directly between hyaluronic acid and one or more collagens or recombinant collagen fragments at an amino group of the collagen molecule and a carboxylic acid group of hyaluronic acid. In some embodiments, crosslinks between HA and the collagen molecule can be formed using a linking agent, suitable examples of which are disclosed and discussed elsewhere herein. In some embodiments, hyaluronic acid molecules can be crosslinked to other hyaluronic acid molecules (either in addition to the crosslinks between HA and the collagen molecule, or instead of between HA and the collagen molecule). In still further embodiments, one or more of the collagen molecules can be cross-linked to another collagen molecule (either in addition to the crosslinks between HA and the collagen molecule, or instead of between HA and the collagen molecule).

In some embodiments, the composition disclosed herein can comprise a crosslinked macromolecular matrix. In some embodiments, the crosslinked macromolecular matrix can be a homogeneous hydrogel composition prepared by a known method in the art under conditions in which collagen and HA are soluble in an aqueous solution.

In some embodiments of the composition disclosed herein, the collagen can comprise no hydroxyproline (Hyp) residues. In some embodiments, the collagen can comprise about 1% to about 20% hydroxyproline (Hyp) residues, based on the total number of prolines in the collagen. In some embodiments, the collagens described herein can include no hydroxyproline to about 20%, about 8% to about 20%, about 10% to about 20%, or about 12% to about 20% hydroxyproline. In some embodiments, the composition disclosed herein can include about 5%, about 6%, about 7%, about 8%, about 9%, about 10%, about 11%, about 12%, about 13%, about 14%, about 15%, about 16%, about 17%, about 18%, about 19%, or about 20% hydroxyproline. Hydroxyproline content can be measured by methods known in the art, for example by liquid chromatography-mass spectrometry (LC-MS).

In some embodiments of the composition disclosed herein, the collagen does not include any hydroxyl groups. In some embodiments of the composition disclosed herein, the collagen can comprise about 1 to about 120 hydroxyl groups. The number of hydroxyl groups can be measured by methods known in the art, for example by mass spectrometry.

In some embodiments, the collagen does not include any amine groups. In some embodiments, the collagen can comprise about 1 to about 100 amine groups. The number of amine groups can be measured by methods known in the art, for example by mass spectrometry and colorimetric TNBSA reagent assay.

In some embodiments, the collagen does not include any carboxylic acid groups. In some embodiments, the collagen can comprise about 1 to about 80 carboxylic acid groups. The number of carboxylic acid groups can be measured by methods known in the art.

The compositions disclosed herein can be prepared by a method comprising crosslinking hyaluronic acid and collagen. In some embodiments, the hyaluronic acid and collagen can be crosslinked under suitable conditions.

In some embodiments, the composition has a viscosity of from about 1 cps to about 100,000 cps. In some embodiments, the composition has a viscosity of from about 10,000 cps to about 80,000 cps. In some embodiments, the composition has a viscosity of from about 50,000 cps to about 70,000 cps.

In some embodiments, the composition can comprise a collagen that is fully miscible with hyaluronic acid and at least one pharmaceutically acceptable or cosmetically acceptable excipient. In some embodiments, the composition can comprise a recombinant collagen fragment according to SEQ ID NO: 1, and at least one pharmaceutically acceptable or cosmetically acceptable excipient. In further embodiments, the composition can comprise a recombinant collagen fragment having at least about 40%, at least about 45%, at least about 50%, at least about 55%, at least about 60%, at least about 65%, at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to, SEQ ID NO: 1, wherein the recombinant collagen fragment is fully miscible with the hyaluronic acid, and at least one pharmaceutically acceptable or cosmetically acceptable excipient. In still further embodiments, the composition can comprise a recombinant collagen fragment having about 40%, about 45%, about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or 100% identity, or similarity, to SEQ ID NO: 1, wherein the recombinant collagen fragment is fully miscible with the hyaluronic acid, and at least one pharmaceutically acceptable or cosmetically acceptable excipient. In still further embodiments, the composition can comprise a recombinant collagen fragment having about 40%, about 45%, about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or 100% identity, or similarity, to SEQ ID NO: 1, wherein the recombinant collagen fragment has the amino acid sequence set forth in any one of SEQ ID NOs: 975-1002, wherein the recombinant collagen fragment is fully miscible with the hyaluronic acid, and at least one pharmaceutically acceptable or cosmetically acceptable excipient.

In some embodiments, the composition can comprise a collagen that is fully miscible with hyaluronic acid, and at least one excipient that is suitable for use in a dietary supplement, e.g., a nutritional supplement. In some embodiments, the composition can comprise a recombinant collagen fragment having about 40%, about 45%, about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, about 99%, or 100% sequence identity, or similarity, to SEQ ID NO: 1, wherein the recombinant collagen fragment is fully miscible with the hyaluronic acid, and at least one excipient that is suitable for use in a dietary supplement, e.g., a nutritional supplement. In further embodiments, the composition can comprise a recombinant collagen fragment having about 85%, about 86%, about 87%, about 88%, about 89%, about 90%, about 91%, about 92%, about 93%, about 94%, about 95%, about 96%, about 97%, about 98%, about 99%, or 100% sequence identity, or similarity, to SEQ ID NO: 1, wherein the recombinant collagen fragment has the amino acid sequence set forth in any one of SEQ ID NOs: 975-1002, wherein the recombinant collagen fragment is fully miscible with the hyaluronic acid, and at least one excipient that is suitable for use in a dietary supplement, e.g., a nutritional supplement.

In some embodiments, the composition can comprise a hydrolysis product of a collagen fragment, wherein the hydrolysis product can have a sequence that is a portion of SEQ ID NO: 1, and at least one pharmaceutically acceptable or cosmetically acceptable excipient. In some embodiments, the composition comprises a hydrolysis product with a sequence according to one or more of SEQ ID NOs: 2-973, and at least one pharmaceutically acceptable or cosmetically acceptable excipient. In some embodiments, the composition can comprise any of the hydrolysis products set forth in SEQ ID NOs: 2-973, and at least one pharmaceutically acceptable or cosmetically acceptable excipient. In some embodiments, the composition can comprise a recombinant collagen fragment having at least about 40%, least about 45%, least about 50%, least about 55%, least about 60%, least about 65%, at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to, one of SEQ ID NOs: 2-973, and at least one pharmaceutically acceptable or cosmetically acceptable excipient.

In yet another embodiment, the composition can comprise a recombinant collagen fragment according to SEQ ID NO: 1, a hydrolysis product having a sequence according to one of SEQ ID NOs: 2-973, and at least one pharmaceutically acceptable or cosmetically acceptable excipient. In yet a further embodiment, the composition can comprise a recombinant collagen fragment according to SEQ ID NO: 1, a plurality of hydrolysis products having sequences, that can be the same or different, according to any of SEQ ID NOs: 2-973, and at least one pharmaceutically acceptable or cosmetically acceptable excipient. In certain embodiments, the number of hydrolysis products present in the plurality of hydrolysis products in the composition can increase with time, with temperature, pH, or as a result of other conditions that would typically cause a recombinant collagen fragment, such as a recombinant collagen fragment according to SEQ ID NO: 1, to hydrolyze or otherwise breakdown. In other embodiments, the composition can be stabilized with one or more stabilizers so that the concentrations of the recombinant collagen fragment according to SEQ ID NO: 1 and the concentrations each of the fragments in the plurality of fragments, remain substantially constant (i.e., vary by no more than +5% by HPLC over a given time period) or remain constant. In certain embodiments, the recombinant collagen fragment, such as a recombinant collagen fragment according to SEQ ID NO: 1, can be hydrolyzed, such that less than about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, or from about 90% to about 100% of non-hydrolyzed recombinant fragment remains in the composition as measured by HPLC. In other embodiments, the composition can comprise a mixture of a recombinant collagen fragment (e.g., a recombinant collagen fragment according to SEQ ID NO: 1) and a plurality of hydrolyzed products of that recombinant collagen fragment (e.g., a plurality of collagen fragments according to any of SEQ ID NOs: 2-973), such that the weight of the hydrolyzed products in the composition is less than about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, or from about 90% to about 100% of the weight of the collagen-related protein in the composition.

In some embodiments, the present disclosure provides a composition comprising one or more recombinant collagen fragment sequence variants disclosed herein, and at least one pharmaceutically acceptable or cosmetically acceptable excipient. In particular embodiments, the composition can comprise a recombinant collagen fragment sequence variant according to any one of SEQ ID NOs: 975-1002, and at least one pharmaceutically acceptable or cosmetically acceptable excipient. In further embodiments, the composition can comprise a recombinant collagen fragment sequence variant having at least about 40%, least about 45%, least about 50%, least about 55%, least about 60%, least about 65%, at least about 70%, at least about 75%, at least about 80%, at least about 85%, at least about 87.5%, at least about 90%, at least about 92.5%, at least about 95%, at least about 97.5%, at least about 98%, or at least about 99% sequence identity, or similarity to SEQ ID NO: 1, and at least one pharmaceutically acceptable or cosmetically acceptable excipient. In still further embodiments, the composition can comprise a recombinant collagen fragment sequence variant having about 40%, about 45%, about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, or about 99%, identity, or similarity, to SEQ ID NO: 1, and at least one pharmaceutically acceptable or cosmetically acceptable excipient. In still further embodiments, the composition can comprise a recombinant collagen fragment sequence variant having about 40%, about 45%, about 50%, about 55%, about 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98%, or about 99% sequence identity, or similarity SEQ ID NO: 1, and at least one excipient that is suitable for use in a dietary supplement, e.g., a nutritional supplement.

In some embodiments, the composition can comprise a recombinant collagen fragment having an amino acid chain length from about 150 amino acids, about 200 amino acids, about 250 amino acids, about 300 amino acids, about 350 amino acids to about 600 amino acids that overlaps with the amino acid sequence in SEQ ID NO: 1. In some embodiments, the composition comprises a recombinant collagen fragment having a length of about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids. In a particular embodiment, the composition comprises a recombinant collagen fragment having an amino acid chain length of 528 amino acids. In a particular embodiment, the composition comprises a recombinant collagen fragment having an amino acid chain length of 546 amino acids.

In some embodiments, the composition can comprise a recombinant collagen fragment sequence variant according to any one of SEQ ID NOs: 975-1002, having a length of about 150 amino acids, about 200 amino acids, about 250 amino acids, about 300 amino acids, about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids. In a particular embodiment, the composition comprises a recombinant collagen fragment having an amino acid chain length of 528 amino acids. In a particular embodiment, the composition comprises a recombinant collagen fragment having an amino acid chain length of 546 amino acids.

In some embodiments, the composition can comprise a recombinant collagen fragment or sequence variant thereof having an amino acid chain length from about 150 amino acids to about 600 amino acids and can have at least about 40%, least about 45%, least about 50%, least about 55%, least about 60%, least about 65%, at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to SEQ ID NO: 1. In some embodiments, the such a collagen fragment described herein can have a length of about 150 amino acids, about 200 amino acids, about 250 amino acids, about 300 amino acids, about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids.

In some embodiments, the composition can comprise a hydrolysis product of a collagen fragment, wherein the hydrolysis product can have an amino acid chain length from about 10 amino acids to about 75 amino acids. In some embodiments, the composition can comprise a hydrolysis product of a collagen fragment having an amino acid chain length from about 20 amino acids to about 50 amino acids. In some embodiments, the hydrolysis product described herein can have a length of about 10 amino acids, about 15 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, or about 75 amino acids.

In some embodiments the composition can comprise from about 5 ppm to about 500 ppm of a recombinant collagen fragment or sequence variant thereof. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of a recombinant collagen fragment. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of a recombinant collagen fragment sequence variant. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of a recombinant collagen fragment with a sequence according to SEQ ID NO: 1. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of one or more hydrolysis products of a recombinant collagen fragment. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of one or more hydrolysis products of a recombinant collagen fragment with a sequence according to SEQ ID NO: 1. In some embodiments, the composition can comprise about 5 ppm, about 10 ppm, about 25 ppm, about 50 ppm, about 100 ppm, about 150 ppm, about 200 ppm, about 250 ppm, about 300 ppm, about 350 ppm, about 350 ppm, about 400 ppm, about 450 ppm, or about 500 ppm of a recombinant collagen sequence variant according to any one of SEQ ID NOs: 987-1015. In some embodiments, the composition can comprise a mixture of a recombinant collagen fragment or variant thereof at a concentration from about 5 ppm to about 500 ppm, and one or more hydrolyzed products of that recombinant collagen fragment at a concentration from about 5 ppm to about 500 ppm. In some embodiments, the composition can comprise a mixture of a recombinant collagen fragment with a sequence according to SEQ ID NO: 1, or a variant thereof, at a concentration from about 5 ppm to about 500 ppm, and one or more hydrolyzed products of that recombinant collagen fragment at a concentration from about 5 ppm to about 500 ppm.

In some embodiments, the composition can be prepared with about 0.0001% to about 20% by volume of an about 0.5% to about 25% by weight recombinant collagen fragment solution. In some of these embodiments, the composition can comprise about 0.0001%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by volume of a recombinant collagen fragment solution. In some of these embodiments, the recombinant collagen fragment solution comprises about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of the recombinant collagen fragment or about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, or about 40% by weight of the recombinant collagen fragment sequence variant.

In some of these embodiments, the recombinant collagen fragment solution comprises about 0.0001%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% or about 40% by weight of a collagen fragment with a sequence according to SEQ ID NO: 1. In some of these embodiments, the recombinant collagen fragment solution comprises about 0.0001%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, or about 40% by weight of one or more hydrolyzed products of a recombinant collagen fragment. In some of these embodiments, the recombinant collagen fragment solution comprises about 0.0001%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25%, or about 40% by weight of one or more hydrolyzed products of a recombinant collagen fragment with sequences according to one or more of SEQ ID NOs: 2-973. In some of these embodiments, the recombinant collagen fragment sequence variant solution comprises about 0.0001%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25%, or about 40% by weight of one or recombinant collagen fragment sequence variants with sequences according to one or more of SEQ ID NOs: 975-1002.

In some of these embodiments, the composition can be prepared using about 0.0001% to about 35% by volume of an about 0.0005% to about 40% by weight of a mixture of a recombinant collagen fragment and one or more hydrolyzed products of that recombinant collagen fragment. In some of these embodiments, the composition can be prepared using about 0.0001% to about 35% by volume of an about 0.0005% to about 40% by weight of a mixture of a recombinant collagen fragment with a sequence according to SEQ ID NO: 1 and one or more hydrolyzed products of that recombinant collagen fragment.

In some embodiments, the composition can be prepared using about 0.0001% to about 35% by volume of an about 0.0005% to about 40% by weight solution comprising a mixture of recombinant collagen fragments with a sequence according to SEQ ID NO: 1 and one or more hydrolyzed products of the recombinant collagen fragment. In some embodiments, the composition can be prepared using about 0.0001% to about 35% by volume of an about 0.0005% to about 40% by weight solution comprising a mixture of a recombinant collagen fragment sequence variants with sequences according to any one of SEQ ID NOs: 2-973. In some embodiments, the composition can be prepared with about 0.0001% to about 35% by volume of an about 0.0005% to about 40% by weight solution comprising a mixture of a recombinant collagen fragment with a sequence according to SEQ ID NO: 1, and one or more hydrolyzed products of the recombinant collagen fragment and a recombinant collagen fragment sequence variant solution comprising sequence variants with sequences according to any one of SEQ ID NOs: 975-1002.

In some of these embodiments, the recombinant collagen fragment and recombinant collagen fragment sequence variant solution comprises about 0.0001%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25%, or about 40% by weight of one or more hydrolyzed products of a recombinant collagen fragment with sequences according to one or more of SEQ ID NOs: 2-973.

In some embodiments, the composition can comprise from about 0.0005% to about 25% by weight of a recombinant collagen fragment, from about 0.001% to about 25% by weight of a recombinant collagen fragment, from about 0.01% to about 25% by weight of a recombinant collagen fragment, from about 0.1% to about 25% by weight of a recombinant collagen fragment, from about 0.5% to about 20% by weight of a recombinant collagen fragment, from about 0.7% to about 17% by weight of recombinant collagen fragment, from about 1% to about 15% by weight of recombinant collagen fragment, from about 2% to about 12% by weight of recombinant collagen fragment, from about 2% to about 10% by weight of recombinant collagen fragment, from about 3% to about 9% by weight of recombinant collagen fragment, from about 4% to about 8% by weight of recombinant collagen fragment, or from about 5% to about 7% by weight of recombinant collagen fragment.

In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a recombinant collagen fragment. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a collagen fragment with a sequence according to SEQ ID NO: 1.

In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of one or more hydrolysis products of a recombinant collagen fragment. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of one or more hydrolysis products of a recombinant collagen fragment with sequences according to one or more of SEQ ID NOs: 2-973.

In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a mixture of a recombinant collagen fragment and one or more hydrolysis products of that recombinant collagen fragment. In some embodiments, the cosmetic composition can about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a mixture of a recombinant collagen fragment with a sequence according to SEQ ID NO: 1 and one or more hydrolysis products of that recombinant collagen fragment.

In some embodiments, the composition can comprise from about 0.0005% to about 25% by weight of a recombinant collagen fragment sequence variant, from about 0.001% to about 25% by weight of a recombinant collagen fragment sequence variant, from about 0.01% to about 25% by weight of a recombinant collagen fragment sequence variant, from about 0.1% to about 25% by weight of a recombinant collagen fragment sequence variant, from about 0.5% to about 20% by weight of a recombinant collagen fragment sequence variant, from about 0.7% to about 17% by weight of recombinant collagen fragment sequence variant, from about 1% to about 15% by weight of recombinant collagen fragment sequence variant, from about 2% to about 12% by weight of recombinant collagen fragment sequence variant, from about 2% to about 10% by weight of recombinant collagen fragment sequence variant, from about 3% to about 9% by weight of recombinant collagen fragment sequence variant, from about 4% to about 8% by weight of recombinant collagen fragment sequence variant, or from about 5% to about 7% by weight of recombinant collagen fragment sequence variant.

In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a recombinant collagen fragment sequence variant. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a collagen fragment sequence variant with a sequence according to any one of SEQ ID NOs: 975-1002.

In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a mixture of a recombinant collagen fragment and one or more recombinant collagen fragment sequence variants. In some embodiments, the cosmetic composition can about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of a mixture of a recombinant collagen fragment with a sequence according to SEQ ID NO: 1 and one or more collagen fragment sequence variants with a sequence according to any one of SEQ ID NOs: 975-1002.

In some embodiments, the composition can comprise a recombinant collagen fragment having the amino acid sequence set forth in SEQ ID NO: 1, one or more recombinant collagen fragments with sequences according to one or more of SEQ ID NOs: 2-973, and/or one or more collagen fragment sequence variants with a sequence according to any one of SEQ ID NOs: 975-1002, and hyaluronic acid, wherein the recombinant collagen fragments or variants are fully miscible with the hyaluronic acid. In some embodiments, the composition can comprise a recombinant collagen fragment having an amino acid sequence identity of at least about 40% to SEQ ID NO: 1, and/or one or more collagen fragment sequence variants having an amino acid sequence identity of at least about 40% to the amino acid sequence set forth in in any one of SEQ ID NOs: 975-1002, and hyaluronic acid, wherein the recombinant collagen fragments or variants are fully miscible with the hyaluronic acid. In some embodiments, the hyaluronic acid can be a biphasic crosslinked hyaluronic acid comprising crosslinked HA and uncrosslinked HA.

In some embodiments, the composition can comprise a recombinant collagen fragment having the amino acid sequence set forth in SEQ ID NO: 1, one or more recombinant collagen fragments with sequences according to one or more of SEQ ID NOs: 2-973, and/or one or more collagen fragment sequence variants with a sequence according to any one of SEQ ID NOs: 975-1002, and polyvinylpyrrolidone, wherein the recombinant collagen fragments or variants are fully miscible with the polyvinylpyrrolidone. In some embodiments, the composition can comprise one or more recombinant collagen fragments having an amino acid sequence identity of at least about 40% to SEQ ID NO: 1, and/or one or more collagen fragment sequence variants having an amino acid sequence identity of at least about 40% to the amino acid sequence set forth in in any one of SEQ ID NOs: 975-1002, and polyvinylpyrrolidone, wherein the recombinant collagen fragments or variants are fully miscible with the polyvinylpyrrolidone.

In some embodiments, the composition can comprise a recombinant collagen fragment having the amino acid sequence set forth in SEQ ID NO: 1, one or more recombinant collagen fragments with sequences according to one or more of SEQ ID NOs: 2-973, and/or one or more collagen fragment sequence variants with a sequence according to any one of SEQ ID NOs: 975-1002, and sodium carboxymethylcellulose, wherein the recombinant collagen fragments or variants are fully miscible with the sodium carboxymethylcellulose. In some embodiments, the composition can comprise one or more recombinant collagen fragments having an amino acid sequence identity of at least about 40% to SEQ ID NO: 1, and/or one or more collagen fragment sequence variants having an amino acid sequence identity of at least about 40% to the amino acid sequence set forth in in any one of SEQ ID NOs: 975-1002, and sodium carboxymethylcellulose, wherein the recombinant collagen fragments or variants are fully miscible with the sodium carboxymethylcellulose.

In some embodiments, the compositions described herein comprise hyaluronic acid at a concentration of at least about 1 mg/mL to at least about 100 mg/mL, at least about 1 mg/mL to at least about 50 mg/mL, at least about 5 mg/mL to at least about 25 mg/mL, at least about 6 mg/mL to at least about 24 mg/mL, at least about 7 mg/mL to at least about 23 mg/mL, at least about 8 mg/mL to at least about 22 mg/mL, at least about 9 mg/mL to at least about 21 mg/mL, or at least about 10 mg/mL to at least about 20 mg/mL, and any range therebetween. In some embodiments, the composition described herein comprises hyaluronic acid at a concentration of about 1 mg/mL to about 100 mg/mL, about 1 mg/mL to about 50 mg/mL, about 5 mg/mL to about 25 mg/mL, about 6 mg/mL to about 24 mg/mL, about 7 mg/mL to about 23 mg/mL, about 8 mg/mL to about 22 mg/mL, about 9 mg/mL to about 21 mg/mL, or about 10 mg/mL to about 20 mg/mL, and any range therebetween. In some embodiments, the composition of the present disclosure comprises hyaluronic acid at concentration of about 1 mg/ml, about 2 mg/ml, about 3 mg/ml, about 4 mg/ml, about 5 mg/ml, about 6 mg/mL, about 7 mg/mL, about 8 mg/mL, about 9 mg/mL, about 10 mg/mL, about 15 mg/mL, about 20 mg/mL, about 25 mg/mL, about 50 mg/mL or about 100 mg/ml. In some embodiments, the composition described herein comprises hyaluronic acid at a concentration of about 20 mg/mL to about 25 mg/mL. In a particular embodiment, the composition described herein comprises hyaluronic acid at a concentration of about 24 mg/mL. In some embodiments, the present disclosure provides a composition comprising collagen and hyaluronic acid, wherein the collagen is fully miscible with the hyaluronic acid.

In certain embodiments, the compositions disclosed herein include one or more collagens or recombinant collagen fragments disclosed herein, cross-linked with hyaluronic acid. The collagen can be directly crosslinked to the HA, i.e., through an amide or ester functionality, or can be linked to the HA through linker. In some embodiments, the composition disclosed herein comprises a hyaluronic acid component which includes hyaluronic acid that has reacted to become part of a crosslinked macromolecular matrix. In some embodiments, the composition according to the present disclosure comprises a collagen component comprising one or more recombinant collagen fragments that has reacted to become part of a crosslinked macromolecular matrix. In addition to the crosslinking between hyaluronic acid and collagen, hyaluronic acid and/or collagen can be self-crosslinked.

In some embodiments, the composition can be a solution. In some embodiments, the composition can be a gel. In some embodiments, the composition can be a film. In some embodiments, the composition can be a fiber or plurality of fibers. In some embodiments, the composition can be a coating. In some embodiments, the composition can be powder. In some embodiments, the composition can be a slurry. In some embodiments, the composition can be particles. In some embodiments, the composition can be a foam.

In some embodiments, the compositions described herein further comprise a pharmaceutically active agent. Exemplary active agents include, but are not limited to, sunscreens, desquamating agents, moisturizing agents, depigmenting agents, pro-pigmentants, alpha-hydroxy acids, antibacterial agents, antiradical agents, anti-pollution agents, anti-inflammatoires, anesthetics, retinoids, extracts of algae, mushrooms, vegetables, yeasts, bacteria, hydrolyzed, partially hydrolyzed or unhydrolyzed proteins, enzymes, hormones, vitamins and their derivatives, flavonoids and isoflavones, and mixtures thereof. Additional exemplary pharmaceutical agents include, but are not limited to, anesthetics, e.g., lidocaine, bupivacaine, mapivacaine, and related compounds. Additional exemplary pharmaceutical agents include, but are not limited to, non-steroidal anti-inflammatory agents (NSAIDs), e.g., flurbiprofen, ibuprofen, naproxen, indomethacin and related compounds. In some embodiments, the composition can further comprise one or more anti-mitotic drugs including colchicine, taxol, and related compounds. In some embodiments, the composition can further comprise one or more topical antiseptics such as, e.g., benzoyl peroxide. In some embodiments, the composition can further comprise one or more polysaccharides produced by microalgae, e.g., alguronic acid. In some embodiments, the composition can further comprise one or more bioactive peptides such as, e.g., palmitoyl tripeptide-1 and palmitoyl tetrapeptide-7. In some embodiments, the composition can further comprise one or more polysaccharides produced by microalgae, e.g., alguronic acid.

### E. Methods of Use

### Dermal Filler

In some embodiments, the present disclosure provides a dermal filler comprising the compositions comprising collagen and hyaluronic acid, disclosed herein. In some embodiments, the dermal filler disclosed herein can comprise a collagen of any known type that is miscible with HA. In some embodiments, the dermal filler provided herein can comprise a collagen of any known type that is fully miscible with HA. In some embodiments, administration of the dermal filler described herein can increase the production of collagen Type I. In some embodiments, administration of the dermal filler described herein can increase the production of collagen Type III. In some embodiments, administration of the dermal filler described herein can increase the production of any combination of collagen Type I, collagen Type III, or ECM.

In some embodiments, the dermal filler disclosed herein can comprise one or more recombinant collagen fragments disclosed herein. In particular embodiments, the dermal filler can comprise a recombinant collagen fragment according to SEQ ID NO: 1, and hyaluronic acid. In further embodiments, the dermal filler can comprise a recombinant collagen fragment having at least about 40%, least about 45%, least about 50%, least about 55%, least about 60%, least about 65%, at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to, SEQ ID NO: 1, and hyaluronic acid. In still further embodiments, the composition can comprise a recombinant collagen fragment having about 40%, about 45%, about 50%, about 55%, about 60%, about 65%, about 70%, about 80%, about 85%, about 86%, about 87%, about 88%, about 89%, about 90%, about 91%, about 92%, about 93%, about 94%, about 95%, about 96%, about 97%, about 98%, about 99%, or 100% identity, or similarity, to SEQ ID NO: 1, and hyaluronic acid. In some embodiments, administration of the dermal filler comprising a recombinant collagen fragment according to SEQ ID NO: 1 and hyaluronic acid, described herein, can increase the production of collagen Type I. In some embodiments, administration of the dermal filler comprising a recombinant collagen fragment according to SEQ ID NO: 1, and hyaluronic acid, described herein, can increase the production of collagen Type III. In some embodiments, administration of the dermal filler comprising a recombinant collagen fragment according to SEQ ID NO: 1 and hyaluronic acid, described herein, can increase the production of ECM. In some embodiments, administration of the dermal filler comprising a recombinant collagen fragment according to SEQ ID NO: 1 and hyaluronic acid, described herein, can increase the production of any combination of collagen Type I, collagen Type III, and ECM.

In some embodiments, the dermal filler can comprise a hydrolysis product of a collagen fragment, wherein the hydrolysis product can have a sequence that is a portion of SEQ ID NO: 1, and hyaluronic acid. In some embodiments, the composition comprises a hydrolysis product with a sequence according to one or more of SEQ ID NOs: 2-973, and hyaluronic acid. In some embodiments, the composition can comprise any of the hydrolysis products set forth in SEQ ID NOs: 2-973, and hyaluronic acid. In some embodiments, the composition can comprise a recombinant collagen fragment having at least about 40%, least about 45%, least about 50%, least about 55%, least about 60%, least about 65%, at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to, one of SEQ ID NOs: 2-973, and hyaluronic acid.

In some embodiments, the dermal filler can comprise a hydrolysis product of a collagen fragment, wherein the hydrolysis product can have a sequence that is a portion of SEQ ID NO: 1, and hyaluronic acid, described herein, can increase the production of collagen Type I. In some embodiments, administration of the dermal filler comprising a hydrolysis product of a collagen fragment, wherein the hydrolysis product can have a sequence that is a portion of SEQ ID NO: 1, and hyaluronic acid, described herein, can increase the production of collagen Type III. In some embodiments, administration of the dermal filler comprising a hydrolysis product of a collagen fragment, wherein the hydrolysis product can have a sequence that is a portion of SEQ ID NO: 1, and hyaluronic acid, described herein, can increase the production of both coallgen Type I and collagen Type III.

In yet another embodiment, the dermal filler can comprise a recombinant collagen fragment according to SEQ ID NO: 1, a hydrolysis product having a sequence according to one of SEQ ID NOs: 2-973, and hyaluronic acid. In yet a further embodiment, the dermal filler can comprise a recombinant collagen fragment according to SEQ ID NO: 1, a plurality of hydrolysis products having sequences that can be the same or different, according to any of SEQ ID NOs: 2-973, and hyaluronic acid. In certain embodiments, the number of hydrolysis products present in the plurality of hydrolysis products in the dermal filler can increase with time, with temperature, pH, or as a result of other conditions that would typically cause a recombinant collagen fragment, such as a recombinant collagen fragment according to SEQ ID NO: 1, to hydrolyze or otherwise breakdown. In other embodiments, the dermal filler can be stabilized with one or more stabilizers so that the concentrations of the recombinant collagen fragment according to SEQ ID NO: 1 and the concentrations each of the fragments in the plurality of fragments, remain substantially constant (i.e., vary by no more than +5% by HPLC over a given time period) or remain constant. In certain embodiments, the recombinant collagen fragment, such as a recombinant collagen fragment according to SEQ ID NO: 1, can be hydrolyzed, such that less than about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, or from about 90% to about 100% of non-hydrolyzed recombinant fragment remains in the composition as measured by HPLC. In other embodiments, the dermal filler can comprise a mixture of a recombinant collagen fragment (e.g., a recombinant collagen fragment according to SEQ ID NO: 1) and a plurality of hydrolyzed products of that recombinant collagen fragment (e.g., a plurality of collagen fragments according to any of SEQ ID NOs: 2-973), and hyaluronic acid, such that the weight of the hydrolyzed products in the composition is less than about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90%, or from about 90% to about 100% of the weight of the collagen-related protein in the dermal filler.

In yet another embodiment, the dermal filler can comprise a recombinant collagen fragment according to SEQ ID NO: 1, a hydrolysis product having a sequence according to one of SEQ ID NOs: 2-973, and hyaluronic acid. In some embodiments, administration of the dermal filler comprising a recombinant collagen fragment according to SEQ ID NO: 1, a hydrolysis product having a sequence according to one of SEQ ID NOs: 2-973, and hyaluronic acid, described herein, can increase the production of collagen Type I. In some embodiments, administration of a dermal filler a recombinant collagen fragment according to SEQ ID NO: 1, a hydrolysis product having a sequence according to one of SEQ ID NOs: 2-973, and hyaluronic acid, described herein, can increase the production of collagen Type III. In some embodiments, administration of the dermal filler comprising a recombinant collagen fragment according to SEQ ID NO: 1 and hyaluronic acid, described herein, can increase the production of both collagen Type I and collagen Type III.

In some embodiments, the dermal filler can comprise a collagen fragment sequence variant having the amino acid sequence according to any one of SEQ ID NO:s 975-1002, and hyaluronic acid.

In some embodiments, the dermal filler can further comprise a bioabsorbable polymer that is selected from the group consisting of solid homopolymers of poly(e-caprolactone), solid homopolymers of poly(p-dioxanone), solid homopolymers of poly(trimethylene carbonate), solid copolymers of a plurality of e-caprolactone repeating units and third lactone repeating units, solid copolymers of a plurality of trimethylene carbonate repeating units and second lactone repeating unit; wherein the third lactone repeating units are selected from the group consisting of glycolide repeating units, lactide repeating units, trimethylene carbonate repeating units, p-dioxanone repeating units, 1,4-dioxepan-2-one repeating units, 1-5-dioxepan-2-one repeating units and combinations thereof.

In some embodiments the dermal filler can further comprise one or more polymers including but not limited to aliphatic polyesters, polyorthoesters, polyoxyesters, polyanhydrides, polycarbonates, polyurethanes, polyamides, polyester amides, hydrolyzable amines, polyalkylene oxides, polyethylene oxide (PEO), poloxamers, functionalized isoflavonoids, amino acids, and combinations thereof.

In some embodiments the dermal filler can further comprise one or more polymers selected from the group consisting of poly(hydroxyethyl methacrylate), poly(sulfobetaine), poly(dimethylsiloxane), poly(caprolactone), poly(p-dioxanone), poly(trimethylene carbonate), poly(glycolide), poly(L-lactide), poly(D,L-lactide), poly(L-lactide-co-glycolide), poly(D,L-lactide-co-glycolide), poly(L-lactide-co-caprolactone), poly(L-lactide-co-D,L-lactide), poly(caprolactone-co-trimethylene carbonate), poly(lactide-co-trimethylene carbonate), poly(caprolactone-co-p-dioxanone), poly(trimethylene carbonate-co-p-dioxanone), poly(caprolactone-co-lactide), poly(lactide-co-1,5-dioxepan-2-one), and poly(1,5-dioxepan-2-one-co-p-dioxanone), poly(lactide-co-1,4-dioxepan-2-one), poly(1,4-dioxepan-2-one-co-p-dioxanone), poly(glycolide-co-caprolactone-co-dioxanone), poly(lactide-co-caprolactone-co-dioxanone), poly(dioxanone-co-D,L-lactide), poly(dioxanone-co-glycolide), poly(dioxanone-co-caprolactone), poly(dioxanone-co-L-lactide), poly(dioxanone-co-D,L-lactide-co-caprolactone), poly(dioxanone-co-glycolide-co-caprolactone), poly(dioxanone-co-L-lactide-co-D,L-lactide), poly(dioxanone-co-L-lactide-co-glycolide), poly(dioxanone-co-L-lactide-do-caprolactone), poly(dioxanone-co-D,L-lactide-co-glycolide), poly(dioxanone-co-D,L-lactide-co-caprolactone), poly(dioxanone-co-glycolide-co-caprolactone, poly(dioxanone-co-L-lactide-co-glycolide-co-caprolactone), poly(dioxanone-co-D,L-lactide-co-glycolide-co-caprolactine), and combinations thereof.

In some embodiments the dermal filler can further comprise PEG in combination with one or more polymers provided herein, examples of which include, but are not limited to, PEG/poly(L-lactide), PEG/poly(D,L-lactide), PEG/poly(caprolactone), PEG/poly(L-lactide-co-glycolide), PEG/poly(sulfobetaine methacrylate), PEG diacrylates, and PEG dimethacrylates.

In some embodiments the dermal filler can further comprise further comprise biodegradable glasses or ceramics, such as calcium phosphates, and other biocompatible metal oxides (i.e., CaO) or a combination of biodegradable ceramics, glasses, and polymers.

In some embodiments, administration of the dermal filler comprising a collagen fragment sequence variant having the amino acid sequence according to any one of SEQ ID NO: s 975-1002, and hyaluronic acid, described herein, can increase the production of collagen Type I. In some embodiments, administration of the dermal filler comprising a collagen fragment sequence variant having the amino acid sequence according to any one of SEQ ID NO:s 975-1002, and hyaluronic acid, described herein, can increase the production of collagen Type III. In some embodiments, administration of the dermal filler comprising a collagen fragment sequence variant having the amino acid sequence according to any one of SEQ ID NO:s 975-1002, and hyaluronic acid, can increase the production of both collagen Type I and collagen Type III.

In some embodiments, the dermal filler can comprise a recombinant collagen fragment according to SEQ ID NO: 1, a hydrolysis product having a sequence according to one of SEQ ID NOs: 2-973, a collagen fragment sequence variant having the amino acid sequence according to any one of SEQ ID NO:s 975-1002, and hyaluronic acid. In some embodiments, administration of the dermal filler comprising a recombinant collagen fragment according to SEQ ID NO: 1, a hydrolysis product having a sequence according to one of SEQ ID NOs: 2-973, a collagen fragment sequence variant having the amino acid sequence according to any one of SEQ ID NO:s 975-1002, and hyaluronic acid, described herein, can increase the production of both collagen Type I and collagen Type III.

In some embodiments, the dermal filler is a heat stable, sterile, soft tissue filler. In some embodiments, the dermal filler is heat stable up to at least about 125° C. In some embodiments, the dermal filler is heat stable at temperatures ranging up to at least about 135° C. As used herein, the dermal filler is “heat stable,” which means that the dermal filler is substantially stable when subjected to a heat treatment such as steam sterilization at normal pressure or under pressure (autoclaving). The stability of a dermal filler disclosed herein can be evaluated by methods known in the art. The heat stable dermal filler disclosed herein can retains one or more desired rheological characteristics present before the heat treatment. The heat stability of the dermal filler disclosed herein can prevent degradation of the collagen, collagen fragments, and/or collagen variants comprised within the dermal filler. In some embodiments, the dermal filler can be terminally sterilized with different sterilization techniques. In some embodiments, the dermal filler can be sterilized by autoclave, by gamma radiation, by e-Beam radiation, or by any method known in the art.

In some embodiments, the dermal filler is injectable. Typically, an injectable dermal filler is administered by subcutaneous or intradermal injection. The term “effective amount” refers to the amount of the injectable dermal filler sufficient to effect beneficial or desired cosmetic or therapeutic results. In some embodiments, the dermal filler is injected in a volume of about 0.1 mL, about 0.2 mL, about 0.3 mL, about 0.4 mL, about 0.5 mL, about 0.6 mL, about 0.7 mL, about 0.8 mL, about 0.9 mL, about 1.0 mL, about 1.5 mL, about 2.0 mL, about 2.5 mL, about 3.0 mL, about 3.5 mL, about 4.0 mL, about 4.5 mL, about 6.0 mL, about 6.5 mL, about 7.0 mL, about 7.5 mL, about 8.0 mL, about 8.5 mL, about 9.0 mL, about 9.5 mL, about 10.0 mL, about 20.0 mL, about 30.0 mL, about 40.0 mL, about 50.0 mL, about 60.0 mL, about 70.0 mL, about 80.0 mL, about 90.0 mL, about 100.0 mL, and any amounts therebetween.

In some embodiments, the dermal filler can further comprise calcium phosphate particles. Suitable calcium phosphate particles include but are not limited to calcium hydroxyapatite, calcium fluoroapatite, calcium chloroapatite, calcium carbonate apatite, tetracalcium phosphate, calcium pyrophosphate, tricalcium phosphate and octacalcium phosphate particles, and combinations thereof. In some embodiments, the dermal filler can further comprise calcium hydroxyapatite particles. In some embodiments, the dermal filler can further comprise carboxymethyl cellulose. In some embodiments, the dermal filler can further comprise polylactic acid.

In some embodiments, the particles can have a particle size in a range from about 10 μm to about 60 μm, and in particular embodiments from about 25 μm to about 45 μm. In some embodiments, the particles can have a particle size of less than 60 μm, less than 45 μm, less than 35 μm, or less than 25 μm.

In some embodiments, the dermal fillers disclosed herein are suitable for injection through a fine gauge needle. In some embodiments, the injectable dermal filler is suitable for administration with a needle having a size from about 16 Gauge (Ga) to about 33 Ga. In some embodiments, a suitable needle has a size of from about 22 Ga to about 30 Ga.

In some embodiments, the dermal fillers disclosed herein can be provided in ready-to-use prefilled syringe. Without wishing to be bound by a particular theory, it is believed that a synergistic effect of the components in the presently described dermal fillers results in formulation in which calcium phosphate particles, e.g., calcium hydroxyapatite particles, are stably suspended over time without undergoing phase separation.

In some embodiments, the dermal fillers described herein can be injected into any area of the body or face where an increase in tissue volume is desired. For example, suitable areas of the body and face include glabellar lines, nasolabial folds, chin folds, marionette lines, buccal commissures, peri-oral wrinkles, crow's feet, cutaneous depressions, scars, temples, subdermal support of the brows, malar and buccal fat pads, tear troughs, nose, lips, cheeks, perioral region, infraorbital region, facial asymmetries, jawlines, and chin. For example, suitable areas of the body include buttocks, breasts, calves, flanks, or any other area in the body where tissue volume is desired for a cosmetic effect, or to address body asymmetries. Further, an increase in tissue volume may have a therapeutic benefit. For example, therapeutic benefit can be achieved if the dermal fillers described herein are injected into the vocal cords, bladder wall or neck, or any other anatomic tubular structure, sphincter or valve that, when augmented by the dermal fillers described herein, would provide enhanced function.

In some embodiments, the dermal filler disclosed herein further comprises a pharmaceutically active agent. In some embodiments, the pharmaceutically active agent comprises an anesthetic agent. In some embodiments, the anesthetic agent is lidocaine, bupivacaine, mepivacaine, or any combination thereof.

### Medical or Cosmetic Treatments

In some embodiments, the present disclosure provides a method of medical or cosmetic treatment comprising administering or applying an effective amount of the composition described herein to a subject in need thereof. In some embodiments, the treatment comprises tissue augmentation. In some embodiments, the treatment comprises filling wrinkles or increasing volume in the face. In some embodiments, the treatment comprises forming a cosmetic implant in a patient.

The compositions disclosed herein are contemplated for any condition or disease in which administration or application of mixtures of collagen and hyaluronic acid can beneficial. Such conditions include but are not limited to: orthopedic conditions where internal cushioning is desired (for example between bone-to-bone contacts), osteoarthritis, arthritis, age-related ocular conditions, and others. In some embodiments, the composition disclosed herein can be administered to the eyes, blood vessels, nerves, cartilage, bones, breasts, vocal cords, feet, or tympanic membranes, joints, spinal discs, buttocks, hips, calves, or hands of the subject.

In some embodiments, the treatment comprises treating an inflammatory skin disease or mucous membrane disease.

In some embodiments, the treatment comprises treating lipoatrophy or other natural or surgical loss of tissue volume.

### Increasing Collagen Production in Cells

The present inventors have surprisingly discovered that the collagen fragments described in this disclosure can induce the production of collagen Type I and collagen Type III in cells such as fibroblasts. Thus, when contacting cells such as fibroblasts, or to other appropriate cells, the collagen fragment described here (i.e., a collagen fragment having the amino acid sequence of SEQ ID NO: 1, or a composition comprising such a fragment and hyaluronic acid) can advantageously induce collagen formation in the cells on which it is contacted.

Accordingly, in some embodiments, the present disclosure provides methods of increasing collagen production in cells, comprising contacting cells with the composition described herein. Such contact can consist of directly exposing the cells to the composition in an amount sufficient to induce the cells to produce greater amounts of collagen. In some embodiments, the method comprises administering or applying the composition comprising the collagen or collagen fragment, hyaluronic acid and at least one pharmaceutically acceptable excipient. In some embodiments, the composition can be applied to cells in their natural context, such as in an organism or tissue. In some embodiments, the composition can be topically applied to skin. In some embodiments, the composition can be topically applied to human skin. In some embodiments, as described above, the composition can be injected.

In some embodiments, the cells can be fibroblasts. In some embodiments, the cells can be fibroblasts in their native context (e.g., within human skin). In some embodiments, the cells can be primary human fibroblasts. In some embodiments, the cells can be muscle cells, transformed human cells, cardiomyocytes, epithelial cells, endothelial cells, stem cells, induced pluripotent stem cells, disc nucleus pulposus cells, synoviocytes, osteoblasts, chondrocytes, keratinocytes, or keratocytes. In some embodiments, the cells can be cells that produce insulin. In some embodiments, the cells can be pancreatic beta cells.

In some embodiments, the compositions disclosed herein can be applied to a wound, such as a cut, laceration, gash, tear, scrape, abrasion, burn, or scratch, to increase collagen production at the wound site and/or its surrounding tissues. As part of the wound healing process, fibroblasts migrate to a wound site and produce collagen that is necessary for wound repair. The cells eventually fill the wound cavity with a network of interlacing threads of collagen which in due time, arrange themselves in firm bands and form the permanent new tissue. Accordingly, compositions comprising the collagens, collagen fragments, and collagen variants described herein can be applied to a wound, such as a cut, laceration, gash, tear, scrape, abrasion, burn, or scratch, to increase collagen production at the wound site and/or its surrounding tissues.

In some embodiments, the wound is a chronic wound. Chronic wounds include, but are not limited, to diabetic foot ulcers, venous leg ulcers, and pressure ulcers.

In certain embodiments, the present disclosure provides methods of applying compositions comprising a collagen or recombinant collagen fragment, hydrolysis products of a recombinant collagen fragment, or combinations thereof, and hyaluronic acid, to a wound, such as a cut, laceration, gash, tear, scrape, abrasion, burn, or scratch. In certain embodiments, the wound can be a wound in a human subject. In some embodiments, the method comprises providing a composition for and promoting collagen production and wound healing in the skin of a subject in need thereof, wherein the composition comprises a recombinant collagen fragment described herein (e.g., a fragment comprising SEQ ID NO: 1). In some embodiments, the method comprises providing a composition for and promoting collagen production and wound healing in the skin of a subject in need thereof, wherein the composition comprises one or more hydrolyzed products of a recombinant collagen fragment described herein (e.g., hydrolyzed products comprising one or more of SEQ ID NOs: 2-973). In some embodiments, the method comprises providing a composition for and promoting collagen production and wound healing in the skin of a subject in need thereof, wherein the composition comprises a mixture of a recombinant collagen fragment and one or more hydrolyzed products of that recombinant collagen fragment, as described herein (e.g., a mixture of a fragment comprising SEQ ID NO: 1, and hydrolyzed products comprising SEQ ID NOs: 2-973).

In some embodiments, the method can comprise administering a collagen fragment with the amino acid sequence of SEQ ID NO: 1, and hyaluronic acid, to cells. In some embodiments, the method can comprise administering a hydrolysis product resulting from the hydrolysis of a collagen fragment with the amino acid sequence of SEQ ID NO: 1 to cells (e.g., administering one or more hydrolysis products with sequences according to SEQ ID NOs: 2-973). In some embodiments the method comprises administering a recombinant collagen fragment described herein and one or more hydrolysis products of that recombinant collagen fragment to cells. In some embodiments the method can comprise administering a collagen fragment with the amino acid sequence of SEQ ID NO: 1, and one or more hydrolysis products of that fragment that have sequences according to SEQ ID NOs: 2-973 to cells. In some embodiments the method can comprise administering a collagen fragment with the amino acid sequence of any one of SEQ ID NOs: 975-1002, and hyaluronic acid, to cells.

In some embodiments, the method can increase the production of collagen Type I. In some embodiments, the method can increase the production of collagen Type III. In some embodiments, the method can increase the production of ECM. In some embodiments, the method can increase the production of any combination of collagen Type I, collagen Type III, and ECM.

### Skin Care

In some embodiments, the compositions described herein can be used to improve the aesthetic appearance of the skin and/or its appendages, for example, the surface appearance and/or texture of the skin. In some embodiments, the compositions described herein can be formulated for use on the body and face, hands, and feet, including as treatment for the eye area, for nails, and hair. The term “surface appearance” means the visual and/or tactile irregularities in the skin and/or in the scalp, including wrinkles and fine lines, expression lines on the forehead and in the space between the eyebrows, wrinkles and/or fine lines around the mouth, and/or slackening in the area around the lips and the top lip area (area located between the top lip and the nose), heterogeneity of the skin tone (liver spots, actinic lentigos), appearance and/or visibility of the pores, papery appearance of the skin, defects in the skin microrelief such as chicken pox or acne scars, imperfections of greasy skin (shiny appearance, etc.). The term “skin texture” can mean slack, flabby, less firm, less elastic skin, and/or skin that has sagged.

In some embodiments, the compositions described herein can be used to improve the aesthetic appearance of the skin, including improvement in the appearance of expression lines. Expression lines are produced by the effect of the stress exerted on the skin by the underlying muscles. Age and environmental factors such as exposure to sunlight, can deepen expression lines and make them permanent. Expression lines are characterized by the presence of grooves in the area around the orifices formed by the nose (nasal grooves), the mouth (perioral lines and so-called bitterness lines) and the eyes (crow's feet wrinkles), around which the skin muscles are located, and between the eyebrows (Glabella or lion wrinkles) and on the forehead.

In some embodiments, the compositions described herein can be used to improve the aesthetic appearance of the skin and/or visibility of the pores. Visibility of the pores can be due to an excess of sebum, aging, loss of firmness, slackening, stress, fatigue, unsuitable hygiene, climatic factors, or any combination thereof. The compositions described herein can tighten the pores, making them less visible.

In some embodiments, the compositions described herein can be used to improve a papery appearance of the skin and the behavior of the skin to the touch. Specifically, older skin can visually take on the appearance of cigarette paper, giving it an appearance similar to that of a sheet of Papyrus. The papery appearance of the skin can be seen on the face and on the back of the hands of the elderly.

In some embodiments, the compositions described herein can be a composition for protecting, treating, or caring for the face, for the hands, for the feet, or for the body, for example, day creams, night creams, makeup remover creams, anti-sun compositions, body milks for skin protection or care, after-sun milks, skincare lotions, gels, foams, artificial tanning compositions, and aftershave compositions. In some embodiments, the compositions described herein can be formulated, for example, as solutions, suspensions, lotions, creams, serums, gels, balms, gels, oils, oil in creams, micellar waters, face mists, face essences, blemish balm or complexion corrector formulas, toners (water and/or alcohol based), paints, polishes, sticks, pencils, sprays, aerosols, ointments, cleansing liquid washes, solid bars, shampoos, hair conditioners, hair styling products, pastes, foams, powders, mousses, balms, shaving creams, wipes, strips, patches, wound dressings, adhesive bandages, hydrogels, film-forming products, facial and skin masks, cosmetics (e.g., foundations, eye liners, eye shadows), exfoliators, deodorants and anti-perspirants, and the like. Exemplary formulations are provided herein.

In some embodiments, the composition described herein can be a cosmetic composition and the at least one excipient can be a cosmetically acceptable excipient. Cosmetically acceptable excipients are excipients suitable for use in a cosmetic product. Exemplary cosmetically acceptable excipients are described below.

In some embodiments, the cosmetic composition described herein can comprise a hydrolysis product of a recombinant collagen fragment and at least one excipient, e.g., a cosmetically acceptable excipient. In some embodiments, the cosmetic compositions described herein can comprise a recombinant collagen fragment, one or more hydrolyzed products of that recombinant collagen fragment, and at least one excipient, e.g., a cosmetically acceptable excipient.

In some embodiments, the cosmetic composition can comprise ingredients commonly used in cosmetics, e.g., skin care, eye care, nail care, and hair care products. These ingredients can include, but are not limited to, soaps, antimicrobials, anti-inflammatories, moisturizers, waxy alcohols, hydration agents, moisturizers, penetration enhancers, emulsifiers, natural or synthetic oils, solvents, fats, surfactants, detergents, gelling agents, emollients, antioxidants, fragrances, paints, polishes, fillers, thickeners, waxes, odor absorbers, dyestuff, coloring agents, powders, viscosity-controlling agents, analgesics, anesthetics, anti-itch agents, botanical extracts, conditioning agents, darkening or whitening agents, humectants, mica, minerals, polyphenols, silicones or silicone derivatives such as dimethicone, sun blocks, vitamins, phytomedicinals, alcohols, such as denatured alcohols and ethanol, polyols, polyolethers, and other ingredients listed in the International Cosmetic Ingredient Dictionary and Handbook, 13th Ed. (2009), the entirety of which is incorporated herein by reference. In certain embodiments, a given ingredient can perform more than one function and can belong to more than one class.

In some embodiments, the composition described herein can be a therapeutic composition and the at least one excipient can be a therapeutically acceptable excipient. Therapeutic compositions can be useful for treating one or more conditions such as reducing or preventing the formation of scar tissue, promoting healing, promoting tissue regeneration, minimizing local inflammation, minimizing tissue rejection, and/or enhancing skin and/or hair graft integration. Therapeutically acceptable excipients are excipients that can serve as a vehicle or medium for an active substance and include excipients commonly used in therapeutic compositions, i.e., compositions useful for treating one or more conditions. Exemplary therapeutically acceptable excipients are described below.

In some embodiments, the composition described herein can be a dietary composition or dietary supplement, and the at least one excipient can be, for example, a food or drink additive. A “dietary supplement” is a preparation intended to supplement the diet and can be useful for providing nutrients or additives that may be missing or may not be consumed in sufficient quantities in a person's diet. In some embodiments, the dietary supplement can be provided in the form of any commonly used solid or liquid dosage form for oral administration including, without limitation, a capsule, a tablet, a pill, a powder, a granule or powder, a soft and hard gelatin capsule, and/or as a gummy. Suitable excipients include, but are not limited to, lactose or milk sugar as well as high molecular weight polyethyleneglycols, and the like. Exemplary dietary compositions are described below.

The compositions described herein can also include one or more of the following additional components. Exemplary contemplated additional ingredients are set forth below; however, this disclosure is not limited to these exemplary additional ingredients.

In some embodiments, the composition described herein can further comprise one or more anti-wrinkle agents. An anti-wrinkle agent is a compound which produces an increase in the synthesis and/or in the activity of certain enzymes of the skin, when the composition is brought into contact with an area of wrinkled skin, e.g., on the body or face, including the eye area, which reduces the outward appearance of the wrinkles and/or fine lines. Exemplary anti-wrinkle agents include, but are not limited to, desquamating agents, antiglycation agents, nitric oxide synthase inhibitors, muscle relaxants and/or dermo-decontracting agents, agents for combating free radicals, and mixtures thereof.

Additional exemplary anti-wrinkle agents that can be included in the composition described herein include, but are not limited to, adenosine and its derivatives, retinol and its derivatives (e.g., retinyl palmitate), ascorbic acid and its derivatives (e.g., magnesium ascorbyl phosphate and ascorbyl glucoside), tocopherol and its derivatives (e.g., tocopheryl acetate), nicotinic acid and its precursors (e.g., nicotinamide), ubiquinone, glutathione and its precursors (e.g., L-2-oxothiazolidine-4-carboxylic acid), C-glycoside compounds (also known as C-glycosyl compounds) and their derivatives (e.g., a β-C-xylosyl derivative with the trade name PRO-XYLANE, plant extracts (e.g., rock samphire extracts and olive leaf extracts), plant proteins and their hydrolysates (e.g., rice or soybean protein hydrolysates), algal extracts (e.g., laminarian extracts), bacterial extracts, sapogenins (e.g., diosgenin), Dioscorea extracts (e.g., wild yam extracts), α-hydroxy acids, β-hydroxy acids (e.g., salicylic acid and 5-(n-octanoyl) salicylic acid), oligopeptides and pseudodipeptides and their acylated derivatives (e.g., {2-[acetyl (3-(trifluoromethyl) phenyl) amino]-3-methylbutyrylamino}acetic acid), lipopeptides e.g., MATRIXYL 3000 available from Croda), lycopene, manganese, magnesium salts (e.g., gluconates), and combinations of any of the foregoing.

Exemplary adenosine derivatives include, but are not limited to, 2′-deoxyadenosine; 2′,3′-iso-propylideneadenosine; toyocamycin, 1-methyladenosine; N-6-methyladenosine, adenosine N-oxide, 6-methyl-mercaptopurine riboside, and 6-chloropurine riboside. Other adenosine derivatives comprise adenosine receptor agonists, including phenylisopropyladenosine (“PIA”)/1-methylisoguanosine, Ns-cyclohexyladenosine (CHA), N6-cyclopentyladenosine (CPA), 2-chloro-Ns-cyclopentyl-adenosine, 2-chloroadenosine, N6-phenyladenosine, 2-phenylaminoadenosine, MECA, Ne-phenethyladenosine, 2-p-(2-carboxyethyl) phenethylamino-5′-N-ethylcarboxamido-adenosine (CGS-21680), (N-ethylcarboxamido) adenosine-S-(NECA), 5′-(N-cyclopropylcarboxamido) adenosine, DPMA (PD 129,944), and metrifudil.

In some embodiments, the composition described herein can comprise one or more adenosine derivatives that increase the intracellular concentration of adenosine, such as erythro-9-(2-hydroxy-3-nonyl) adenine (“EHNA”), iodotubercidin, or combinations thereof. Additional adenosine derivatives contemplated herein include adenosine salts and alkyl esters of adenosine.

In some embodiments, the composition can further comprise one or more pearlescent agents. Pearlescent agents are iridescent particles of any shape produced in particular by certain shellfish in their shells. Alternatively, pearlescent agents can be synthesized, i.e., man made. The pearlescent agents can be chosen from white pearlescent agents such as, but not limited to, mica covered with titanium oxide or with bismuth oxychloride, colored pearlescent agents such as, but not limited to, pearlescent agents based on bismuth oxychloride, titanium oxide-coated mica covered with iron oxides, titanium oxide-coated mica covered with in particular ferric blue or chromium oxide, or titanium oxide-coated mica covered with an organic pigment.

In some embodiments, the composition can further comprise one or more hydroxy acids. Examples of hydroxy acids include beta hydroxy acids such as salicylic acid, acetylsalicylic acid, and the like. Additional exemplary hydroxy acids suitable for use in the composition include citric acid, glycolic acid, hydroxycaproic acid, hydroxycaprylic acid, lactic acid, malic acid, tartaric acid, polyhydroxy acids including gluconolactone, and any combination thereof.

In some embodiments, the composition can further comprise one or more emulsifiers. An emulsifier keeps unlike ingredients (such as oil and water) from separating in an emulsion. Suitable emulsifiers include but are not limited to: polysorbates, laureth-4, potassium cetyl sulfate, glyceryl caprylate, and any combinations thereof.

In some embodiments, the composition can further comprise one or more chelating agents. Chelating agents bind with metal ions and prevent them from chemically reacting with other substances in a formulation. Suitable chelating agents include but are not limited to: sodium phytate, disodium EDTA, tetrasodium EDTA, tetrasodium glutamate diacetate, and trisodium ethylenediamine disuccinate.

In addition to a chelating agent, tetrasodium glutamate diacetate can also act as a stabilizer in any of the compositions described herein.

In some embodiments, the composition can further comprise one or more antimicrobials. Suitable antimicrobials include but are not limited to: caprylyl glyceryl ether, benzalkonium chloride, benzethonium chloride, and chloroxylenol (PCMX), tea tree oil, witch hazel, rosemary oil, lemon oil, and any combination thereof.

In some embodiments, the composition can further comprise one or more humectants (water-retaining agents) to improve the level of moisture in the skin. Non-limiting examples of suitable humectants for use in the compositions described herein are described in WO 98/22085, WO 98/18444, and WO 97/01326 and include: amino acids and derivatives thereof such as proline and arginine aspartate, 1,3-butylene glycol, propylene glycol, pentylene glycol, water, codium tomentosum extract, creatinine, diglycerol, biosaccharide gum-1, glucamine salts, glucuronic acid salts, glutamic acid salts, polyethylene glycol ethers of glycerine (e. g. glycereth 20), glycerine, glycerol monopropoxylate, glycogen, hexylene glycol, honey, hydrogenated starch hydrolysates, hydrolyzed mucopolysaccharides (such as xanthan gum and biosaccharide gum-1), inositol, keratin amino acids, glycosaminoglycans, methoxy PEG 10, methyl gluceth-10 and -20, methyl glucose, 3-methyl-1,3-butanediol, N-acetyl glucosamine salts, polyethylene glycol and derivatives thereof (such as PEG 15 butanediol, PEG 4, PEG 5 pentaerythitol, PEG 6, PEG 8, PEG 9), propanediol, pentaerythitol, 1,2 pentanediol, PPG-1 glyceryl ether, 2-pyrrolidone-5-carboxylic acid (including salts and esters thereof), saccharide isomerate, sericin, silk amino acids, sodium acetylhyaluronate, sodium hyaluronate, sodium poly-aspartate, sodium polyglutamate, caprylyl glycol, sorbeth 20, sorbeth 6, sugar and sugar alcohols and derivatives thereof such as glucose, mannose and polyglycerol sorbitol, trehalose, triglycerol, trimethyolpropane, tris (hydroxymethyl) amino methane salts, and yeast extract, and mixtures thereof.

Additional humectants suitable for use herein include polyhydric alcohols selected from the group consisting of glycerin, diglycerin, glycerol, erythritol, arabitol, xylitol, ribitol, mannitol, sorbitol, galactitol, fucitol, maltitol, mannose, inositol, triethyleneglycol, sodium pyrrolidone carboxylic acid (PCA), zinc PCA and derivatives and mixtures thereof.

In some embodiments, the composition can further comprise a gelling agent comprising a polyacrylamide-based polymer. The polyacrylamide-based polymer can, aside from being polyacrylamide itself, be a derivative thereof, and can be a mixture of a plurality of types of polymers and can also be a copolymer with acrylamide and its derivatives as monomers. A gelling agent can be used to provide a good appearance to the skin, provide a sense of coolness to the skin, and provide a sense of refreshment without stickiness to the skin. In some embodiments, a gelling agent can include one or more pigments, or one or more fillers, including inorganic pigments, including extender pigments, coloring pigments, and whitening pigments, organic pigments, pearlescent gloss pigments, macromolecular powders, functional pigments, talc, mica, kaolin, calcium carbonate, magnesium carbonate, silicic anhydride, aluminum silicate, magnesium silicate, calcium silicate, aluminum oxide, barium sulfate, red iron oxide, yellow iron oxide, black iron oxide, chrome oxide, ultramarine blue, prussian blue, carbon black, zinc oxide, mica titanium, fish scale flakes, bismuth oxychloride, boron nitride, nylon powder, silk powder, carbomer, tar pigments, natural pigments and titanium oxide, such as amorphous or rutile type and/or anatase type crystals.

In some embodiments, the composition can comprise a pigment and/or filler that is resistant to water and an oil and can further include any conventionally used water-repellent and/or oil-repellent agent to confer water repellence and oil repellence to pigments, for example, fluorine compounds. Representative fluorine compounds which are conventionally used and can act as water-repellent and oil-repellent agents include compounds having perfluoroalkyl groups such as perfluororalkyl phosphates, perfluoroalkyl silanes, perfluoroalkyl silazanes, polyhexafluoropropylene oxides, perfluoroalkyl-group-containing organosiloxanes, per-fluoropolyethers, perfluoro alcohols, perfluoroalkylacrylate polymers, and derivatives thereof. Perfluoroalkyl phosphates can provide a uniform and stable dispersement of pigments within the formulation of a gel composition, and perfluoroalkyl silanes can have exceptional compatibility with other cosmetic ingredients. Additionally, the perfluoroalkyl phosphate-diethanol amine salt marketed by Asahi Glass as AsahiGuard AG530, and perfluoroalkyl silane coupling agents, such as LP-IT and LP-4T of Shin-Etsu Silicone, can be used.

Representative gelling agents include, but are not limited to, those marketed by Seppic under the trade names Sepigel 305, Sepigel 501, and Sepigel 600. Sepigel 305 is a mixture containing approximately 40% polyacrylamide, approximately 24% —C₁₃-C₁₄ isoparaffin and approximately 6% Laureth-7 (here, Laureth-7 is a non-ionic surfactant having the formula C₁₂H₂₅—(OCH₂CH₂)ₙ—OH, wherein n has an average value of 7). Sepigel 600 is a mixture of a acrylamide/acrylamide-2-propane sulfonate copolymer, isohexadecane and polysorbate 80 (polyoxyethylene sorbitan mono-oleate (20 EO)). A suitable gelling agent comprising a polyacrylamide-based polymer is disclosed in EP 0 503 853 (Scott Bader Company Ltd.), the disclosure of which is incorporated by reference herein.

In some embodiments, the composition can further comprise hyaluronic acid (HA). In some embodiments, the HA can be in an uncrosslinked state. In some embodiments, the HA can be in a crosslinked state. Like collagen, HA is an important structural component of human tissues. Hyaluronan, also known as hyaluronic acid (HA) is a non-sulfated glycosaminoglycan that is distributed widely throughout the human body in connective, epithelial, and neural tissues. Hyaluronan is abundant in the different layers of the skin, where it has multiple functions such as, e.g., to ensure good hydration, to assist in the organization of the ECM, to act as a filler material; and to participate in tissue repair mechanisms. However, with age, the quantity of hyaluronan present in the skin decreases.

In some embodiments, the composition described herein can be a dermal filler composition, e.g., an injectable dermal filler composition. Dermal filler compositions can be suitable for use on the face and body, including, e.g., around the eyes, on or around the cheeks, on or around the décolletage, on or around the hands, on or around the nails, on or around the ears, including on the earlobes, on or around the legs, and on or around the feet.

In some embodiments, the composition described herein can be used with a microneedle array, such as an array included in a sheet or patch. Microneedle arrays can comprise a plurality of microneedles that are of a length sufficient to penetrate the skin across the stratum corneum and into the viable epidermis. It some embodiments it can be desirable to deliver collagento the area of epidermal/dermal junction for cosmetic or therapeutic purposes.

In some embodiments, the composition described herein can be used with microneedle sheets or patches. Microneedles and microneedle patches are suitable for delivering collagen into the epidermis and dermis of human skin on the face and body, including, e.g., the eyes, the cheeks, the lips, the décolletage, and the hands. In some embodiments, the microneedles for delivering compositions into the epidermis and dermis in a targeted manner are injectable microneedles, drug coated metal microneedles, or microneedles having dissolvable tips. Exemplary methods and disclosures regarding microneedles can be found in, for example, Aditya et al., Kinetics of collagen microneedle drug delivery system, Journal of Drug Delivery Science and Technology, vol. 52, pp. 618-623 (August 2019) and Sun et al., Transdermal Delivery of Functional Collagen Via Polyvinylpyrrolidone Microneedles, Ann. Biomed. Eng., 43(12):2978-2990 (2015), each of which is incorporated by reference in its entirety. In some embodiments, the composition described herein can be applied to the skin prior to, during, or following any skin resurfacing procedure. Skin resurfacing procedures refer to a variety of skin treatments designed to revitalize skin. Skin resurfacing procedures include laser skin resurfacing, chemical peels, microdermabrasion, dermabrasion, microneedling, and Intense Pulsed Light (IPL) technology photorejuvanation. In some embodiments, the composition can further comprise a waxy lipid, e.g., a ceramide. Ceramides help create a barrier to prevent permeability, which helps prevent dryness and irritation and can also protect the epidermis from environmental damage.

In some embodiments, the composition can further comprise vitamin A or a vitamin A derivative. Examples of vitamin derivatives include, but are not limited to, retinoids such as retinal, retinoic acid, retinoate, retinyl ester, retinol, tretinoin, isotretinoin, adapalene, tazarotene, and the like. The term “retinoids” includes cis and trans derivatives of retinoids (e.g., all-trans-retinoic acid, 13-cis-retinoic acid, 13-trans retinoic acid, and 9-cis-retinoic acid).

In some embodiments, the composition can further comprise vitamin C or its derivatives, e.g., ascorbic acid, ascorbate (e.g., tetrahexyldecyl ascorbate), and the like.

In some embodiments, the composition can further comprise vitamin B, e.g., biotin, (i.e., vitamin B7), niacinamide, and the like.

In some embodiments, the composition can further comprise vitamin E, e.g., α-, β-, γ-, and σ-tocopherols and their related corresponding tocotrienols), and the like.

In some embodiments, the composition can further comprise vitamin K and derivatives thereof.

Any vitamin, vitamin analog, or derivative thereof that can be suitably formulated as a topical composition is contemplated for the present disclosure.

In some embodiments, the composition disclosed herein can further comprise one or more thickening agents. A thickening agent, i.e., structure builder, is able to suspend pigments and/or build viscosity in the composition. Thickeners and/or structure builders suitable for the present compositions include, but are not limited to, organically modified clays, fumed silica, trihydroxystearin, silicone gels or silicone elastomers, ammonium acryloyldimethyltaurate/VP copolymer, acrylates/C10-30 alkyl acrylate crosspolymer, and mixtures thereof.

Suitable organically modified clays include, but are not limited to, organically modified versions of hectorite, bentonite, smectite and montmorillonite clay (such as those sold under tradename BENTONE® from Elementis Specialties, TIXO-GEL® from Sud-Chemie, and CLAYTONER from Southern Clay Products). Hydrophilically modified fumed silicas include, but are not limited to, WACKER HDK® N20 and T30 grades (Wacker-Chemie AG), and hydrophilic grades under tradename of AEROSIL® (Evonik). Silicone gels or silicone elastomers include, but are not limited to, the “KSG” thickening series (KSG-15, KSG-16. KSG-18, KSG-41, KSG-42, KSG-43, KSG-44) from Shin-Etsu Silicones, DOW CORNING®9040, 9041, 9045, and 9546 silicone elastomer blends from Dow Corning, SFE839™, and Velvesil™ silicone gels from Momentive Performance Materials, and WACKER-BELSIL® RG-100 from Wacker-Chemie AG.

In some embodiments, the compositions disclosed herein can further comprise one or more lipo-soluble/lipo-dispersible film-forming agent. Lipo-soluble/lipo-dispersible film-forming agents suitable for use herein include, but are not limited to, organic silicone resins (e.g., trimethylsiloxysilicate such as SRI 000 from GE Silicones) and copolymers of organic silicone resins (e.g., diisostearyl trimethylolpropane siloxy silicate such as SF1318 from GE Silicones); fluorinated silicone resins; acrylic and/or vinyl based polymers or copolymers, including silicone and/or fluorinated versions (e.g., the “KP” series of silicone acrylates from Shin-Etsu Silicones, and 3M™ Silicones “Plus” Polymer VS70 and SA70); polyurethanes (e.g., the hydroxyester triglyceride derived Poly Derm® series from Alzo International); polyesters (e.g., the Lexorez® series of polymeric polyesters from Inolex Chemical Company); and mixtures thereof.

In some embodiments, the composition disclosed herein can further comprise one or more coloring agents. Coloring agents suitable for use herein include all inorganic and organic colors/pigments, including mineral or pearl pigments suitable for use in cosmetic compositions. Such coloring agents include those either with or without a surface coating or treatment. Coloring agents can intensify coloration, and/or light scattering, and/or light reflecting effects of the composition.

In some embodiments, the composition disclosed herein can further comprise one or more sunscreens, e.g., mineral and/or physical sunscreens. Sunscreens can block UVA and/or UVB radiation. Exemplary UVA sunscreen agents include, but are not limited to, avobenzone, terephthalylidene dicamphor sulfonic acid, bis-disulizole disodium, disodium phenyl dibenzimidazole tetrasulfonate, diethylamino hydroxybenzoyl hexylbenzoate, bis-diethylamino hydroxybenzoyl benzoate, bis-benzoxazolylphenyl ethylhexylamino triazine, and combinations thereof.

Exemplary UVB sunscreen agents include, but are not limited to, octocrylene, octinoxate, octisalate, homosalate, ensulizole, ethylhexyl triazone, enzacamene, amiloxate, diethylhexyl butamido triazine, benzylidene malonate polysiloxane, padimate-O, trolamine salicylate, cinoxate, p-aminobenzoic acid and derivatives thereof, and combinations thereof.

Exemplary sunscreen agents that absorb both UVA and UVB radiation such as, for example, oxybenzone, meradimate, titanium dioxide, zinc oxide, bis-octrizole, bemotrizinol, drometrizole trisiloxane, sulisobenzone, dioxybenzone, or combinations thereof.

Specific Suitable sunscreens include but are not limited to p-aminobenzoic acid, its salts and its derivatives (ethyl, isobutyl, glyceryl esters, p-dimethylaminobenzoic acid, anthranilates (i.e., o-aminobenzoates, methyl, menthyl, phenyl, benzyl, phenylethyl, linallyl, terpinyl, and cyclohexenyl esters), salicylates (amyl, phenyl, benzyl, menthyl, glyceryl, and dipropylene glycol esters), cinnamic acid derivatives (methyl and benzyl esters, alpha-phenyl cinnamonitrile, butyl cinnamoyl pyruvate), dihydroxycinnamic acid derivatives (umbelliferone, methylumbelliferone, methylaceto umbelliferone), trihydroxycinnamic acid derivatives (esculetin, methyl eSculletin, daphnetin, and the glucosides, esculin and daphnin), hydrocarbons (diphenylbutadiene, stilbene), dibenzalacetone and benzalacetophenone, naphtholsulfonates (sodium salts of 2-naphthol-3,3-disulfonic and of 2-naphthol-6,8-disulfonic acids), cihydroxynaphthoic acid and its salts, o- and phydroxybiphenyldisulfonates, coumarin derivatives (7 hydroxy, 7-methyl, 3-phenyl), diazoles (2-acetyl-3-bromoindazole, phenyl benzoxazole, methylnaphthoxalole, various arylbenzothiazoles), quinine salts (bisulfate, sulfate, chloride, oleate, and tannate), quinoline derivatives (8-hydroxyquinoline salts, 2-phenylquinoline), hydroxy- or methoxy substituted benzophenones, uric and vilouric acids, tannic acid and its derivatives (e.g., hexaethylether), (butyl carbityl) (6-propyl piperonyl) ether, hydroquinone, benzophenones (oxybenzene, sulisobenzone, dioxybenzone, benzoresorcinol, 2,2,4,4′-tetrahydroxybenzophenone, 2,2′-dihydroxy4,4′-dimethoxybenzophenone, octabenzone, 4-isopropyhldibenzoylmethane, butylmethoxydibenzoylmethane, etocrylene, and 4-isopropyl-di-benzoylmethane, titanium dioxide, iron oxide, zinc oxide, and mixtures thereof. Other cosmetically-acceptable sunscreens and concentrations (percent by weight of the total cosmetic sunscreen composition) include diethanolamine methoxycinnamate (10% or less), ethyl-bis(hydroxypropyl)aminobenzoate (5% or less), glyceryl aminobenzoate (3% or less), 4-isopropyl dibenzoylmethane (5% or less), 4-methylbenzylidene camphor (6% or less), terephthalylidene dicamphor sulfonic acid (10% or less), and sulisobenzone (also called benzophenone-4, 10% or less). In some embodiments, the composition disclosed herein can further comprise D-aspartic acid and/or D-alanine and any salts thereof. As used herein, the term “derivatives” of D-aspartic acid and D-alanine indicates D-aspartic acid and D-alanine molecules that are covalently bound to any organic group via their amino groups, carboxyl groups, or side chains, provided that the effect on promoting collagen production of D-aspartic acid and D-alanine is not impaired. Exemplary organic groups include, but are not limited to, protective groups, such as N-phenylacetyl group, and 4,4′-dimethoxytrityl (DMT) group; biopolymers, such as a protein, a peptide, a saccharide, a lipid, and a nucleic acid; synthetic polymers, such as a polystyrene, a polyethylene, a polyvinyl, a polypropylene, and a polyester; and functional groups such as an ester group. The ester group can comprise, for example, an aliphatic ester, such as methyl ester, and ethyl ester; and an aromatic ester.

In some embodiments, the composition can further comprise one or more general skin care additives such as, e.g., conditioning agents such silicones. In some embodiments, the composition can further comprise one or more shark liver oils, e.g., squalane and/or squalene. In some embodiments, the composition can further comprise one or more polysaccharides produced by microalgae, e.g., alguronic acid.

In some embodiments, the composition can further comprise at least one preservative. In some embodiments, the at least one preservative can be quarternary ammonium compounds, halogenated phenols, sorbic acid, potassium sorbate, benzoic acid, sodium benzoate, sodium citrate, sodium anisate, caprylhydroxamic acid, sodium levulinate, phenoxyethanol, or combinations thereof.

The compositions described herein can have a pH in the range of about 4 to about 8, from about 4.7 to about 5.5, from about 5 to about 7, from about 6 to about 7, from about 6.1 to about 6.8, or from about 6.4 to 6.6.

### Soaps

In some embodiments, the cosmetic composition described herein can be a cleansing composition or soap, including traditional soaps in the form of solid bar and liquid soaps in the form of cleaners, makeup removers, body washes, milks, creams, foams cream gels, or gels that can be packaged in tubes, bottles, pump bottles, aerosol shower foams or foam pump bottles. Soaps can be used in a cosmetic process for cleaning the dirt residues of human keratinous materials in the presence of water, massed to form a foam and the formed foam and the soil residues are removed by rinsing with water, and can be used on any part of the body and face disclosed herein, including, for example, on the skin of the body, face, hands, lips, eyelids, nails, hair, eyelashes and/or eyebrows.

Traditionally, solid soaps include alkali metal fatty acid salts and potassium fatty acid soaps, and liquid soaps include four main families of detergent formulation: (1) those based on lauryl sulfate; those based on alpha-olefin sulfonate, (3) those based on a mixture of synthetic anionic, amphoteric and/or nonionic surfactants; (4) mixed formulations based on soaps and synthetic surfactants. Liquid soaps generally contain a thickening system chosen, for example, from electrolytes such as sodium chloride, potassium chloride or potassium sulphate; alkanolamides such as cocamide DEA or cocamide MEA; esters of polyethylene glycol and monoacid or stearic acid such as polyethylene glycol distearate 6000 or mixtures thereof and are contained in a cosmetically acceptable aqueous medium. However, both solid and liquid soaps can comprise any suitable additional ingredients such as those listed below herein, in any combination.

In some embodiments, the cleansing composition or soap described herein can further comprise one or more cellulosic compounds, or polysaccharide compound having in its structure chains of glucose residues linked by β-1,4 bonds, one or more fatty acids comprising a linear or branched, saturated or unsaturated alkyl chain having from 6 to 30 carbon atoms or 12 to 22 carbon atoms, one or more fatty acids, including lauric acid, myristic acid, palmitic acid and stearic acid, linolenic acid, and mixtures thereof, and one or more mineral bases, including alkali metal hydroxides (sodium hydroxide and potassium hydroxide), metal hydroxides or ammonia or organic bases such as triethanolamine, monethanolamine, monoisopropanolmaine, N-methylglucamine, lysine and arginine.

In some embodiments, the composition described herein can further comprise one or more anionic surfactants or salts, including alkali metal salts such as sodium salts, ammonium salts, amine salts, aminoalcohol salts or salts. alkaline earth metals, for example, magnesium, of the following types: alkyl sulphates, alkyl ether sulphates, alkyl amido ether sulphates, alkyl aryl polyether sulphates, monoglyceride sulphates; alkylsulfonates, alkylamidesulfonates, alkylarylsulphonates, α-olefin-sulfonates, paraffin-sulfonates; alkylsulfosuccinates, alkylethersulfosuccinates, alkylamide-sulfosuccinates; alkylsulfoacetates; acylsarcosinates; and the acylglutamates, the alkyl and acyl groups of all these compounds having from 6 to 24 carbon atoms and the aryl group denoting a phenyl or benzyl group, C₆-C₂₄ alkyl esters of polyglycoside carboxylic acids such as alkyl glucoside citrates, alkyl polyglycoside tartrates and alkyl polyglycoside sulfosuccinates, alkylsulfosuccinamates, acylisethionates and N-acyltaurates, the alkyl or acyl group of all these compounds having from 12 to 20 carbon atoms, and/or acyllactylates whose acyl group contains from 8 to 20 carbon atoms and their mixtures. In some embodiments, alkyl-D-galactoside uronic acids, polyoxyalkylenated (C₆-C₂₄) ether carboxylic acids, polyoxyalkylenated (C₆-C₂₄) aryl (C₆-C₂₄) polyoxyalkylenated ether carboxylic acids, polyoxyalkylenated (C₆-C₂₄) alkyl amidoether carboxylic acids, in particular those containing from 2 to 50 ethylene oxide groups; and their alkali metal, ammonium, amine, aminoalcohol or alkaline earth metal salts can also be suitable.

Suitable C₆-C₂₄ alkyl ether sulphate salts containing from 1 to 30 ethylene oxide groups, include alkali metals or alkaline earth metals, ammonium, amine or amino alcohol salts, sodium salts and oxyethylenated (C₁₂-C₁₄) alkyl ethersulfates having an average number of ethylene oxide groups of between 1 and 4 and including sodium laureth sulfate (CTFA name) such as the commercial product sold under the name TEXAPON AOS 225 UP TEXAPON N702 TEXAPON NSW marketed by COGNIS or EMPICOL ESB3/FL2, EMPICOL ESB3/FL3, EMPICOL ESB70/FL2 sold by the company Huntsman.

Suitable amphoteric surfactants include, but are not limited to, derivatives of secondary or tertiary aliphatic amines, in which the aliphatic group is a linear or branched chain comprising from 8 to 22 carbon atoms. The amphoteric surfactants can contain at least one water-soluble anionic group such as a carboxylate, sulfonate, sulfate, phosphate or phosphonate group, (C₈-C₂₀) alkylbetaines, sulphobetaines, (C₈-C₂₀) alkylamido (C₆-C₈) alkylbetaines or (C₈-C₂₀) alkylamidoalkyl (C₆-C₈) sulfobetaines, and mixtures thereof.

Suitable amine derivatives include the products sold under the name MIRANOL®, as described in U.S. Pat. Nos. 2,528,378 and 2,781,354 and filed in the CTFA dictionary, 3rd edition, 1982, under the terms Amphocarboxy-glycinate and Amphocarboxypropionate denominations. Additional suitable amine derivatives include those classified in the CTFA dictionary, 5th edition, 1993, under the names cocoamphodiacetate disodium, lauroamphodiacetate disodium, caprylamphodiacetate disodium, capryloamphodiacetate disodium, cocoamphodipropionate disodium, lauroamphodipropionate disodium, caprylamphodipropionate disodium, capryloamphodipropionate disodium, acid lauroamphodipropionic, cocoamphodipropionic acid, and cocoamphodiacetate sold under the trade name MIRANOL® C2M concentrated by Rhodia. Suitable alkyl (C₈-C₂₀) betaines include cocamidopropyl betaine and cocobetaine such as the commercial products MIRATAINE BB/FLA from RHODIA or EMPIGEN BB/FL from Huntsman.

In some embodiments, the cleansing compositions or soaps described herein can be sulfate-free, and can include a sulfate-free surfactant system.

In some embodiments, the cleansing composition or soap described herein can further comprise one or more thickeners of the nonionic cellulosic compound type. Suitable cellulosic compounds include, but are not limited to, nonionic cellulose ethers, including methylcelluloses and ethylcelluloses; hydroxyalkylcelluloses such as hydroxymethylcelluloses, hydroxyethylcelluloses and hydroxypropylcelluloses; mixed hydroxyalkyl-alkylcellulose celluloses such as hydroxypropyl-methylcelluloses, hydroxyethyl-methylcelluloses, hydroxyethylethylcelluloses and hydroxybutyl-methylcelluloses and hydroxyalkylcelluloses modified with an alkyl chain. Suitable hydroxypropyl methylcelluloses include the commercial products METHOCEL E, F, J and K sold by Dow Corning and even more particularly METHOCEL E 4MQG or METHOCEL F 4M. Suitable cellulosic ingredients can be in a crystalline form, a microcrystalline form, or a mixture thereof.

In some embodiments, the cleansing composition or soap described herein can further comprise one or more additional thickeners including electrolytes such as sodium chloride, potassium chloride or potassium sulphate; alkanolamides such as cocamide DEA or cocamide MEA; esters of polyethylene glycol and monoacid or stearic acid such as polyethylene glycol distearate 6000 or mixtures thereof, polysaccharide biopolymers such as xanthan gum, guar gum, alginates, synthetic polymers such as polyacrylics such as CARBOPOL 980, CARBOPOL 1382 marketed by NOVEON, acrylate/acrylonitrile copolymers such as HYPAN SS201 marketed by KINGSTON, clays such as smectites, modified or unmodified hectorites such as BENTONE products marketed by Rheox, LAPONITE products marketed by Southern Clay Products, VEEGUM HS product marketed by RT Vanderbilt, and mixtures thereof.

In some embodiments, the cleansing composition or soap described herein can further comprise one or more nonionic surfactants. These are well-known compounds (see regard “Handbook of Surfactants” by MR PORTER, Blackie & Son editions (Glasgow and London), 1991, pp 116-178), and can be chosen from alcohols, alpha-diols, (C₁-C₂₀) alkyl phenols or polyethoxylated, polypropoxylated or polyglycerolated fatty acids, having a fatty chain comprising, for example, from 8 to 18 atoms, the number of ethylene oxide groups or propylene oxide can range from 2 to 50 and the number of glycerol groups can range from 2 to 30, copolymers of ethylene oxide and propylene, condensates of ethylene oxide and propylene oxide on fatty alcohols; polyethoxylated fatty amides having from 2 to 30 moles of ethylene oxide, polyglycerolated fatty amides comprising on average 1 to 5 glycerol groups; polyethoxylated fatty amines having 2 to 30 moles of ethylene oxide, ethoxylated sorbitan fatty acid esters having 2 to 30 moles of ethylene oxide; sucrose fatty acid esters, polyethylene glycol fatty acid esters, (C₆-C₂₄) alkyl polyglycosides, N-alkyl (C₆-C₂₄) glucamine derivatives, amine oxides such as that the oxides of alkyl (C₁₀-C₁₄) amines or the oxides of N-acyl (C₁₀-C₁₄)-aminopropylmorpholine, and mixtures thereof.

Additional suitable nonionic surfactants include, but are not limited to, alkyl polyglucosides (APG), maltose esters, polyglycerolated fatty alcohols, glucamine derivatives, for instance 2-ethylhexyloxycarbonyl-N-methylglucamine, and mixtures thereof. Suitable alkylpolyglucosides include those that contain an alkyl group comprising from 6 to 30 carbon atoms, and a hydrophilic group (glucoside). Exemplary alkylpolyglucosides include decylglucoside (alkyl-C9/C11-polyglucoside (1.4)), including the product sold under the name Mydol 10® by the company Kao Chemicals, the product sold under the name Plantaren 2000 UP® by the company Cognis, and the product sold under the name Oramix NS 10® by the company SEPPIC, and caprylyl/capryl glucosides, including the product sold under the name Oramix CG 110® by the company SEPPIC; laurylglucoside, sold as Plantaren 1200 N® and Plantacare 1200® by the company Cognis, and cocoglucoside, for instance the product sold under the name Plantacare 818/UP® by the company Cognis.

Suitable maltose derivatives include those described in document EP-A-566 438, such as O-octanoyl-6′-D-maltose or O-dodecanoyl-6′-D-maltose described in document FR-2 739 556.

In some embodiments, the cleansing composition or soap can be formulated in a cosmetically acceptable aqueous medium. Suitable cosmetically acceptable aqueous media can include, in addition to water, one or more solvents such as lower alcohols containing from 1 to 6 carbon atoms, such as ethanol; polyols such as glycerine; glycols such as butylene glycol, isoprene glycol, propylene glycol, polyethylene glycols such as PEG-8, sorbitol, sugars such as glucose, fructose, maltose, lactose, sucrose, and mixtures thereof. The amount of solvent(s) in the composition disclosed herein can range from 0.1 to 95% by weight.

In some embodiments, the cleansing composition or soap described herein can further comprise one or more cationic polymers of the polyquaternium type, which can provide softness and lubricity to a foaming composition. Suitable cationic polymers include Polyquaternium 5 such as the product MERQUAT 5 marketed by the company CALGON, Polyquaternium 6 such as the product SALCARE SC 30 marketed by the company CIBA, and the product MERQUAT 100 marketed by the company CALGON, Polyquaternium 7 such as the MERQUAT S, MERQUAT 2200 and MERQUAT 550 products marketed by the company CALGON, and the SALCARE SC 10 product marketed by the company CIBA, Polyquaternium 10 such as the product Polymer JR400 marketed by the company Amerchol, Polyquaternium 11 such as GAFQUAT 755, GAFQUAT 755N and GAFQUAT 734 products marketed by ISP, Polyquaternium 15 such as the product ROHAGIT KF 720 F marketed by the company ROHM, Polyquaternium 16 such as LUVIQUAT FC905, LUVIQUAT FC370, LUVIQUAT HM552 and LUVIQUAT FC550 products marketed by BASF, Polyquaternium 22 such as the product Merquat 280 sold by the company Calgon, Polyquaternium 28 such as the product STYLEZE CC10 marketed by the company ISP, Polyquaternium 39 such as the MERQUAT PLUS 3330 product marketed by Calgon, Polyquaternium 44 such as the product LUVIQUAT CARE sold by the company BASF, Polyquaternium 46 such as the product LUVIQUAT HOLD marketed by the company BASF, Polyquaternium 47 such as the product MERQUAT 2001 marketed by Calgon, and cationic guars such as the product Jaguar marketed by the company Rhodia can also be used as cationic polymer.

In some embodiments, the cleansing composition or soap described herein can further comprise one or more adjuvants or additives used in cosmetic compositions. Suitable adjuvants or additives include but are not limited to oils, active agents, perfumes, preservatives, sequestering agents, pearlescent or opacifying agents, pigments, pearlescent agents, mineral or organic fillers such as talc, kaolin, silica powders or of polyethylene, soluble dyes, or any combination thereof.

Examples of oils include vegetable oils (jojoba, avocado, sesame, sunflower, corn, soy, safflower, grape seed), mineral oils (vaseline, isoparaffins optionally hydrogenated), synthetic oils (isopropyl myristate, cetearyl octanoate, polyisobutylene, ethyl hexyl palmitate, alkyl benzoates), volatile or non-volatile silicone oils such as polydimethylsiloxanes (PDMS) and cyclodimethylsiloxanes or cyclomethicones, and fluorinated or fluorosilicone oils and mixtures thereof.

Exemplary active agents include sunscreens, desquamating agents, moisturizing agents, depigmenting agents, pro-pigmentants, alpha-hydroxy acids, antibacterial agents, antiradical agents, anti-pollution agents, anti-inflammatoires, retinoids, extracts of algae, mushrooms, vegetables, yeasts, bacteria, hydrolysed, partially hydrolyzed or unhydrolyzed proteins, enzymes, hormones, vitamins and their derivatives, flavonoids and isoflavones, and mixtures thereof.

The cleansing composition or soap described herein can have a pH ranging from 6 to 10 depending on the application chosen. The adjustment of the pH to the desired value can be done conventionally by adding a base (organic or inorganic) in the composition, for example ammonia or a primary, secondary or tertiary (poly) amine such as monoethanolamine, diethanolamine, triethanolamine, isopropanolamine or 1,3-propanediamine, or by addition of a mineral or organic acid, such as a carboxylic acid, for example, citric acid. In the context of shower gels, the pH can vary from 8 to 10.

In some embodiments, the cleansing composition described herein can further comprise optional additives such as colorants, fragrances, antibacterials, preservatives, antioxidants, beads (fragrance, exfoliating or moisturizing), mica, glitter, shea butter, shea butter beads, opacifying agents, pearlizing agents and other such ingredients. In some embodiments, the composition has high clarity (about 2 to about 25 NTU's), a targeted viscosity (about 4,000 to about 10,000 centipoise) for ease of dispensing from an orifice in the range of about ¼ to about ⅛, and a yield value (about 3 to about 15 Pascals) that allows the composition to suspend a variety of additives with a uniformity of distribution and enhanced stability (for example, about 8 months at 120° F. (49° C.), and any mixture thereof.

In some embodiments, the cleansing composition or soap can further comprise one or more moisturizers/emollients. Moisturizers can be included in bar or liquid soap compositions to provide conditioning benefits to the skin. The term “moisturizer” describes a material which imparts a smooth and soft feeling to the skin surface.

There are two ways of reducing water loss from the stratum corneum. One is to deposit on the surface of the skin an occlusive layer which reduces the rate of evaporation. The second method is to add nonocclusive hygroscopic substances to the stratum corneum which will retain water and make this water available to the stratum corneum to alter its physical properties and produce a cosmetically desirable effect. Nonocclusive moisturizers also function by improving the lubricity of the skin.

Both occlusive and nonocclusive moisturizers are contemplated for use in the compositions described herein. Exemplary moisturizers include long chain fatty acids, liquid water-soluble polyols, glycerin, propylene glycol, sorbitol, polyethylene glycol, ethoxylated/propoxylated ethers of methyl glucose (e.g., methyl gluceth-20), ethoxylated/propoxylated ethers of lanoline alcohol (e.g., Solulan-75® available from the Amerchol Co.) coconut and tallow fatty acids, liquid water-soluble polyols (e.g., glycerin, propylene glycol, butylene glycol, hexylene glycol, polypropylene glycol and polyethylene glycol).

Nonocclusive moisturizers can naturally occur in the stratum corneum of the skin, such as sodium pyrrolidone carboxylic acid, lactic acid, urea, L-proline, guanidine and pyrrolidone. Examples of other nonocclusive moisturizers include hexadecyl, myristyl, isodecyl or isopropyl esters of adipic, lactic, oleic, stearic, isostearic, myristic or linoleic acids, as well as many of their corresponding alcohol esters (sodium isostearoyl-2-lactylate, sodium capryl lactylate), hydrolyzed protein and other collagen-derived proteins, aloe vera gel and acetamide MEA (N-acetyl ethanolamine). Other examples of both occlusive and nonocclusive types of moisturizers are disclosed in “Emollients—A Critical Evaluation,” by J. Mausner, Cosmetics & Toiletries, May 1981, incorporated herein by reference.

Exemplary occlusive moisturizers include petrolatum, mineral oil, beeswax, silicones, lanolin and oil-soluble lanolin derivatives, saturated and unsaturated fatty alcohols such as behenyl alcohol, squalene and squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

### Hair Care

In some embodiments, the composition be a hair care composition comprising ingredients commonly used for hair care products. These ingredients can include, but are not limited to, cleansing agents, lathering agents, hydration agents, surfactants, detergents, gelling agents, fragrances, botanical extracts, conditioning agents, humectants, silicones or silicone derivatives, thickening agents, sun blocks, vitamins, alcohols, polyols, polyolethers, and other commonly used ingredients in shampoos, conditioners, and styling agents. Hair care products generally include one or more surfactants, one or more viscosity adjusting agents, one or more preservatives, and one or more fragrances, and any of the ingredients listed below or combinations thereof.

Generally, hair can be damaged and embrittled by the action of external atmospheric agents such as light and bad weather, but also by mechanical or chemical treatments, such as brushing, combing, dyeing, bleaching, permanent-waving and/or relaxing, and over time, hair can become dry, coarse, dull, and/or fragile. To overcome these drawbacks, it is common practice to use care compositions that condition the hair appropriately, giving it satisfactory cosmetic properties, especially in terms of smoothness, sheen, softness, suppleness, lightness, a natural feel and good disentangling properties. These hair care compositions can be, for example, shampoos, conditioning shampoos, hair conditioners, masks, sera, gels, hair lotions and creams that can be rinsed-out or leave-in compositions. In various embodiments, these compositions generally comprise a combination of cationic conditioning agents such as cationic surfactants, cationic polymers, silicones and/or fatty substances, such as fatty alcohols, in order to give the hair satisfactory cosmetic properties, in terms of softness, smoothness and suppleness. Exemplary compositions can comprise silicones, which are known to improve the cosmetic properties of hair in terms of smoothness and flexibility (as shown in U.S. Pat. No. 5,374,421, each of which is incorporated herein by reference).

In some embodiments, the hair care composition can further comprise one or more non-amino polyalkylsiloxanes, one or more oxyethylenated polymers in the presence of fatty alcohols, one or more non-amino polyalkylsiloxanes comprising at least one alkyl chain having at least 12 carbon atoms, one or more oxyethylenated polymers, and/or one or more fatty alcohols. Exemplary oxyethylenated polymers can have a weight-average molecular mass greater than or equal to 106. In some embodiments, the oxyethylenated polymers can be chosen from the compounds having the formula H(OCH₂CH₂)_{z}OH, wherein, z is an integer greater than or equal to 30,000. In certain embodiments, z can range from 5,000 to 120,000, or from 40,000 to 95,000. In some embodiments, the oxyethylenated polymer can be PEG-45M (z=45,000) such as the product sold under the name Polyox WSR N 60 K by the company Amerchol, and PEG-90M (z=90 000), and mixtures thereof.

In some embodiments, the hair care composition can further comprise one or more fatty alcohols. The term “fatty alcohol” means any saturated or unsaturated, linear or branched alcohol comprising at least 8 carbon atoms, and which is not oxyalkylenated. Exemplary fatty alcohols are solid at room temperature (25° C.) and at atmospheric pressure (1.013*10₅ Pa). Exemplary fatty alcohols include cetyl alcohol, stearyl alcohol, oleyl alcohol, behenyl alcohol, linoleyl alcohol, palmitoleyl alcohol, arachidonyl alcohol, erucyl alcohol, cetylstearyl (or cetearyl) alcohol, and mixtures thereof.

In some embodiments, the hair care composition can further comprise one or more conditioning agents, including cationic surfactants, cationic polymers, and mixtures thereof. The term “cationic surfactant” means a surfactant that is positively charged when it is contained in the composition described herein. Suitable cationic surfactants can be chosen from primary, secondary, or tertiary fatty amines, optionally polyoxyalkylenated, or salts thereof, and quaternary ammonium salts, and mixtures thereof. An exemplary fatty amine is stearamidopropyl dimethylamine. Exemplary quaternary ammonium salts include tetraalkylammonium salts, including dialkyldimethylammonium or alkyltrimethylammonium salts in which the alkyl group contains approximately from 16 to 22 carbon atoms, in particular behenyltrimethylammonium, distearyldimethylammonium, cetyltrimethylammonium or benzyldimethylstearylammonium salts, or, on the other hand, the palmitylamidopropyltrimethylammonium salt, the stearamidopropyltrimethylammonium salt, the stearamidopropyldimethylcetearylammonium salt, or the stearamidopropyldimethyl(myristyl acetate) ammonium salt sold under the name CERAPHYL 70 by the company Van Dyk.

The term “cationic polymer” means any polymer containing cationic groups and/or groups that can be ionized to cationic groups, which can be non-siliceous. Exemplary cationic polymers include any known for styling the hair, for example, those described in patent application EP-A-0 337 354 and in French patent applications FR-A-2 270 846, 2 383 660, 2 598 611, 2 470 596 and 2 519 863, each of which is incorporated by reference in its entirety. Additional exemplary cationic polymers include those containing units comprising primary, secondary, tertiary and/or quaternary amine groups that can either form part of the main polymer chain or can be borne by a side substituent directly connected thereto. Suitable cationic polymers can have a weight-average molecular mass of greater than 10₅, including polymers of polyamine, polyaminoamide and polyquaternary ammonium type, including those described in French patents 2 505 348 and 2 542 997, each of which is incorporated by reference in its entirety.

In some embodiments, the hair care composition can further comprise a “non-sulfate” cleansing agent, lathering agent, or surfactant agent. Suitable “non-sulfate” agents include but are not limited to: sodium lauroyl methyl isethionate propanediol, sodium methyl oleoyl taurate, and sodium cocoyl isethionate.

In some embodiments, the hair care composition can further comprise any of the following ingredients or mixtures thereof: quaternary ammonium compound synthetically derived from rapeseed, quaternary polymers of vinylpyrrolidone and/or of vinylimidazole, for instance the products sold under the names LUVIQUAT FC 905, FC 550 and FC 370 and LUVIQUAT Excellence by the company BASF, Cationic polysaccharides, including cationic celluloses, including cellulose ether derivatives comprising quaternary ammonium groups, cationic cellulose copolymers or cellulose derivatives grafted with a water-soluble quaternary ammonium monomer, and cationic galactomannan gums. Exemplary cellulose ether derivatives comprising quaternary ammonium groups are described in French patent 1 492 597. These polymers are also defined in the CTFA dictionary as quaternary ammoniums of hydroxyethylcellulose that have reacted with an epoxide substituted with a trimethylammonium group. Cationic cellulose copolymers or the cellulose derivatives grafted with a water-soluble quaternary ammonium monomer are described in U.S. Pat. No. 4,131,576, such as hydroxyalkyl celluloses, for instance hydroxymethyl, hydroxyethyl or hydroxypropyl celluloses grafted especially with a methacryloylethyltrimethylammonium, methacrylamidopropyltrimethylammonium or dimethyldiallylammonium salt. Suitable associative celluloses such as alkylhydroxyethylcelluloses quaternized with C₈-C₃₀ fatty chains, such as the product QUATRISOFT LM 200, sold by the company Amerchol/Dow Chemical (INCI name Polyquaternium-24) and the products CRODACEL QM (INCI name PG-Hydroxyethylcellulose cocodimonium chloride), CRODACEL QL (C₁₂ alkyl) (INCI name PG-Hydroxyethylcellulose lauryldimonium chloride) and CRODACEL QS (C₁₈ alkyl) (INCI name PG-Hydroxyethylcellulose stearyldimonium chloride) sold by the company Croda. Other suitable fatty-chain hydroxyethylcellulose derivatives include the commercial products SOFTCAT Polymer SL such as SL-100, SL-60, SL-30 and SL-5 from the company Amerchol/Dow chemical of INCI name Polyquaternium-67. Suitable cationic galactomannan gums are described in U.S. Pat. Nos. 3,589,578 and 4,031,307. Suitable cellulosic ingredients can be in a crystalline form, a microcrystalline form, or a mixture thereof.

In some embodiments, the hair care composition can further comprise one or more cationic proteins or cationic protein hydrolysates, polyalkyleneimines, including polyethyleneimines, polymers containing vinylpyridine or vinylpyridinium units, condensates of polyamines and of epichlorohydrin, quaternary polyureylenes and chitin derivatives, animal protein hydrolysates bearing trimethylbenzylammonium groups, such as the products sold under the name Crotein BTA by the company Croda and referred to in the CTFA dictionary as Benzyltrimonium hydrolyzed animal protein, protein hydrolysates bearing quaternary ammonium groups on the polypeptide chain, the said ammonium groups comprising at least one alkyl radical having from 1 to 18 carbon atoms.

In some embodiments, the hair care composition can further comprise one or more quaternized plant proteins such as from wheat, corn or soybean proteins, for example, quaternized wheat proteins, including those sold by the company Croda under the names Hydrotriticum WQ or QM, referred to in the CTFA dictionary as Cocodimonium hydrolysed wheat protein, Hydrotriticum QL, referred to in the CTFA dictionary as Laurdimonium hydrolysed wheat protein, or else Hydrotriticum QS, referred to in the CTFA dictionary as Steardimonium hydrolysed wheat protein.

In some embodiments, the hair care composition can further comprise one or more polyamines such as POLYQUART R H sold by Cognis, referred to under the name polyethylene glycol tallow polyamine in the CTFA dictionary. Additional suitable polymers include those sold especially under the name Lupamin by the company BASF, and the products sold under the names Lupamin 9095, Lupamin 5095, Lupamin 1095, Lupamin 9030 and Lupamin 9010.

In some embodiments, the hair care composition can further comprise one or more fatty substances that are liquid at room temperature (25° C.) and at atmospheric pressure (1.013*10⁵ Pa). The term “fatty substance” means an organic compound that is insoluble in water at ordinary temperature (25° C.) and at atmospheric pressure (1.013*10⁵ Pa) (solubility of less than 5%, less than 1%, or less than 0.1%). Fatty substances are generally soluble in organic solvents under the same temperature and pressure conditions, for example, in chloroform, dichloromethane, carbon tetrachloride, ethanol, benzene, toluene, tetrahydrofuran (THF), liquid petroleum jelly or decamethylcyclopentasiloxane. The liquid fatty substances of the present disclosure can be nonpolyoxyethylenated and nonpolyglycerolated. The term “oil” means a “fatty substance” that is liquid at room temperature (25° C.) and at atmospheric pressure (1.013*10⁵ Pa). The term “non-silicone oil” means an oil not containing any silicon atoms (Si) and the term “silicone oil” means an oil containing at least one silicon atom. The liquid fatty substances can be chosen from non-silicone oils such as in particular C₆-C₁₆ liquid hydrocarbons, liquid hydrocarbons containing more than 16 carbon atoms, non-silicone oils of animal origin, triglycerides of plant or synthetic origin, fluoro oils, liquid fatty acid and/or fatty alcohol esters other than triglycerides, and mixtures thereof. The liquid hydrocarbons can be linear, branched or optionally cyclic, including hexane, cyclohexane, undecane, dodecane, tridecane or isoparaffins, such as isohexadecane, isodecane or isododecane, and mixtures thereof. Suitable linear or branched liquid hydrocarbons of mineral or synthetic origin containing more than 16 carbon atoms can be chosen from liquid paraffins, petroleum jelly, liquid petroleum jelly, mineral oil, polydecenes and hydrogenated polyisobutene such as PARLEAM and mixtures thereof. Hydrocarbon-based oils of animal origin, such as perhydrosqualene, can be used.

Exemplary triglycerides of vegetable or synthetic origin can be chosen from liquid fatty acid triglycerides comprising from 6 to 30 carbon atoms, for instance heptanoic or octanoic acid triglycerides, or alternatively, more particularly from those present in plant oils, for instance coconut oil, sunflower oil, corn oil, soybean oil, marrow oil, grapeseed oil, sesame seed oil, hazelnut oil, apricot oil, macadamia oil, arara oil, castor oil, avocado oil, jojoba oil, shea butter oil or synthetic caprylic/capric acid triglycerides, such as those sold by the company Stearineries Dubois or those sold under the names MIGLYOL 810, 812 and 818 by the company Dynamit Nobel, and mixtures thereof. Suitable fluoro oils include perfluoromethylcyclopentane and perfluoro-1,3-dimethylcyclohexane, such as FLUTEC PC1 and FLUTEC PC3 by the company BNFL Fluorochemicals; perfluoro-1,2-dimethylcyclobutane; perfluoroalkanes such as dodecafluoropentane and tetradecafluorohexane, sold under the names PF 5050 and PF 5060 by the company 3M, or bromoperfluorooctyl sold under the name FORALKYL by the company Atochem; nonafluoromethoxybutane and nonafluoroethoxyisobutane; perfluoromorpholine derivatives such as 4-trifluoromethyl perfluoromorpholine sold under the name PF 5052 by the company 3M.

Suitable monoesters include dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso) stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates; 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate, and mixtures thereof.

In some embodiments, the hair care composition can further comprise diethyl sebacate, diisopropyl sebacate, diisopropyl adipate, di(n-propyl) adipate, dioctyl adipate, diisostearyl adipate, dioctyl maleate, glyceryl undecylenate, octyldodecyl stearoyl stearate, pentaerythrityl monoricinoleate, pentaerythrityl tetraisononanoate, pentaerythrityl tetrapelargonate, pentaerythrityl tetraisostearate, pentaerythrityl tetraoctanoate, propylene glycol dicaprylate, propylene glycol dicaprate, tridecyl erucate, triisopropyl citrate, triisostearyl citrate, glyceryl trilactate, glyceryl trioctanoate, trioctyldodecyl citrate, trioleyl citrate, propylene glycol dioctanoate, neopentyl glycol diheptanoate, diethylene glycol diisononanoate, and polyethylene glycol distearates, and mixtures thereof.

In some embodiments, the hair care composition can further comprise one or more fatty esters, one or more sugar esters, and/or one or more diesters of C₆-C₃₀, such as C₁₂-C₂₂ fatty acids. The term “sugar” means oxygen-bearing hydrocarbon-based compounds containing several alcohol functions, with or without aldehyde or ketone functions, and which comprise at least 4 carbon atoms. Suitables sugars can include monosaccharides, oligosaccharides or polysaccharides, for example, sucrose (or saccharose), glucose, galactose, ribose, fucose, maltose, fructose, mannose, arabinose, xylose and lactose, and derivatives thereof, such as alkyl derivatives, such as methyl derivatives, for instance methylglucose. Suitable esters can include oleates, laurates, palmitates, myristates, behenates, cocoates, stearates, linoleates, linolenates, caprates, arachidonates or mixtures thereof, such as, oleate/palmitate, oleate/stearate, or palmitate/stearate mixed esters. Suitable monoesters and diesters also include mono- or di-oleate, -stearate, -behenate, -oleopalmitate, -linoleate, -linolenate or -oleostearate of sucrose, of glucose or of methylglucose, including the product sold under the name GLUCATE DO by the company Amerchol, which is a methylglucose dioleate. Additional exemplary esters or mixtures of esters of sugar and of fatty acid that can also be mentioned include: the products sold under the names F160, F140, F110, F90, F70 and SL40 by the company Crodesta, respectively denoting sucrose palmitate/stearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose mono laurate; the products sold under the name Ryoto Sugar Esters, for example reference B370 and corresponding to sucrose behenate formed from 20% monoester and 80% diester-triester-polyester; the sucrose mono-dipalmitate/stearate sold by the company Goldschmidt under the name TEGOSOFT PSE.

In some embodiments, the hair care composition can further comprise a pH modifying agent such as citric acid and/or sodium hydroxide. Any commonly used pH-modifying agent for hair care compositions is contemplated for use herein.

In various embodiments, the hair care composition can further comprise any of the following ingredients, and/or mixtures thereof: sodium lauroyl methyl isethionate (a cleansing and lathering agent), liquid fatty substances including silicone oils different from the non-amino polyalkylsiloxanes discussed above herein, and organomodified polysiloxanes comprising at least one functional group chosen from amino groups, aryl groups and alkoxy groups. Organopolysiloxanes are defined in greater detail in Walter Noll's Chemistry and Technology of Silicones (1968), Academic Press, the entirety of which is hereby incorporated by reference. They can be volatile or non-volatile. Suitable cyclic polydialkylsiloxanes include octamethylcyclotetrasiloxane sold under the name VOLATILE SILICONE 7207 by Union Carbide or SILBIONE 70045 V2 by Rhodia, decamethylcyclopentasiloxane sold under the name VOLATILE SILICONE 7158 by Union Carbide, and SILBIONE 70045 V5 by Rhodia, and mixtures thereof. Cyclocopolymers of the dimethylsiloxane/methylalkylsiloxane type, such as VOLATILE SILICONE FZ 3109 sold by the company Union Carbide are also suitable. Exemplary cyclic polydialkylsiloxanes with organosilicon compounds include octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane, linear volatile polydialkylsiloxanes such as those sold under the name SH 200 by the company Toray Silicone. Silicones coming within this category are also described in the paper published in Cosmetics and Toiletries, Vol. 91, January 76, pp. 27-32, Todd & Byers, “Volatile Silicone Fluids for Cosmetics,” which is incorporated by reference herein.

Exemplary suitable non-volatile polydialkylsiloxanes include polydimethylsiloxanes having trimethylsilyl end groups such as the SILBIONE oils of the 47 and 70 047 series or the MIRASIL oils sold by Rhodia, such as, for example, the oil 70 047 V 500 000; the oils of the MIRASIL series sold by Rhodia; the oils of the 200 series from the company Dow Corning, such as DC200 with a viscosity of 60 000 mm²/s; the VISCASIL oils from General Electric and certain oils of the SF series (SF 96, SF 18) from General Electric, the polydimethylsiloxanes having dimethylsilanol end groups known under the name dimethiconol (CTFA), such as the oils of series 48 from the company Rhodia.

Exemplary organomodified silicones include polyalkylarylsiloxanes, and products sold under the following names: SILBIONE oils of the 70 641 series from Rhodia; the oils of the RHODORSIL 70 633 and 763 series from Rhodia; the oil Dow Corning 556 Cosmetic Grade Fluid from Dow Corning; the silicones of the PK series from Bayer, such as the product PK20; the silicones of the PN and PH series from Bayer, such as the products PN1000 and PH1000, certain oils of the SF series from General Electric, such as SF 1023, SF 1154, SF 1250 and SF 1265, the products sold under the names GP 4 Silicone Fluid and GP 7100 by Genesee or the products sold under the names Q2 8220 and Dow Corning 929 or 939 by the company Dow Corning.

In various embodiments, the hair care composition can further comprise one or more additional surfactants different from the cationic surfactants described above, including anionic surfactants, amphoteric or zwitterionic surfactants, nonionic surfactants and mixtures thereof. Exemplary anionic surfactants include alkyl sulfates, alkyl ether sulfates, alkylamido ether sulfates, alkylarylpolyether sulfates, monoglyceride sulfates, alkylsulfonates, alkylamidesulfonates, alkylarylsulfonates, α-olefin sulfonates, paraffin sulfonates, alkylsulfosuccinates, alkylether sulfosuccinates, alkylamide sulfo succinates, alkylsulfoacetates, acylsarcosinates, acylglutamates, alkylsulfosuccinamates, acylisethionates and N—(C₁-C₄)alkyl N-acyltaurates, salts of alkyl monoesters and of polyglycoside-polycarboxylic acids, acyllactylates, D-galactoside uronic acid salts, alkyl ether carboxylic acid salts, alkylaryl ether carboxylic acid salts, alkylamido ether carboxylic acid salts; and the corresponding non-salified forms of all these compounds; the alkyl and acyl groups of all these compounds (unless otherwise mentioned) generally comprising from 6 to 24 carbon atoms and the aryl group generally denoting a phenyl group. Anionic surfactants in salt form can include alkali metal salts such as the sodium or potassium salt, the sodium salt, ammonium salts, amine salts and amino alcohol salts or alkaline-earth metal salts such as the magnesium salt. Exemplary amino alcohol salts include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Suitable anionic surfactants also include mild anionic surfactants, i.e. anionic surfactants without a sulfate function, including polyoxyalkylenated alkyl ether carboxylic acids; polyoxyalkylenated alkylaryl ether carboxylic acids; polyoxyalkylenated alkylamido ether carboxylic acids, in particular those comprising 2 to 50 ethylene oxide groups; alkyl-D-galactoside uronic acids; acylsarcosinates, acylglutamates; and alkylpolyglycoside carboxylic esters such as those sold under the name AKYPO RLM 45 CA from Kao.

Exemplary suitable amphoteric or zwitterionic surfactant(s) can be secondary or tertiary aliphatic amine derivatives, optionally quaternized, in which the aliphatic group is a linear or branched chain containing from 8 to 22 carbon atoms, where the amine derivatives contain at least one anionic group, for example a carboxylate, sulfonate, sulfate, phosphate or phosphonate group, such as (C₈-C₂₀)alkylbetaines, sulfobetaines, (C₈-C₂₀)alkylamido(C₃-C₈)alkylbetaines or (C₈-C₂₀)alkylamido(C₆-C₈)alkylsulfobetaines. Any suitable secondary or tertiary aliphatic amine derivative can be present in coconut oil or in hydrolysed linseed oil, or the like. Representative compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of example, the cocoamphodiacetate sold by the company Rhodia under the trade name MIRANOL C2M Concentrate, and sodium diethylaminopropyl cocoaspartamide and sold by the company Chimex under the name Chimexane HB are suitable for use in the disclosed compositions.

Suitable nonionic surfactants are described in the Handbook of Surfactants by M. R. Porter, published by Blackie & Son (Glasgow and London), 1991, pp. 116-178, and include fatty alcohols, fatty α-diols, fatty (C₁-C₂₀)alkylphenols and fatty acids, which can be ethoxylated, propoxylated or glycerolated and contain at least one fatty chain comprising from 8 to 18 carbon atoms, the number of ethylene oxide or propylene oxide groups ranging from 1 to 200, and the number of glycerol groups ranging from 1 to 30. Condensates of ethylene oxide and of propylene oxide with fatty alcohols, ethoxylated fatty amides having from 1 to 30 ethylene oxide units, polyglycerolated fatty amides comprising on average from 1 to 5 glycerol groups, and in particular from 1.5 to 4, ethoxylated fatty acid esters of sorbitan containing from 1 to 30 ethylene oxide units, fatty acid esters of sucrose, fatty acid esters of polyethylene glycol, (C₆-C₂₄)alkylpolyglycosides, oxyethylenated plant oils, N—(C₆-C₂₄)alkylglucamine derivatives, amine oxides such as (C₁₀-C₁₄)alkylamine oxides or N—(C₁₀-C₁₄)acylaminopropylmorpholine oxides are also suitable for use in the compositions disclosed herein.

Additional suitable nonionic surfactants include alkyl polyglucosides (APG), maltose esters, polyglycerolated fatty alcohols, glucamine derivatives, for instance 2-ethylhexyloxycarbonyl-N-methylglucamine, and mixtures thereof. Alkylpolyglucosides that are those containing an alkyl group comprising from 6 to 30 carbon atoms and containing a hydrophilic group (glucoside). Exemplary alkylpolyglucosides include decylglucoside (alkyl-C9/C11-polyglucoside (1.4)), including the product sold under the name Mydol 10® by the company Kao Chemicals, the product sold under the name Plantaren 2000 UP® by the company Cognis, and the product sold under the name Oramix NS 10® by the company SEPPIC, and caprylyl/capryl glucosides, including the product sold under the name Oramix CG 110® by the company SEPPIC; laurylglucoside, sold as Plantaren 1200 N® and Plantacare 1200® by the company Cognis, and cocoglucoside, for instance the product sold under the name Plantacare 818/UP® by the company Cognis.

Suitable maltose derivatives include those described in document EP-A-566 438, such as O-octanoyl-6′-D-maltose or O-dodecanoyl-6′-D-maltose described in document FR-2 739 556. Each of these documents is incorporated by reference in its entirety.

In some embodiments, the collagen protein in the composition can be chemically modified with a hair colorant to provide a permanent, semipermanent, or temporary hair dye solution. Hair colorants, as herein defined, can be any dye, pigment, nanoparticles, and the like that can be used to change the color of hair. Suitable hair coloring agents include, but are not limited to dyes, such as 4-hydroxypropylamino-3-nitrophenol, 4-amino-3 nitrophenol, 2-amino-6-chloro-4-nitrophenol, 2-amino-6-chloro-4-nitrophenol, 2-nitro-paraphenylenediamine, N,N-hydroxy-ethyl-2-nitro-phenylenediamine, 4-nitro-indole, 2-nitro-5-glycerylmethylaniline, 3-methylamino-4-nitrophenoxyethanol, para-phenylenediamine, hydrogen peroxide, ammonia, DMDM hydantoin, methylparabens, propylparabens, lead acetate, and resorcinol.

In some embodiments, the hair care composition can be formulated in a cosmetically acceptable medium. The term “cosmetically acceptable medium” means a medium that is compatible with human keratin fibers, such as the hair. A cosmetically acceptable medium can be formed from water or from a mixture of water and one or more cosmetically acceptable solvents chosen from lower alcohols, such as ethanol and isopropanol; polyols and polyol ethers, including 2-butoxyethanol, propylene glycol, propylene glycol monomethyl ether, diethylene glycol monoethyl ether and monomethyl ether, and mixtures thereof.

In some embodiments, the hair care composition can further comprise any of the following additives, or mixtures thereof: solid fatty substances different from fatty alcohols such as waxes, anionic, nonionic or amphoteric polymers or mixtures thereof, antidandruff agents, anti-seborrhoea agents, agents for preventing hair loss and/or for promoting hair regrowth, vitamins and provitamins including panthenol, sunscreens, mineral or organic pigments, sequestrants, plasticizers, solubilizers, acidifying agents, mineral or organic thickeners, especially polymeric thickeners different from oxyethylenated polymers, opacifiers or nacreous agents, antioxidants, hydroxy acids, fragrances and/or preserving agents.

### Nail Care

In some embodiments, the cosmetic composition can comprise ingredients commonly used in nail care products. Nail care products include, but are not limited to, nail treatments including nail strengtheners, top coats, and base coats, nail polishes, nail polish removers, hands skincare, feet skincare, drying agents, and corrector pens including nail polish removers.

In some embodiments, the nail care composition can be a nail treatment composition. A nail treatment composition can comprise a composition for treating ingrown nails or nail deformities, a composition for topical treatment of nail infections, including fungal infections, nail strengtheners, top coats, base coats, polish removers, or any combination thereof. In some embodiments, a nail treatment composition can be formulated as a topical nail lacquer or polish, creams, solutions, suspensions, lotions, serums, gels, balms, gels, oils, oil in creams, and/or scrubs for treating the hands and/or the feet.

In some embodiments, the nail treatment composition can be nail strengthener. Nail strengtheners can treat fingernails and toenails to both harden, strengthen, and promote growth of the nails, to prevent or minimize breaking, cracking, splitting and peeling, and can comprise any known composition used to prevent and heal quarter cracks while increasing the growth of horse hooves, which have a similar protein consistency to human fingernails and toenails, including: lanolin, butter, beeswax, rosin, copper acetate, and turpentine. In some embodiments, the nail treatment composition can further comprise titanium Dioxide for example, TI-PURE R900 from E.I. DuPont.

In some embodiments, the nail treatment composition can be a base coat. In some embodiments, a base coat can be a liquid composition comprising at least one polymer which provides adhesion, for example a polymer co-polymerized from methyl methacrylate (MMA) and methacrylic acid (MAA) to form a polymer composed of polymethyl methacrylate (PMMA) and polymethacrylic acid (PMAA), in which the MAA monomer fraction can vary from 0 to 100%. In some embodiments, suitable polymers for use as a base coat include hydroxyethylmethacrylate (HEMA), hydroxypropylmethacrylate (HPMA), ethyl methacrylate (EMA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/succinate, 1,3-glycerol dimethacrylate/succinate adduct, phthalic acid monoethyl methacrylate, and mixtures thereof. In some aspects, a base coat can further comprise a non-reactive, solvent-dissolvable, film-forming polymer such as a cellulose ester, for example cellulose acetate alkylate, cellulose acetate butyrate, or cellulose acetate propionate. The above exemplary ingredients are not limiting.

In some embodiments, the nail treatment composition can be a top coat, for example a quick drying top coat. Top coats can include a base resin which is cellulose acetate butyrate, a film former which is a methacrylate polymer, thermally curable or photocurable monomers which are monofunctional methacrylates and cross-linkers (i.e., difunctional and trifunctional methacrylates), a thermal initiator or photoinitiator(s) together in a solution of aliphatic esters and alcohol, and a photoreactive coating. Suitable solvents include acetates and alcohols, particularly ethyl acetate, butyl acetate, and isopropyl alcohol. The photoreactive coating can include photoreactive monomers including methacrylate monomers such as: cyclohexyl methacrylate, n-decyl methacrylate, 2-ethyl hexyl methacrylate, ethyl methacrylate, hydroxy propyl methacrylate, isobornyl methacrylate, 2-methoxy ethyl methacrylate; difunctional and trifunctional methacrylate monomers, cross-linking agents such as diurethane dimethacrylate, ethylene glycol dimethacrylate, 1,10 decanediol dimethacrylate, 1,6-hexanediol dimethacrylate, and trimethylolpropane trimethacrylate. Commercially available photoinitiators suitable for use include but are not limited to benzoin methyl ether, 2-hydroxy-2-methyl-1-phenyl-1-propanone (“Darocur 1173”), diethoxyacetophenone, and benzyl diketal. Photoinitiators are oligomeric mixtures of phenyl propanones such as a mixture of 2,4,6-trimethylbenzophenone and 4-methylbenzophenone and a mixture of oligo-[2-hydroxy-2-methyl-1[4-(1-methylvinyl)phenyl]propanone] and 2-hydroxy-2-methyl-phenyl propanone that are sold by Sartomer under the names “Esacure KIP 100F” and “Esacure TZT Photoinitiator.”

In some embodiments, the nail care composition can be a nail polish or nail enamel composition. In some embodiments, the nail polish or nail enamel composition can further comprise a nitrocellulose film former, a latex film former, a polycarbodiimide film former, a low volatile organic compound (VOC), and polycarbodiimide film formers. Polycarbodiimides can include polymers with a plurality of carbodiimide groups appended to the polymer backbone. For example, U.S. Pat. No. 5,352,400 (the disclosure of which is incorporated by reference herein) discloses polymers and co-polymers derived from alpha-methylstyryl-isocyanates. Suitable polycarbodiimide compounds include, but are not limited to, those commercially sold by the suppliers Nisshinbo (including those known by the name under the CARBODILITE series, V-02, V02-L2, SV-02, E-02, V-10, SW-12G, E-03A), Picassian, and 3M.

In some embodiments, the nail polish or nail enamel composition can further comprise one or more latex polymers, including carboxyl functional acrylate latex polymers, carboxyl functional polyurethane latex polymers, carboxyl functional silicone latex polymers, carboxyl functional non-acrylate latex polymers and mixtures thereof. In various embodiments, suitable latex polymers can be film-forming latex polymers or non-film-forming latex polymers. In some embodiments, the latex polymers can be carboxyl functional acrylate latex polymers, such as those resulting from the homopolymerization or copolymerization of ethylenically unsaturated monomers chosen from vinyl monomers, (meth)acrylic monomers, (meth)acrylamide monomers, mono- and dicarboxylic unsaturated acids, esters of (meth)acrylic monomers, and amides of (meth)acrylic monomers The term “(meth)acryl” and variations thereof, as used herein, means acryl or methacryl. The (meth)acrylic monomers can be chosen from, for example, acrylic acid, methacrylic acid, citraconic acid, itaconic acid, maleic acid, fumaric acid, crotonic acid, and maleic anhydride. The esters of (meth)acrylic monomers can be, by way of non-limiting example, C1-C8 alkyl (meth)acrylates such as methyl (meth)acrylate, ethyl (meth)acrylate, propyl (meth)acrylate, isopropyl (meth)acrylate, butyl (meth)acrylate, tert-butyl (meth)acrylate, pentyl(meth) acrylate, isopentyl (meth)acrylate, neopentyl (meth)acrylate, hexyl (meth)acrylate, isohexyl (meth)acrylate, 2-ethylhexyl (meth)acrylate, cyclohexyl (meth)acrylate, isohexyl (meth)acrylate, heptyl (meth)acrylate, isoheptyl (meth)acrylate, octyl (meth)acrylate, isooctyl (meth)acrylate, allyl (meth)acrylate, and combinations thereof. The amides of (meth)acrylic monomers can, for example, be made of (meth)acrylamides, and especially N-alkyl (meth)acrylamides, in particular N—(C1-C12) alkyl (meth)acrylates such as N-ethyl (meth)acrylamide, N-t-butyl (meth)acrylamide, N-t-octyl (meth)acrylamide, N-methylol (meth)acrylamide and N-diacetone (meth)acrylamide, and any combination thereof.

The vinyl monomers can include, but are not limited to, vinyl cyanide compounds such as acrylonitrile and methacrylonitrile; vinyl esters such as vinyl formate, vinyl acetate, vinyl propionate, vinyl neodecanoate, vinyl pivalate, vinyl benzoate and vinyl t-butyl benzoate, triallyl cyanurate; vinyl halides such as vinyl chloride and vinylidene chloride; aromatic mono- or divinyl compounds such as styrene, .alpha.-methylstyrene, chlorostyrene, alkylstyrene, divinylbenzene and diallyl phthalate, as well as para-styrensulfonic, vinylsulfonic, 2-(meth)acryloyloxyethylsulfonic, 2-(meth)acrylamido-2-methylpropylsulfonic acids, and mixtures thereof.

The list of monomers herein is not limiting, and it should be understood that it is possible to use any monomer known to those skilled in the art which includes acrylic and/or vinyl monomers (including monomers modified with a silicone chain).

In some non-limiting exemplary embodiments, carboxyl functional acrylate latex polymers can be chosen from aqueous dispersions of Methacrylic Acid/Ethyl Acrylate copolymer (INCI: Acrylates Copolymer, such as LUVIFLEX SOFT by BASF), PEG/PPG-23/6 Dimethicone Citraconate/C10-30 Alkyl PEG-25 Methacrylate/Acrylic Acid/Methacrylic Acid/Ethyl Acrylate/Trimethylolpropane PEG-15 Triacrylate copolymer (INCI: Polyacrylate-2 Crosspolymer, such as Fixate Superhold™ by Lubrizol), Styrene/Acrylic copolymer (such as Acudyne Shine by Dow Chemical), Ethylhexyl Acrylate/Methyl Methacrylate/Butyl Acrylate/Acrylic Acid/Methacrylic Acid copolymer (INCI: Acrylates/Ethylhexyl Acrylate Copolymer, such as Daitosol 5000SJ, Daito Kasei Kogyo), Acrylic/Acrylates Copolymer (INCI name: Acrylates Copolymer, such as Daitosol 5000AD, Daito Kasei Kogyo), Acrylates Copolymers, such as those known under the tradenameDermacryl AQF (Akzo Nobel), under the tradename LUVIMER MAE (BASF), or under the tradename BALANCE CR (AKZO NOBEL), Acrylates/Hydroxyesters Acrylates Copolymer, known under the tradename ACUDYNE 180 POLYMER (Dow Chemical), Styrene/Acrylates Copolymer, known under the tradename Acudyne Bold from Dow Chemical, Styrene/Acrylates/Ammonium Methacrylate Copolymer, known under the tradename SYNTRAN PC5620 CG from Interpolymer, and mixtures thereof.

In some embodiments, the nail care composition can comprise a nail polish remover. Nail polish removers can include a polyhydric alcohol compound including glycerin, glycols, polyglycerin, esters of polyhydric alcohols, and mixtures thereof. The glycol can contain from 2 to 12 carbon atoms, such as, for example, glycerin, propylene glycol, butylene glycol, propane diol, hexylene glycol, polyglycerin, dipropylene glycol and diethylene glycol. Suitable esters of polyhydric alcohol include liquid esters of saturated or unsaturated, linear or branched C1-C26 polyhydric alcohols. Examples of suitable esters of polyhydric alcohol include, but are not limited to, esters of dihydroxy, trihydroxy, tetrahydroxy or pentahydroxy alcohols. The ester of polyhydric alcohol can be a glyceryl ester such as, glyceryl triglycolate, glyceryl tricitrate, glyceryl trilactate, glyceryl trilactate, glyceryl tributanoate, glyceryl triheptanoate, glyceryl trioctanoate, etc.

A nail polish remover can further comprise a low carbon alcohol, a containing from 1 to 8 carbon atoms. The low carbon alcohol can contain from 2 to 6 carbon atoms, such as from 2 to 5 carbon atoms. Examples of low carbon alcohols include, but are not limited to, ethanol, propanol, butanol, pentanol, isopropanol, isobutanol, and isopentanol. A nail polish remover can further comprise a high boiling point ester compound including, but are not limited to, carbonate esters, adipates, sebacates and succinates. Exemplary high boiling point ester compounds include, but are not limited to, alkylene carbonates such as propylene carbonate, dimethyl succinate, diethyl succinate, dimethyl glutarate, diethyl glutarate, dimethyl sebacate, diethyl sebacate, diisopropyl sebacate, bis(2-ethylhexyl) sebacate, dimethyl adipate, diisopropyl adipate, di-n-propyl adipate, dioctyl adipate, bis(2-ethylhexyl) adipate, diisostearyl adipate, ethyl maleate, bis(2-ethylhexyl) maleate, triisopropyl citrate, triisocetyl citrate, triisostearyl citrate, trioctyldodecyl citrate and trioleyl citrate.

A nail polish remover can further comprise a thickening agent including but not limited to: nonionic, anionic, cationic, amphiphilic, and amphoteric polymers, and other known rheology modifiers, such as cellulose-based thickeners such as hydroxyethylcellulose, hydroxypropylcellulose, methylcellulose, and ethylhydroxyethylcellulose. Certain notable cellulose derivatives include hydroxyl-modified cellulose polymers such as Hydroxyethylcellulose, e.g., those having a molecular weight over 500,000 daltons such as NATROSOL 250 HHR and Hydroxypropyl cellulose, e.g., KLUCEL MF—both available from Ashland of Covington, Ky. The thickening agent can be a polysaccharide such as fructans, glucans, galactans and mannans or heteropolysaccharides such as hemicellulose, pullulan or branched polysaccharides such as gum arabic and amylopectin, or mixed polysaccharides such as starch. The thickening agent can be an acrylic thickening agent (acrylic thickener) or an acrylamide thickening agent (acrylamide thickener). The thickening agent can comprise at least one monomer performing a weak acid function such as acrylic acid, methacrylic acid, itaconic acid, crotonic acid, maleic acid and/or fumaric acid. The thickening agent can comprise a monomer performing a strong acid function such as monomers having a function of the sulfonic acid type or phosphonic acid type, such as 2-acrylamido-2-methylpropane sulfonic acid (AMPS). The thickening agent can comprise a crosslinking agent such as methylene bisacrylamide (MBA), ethylene glycol diacrylate, polyethylene glycol dimethacrylate, diacrylamide, cyanomethacrylate, vi nyloxyethacrylate or methacrylate, formaldehyde, glyoxal, and compositions of the glycidylether type such as ethyleneglycol diglycidylether, or epoxides. Suitable acrylic thickeners are disclosed in U.S. patent application publication nos. 2004/0028637 and 2008/0196174, both of which are incorporated herein by reference. In some embodiments, the thickening agent can comprise an organoclay (hydrophobically treated clay) or a hydrophilic clay.

In some embodiments, the thickening agent can comprise an abrasive compound (abrasive system). An “abrasive compound” is a compound capable of providing abrasion or mechanical exfoliation. The abrasive particles can comprise perlite, pumice, zeolites, hydrated silica, calcium carbonate, dicalcium phosphate dihydrate, calcium pyrophosphate, alumina, sodium bicarbonate, polylactic acid, as well as synthetic polymeric materials such as polyethylene, polypropylene, polyethylene terephthalate, polymethlyl methacrylate or nylon. In certain embodiments a moderately hard abrasive includes perlite, such as a cosmetic grade perlite available from Imerys under the name IMERCARE 270P-Scrub. In certain embodiments a soft abrasive is a sugar, a ground fruit kernel or shell powders such as apricot kernel, coconut husk, or spherical waxes (for example, carnauba jojoba); argan shell powder, and the like.

In some embodiments, the nail care composition can further comprise an additive commonly used in cosmetic compositions and known to a person skilled include solvents, preservatives, fragrances, oils, waxes, surfactants, antioxidants, agents for combating free radicals, wetting agents, dispersing agents, antifoaming agents, neutralizing agents, stabilizing agents, active principles chosen from essential oils, UV screening agents, sunscreens, moisturizing agents, vitamins, proteins, ceramides, plant extracts, fibers, and the like, and their mixtures.

### Therapeutic Compositions

In some embodiments, the composition described herein can be a therapeutic composition that is useful for treating one or more conditions. In some embodiments the composition described herein can be formulated to be a dry film, a powder, a gel, or a solution in water or buffer. For example, and in some embodiments, application of the compositions described herein can promote wound healing, reduce or prevent the formation of scar tissue, promote tissue regeneration, minimize local inflammation, minimize tissue rejection, and/or enhance graft integration. In some embodiments, the composition can be formulated as an injectable material, e.g., a hydrogel. Collagen hydrogels present a large, uniform surface area, and can serve as a delivery system for collagen and, optionally, one or more additional therapeutic agents. Injectable collagen materials can also form scaffolds or networks capable of both replacing tissue function and supporting tissue regeneration. In certain embodiments, the composition can be topically applied. In certain embodiments, the composition can be dermally, intradermally, or subcutaneously injected.

In some embodiments, the therapeutic compositions described herein can comprise one or more additional therapeutic agents and/or prophylactic agents—other than the collagen fragments described elsewhere herein. The one or more additional therapeutic and/or prophylactic agents can be a small molecule active agent or a biomolecule, such as an enzyme or protein, polypeptide, or nucleic acid.

Non-limiting examples of additional therapeutic and/or prophylactic agents include anti-cancer agents, antimicrobial agents (including anti-viral agents, antibacterial agents, anti-fungal agents, and anti-parasitic agents), antioxidants, local analgesics, local anesthetics, anti-inflammatory agents, cytokines, immunosuppressant agents, anti-allergenic agents, essential nutrients, growth factors (such as fibroblast growth factor, hepatocyte growth factor, platelet-derived growth factor, vascular endothelial cell growth factor, and insulin-like growth factor), and combinations thereof. Specific dosages of the additional therapeutic and/or prophylactic agents can be readily determined by those of skill in the art. See Ansel, Howard C. et al., Pharmaceutical Dosage Forms and Drug Delivery Systems (6th ed.) Williams and Wilkins, Malvern, PA (1995).

In other embodiments, the composition described herein can be used in combination with cell delivery, for example, the delivery of stem cells, pluripotent cells, somatic cells, and combinations thereof.

Although the present disclosure contemplates that the therapeutic and/or prophylactic agents will be an integral part of the composition described herein, in some embodiments, the therapeutic and/or prophylactic agents can be administered prior to, in conjunction with, or subsequent to administration of the therapeutic composition described herein. In other words, in some embodiments, the one or more additional therapeutic and/or prophylactic agents may not be integrally incorporated into the present compositions but be provided separately from the present compositions.

Non-limiting examples of suitable local anesthetics capable of being included in the present compositions include, but are not limited to, ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxy tetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, psuedococaine, pyrrocaine, ropivacaine, salicyl alcohol, tetracaine, tolycaine, trimecaine, zolamine, and any combination thereof.

Non-limiting examples of suitable antiviral agents include ganciclovir and acyclovir. Non-limiting examples of suitable antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, and tetracyclines. Additional exemplary antimicrobial agents include iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

Non-limiting examples of suitable anti-inflammatory agents include steroidal active agents include glucocorticoids, progestins, mineralocorticoids, and corticosteroids. Exemplary non-steroidal anti-inflammatory drug include ketorolac, ibuprofen, nepafenac, diclofenac, aspirin, and naproxen. Other exemplary anti-inflammatory agents include triamcinolone acetonide, fluocinolone acetonide, prednisolone, dexamethasone, loteprendol, fluorometholone, and dipotassium glycyrrhizate.

In some embodiments, the composition can further comprise one or more additional pharmaceutically active agents. Exemplary pharmaceutical agents can include non-steroidal anti-inflammatory agents (NSAIDs), e.g., flurbiprofen, ibuprofen, naproxen, indomethacin, and related compounds. In some embodiments, the composition can further comprise one or more anti-mitotic drugs including colchicine, taxol and related compounds. In some embodiments, the composition can further comprise one or more topical antiseptics such as, e.g., benzoyl peroxide. In some embodiments, the composition can further comprise one or more polysaccharides produced by microalgae, e.g., alguronic acid.

In some embodiments, the composition can further comprise one or more immune-modulating drugs. Exemplary immune-modulating drugs include imiquimod, cyclosporine, tacrolimus, and rapamycin.

In some embodiments, the composition can further comprise one or more cytokines. Exemplary suitable cytokines include, but are not limited to, IL-10, TGF-β, IL-25, and IL-35. In certain embodiments, the cytokines can induce Treg activation (e.g., IL-25) and suppress Thl7 activation (e.g., IL-10) in order to minimize rejection.

In some embodiments, the compositions described herein further comprises at least one eukaryotic cell type. Some exemplary eukaryotic cell types include stem cells, mesenchymal stem cells, islet cells, keratinocytes, fibroblasts, melanocytes, adipocytes, immune cells such as T lymphocytes, B lymphocytes, natural killer cells, and dendritic cells, or combinations thereof. In some embodiments, the stem cells can be adipose-derived mesenchymal stem cells. Functional characteristics of mesenchymal stem cells that can benefit wound healing include their ability to migrate to the site of injury or inflammation, participate in regeneration of damaged tissues, stimulate proliferation and differentiation of resident progenitor cells, promote recovery of injured cells through growth factor secretion and matrix remodeling, and exert unique immunomodulatory and anti-inflammatory effects (See e.g., Phinney O G et al., Stem Cells, 25:2896-2902 (2007); Chamberlain G et al, Stem Cells, 25:2739-2749(2007); Dazzi F et al., Curr Opin Oncol. 19:650-655 (2007). Each of these references is incorporated by reference in its entirety.

In some embodiments, the eukaryotic cell can be responsible for increasing the structural integrity of connective tissue and/or promote healing. In some embodiments, the eukaryotic cell, such as a fibroblast, can be responsible for enhancing or promoting the growth or connection of cells or tissues.

In some embodiments, the recombinant collagen composition can be in contact with 1) an implanted hair graft and 2) tissue into which the hair graft is implanted. Recipient sites for hair grafts include the scalp, the facial region, the armpit or chest region or the pubic region. Specific areas of the facial region include eyebrows, eyelid, mustache, sideburn, chin and cheeks. The recipient site can be any area of skin wherein the appearance of hair is desired by the subject. In some embodiments, the contact between the composition described herein and the implanted hair with the surrounding tissues of the recipient site promotes nutritional perfusion from the surrounding tissues into the graft and increases the survival rate of the graft compared to a graft implanted without the use of the composition described herein. In certain embodiments, the contact between the composition described herein and the implanted hair, and between the composition described herein and the surrounding tissues of the recipient site promotes vascularization around the implanted grafts, such that the survival rate of the graft is increased compared to a graft implanted without the use of the composition described herein.

### Dietary Compositions

In some embodiments, the composition described herein can be a dietary composition useful for useful for providing collagen to a subject in need thereof. For example, and in some embodiments, consumption of the dietary compositions described herein can provide health and/or skin benefits, such as increasing collagen intake, relieving joint pain, and improving skin health. In certain embodiments, the composition can be in the form of a powder, a capsule, a liquid, or any other suitable form.

In some embodiments, the dietary compositions can comprise one or more nutritional ingredients such as: ascorbic acid, biotin, chromium nicotinate, copper citrate, D-calcium pantothenate, cyanocobalamin, flax seed, Linum usitatissimum, folic acid, fructooligosaccharide (fiber), magnesium oxide, manganese citrate, maltodextrin, medium chain triglycerides, flavor, niacinamide, potassium citrate potassium iodide, riboflavin, sugar cane (Saccharum officinarum), sodium molybdate dihydrate, sodium selenate (selenium), soy protein isolate, Stevia leaf extract/Stevia rebaudiana, thiamin HCl, tricalcium phosphate, vitamin a palmitate, vitamin D3, xanthan gum, zinc citrate, cellulose gum, guar gum, pyridoxine hydrochloride, salt, tocopherol, antioxidants, e.g., resveratrol, CoQ10, acai berry, lycopene and pomegranate, natural or artificial sweeteners, e.g., glucose, sucrose, fructose, saccharides, cyclamates, aspartamine, sucralose, aspartame, acesulfame K, or sorbitol, flavoring, such as a flavored extract, volatile oil, chocolate flavoring (e.g., non-caffeinated cocoa or chocolate, chocolate substitutes such as carob), peanut butter flavoring, cookie crumb, vanilla, or any commercially available flavoring, and any combination thereof.

### Exemplary Formulations

In some embodiments, as mentioned above, the composition described herein can be in the form of an alcohol- or water-based toner. Exemplary toner formulations are set forth below. The collagen, collagen fragment, or dyed collagen fragment set forth in the examples below can be crosslinked or free of crosslinks. In some embodiments, the HA set forth in the examples below can be crosslinked or free of crosslinks.

| INGREDIENTS: | % WT/WT |
|---|---|
| Water (Aqua) | Remainder Ingredient |
| Recombinant collagen fragment | about 0.0005% to about 25% |
| disclosed herein | |
| Alcohol Denat. | 10-20% |
| Pentylene Glycol | 5-10% |
| Glycerin | 1-5% |
| Gluconolactone | 0.1-1% |
| Dipotassium Glycyrrhizate | 0.1-1% |
| Sodium Citrate | 0.1-1% |
| Sodium Benzoate | 0.1-1% |
| HA | 0.1-3% |

| INGREDIENTS: | % WT/WT |
|---|---|
| Water (Aqua) | Remainder Ingredient |
| Recombinant collagen fragment | about 0.0005% to about 25% |
| disclosed herein | |
| Niacinamide | 1-5% |
| Pentylene Glycol | 1-5% |
| Propanediol | 1-5% |
| Glycerin | 1-5% |
| Biosaccharide Gum-1 | 0.1-1% |
| Glyceryl Caprylate | 0.1-1% |
| Sodium Anisate | 0.1-1% |
| Sodium Hydroxide | 0.1-1% |
| Caprylhydroxamic Acid | 0.1-1% |
| Acrylates/C10-30 Alkyl Acrylate | 0.1-1% |
| Crosspolymer | |
| Sodium Levulinate | 0.1-1% |
| Caprylyl Glycol | 0.1-1% |
| HA | 0.1-3% |

In some embodiments as mentioned above, the composition described herein can be in the form of a cream, a gel, or a serum. Exemplary cream, gel, and serum formulations are set forth below.

### Cream

| INGREDIENTS: | % WT/WT |
|---|---|
| Water (Aqua) | Remainder Ingredient |
| Recombinant collagen | about 0.0005% to about 25% |
| fragment disclosed herein | |
| Cetearyl Alcohol | 5-10% |
| Glycerin | 1-5% |
| Squalane | 1-5% |
| Butyrospermum Parkii (Shea) | 1-5% |
| Butter | |
| Glyceryl Caprylate | 1-5% |
| Microcrystalline Cellulose | 1-5% |
| Glyceryl Stearate Citrate | 0.1-1% |
| Tocopheryl Acetate | 0.1-1% |
| Cetearyl Glucoside | 0.1-1% |
| Sodium Stearoyl Glutamate | 0.1-1% |
| Cellulose Gum | 0.1-1% |
| Xanthan Gum | 0.1-1% |
| Caprylhydroxamic Acid | 0.1-1% |
| Sodium Phytate | 0.01-0.1% |
| HA | 0.1-3% |

### Gel

| INGREDIENTS: | % WT/WT |
|---|---|
| Water (Aqua) | Remainder Ingredient |
| Recombinant collagen fragment | about 0.0005% to about 25% |
| disclosed herein | |
| Sodium Phytate | 0.1-1% |
| Sodium Hydroxide | 0.1-1% |
| Carbomer | 0.1-1% |
| Phenoxyethanol | 0.1-1% |
| HA | 0.1-3% |

### Serum

| INGREDIENTS: | % WT/WT |
|---|---|
| Water (Aqua) | Remainder Ingredient |
| Recombinant collagen fragment | about 0.0005% to about 25% |
| disclosed herein | |
| Pentylene Glycol | 1-5% |
| Niacinamide | 1-5% |
| Dimethicone | 1-5% |
| Propanediol | 1-5% |
| Tocopherol | 0.1-1% |
| HA | 0.1-3% |
| Linoleic Acid | 0.1-1% |
| Ammonium | 0.1-1% |
| Acryloyldimethyltaurate/VP | |
| Copolymer | |
| Acrylates/C10-30 Alkyl Acrylate | 0.1-1% |
| Crosspolymer | |
| Caprylyl Glyceryl Ether | 0.1-1% |
| Tetrasodium Glutamate Diacetate | 0.01-0.1% |
| Sodium Hydroxide | 0.01-0.1% |
| Phenoxyethanol | 0.01-0.1% |
| Linolenic Acid | 0.001-0.0 |

In some embodiments as mentioned above, the composition described herein can be a shampoo or conditioner. Exemplary shampoo or conditioner formulations are set forth below.

### Shampoo

| INGREDIENTS: | % WT/WT |
|---|---|
| Water (Aqua) | Remainder Ingredient |
| Recombinant collagen fragment | about 0.0005% to about 25% |
| disclosed herein | |
| Cocamidopropyl Betaine | 5-10% |
| Sodium Lauroyl Methyl Isethionate | 5-10% |
| Propanediol | 1-5% |
| Sodium Methyl Oleoyl Taurate | 1-5% |
| Sodium Cocoyl Isethionate | 1-5% |
| Trisodium Ethylenediamine | 0.1-1% |
| Disuccinate | |
| Caprylhydroxamic Acid | 0.1-1% |
| Panthenol | 0.1-1% |
| Citric Acid | 0.1-1% |
| Caprylyl Glycol | 0.1-1% |
| Sodium Benzoate | 0.1-1% |
| HA | 0.1-3% |

### Conditioner

| INGREDIENTS: | % WT/WT |
|---|---|
| Water (Aqua) | Remainder Ingredient |
| Recombinant collagen fragment | about 0.0005% to about 25% |
| disclosed herein | |
| Cetearyl Alcohol | 5-10% |
| Glyceryl Caprylate | 1-5% |
| Behentrimonium Methosulfate | 1-5% |
| Glycerin | 1-5% |
| Caprylhydroxamic Acid | 0.1-1% |
| Panthenol | 0.1-1% |
| Hydroxyethylcellulose | 0.1-1% |
| Cocos Nucifera (Coconut) Oil | 0.1-1% |
| Citric Acid | 0.01-0.1% |
| HA | 0.1-3% |

In some embodiments as mentioned above, the composition described herein can be a nailcare composition. An exemplary nailcare formulation is set forth below.

### Nail Polish

| INGREDIENTS: | % WT/WT |
|---|---|
| Water (Aqua) | Remainder Ingredient |
| Dyed Recombinant collagen | about 0.0005% to about 25% |
| fragment disclosed herein | |
| Ethyl acetate | 0.1-1% |
| Butyl acetate | 0.1-1% |
| Isopropyl alcohol, and | 0.1-1% |
| diacetone alcohol. | |
| Nitrocellulose | 0.1-1% |
| Acetyl tributyl citrate | 0.1-1% |
| Adipic acid/neopentyl glycol | 0.1-1% |
| Neopentyl glycol | 0.1-1% |
| Plasticizer | 0.1-1% |
| HA | 0.1-3% |

### F. Methods of Production

In some embodiments, the present disclosure provides methods of preparing a composition comprising collagen and hyaluronic acid, wherein the collagen is fully miscible with the hyaluronic acid, the method comprising: a) dissolving hyaluronic acid in an NaOH solution; b) adjusting the pH by adding HCl; and c) adding collagen.

In some embodiments, the present disclosure provides methods of preparing a composition comprising collagen and hyaluronic acid, wherein the collagen is fully miscible with the hyaluronic acid, the method comprising (a) dissolving hyaluronic acid in water, (b) dissolving collagen in water, and (c) mixing the two solutions. In some embodiments, the methods further comprise: d) optionally crosslinking the hyaluronic acid and the collagen in the solution by adding a coupling agent.

In some embodiments, the present disclosure provides methods of preparing a composition comprising collagen and hyaluronic acid, wherein the collagen is fully miscible with the hyaluronic acid, the method comprising (a) dissolving hyaluronic acid in water or an NaOH solution, b) optionally crosslinking the hyaluronic acid by adding a coupling agent before reducing the pH with HCl, (c) dissolving collagen in water, and (d) mixing the two solutions.

In some embodiments, the methods further comprise: e) adding sodium salt to cause a gel to form; and f) allowing the gel to set. In some embodiments, the methods further comprise: g) sterilizing the gel. In some embodiments, the methods further comprise: h) dispensing the gel into a syringe under aseptic conditions.

In some embodiments, the present disclosure provides methods of preparing a composition comprising collagen and hyaluronic acid comprising dissolving hyaluronic acid in a salt buffer such as PBS and adding collagen.

In some embodiments, the present disclosure provides methods of preparing a composition comprising collagen and hyaluronic acid, wherein the solution comprises NaCl.

In some embodiments the method further comprises adding calcium hydroxyapatite, carboxymethyl cellulose, polylactic acid, or any combination thereof to the composition or gel.

In some embodiments the method further comprises adding any suitable biocompatible polymer known in the art. Suitable polymers include but are not limited to aliphatic polyesters, polyorthoesters, polyoxyesters, polyanhydrides, polycarbonates, polyurethanes, polyamides, polyester amides, hydrolyzable amines, polyalkylene oxides, polyethylene oxide (PEO), poloxamers, functionalized isoflavonoids, amino acids, and combinations thereof.

Additional suitable polymers include but are not limited to: poly(hydroxyethyl methacrylate), poly(sulfobetaine), poly(dimethylsiloxane), poly(caprolactone), poly(p-dioxanone), poly(trimethylene carbonate), poly(glycolide), poly(L-lactide), poly(D,L-lactide), poly(L-lactide-co-glycolide), poly(D,L-lactide-co-glycolide), poly(L-lactide-co-caprolactone), poly(L-lactide-co-D,L-lactide), poly(caprolactone-co-trimethylene carbonate), poly(lactide-co-trimethylene carbonate), poly(caprolactone-co-p-dioxanone), poly(trimethylene carbonate-co-p-dioxanone), poly(caprolactone-co-lactide), poly(lactide-co-1,5-dioxepan-2-one), and poly(1,5-dioxepan-2-one-co-p-dioxanone), poly(lactide-co-1,4-dioxepan-2-one), and poly(1,4-dioxepan-2-one-co-p-dioxanone), poly(glycolide-co-caprolactone-co-dioxanone), poly(lactide-co-caprolactone-co-dioxanone), poly(dioxanone-co-D,L-lactide), poly(dioxanone-co-glycolide), poly(dioxanone-co-caprolactone), poly(dioxanone-co-L-lactide), poly(dioxanone-co-D,L-lactide-co-caprolactone), poly(dioxanone-co-glycolide-co-caprolactone), poly(dioxanone-co-L-lactide-co-D,L-lactide), poly(dioxanone-co-L-lactide-co-glycolide), poly(dioxanone-co-L-lactide-do-caprolactone), poly(dioxanone-co-D,L-lactide-co-glycolide), poly(dioxanone-co-D,L-lactide-co-caprolactone), poly(dioxanone-co-glycolide-co-caprolactone, poly(dioxanone-co-L-lactide-co-glycolide-co-caprolactone), poly(dioxanone-co-D,L-lactide-co-glycolide-co-caprolactine), and combinations thereof.

In some embodiments, the method further comprises adding a bioabsorbable polymer that is selected from the group consisting of solid homopolymers of poly(e-caprolactone), solid homopolymers of poly(p-dioxanone), solid homopolymers of poly(trimethylene carbonate), solid copolymers of a plurality of e-caprolactone repeating units and third lactone repeating units, solid copolymers of a plurality of trimethylene carbonate repeating units and second lactone repeating unit; wherein the third lactone repeating units are selected from the group consisting of glycolide repeating units, lactide repeating units, trimethylene carbonate repeating units, p-dioxanone repeating units, 1,4-dioxepan-2-one repeating units, 1-5-dioxepan-2-one repeating units and combinations thereof.

In some embodiments the method further comprises adding an anesthetic agent to the composition or gel.

In some embodiments the method further comprises drying the composition by any suitable option such as oven drying, lyophilizing or spray drying. In some embodiments the dry product can be a coating, a film or a powder which can be re-hydrated to a gel as desired for application.

The methods described herein can include exposing the collagens and collagen fragments described herein to crosslinking agents (chemical crosslinking agents, e.g., gluteraldehyde, etc.) or to crosslinking conditions (e.g., heat, radiation, etc.), such that the collagen does contain non-endogenously imposed crosslinks.

In some embodiments, methods of crosslinking hyaluronic acid and one or more recombinant collagen fragments comprise: dissolving a hyaluronic acid and a collagen in an aqueous solution to form an aqueous pre-reaction solution, wherein the aqueous pre-reaction solution further comprises a salt or has a low pH; and modifying the aqueous pre-reaction solution to form a crosslinking reaction mixture comprising: the hyaluronic acid; the recombinant collagen fragment; a water soluble coupling agent; and the salt; and wherein the crosslinking reaction has a higher pH than the aqueous pre-reaction solution; and allowing the crosslinking reaction mixture to react to thereby crosslinking at least the hyaluronic acid and one or more recombinant collagen fragments. In some embodiments, the concentration and the proportion of hyaluronic acid and recombinant collagen fragment can be varied within a wide range. The respective concentrations are at least about 0.01 to 10 wt % for hyaluronic acid and at least about 0.01 to 10 wt % for the recombinant collagen fragment, at least about 0.05 to 10 wt % for hyaluronic acid and at least about 0.1 to 10 wt % for recombinant collagen fragment, at least about 0.05 to 5 wt % for hyaluronic acid and at least about 0.5 to 7 wt % recombinant collagen fragment, or at least about 0.1 to 2 wt % for hyaluronic acid and at least about 1 to 5 wt % recombinant collagen fragment. The respective concentrations are about 0.01 to 10 wt % for hyaluronic acid and about 0.01 to 10 wt % for recombinant collagen fragment, about 0.05 to 10 wt % for hyaluronic acid and about 0.1 to 10 wt % for recombinant collagen fragment, about 0.05 to 5 wt % for hyaluronic acid and about 0.5 to 7 wt % recombinant collagen fragment, or about 0.1 to 2 wt % for hyaluronic acid and about 1 to 5 wt % recombinant collagen fragment.

In some embodiments, a base (e.g., NaOH) is first added to hyaluronic acid to adjust the pH to 9-12. A cross-linker is added to the hyaluronic acid and the reaction is allowed to proceed. One or more recombinant collagen fragments (pH 2-3) are then added. The collagen reacts with the cross-linker to covalently bind to HA. In some embodiments, the cross-linker can be consumed lightly (i.e. not fully reacted) in cross-linking the collagen to HA. A hyaluronic acid solution is added as a lubricant and cross-linker scavenger to react with any excess/unreacted crosslinker. The final formulation can have a core of cross-linked hyaluronic acid with collagen lightly cross-linked and surrounding the hyaluronic acid core. The additional soluble hyaluronic acid consumes the residual cross-linker and adds a lubrication benefit.

In some embodiments, a base (e.g., NaOH) can be first added to hyaluronic acid to adjust the pH to 9-12. A cross-linker can be added to the hyaluronic acid and the reaction is allowed to proceed. The pH can be adjusted to below 7.5 with acid, then one or more recombinant collagen fragments (pH 2-3) can be added. The collagen may react with the cross-linker to covalently bind to HA. In some embodiments, the cross-linker can be consumed lightly (i.e., not fully reacted) in cross-linking the collagen to HA. A hyaluronic acid solution can be added as a lubricant and cross-linker scavenger to react with any excess/unreacted crosslinker. The final formulation can have a core of cross-linked hyaluronic acid with collagen lightly cross-linked infusing the hyaluronic acid core. The additional soluble hyaluronic acid can consume the residual cross-linker and add a lubrication benefit.

Suitable linking agents for the covalent crosslinkage include, but are not limited to, ethylene glycol diglycidyl ether or 1,4-butanediol diglycidyl ether, divinyl sulfone, 1-Ethyl-3-(3-dimethylaminopropyl)carbodiimide (EDC), glycol ether diglycidylether, polyethyleneglycol diglycidylether, polypropylene glycol diglycidyl ether, 1,6-hexanediol diglycidyl ether, 1,2,7,8-diepoxy octane, divinylsulfone. 1,2-bis(2,3-epoxypropyl)-2,3-ethylene, 1-(2,3-epoxypropyl)-2,3-epoxycyclohexane, glycerin triglycidyl ether, resorcinol diglycidyl ether, glycidyl allyl ether, ethylene glycol diglycidylether, any one of 1,6-hexanediol diglycidyl ether, neopentylglycol diglycidyl ether, photocrosslinking reagents, such as ethyleosin, hydrazides, such as bishydrazide, trishydrazide or polyvalent hydrazide compounds. In some embodiments, the linking agent can be an auxiliary crosslinking agent, such as N-hydroxy-succinamide (NHS), NHS (sulfo group-NHS), succinyl oxide, and sodium periodate. In some embodiments, the linking agent can be a carbodiimide class linking agent such as 1-ethyl-3-(3-dimethylaminopropyl) carbodiimide (EDC), 1-ethyl-3-(3-trimethyl amino propyl) carbodiimide (ETC), 1,1′-two sulphur-p-phenylene two (carbodiimide), tert-butylcarbodiimide, and N,N-Diisopropylcarbodiimide (DIC).

In some embodiments, the crosslinkage reaction takes place at a temperature of from about 0° C. to about 100° C., from about 10° C. to about 90° C., from about 20° C. to about 90° C., from about 20° C. to about 80° C., from about 30° C. to about 70° C. or from about 40° C. to about 60° C. The step of crosslinking can be carried out using any means known to those of ordinary skill in the art.

In some embodiments, the crosslinking reaction can take place at a temperature of from about 0° C. to about 100° C., from about 10° C. to about 90° C., from about 20° C. to about 90° C., from about 20° C. to about 80° C., from about 30° C. to about 70° C. or from about 40° C. to about 60° C. The step of crosslinking can be carried out using any means known to those of ordinary skill in the art. In some embodiments, following the crosslinking the pH can be reduced to below 7.5 before the addition of collagen. The addition of collagen can be from about 0.1% by weight to about 25% by weight in water or optionally a buffer such as PBS. Collagen can be added with or without mixing. Without wishing to be bound by a particular theory, it is believed that when collagen is added without mixing, the collagen penetrates the HA gel by diffusion. In embodiments where mixing is employed, any suitable mixing device known to those skilled in the art can be employed.

Those skilled in the art can optimize conditions of crosslinking according to the nature of the hyaluronic acid and collagen fragments and carry out crosslinking to an optimized degree. The degree of cross-linking can be controlled by changing several factors including the molecular weight of the hyaluronic acid, the molecular weight of the recombinant collagen fragments, the concentration in the reaction mixture, the alkali concentration, and the ratio of linking agents.

The composition according to the present disclosure can comprise collagen or fragments thereof generated by yeast or a modified yeast used to produce collagen fragments. Suitable yeast strains and methods of producing recombinant collagen fragments therein are described in International Application No. PCT/US2022/027016, which is incorporated herein in its entirety.

### Skincare

In some embodiments, the composition described herein can be a skincare composition that can be used to treat an area of the skin by topically applying the skincare composition to the area of skin.

In some embodiments, the composition described herein can be a skincare composition that can be used to treat the skin, hair, and nails, by consuming the composition as a dietary supplement.

In some embodiments, administration of the composition described herein to the skin can improve or maintain the quality of skin and reduce or eliminate signs of aging. Signs of aging include, but are not limited to, all outward visibly and tactilely perceptible manifestations as well as any other macro or micro effects due to skin aging. Such signs can be induced or caused by intrinsic factors or extrinsic factors (such as chronological aging and/or environmental damage). These signs can result from processes which include, but are not limited to, the development of textural discontinuities such as wrinkles and coarse deep wrinkles, fine lines, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including undereye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues (e.g., fat and/or muscle), especially those proximate to the skin.

In some embodiments, a composition as described herein can be suitable for use as a dermal filler. A dermal filler composition can replace lost endogenous matrix polymers or enhance/facilitate the function of existing matrix polymers, in order to treat skin conditions due to aging or injury. The dermal filler composition can fill wrinkles, lines, folds, scars, and to enhance dermal tissue, such as, e.g., to plump thin lips, or fill-in sunken eyes or shallow cheeks. Earlier dermal filler products generally were made of collagens. One common matrix polymer used in modern dermal filler compositions is hyaluronan. Because hyaluronan is natural to the human body, it is a generally well tolerated and a fairly low risk treatment for a wide variety of skin conditions.

In some embodiments, a composition as described herein can be suitable for use with a microneedle sheet or patch. Microneedles can provide the composition described herein to any area of the face and body to achieve a cosmetic benefit or wound healing benefit.

In some embodiments, the composition described herein can be a therapeutic composition that can be used to reduce or prevent the formation of scar tissue, promoting healing, promoting tissue regeneration, minimizing local inflammation, minimizing tissue rejection, and/or enhance skin and/or hair graft integration.

In some embodiments, the disclosure provides a method of treating a dermatological condition comprising administering an effective amount of the composition comprising a recombinant collagen fragment to a subject in need thereof. In some embodiments, the dermatological condition can be fine lines, wrinkles, dry skin, excessive pore size, skin dyschromia, reduced elasticity, unwanted hair, skin thinning, purpura, actinic keratosis, pruritus, eczema, acne, rosacea, erythema, telangiectasia, actinic telangiectasia, skin cancer, or rhinophyma.

In some embodiments, the composition can be topically administered to an area of skin. In some embodiments, the area of skin can be selected from the group consisting of a facial surface, scalp, neck, ears, shoulders, chest (including breasts and/or the decolletage), arms, hands, legs, stomach, buttocks, groin, back, feet, and combinations thereof.

In some embodiments, the composition can be topically administered to a facial surface. In some embodiments, the facial surface can be selected from the group consisting of forehead, eyes, a perioral surface, a chin surface, a periorbital surface, a nasal surface, a cheek skin surface, and combinations thereof.

In some embodiments, the disclosure provides a method for improving collagen production in the skin, comprising administering an effective amount of a composition comprising a recombinant collagen fragment.

In some embodiments, the disclosure provides a skincare product comprising the composition described herein for use in reducing the appearance of wrinkles, evening skin tone, providing moisture, reducing the appearance of dark circles under the eyes, increasing the collagen content of skin, increasing skin density, improving skin firmness and elasticity, improving the appearance of lines and wrinkles, smoothing the skin texture, increasing skin radiance and luminosity, improving the appearance of sagging skin, whitening the skin, sun care, color cosmetics, hair care, fragrances, oral care, or any combination thereof.

### EXAMPLES

The examples presented below are provided for the purpose of illustration only and the embodiments described herein should in no way be construed as being limited to these examples. Rather, the embodiments should be construed to encompass any and all variations which become evident as a result of the teaching provided herein.

### Example 1

Polyethylene oxide (PEO) with a molecular weight of 400 kDa was purchased from Sigma (Sigma-Aldrich, St. Louis, MO) (“Polymer 1”). A recombinant collagen fragment with a molecular weight of 50 kDa having SEQ ID NO: 1 was prepared in yeast, as described in PCT/US2022/027016, which is incorporated herein by reference in its entirety (“Polymer 2”). A 5 wt % polyethylene oxide (PEO) solution was prepared by dissolving 2.5 g PEO in 47.5 ml deionized (DI) water and mixing on a stir plate at 400 RPM for 16 hours. A 5% (w/v) solution including the recombinant collagen fragment with the amino acid sequences of SEQ ID NO: 1 was produced by dissolving 5 grams of the 50 kDa protein into 100 ml of water. The two solutions were mixed at a 1:1 (w/w) ratio in a 50 ml conical tube and mixed at 10 RPM for 16 hours. The conical tube was placed vertically on a rack for overnight before assessing the solution stability. The final solution was turbid and slowly phase separated, with white or yellow-ish aggregates settled at bottom of the tube.

### Example 2

A mixture including polyethylene oxide (PEO) and a recombinant collagen fragment with a molecular weight of 50 kDa having SEQ ID NO: 1 was prepared and dried, and visually examined. PEO with a molecular weight of 100 kDa was purchased from Sigma (“Polymer 1”). The remainder of the method was the same as set forth in Example 1. The final solution was phase separated with a hazy solution on top, and hazy and slightly yellow solution on bottom.

### Example 3

A first polyvinylpyrrolidone (PVP) and collagen (recombinant collagen fragment with a molecular weight of 50 kDa having SEQ ID NO: 1) mixture was prepared and dried, and visually examined. Example 3 was prepared using the same method described in Example 1, except that Polymer 1 was replaced with polyvinylpyrrolidone (Sigma; molecular weight of 10 kDa). The final solutions were transparent. About 13 grams (g) of solution was cast into a 100 ml Polytetrafluoroethylene (PTFE) evaporating dish and dried at 45° C. overnight. The dried films were visually transparent.

This example demonstrates that solutions including 10 kDa PVP and the recombinant collagen fragment with a molecular weight of 50 kDa having SEQ ID NO: 1 are fully miscible and can be prepared as a film or article.

### Example 4

A second polyvinylpyrrolidone (PVP) and collagen (recombinant collagen fragment with a molecular weight of 50 kDa having SEQ ID NO: 1) mixture was prepared, dried, and visually examined. Example 4 was prepared using the method described in Example 1, except that Polymer 1 was replaced with polyvinylpyrrolidone (Sigma; molecular weight of 40 kDa). The final solutions were transparent. Films were prepared using the method described in Example 3. The dried films were visually transparent.

This example demonstrates that solutions including 40 kDa PVP and the recombinant collagen fragment with a molecular weight of 50 kDa having SEQ ID NO: 1 are fully miscible and can be prepared as a film or article.

### Example 5

A third polyvinylpyrrolidone (PVP) and collagen (recombinant collagen fragment with a molecular weight of 50 kDa having SEQ ID NO: 1) mixture was prepared and dried, and visually examined. Example 5 was prepared using the method described in Example 1, except that Polymer 1 was replaced with polyvinylpyrrolidone (Sigma; molecular weight of 360 kDa). The final solutions were transparent. Films were prepared using the same method in Example 3. The dried films were visually transparent.

This example demonstrates that solutions including 360 kDa PVP and the recombinant collagen fragment with a molecular weight of 50 kDa having SEQ ID NO: 1 are fully miscible and can be prepared as a film.

### Example 6

A first chitosan and collagen (recombinant collagen fragment with a molecular weight of 50 kDa having SEQ ID NO: 1) mixture was prepared and visually examined. Example 6 was prepared using the method described in Example 1, except that Polymer 1 was replaced with chitosan (molecular weight range of 50 kDa to 190 kD; Sigma). The chitosan solution was pH adjusted to pH 4 using 0.287 ml of acetic acid from Sigma to fully solubilize the solution during mixing. The final solution was phase separated into two layers. The viscosity was determined visually by inspecting the difference between the two layers. The top layer had lower viscosity and was more transparent, whereas the bottom layer had higher viscosity and was more turbid.

### Example 7

A polyethylene glycol and collagen (recombinant collagen fragment with a molecular weight of 50 kDa having SEQ ID NO: 1) mixture was prepared and dried, and visually examined. Example 7 was prepared using the method described in Example 1, except that Polymer 1 was replaced with polyethylene glycol (molecular weight of 35 kDa Sigma). The final solution was phase separated with a transparent and clear solution on the top and a transparent and slightly yellow solution on the bottom.

### Example 8

A first alginic acid and collagen (recombinant collagen fragment with a molecular weight of 50 kDa having SEQ ID NO: 1) mixture was prepared and dried, and visually examined. Examples 8 was prepared using the method described in Example 1, except that Polymer 1 was replaced with medium viscosity alginic acid sodium salt from brown algae (Sigma). The viscosity of 2% alginic acid sodium salt in water at 25° C. was equal to or greater than 2,000 cps. A 2.5 wt % alginic acid solution was prepared by dissolving 2.5 g alginic acid sodium salt in 97.5 ml deionized (“DI”) water and mixing on a stir plate at 400 RPM for 24 hours due to the high viscosity of the solution. The viscosity was determined visually by inspecting the difference between the two layers. The two solutions were mixed at a 1:1 (v/v) ratio in a 50 ml conical tube and mixed at 10 RPM for 16 hours. The conical tube was placed vertically on a rack for overnight before assessing the solution stability. The final solution was transparent. A film was prepared using the method set forth in Example 3. The dried film was visually hazy with uniformly distributed haziness throughout the film.

### Example 9

A second alginic acid and collagen (recombinant collagen fragment with a molecular weight of 50 kDa having SEQ ID NO: 1) mixture was prepared and dried, and visually examined. Example 9 was prepared using the method described in Example 1, except that Polymer 1 was replaced with low viscosity alginic acid sodium salt from brown algae (Sigma). The viscosity of 1% alginic acid sodium salt in water at 25° C. was about 4-12 cps. Both alginic acid sodium salt and collagen stock solutions were 5 wt %. The two solutions were mixed at a 1:1 (v/v) ratio in a 50 ml conical tube and mixed at 10 RPM for 16 hours. The final solution was transparent. A film was prepared using the method set forth in Example 3. The dried film was visually hazy with uniformly distributed haziness throughout the film.

### Example 10

A polyacrylamide and collagen (recombinant collagen fragment with a molecular weight of 50 kDa having SEQ ID NO: 1) mixture was prepared and dried, and visually examined. Example 10 was prepared using the method described in Example 1, except that Polymer 1 was replaced with polyacrylamide (molecular weight range of 5,000 kDa to 6,000 kDa; Sigma). The remainder of the method was the same as set forth in Example 1. The final solution was transparent. A film was prepared using the same method described in Example 3. The dried film was visually transparent.

This example demonstrates that solutions including 5,000 kDa to 6,000 kDa polyacrylamide and the recombinant collagen fragment with a molecular weight of 50 kDa having SEQ ID NO: 1 are miscible and can be prepared as a film or article.

### Example 11

A polyacrylamide and collagen (recombinant collagen fragment with a molecular weight of 50 kDa having SEQ ID NO: 1) mixture was prepared and dried, and visually examined. Example 11 was prepared using the method described in Example 1, except that Polymer 1 was replaced with polyacrylamide (molecular weight of 40 kDa; Sigma). The remainder of the method was the same as set forth in Example 1. The final solution was transparent. A film was prepared using the method described in Example 3, except using an aluminum weighing pan. The dried film was visually transparent.

This example demonstrates that solutions including 40 kDa polyacrylamide and the recombinant collagen fragment with a molecular weight of 50 kDa having SEQ ID NO: 1 are miscible and can be prepared as a film or article.

### Example 12

A first polyvinyl alcohol (PVA) and collagen (recombinant collagen fragment with a molecular weight of 50 kDa having SEQ ID NO: 1) mixture was prepared and visually examined. Example 12 was prepared using the method described in Example 1, except that Polymer 1 was replaced with PVA (molecular weight range of 89 kDa to 98 kDa; Sigma). A 4 wt % PVA solution was prepared by dissolving 2 g PVA in 48 ml DI water and mixing on a stir plate at 400 RPM for 16 hours. The two solutions were mixed at a 1:1 (w/w) ratio in a 50 ml conical tube and mixed at 10 RPM for 16 hours. The final solution was phase separated into two layers. The top layer was turbid and there was a large amount of white powder-like aggregates settled at the bottom.

### Example 13

A second PVA and collagen (recombinant collagen fragment with a molecular weight of 50 kDa having SEQ ID NO: 1) mixture was prepared and visually examined. Example 13 was prepared using the method described in Example 12, except that Polymer 1 was replaced with a 4% PVA (w/v) solution (unknown molecular weight; Fisher Scientific). The remainder of the method was the same as set forth in Example 12. The final solution was phase separated into two layers. The viscosity was determined visually by inspecting the difference between the two layers. The top layer had lower viscosity and was more turbid with small, suspended particles. The bottom layer had higher viscosity and was transparent.

### Example 14

A first hyaluronic acid (HA) and collagen (recombinant collagen fragment with a molecular weight of 50 kDa having SEQ ID NO: 1) mixture was prepared and dried, and visually examined. Example 14 was prepared using the method described in Example 1, except that Polymer 1 was replaced with hyaluronic acid (HA) (molecular weight of 1,000 kDa purchased from Pure Health Botanicals (Saint Charles, IL)). A 2.5 wt % HA solution was prepared by dissolving 5 g HA in 195 ml DI water and mixing with a high shear impeller at 400 RPM for 40 hours due to the high viscosity of the solution. A 5% (w/v) collagen solution including the recombinant collagen fragment of SEQ ID NO: 1 was produced as described in Example 1. The HA solution and collagen solution were mixed at a 2:1 (w/w) ratio in a 50 ml conical tube and mixed at 10 RPM for 16 hours to maintain the dry mass ratio of 1:1. The final solution was transparent. A film was prepared using the method described in Example 3. The dried film was visually hazy with uniformly distributed haziness throughout the film.

### Example 15

A second hyaluronic acid (HA) and collagen (recombinant collagen fragment with a molecular weight of 50 kDa having SEQ ID NO: 1) mixture was prepared and dried, and visually examined. Example 15 was prepared using the method in Example 1, except that Polymer 1 was replaced with non-animal-based HA (molecular weight of 50 kDa; Pure Health Botanicals (Saint Charles, IL) and DSM (Kaiseraugst, Switzerland)). A 5 wt % HA solution was prepared by dissolving 5 g HA in 95 ml DI water and mixing on stir plate at 400 RPM for 16 hours. A 5% (w/v) collagen solution including the recombinant collagen fragment of SEQ ID NO: 1 was produced as described in Example 1. The two solutions were mixed at a 1:1 (w/w) ratio in a 50 ml conical tube and mixed at 10 RPM for 16 hours. The final solution was transparent. A film was prepared using the method described in Example 3. The dried film was visually hazy with uniformly distributed haziness throughout the film. In addition, an aliquot of HA and collagen solution mixture was placed in a fridge at 4° C. overnight and the solution remained transparent.

### Example 16

A first gelatin and HA mixture was prepared and visually examined. Example 16 was prepared using the method in Example 15, except that Polymer 2 was replaced with low molecular weight beef gelatin dietary supplement (Great Lakes Wellness, Grayslake, IL). A 2.2 wt % gelatin solution was prepared by dissolving 1 g gelatin in 45 ml 0.01N HCl and mixing with a high shear impeller at 400 RPM for 16 hours due to the high viscosity of the solution. A 5 wt % 50 kDa HA solution was prepared using the method described in Example 15. The HA solution and gelatin solution were mixed at a 1:2.4 (w/w) ratio in a 50 ml conical tube and mixed at 10 RPM for 16 hours to maintain the dry mass ratio of 1:1. The final solution was phase separated into two layers. The viscosity was determined visually by inspecting the difference between the two layers. The top layer had lower viscosity and was more transparent. The bottom layer had higher viscosity and was more turbid.

### Example 17

A second gelatin and HA mixture was prepared and visually examined. Example 17 was prepared using the method described in Example 16, except that Polymer 2 was replaced with gelatin from porcine skin (molecular weight of 300 kDa; Sigma). A 1.1 wt % gelatin solution was prepared by dissolving 0.5 g gelatin in 45 ml 0.01N HCl and mixing with a high shear impeller at 400 RPM for 16 hours due to the high viscosity of the solution. A 5 wt % 50 kDa HA solution was prepared using the method described in Example 15. The HA solution and gelatin solution were mixed at a 1:4.5 (w/w) ratio in a 50 ml conical tube and mixed at 10 RPM for 16 hours to maintain the dry mass ratio of 1:1. The final solution was phase separated with a large number of white aggregates settled at the bottom and scattered white aggregates suspended or floating at the top.

### Example 18

A HA and collagen Type I mixture was prepared and visually examined. Example 18 was prepared using the method described in Example 15 except that Polymer 2 was replaced with collagen Type I (molecular weight of 414 kDa; Sigma). A 0.7 wt % collagen Type I solution was prepared by dissolving 0.3 g collagen Type I in 45 ml 0.01N HCl and mixing with a high shear impeller at 400 RPM for 16 hours due to the high viscosity of the solution. A 5 wt % 50 kDa HA solution was prepared using the method described in Example 15. The HA solution and collagen Type I solution were mixed together in a 1:7 (w/w) ratio in a 50 ml Falcon conical tube and mixed with Hula mixer at 10 RPM for 16 hours to maintain the dry mass ratio of 1:1. The final solution was phase separated with a large number of white fibrils settled at the bottom and scattered white fibrils suspended or floating at the top.

### Example 19

A cellulase and HA mixture was prepared and visually examined. Example 19 was prepared using the method described in Example 15, except that Polymer 2 was replaced with cellulase (Sunson Industry Group Co., Ltd.). A 5% (w/v) cellulase solution was prepared by dissolving 2.5 g cellulase in 47.5 ml DI water and mixing on stir plate at 400 RPM for 16 hours. A 5 wt % 50 kDa HA solution was prepared using the same method described in Example 15. The two solutions were mixed together in a 1:1 (w/w) ratio in a 50 ml conical tube and mixed at 10 RPM for 16 hours. The final solution was turbid with yellow powders settled at the bottom.

### Example 20

A HA, chitosan, and collagen (recombinant collagen fragment with a molecular weight of 50 kDa having SEQ ID NO: 1) mixture was prepared and visually examined. Example 20 was prepared using the method described in Example 15 except the chitosan that was used in Example 6 was added as Polymer 3. A 5 wt % 50 kDa HA solution was prepared by dissolving 5 g HA in 95 ml DI water and mixing on stir plate at 400 RPM for 16 hours A 5% (w/v) collagen solution including the recombinant collagen fragment of SEQ ID NO: 1 was produced by dissolving 5 grams of the 50 kDa protein into 100 ml of water, as described in Example 1. A 5 wt % chitosan solution was prepared by dissolving 2.5 g chitosan in 47.5 ml DI water and mixing on stir plate at 400 RPM for 16 hours. The chitosan solution was pH adjusted to pH 4 using 0.287 ml acetic acid from Sigma to fully solubilize the solution during mixing. The three solutions were mixed at a 1:1:1 (w/v) ratio in a 50 ml conical tube and mixed at 10 RPM for 16 hours. The final solution was phase separated with large chunks of yellow aggregates suspended or floating in the low viscosity medium.

### Example 21

A mixture of hyaluronic acid and a recombinant collagen fragment with a molecular weight of 50 kDa having SEQ ID NO: 1 (hydroxylated to 22%) was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 21 was prepared using the method in Example 15 except that Polymer 2 was replaced with a 22% hydroxylated collagen with a molecular weight of 50 kDa. The 22% hydroxylated collagen solution had a solid content of 1.3% and pH was about 6. A 5 wt % 50 kDa HA solution (Polymer 1) was prepared by dissolving 5 g HA in 95 ml DI water and mixing on stir plate at 400 RPM for 16 hours. Two solutions were mixed at a 1:1 (w/w) ratio in a 15 ml Falcon tube and mixed at 10 RPM for 16 hours. The final solution was turbid indicating that at this hydroxylation level the two polymers are not miscible in solution.

### Example 22

A mixture of hyaluronic acid and a recombinant collagen fragment with a molecular weight of 50 kDa having SEQ ID NO: 1 (hydroxylated to 44%) was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 22 was prepared using the same method described in Example 21 except using 44% hydroxylated collagen with a molecular weight of 50 kDa as Polymer 2. The 44% hydroxylated collagen solution had a solid content of 1.3% and pH was about 6. A 5 wt % 50 kDa HA solution (Polymer 1) was prepared by dissolving 5 g HA in 95 ml DI water and mixing on stir plate at 400 RPM for 16 hours. The two solutions were mixed at a 1:1 (w/w) ratio in a 15 ml conical tube and mixed at 10 RPM for 16 hours. The final solution was turbid and slowly phase separated, with white aggregates settled at bottom of the tube.

### Example 23

A biphasic crosslinked hyaluronic acid (comprising crosslinked HA and uncrosslinked HA) and collagen (recombinant collagen fragment with a molecular weight of 50 kDa having SEQ ID NO: 1) mixture was prepared and visually examined. Example 23 was prepared using the method described in Example 15, except that Polymer 1 was replaced with biphasic crosslinked HA (particle size of 1.25-2 mm; Beijing Mengbring Bio-Sci-Tec. Co., Ltd.). A 2.4% (w/v) HA solution was used. A solution of 20 wt % of the recombinant collagen fragment of SEQ ID NO: 1 was prepared by dissolving 2 g of the 50 kDa recombinant collagen in 18 ml DI water and mixing at 10 RPM for 4 hours (Polymer 2). The two solutions were mixed together in a 1:1 (w/w) ratio in a 50 ml conical tube and mixed at 10 RPM for 16 hours. The mixed solution was visually transparent.

This example demonstrates that solutions including a biphasic crosslinked HA and the recombinant collagen fragment with a molecular weight of 50 kDa having SEQ ID NO: 1 are fully miscible.

### Example 24

A monophasic crosslinked hyaluronic acid and collagen (recombinant collagen fragment with a molecular weight of 50 kDa having SEQ ID NO: 1) mixture was prepared and visually examined. Example 24 was prepared using the same method in Example 15, except that Polymer 1 was replaced with monophasic crosslinked HA (Bloomage Biotechnology Corp. (Jinan, Shandong, China)). A 2% (w/v) monophasic crosslinked HA solution was used. A 20 wt % collagen solution comprising the recombinant collagen fragment of SEQ ID NO: 1 was prepared by dissolving 2 g of the 50 kDa recombinant collagen in 18 ml DI water and mixing at 10 RPM for 4 hours. The two solutions were mixed at a 1:1 (w/w) ratio in a 50 ml conical tube and mixed at 10 RPM for 16 hours. The solution was visually transparent.

This example demonstrates that solutions including a monophasic crosslinked HA and the recombinant collagen fragment with a molecular weight of 50 kDa having SEQ ID NO: 1 are fully miscible.

### Example 25

A biphasic crosslinked hyaluronic acid (comprising crosslinked HA and uncrosslinked HA) and collagen (recombinant collagen fragment with a molecular weight of 50 kDa having SEQ ID NO: 1) mixture was prepared and visually examined. Example 25 was prepared using the method described in Example 24, except that Polymer 1 was replaced with biphasic crosslinked HA (Restylane). A 2.0% (w/v) biphasic crosslinked HA solution was used. A 40 wt % solution of the recombinant collagen fragment of SEQ ID NO: 1 was prepared by dissolving 4 g of the 50 kDa recombinant collagen in 16 ml DI water and mixing at 10 RPM for 4 hours. The two solutions were mixed at a 1:3, 1:1, 10:3 and 100:3 (w/w) ratio of HA:collagen in a 1 ml Eppendorf tube with a pipette and mixed at 10 RPM for 16 hours. The solutions were transparent visually.

This example demonstrates that solutions including a biphasic crosslinked hyaluronic acid and the recombinant collagen fragment with a molecular weight of 50 kDa having SEQ ID NO: 1 are fully miscible.

### Example 26

A 5% hyaluronic acid and collagen (recombinant collagen fragment with a molecular weight of 50 kDa having SEQ ID NO: 1) mixture was prepared and visually examined. Example 26 was prepared using the method described in Example 15, except that the pH of the 5 wt % 50 kDa HA solution was adjusted to pH 5.2-7.2 with sodium hydroxide prior to the addition of a collagen solution comprising the recombinant collagen fragment of SEQ ID NO: 1. The two solutions were mixed together in a 1:1 (w/w) ratio in a 50 ml tube and mixed at 10 RPM for 16 hours. The solutions were visually transparent. Films were prepared using the method of Example 3. The dried films were visually hazy with uniformly distributed haziness throughout the film.

This example demonstrates that solutions including 5% hyaluronic acid at pH 5.2-7.2 and the recombinant collagen fragment with a molecular weight of 50 kDa having SEQ ID NO: 1 are fully miscible.

### Example 27

A 5% hyaluronic acid and collagen (recombinant collagen fragment with a molecular weight of 50 kDa having SEQ ID NO: 1) mixture was prepared at different pHs and visually examined. Example 27 was prepared using the method described in Example 15, except the pH of the 5 wt % 50 kDa HA solution was adjusted to pH of 9, 10, 11, and 12 with sodium hydroxide prior to the addition of the recombinant collagen fragment of SEQ ID NO: 1. The two solutions were mixed at a 1:1 (w/w) ratio in a 20 ml glass vial and mixed with a stir bar at 400 RPM for 30 minutes due to lower viscosity of the basic solutions. The solutions were then readjusted to pH 7±1 with concentrated hydrochloric acid while mixing with a stir bar at 400 RPM. The solutions were visually transparent prior and after pH adjusted to 7±1.

This example demonstrates that solutions including 5% hyaluronic acid at pH 9, 10, 11, 12, and the recombinant collagen fragment with a molecular weight of 50 kDa having SEQ ID NO: 1 are fully miscible.

### Example 28

A hyaluronic acid and collagen mixture was prepared and visually examined. Example 28 was prepared using the method in Example 15 except that Polymer 1 was replaced with 2% HumaColl21® solution (Geltor). A 2 wt % 50 kDa HA solution was prepared using the method described in Example 15. The two solutions were mixed at a 1:1 (w/w) ratio in a 2 ml glass vial and mixed with on a stir plate at 400 RPM for 1 hour. The final solution was visually transparent.

This example demonstrates that solutions including 2% HumaColl21@solution and the recombinant collagen fragment with a molecular weight of 50 kDa having SEQ ID NO: 1 are fully miscible.

### Example 29

Hyaluronic acid and full-length recombinant collagen and a 50 kDa recombinant collagen fragment are blended, mixed with HA, and visually examined. A full-length recombinant collagen and a 50 kDa recombinant collagen fragment are blended and mixed with HA. The solutions are visually inspected. A blend of 50 kDa recombinant collagen fragment with full-length recombinant collagen and/or hydrolyzed recombinant collagen (or combinations of any of these three) is mixed with HA, and visually examined. Then, the blend of the full-length recombinant collagen and the 50 kDa recombinant collagen fragment and a blend of the blend of the full-length recombinant collagen and a hydrolyzed full-length recombinant collagen are mixed with HA. The solutions are visually inspected.

### Example 30

A poly(2-ethyl-2-oxazoline) (PEOx) solution was mixed with a solution comprising the recombinant collagen fragment with a molecular weight of 50 kDa having SEQ ID NO: 1. Example 30 was prepared using the same method described in Example 1, except that Polymer 1 was replaced with PEOx (molecular weight of 50 kDa; Sigma)1. The remainder of the method was the same as set forth in Example 1. The final solution was visually transparent indicating that the polymers were miscible in solution. A film was prepared using the same method as described in Example 3 except in an aluminum weighing pan. The dried film visually appeared to be phase separated during drying.

### Example 31

A polyethylenimine (PEI) solution was mixed with a solution including the recombinant collagen fragment with a molecular weight of 50 kDa having SEQ ID NO: 1. Example 31 was prepared using the method described in Example 1, except that Polymer 1 was replaced with PEI (molecular weight of 100 kDa; Polysciences (Warrington, Pennsylvania)). A 5 wt % polyethylenimine solution was prepared by dissolving 0.5 g of PEI in 9.5 g of Milli-Q water at 80° C. using a stirrer bar at 450 RPM for 3 hours, or until the solution was completely transparent. The solution was then cooled to room temperature and the pH was adjusted to 7 using 12N HCl. The remainder of the method was the same as set forth in Example 1. The solution was transparent indicating that the polymers were miscible in solution. A film was prepared using the method set forth in Example 3. The dried film was visually opaque.

### Example 32

A first sodium carboxymethylcellulose (NaCMC) and collagen (recombinant collagen fragment with a molecular weight of 50 kDa having SEQ ID NO: 1) mixture was prepared and dried, and visually examined. Example 32 was prepared using the method as described in Example 1, except that Polymer 1 was replaced with sodium carboxymethylcellulose (molecular weight of ˜250 kDa and a degree of substitution of 1.22; Sigma). A 5 wt % sodium carboxymethylcellulose solution was prepared by dissolving 0.5 g of sodium carboxymethylcellulose in 9.5 g DI water and mixing in a conical tube for 16 hours at 5 RPM. The two solutions were mixed at a 1:1 (v/v) ratio in a 50 ml conical tube and mixed at 10 RPM for 16 hours. The final solution was transparent and slightly yellow indicating that the solutions were miscible. A film was prepared using the method set forth in Example 3, except using an aluminum weighing pan. The dried film was visually hazy with uniformly distributed haziness throughout the film.

### Example 33

A second sodium carboxymethylcellulose and collagen (recombinant collagen fragment with a molecular weight of 50 kDa having SEQ ID NO: 1) mixture was prepared and dried, and visually examined. Example 33 was prepared using the same method described in Example 1, except that Polymer 1 was replaced with sodium carboxymethylcellulose (molecular weight of ˜250 kDa and a degree of substitution of 0.79; Sigma). A 2.5 wt % sodium carboxymethylcellulose solution was prepared by dissolving 0.25 g of sodium carboxymethylcellulose in 9.75 g DI water and mixing in a conical tube for 16 hours at 5 RPM. A 2.5 wt % collagen solution comprising the recombinant collagen fragment with amino acid sequence of SEQ ID NO: 1 was prepared by dissolving 0.25 g of the 50 kDa recombinant collagen in 9.75 g DI water. The two solutions were mixed at a 1:1 (v/v) ratio in a 50 ml conical tube and mixed at 10 RPM for 16 hours. The final solution was transparent indicating that the polymers were miscible in solution. A film was prepared using the method set forth in Example 3, except using an aluminum weighing pan. The dried film was visually hazy with uniformly distributed haziness throughout the film.

### Example 34

A third sodium carboxymethylcellulose and collagen (recombinant collagen fragment with a molecular weight of 50 kDa having SEQ ID NO: 1) mixture was prepared and dried, and visually examined. Example 35 was prepared using the method described in Example 1, except that Polymer 1 was replaced with using sodium carboxymethylcellulose (high viscosity of 2,700 cps for 1% aqueous solution and a degree of substitution of 0.87; Sigma). A 2.5 wt % sodium carboxymethylcellulose solution was prepared by dissolving 0.25 g of sodium carboxymethylcellulose in 9.75 g DI water and mixing in a conical tube for 16 hours at 5 RPM. A 2.5 wt % solution comprising the recombinant collagen fragment with the amino acid sequence of SEQ ID NO: 1 was produced by dissolving 0.25 g of the recombinant collagen fragment into 9.75 g of water. The two solutions were mixed at a 1:1 (v/v) ratio in a 50 ml conical tube and mixed at 10 RPM for 16 hours. The final solution was transparent indicating that the polymers were miscible in solution. A film was prepared using the method set forth in Example 3, except using an aluminum weighing pan. The dried film was visually hazy with uniformly distributed haziness throughout the film.

### Example 35 (HA/SEQ ID NO: 1007)

A recombinant collagen fragment having the amino acid sequence of SEQ ID NO: 1007 and HA mixture was prepared, dried, and visually examined. SEQ ID NO: 1007 is 98% identical to SEQ ID NO: 1. Example 35 was prepared using the method as described in Example 15, except that Polymer 2 was replaced with a collagen polymer having amino acid sequence of SEQ ID NO: 1007. A 5 wt % solution comprising the recombinant collagen fragment having amino acid sequence of SEQ ID NO: 1007 was made by dissolving 0.25 g of the recombinant collagen fragment in 4.75 g of water. A 5 wt % 50 kDa HA solution was prepared by dissolving 0.25 g of 50 kDa HA (Polymer 1) in 4.75 g of water. The two solutions were mixed at a 1:1 (w/w) ratio in a 50 ml conical tube and mixed at 10 RPM for 16 hours. The conical tube was placed vertically on a rack overnight to assess the solution stability. The final solution was transparent. A film was prepared using the method set forth in Example 3, except using an aluminum pan. The dried film was visually hazy with uniformly distributed haziness throughout the film.

### Example 36 (HA/SEQ ID NO: 1008)

A recombinant collagen fragment having the amino acid sequence of SEQ ID NO: 1008 and HA mixture was prepared, dried, and visually examined. SEQ ID NO: 1008 is 50% identical to SEQ ID NO: 1. Example 36 was prepared using the method described in Example 15, except that Polymer 2 was replaced with a collagen polymer having amino acid sequence of SEQ ID NO: 1008. A 5 wt % collagen solution comprising the collagen polymer having amino acid sequence of SEQ ID NO: 1008 was prepared by dissolving 0.25 g of the recombinant collagen fragment in 4.75 g of Milli-Q Water (water purified using a Millipore Milli-Q lab water system; “MQ water”) and purified through centrifugation at 3214 relative centrifugal force (RCF) for 1 hour. A 5 wt % 50 kDa HA (Polymer 1) solution was prepared using the same method described in Example 35. The supernatant of the collagen solution and the HA solution were mixed at a 1:1 (w/w) ratio in a 50 ml Falcon tube and mixed at 10 RPM for 16 hours. The solution was transparent indicating that the two polymers were miscible in solution. The dried film was visually hazy.

### Example 37 (HA/SEQ ID NO: 973)

A full-length collagen and HA mixture was prepared, dried, and visually examined. Example 37 was prepared using the method as described in Example 35, except that Polymer 2 was replaced with a recombinant collagen fragment with a molecular weight of 50 kDa and having SEQ ID NO: 973. A 5 wt % collagen solution comprising the recombinant collagen fragment of SEQ ID NO: 973 was prepared by dissolving 0.25 g of the recombinant collagen in 4.75 g of MQ water. A 5 wt % 50 kDa HA solution was prepared using the method described in Example 36. The remainder of the method was the same as set forth in Example 36. The solution was transparent indicating that the polymers were miscible in solution. The dried film was visually hazy with uniformly distributed haziness throughout the film.

### Example 38 (Chondroitin Sulfate/Collagen)

A chondroitin sulfate and collagen (recombinant collagen fragment with a molecular weight of 50 kDa having SEQ ID NO: 1) mixture was prepared, dried, and visually examined. Example 38 was prepared using the method described in Example 1, except that Polymer 1 was replaced with chondroitin sulfate from bovine trachea (molecular weight of 50 kDa; EMD Millipore (Burlington, MA)). A 5 wt % chondroitin sulfate solution was prepared by dissolving 0.5 g of chondroitin sulfate in 9.5 g MQ Water. The remainder of the method was the same as set forth in Example 1. The final solution was transparent indicating that the polymers were miscible in solution. A film was prepared using the same method as described in Example 3, except using an aluminum weighing pan. The dried film was visually hazy with uniformly distributed haziness throughout the film.

### Example 39 (AcHA/Collagen)

An acetylated hyaluronate (AcHA) and collagen (recombinant collagen fragment with a molecular weight of 50 kDa having SEQ ID NO: 1) mixture was prepared, dried, and visually examined. Example 39 was prepared using the method described in Example 15, except that Polymer 1 was replaced with sodium acetylated hyaluronate (Bloomage Biotechnology Corp., Ltd.). A 5 wt % Acetylated Hyaluronic Acid (AcHA) solution was prepared by dissolving 0.25 g of AcHA in 4.75 g of MQ Water. A 5 wt % solution comprising the recombinant collagen fragment of SEQ ID NO: 1 was prepared by dissolving 0.25 g of collagen in 4.75 g of water. The remainder of the method was the same as set forth in Example 15. The solution was transparent indicating that the polymers were miscible in solution. A film was prepared using the same method described in Example 3, except using an aluminum pan. The dried film was visually hazy with uniformly distributed haziness throughout the film.

### Example 40 (Zinc HA/Collagen)

A Zinc hyaluronate (ZnHA) and collagen (recombinant collagen fragment with a molecular weight of 50 kDa having SEQ ID NO: 1) mixture was prepared and dried, and visually examined. Example 40 was prepared using the method described in Example 15, except that Polymer 1 was replaced with (Zinc Hyaluronate (ZnHA) (molecular weight of 170 kDa; Bloomage Biotechnology Corp., Ltd.). A 5 wt % Zinc Hyaluronate (ZnHA) solution was prepared by dissolving 0.25 g of ZnHA in 4.75 g of MQ Water. A 5 wt % solution comprising the recombinant collagen fragment of SEQ ID NO: 1 was prepared by dissolving 0.25 g of collagen in 4.75 g of water. The remainder of the method was the same as set forth in Example 15. The solution was turbid indicating aggregation of particles. A film was prepared using the same method as described in Example 3, except using an aluminum pan. The dried film was transparent indicating that the polymers were miscible.

### Example 41 (HA/Collagen Salt Doping Study)

A hyaluronic acid and collagen (recombinant collagen fragment with a molecular weight of 50 kDa having SEQ ID NO: 1) solution was prepared, dried, and visually examined. Example 41 was prepared using the method as described in Example 15, except that the solution comprising the recombinant collagen fragment with a molecular weight of 50 kDa having SEQ ID NO: 1 was prepared in a NaCl solution. A 5 wt % collagen solution was prepared by dissolving 0.25 g in 4.75 g in a 0.05 mM solution of NaCl. A 5 wt % 50 kDa HA solution was prepared using the same method as described in Example 35. The two solutions were mixed at a 1:1 (w/w) ratio in a 50 ml conical tube and mixed at 10 RPM for 4 hours. The solution was transparent indicating that the polymers were miscible in higher salt content solutions. A film was prepared using the same method as described in Example 3, except using an aluminum pan. The dried film was hazy.

### Example 42 (HA/Collagen Dialysis)

A hyaluronic acid and collagen (recombinant collagen fragment with a molecular weight of 50 kDa having SEQ ID NO: 1) solution was prepared, dried, and visually examined. Example 42 was prepared using the method described in Example 15, except the hyaluronic and collagen solution was dialyzed. A 5 wt % collagen solution comprising the recombinant collagen fragment of SEQ ID NO: 1 was prepared by dissolving 0.25 g of the recombinant collagen in 4.75 g of MQ water. A 5 wt % 50 kDa HA solution was prepared by dissolving 0.25 g of hyaluronic acid powder in 4.95 g of MQ water. The two solutions were mixed at a 1:1 (w/w) ratio in a 50 ml conical tube and mixed at 10 RPM for 4 hours. Then, 9 g of the combined solution was loaded into a 10 ml volume 3.5-5 kDa molecular weight cutoff (MWCO) membrane. A dialysate reservoir was prepared with a Nalgene bottle containing 500 ml of MQ water and a stir bar to keep the surrounding solution gently rotating. The membrane containing the solution was submerged in the dialysate reservoir and dialyzed at room temperature for 16 hours. The solution was pipetted out of the membrane and transferred into a 50 ml conical tube. The solution was transparent indicating that the polymers were miscible in lower salt content solutions. A film was prepared using the same method as described in Example 3, except using an aluminum pan. The dried film was visually hazy with uniformly distributed haziness throughout the film.

### Example 43 (HA and Hydrolyzed Recombinant Collagen Fragment—5 mg/mL Papain)

A hyaluronic acid and hydrolyzed collagen solution (hydrolyzed fragments of recombinant collagen fragment with a molecular weight of 50 kDa having SEQ ID NO: 1) was prepared, dried, and visually examined. A 5 wt % solution comprising the recombinant collagen fragment with the amino acid sequences of SEQ ID NO: 1 was made by dissolving 0.25 g of the collagen in 4.75 g of water. A 10 wt % Papain enzyme solution was made by dissolving 100 mg Papain (200 TU/mg, sourced from BIO-CAT) in 900 μl of water. A hydrolyzed recombinant fragment solution was made by adding 250 μl of 10 wt % Papain enzyme solution into 4.75 ml of 5 wt % recombinant collagen solution and incubating at 60° C. for 2 hours for enzymatic hydrolysis, and then at 90° C. for 10 minutes to deactivate the enzyme activity. After that, the hydrolyzed solution was centrifuged at 3214 relative centrifugal force (RCF) for 20 minutes. The supernatant solution was collected to mix with the following HA solution. A 5 wt % recombinant collagen fragment and HA solution was prepared by dissolving 0.25 g of 50 kDa HA in 4.75 g of water. The two solutions were mixed at a 1:1 (w/w) ratio in a 50 ml conical tube and mixed at 5 RPM for 2 hours. The conical tube was placed vertically on a rack overnight to assess the solution stability. The final solution was transparent. A film was prepared using the method set forth in Example 3 except using a silicone mold. The dried film was visually transparent indicating that the polymers were fully miscible.

### Example 44 (PVP and Hydrolyzed Recombinant Collagen Fragment—5 mg/mL Papain)

A polyvinylpyrrolidone and hydrolyzed recombinant collagen solution (hydrolyzed fragments of recombinant collagen fragment with a molecular weight of 50 kDa having SEQ ID NO: 1) was prepared, dried, and visually examined. Example 44 was prepared using the method described in Example 43, except that Polymer 1 was replaced with polyvinylpyrrolidone (molecular weight of 40 kDa; Sigma). A 5 wt % 40 kDa PVP solution was prepared by dissolving 0.25 g of 40 kDa PVP in 4.75 g of water. The hydrolyzed recombinant collagen solution was prepared using the method described in Example 44. The two solutions were mixed at a 1:1 (w/w) ratio in a 50 ml conical tube and mixed at 5 RPM for 2 hours. The conical tube was placed vertically on a rack overnight to assess the solution stability. The final solution was transparent. A film was prepared using the method set forth in Example 3, except using a silicone mold. The dried film was visually transparent indicating that the polymers were miscible in solution and solid.

### Example 45 (HA and Blend of Recombinant Collagen Fragment and Hydrolyzed Recombinant Collagen Fragment—0.1 mg/mL Papain)

A solution of hyaluronic acid, a recombinant collagen fragment with a molecular weight of 50 kDa having SEQ ID NO: 1, and partially hydrolyzed recombinant collagen fragments was prepared, dried, and visually examined. A 5 wt % solution comprising the recombinant collagen fragment having the amino acid sequence of SEQ ID NO: 1 was made by dissolving 0.25 g of the recombinant collagen fragment in 4.75 g of water. A blend of recombinant collagen fragment and hydrolyzed recombinant collagen fragment solution was prepared by partially hydrolyzing the 5 wt % collagen solution. A 1 wt % Papain enzyme solution was made by dissolving 10 mg Papain (200 TU/mg, sourced from BIO-CAT) in 990 μl of water. A solution of partially hydrolyzed recombinant fragments was made by adding 50 μl of 1 wt % Papain enzyme solution into 4.95 ml of 5 wt % recombinant collagen solution and incubating at 60° C. for 2 hours for enzymatic hydrolysis, and then at 90° C. for 10 minutes to deactivate the enzyme activity (Polymer 2). A 5 wt % 50 kDa HA (Polymer 1) solution was prepared by dissolving 0.25 g of 50 kDa HA in 4.75 g of water. The two solutions were mixed at a 1:1 (w/w) ratio in a 50 ml conical tube and mixed at 5 RPM for 2 hours. The conical tube was placed vertically on a rack overnight to assess the solution stability. The final solution was transparent indicating that the polymers were miscible in solution. A film was prepared using the method set forth in Example 3, except using a silicone mold. The dried film was visually hazy with uniformly distributed haziness throughout the film.

### Example 46 (PVP and Blend of Recombinant Collagen Fragment and Hydrolyzed Recombinant Collagen Fragment—0.1 mg/mL Papain)

A solution of polyvinylpyrrolidone, a recombinant collagen fragment with a molecular weight of 50 kDa having SEQ ID NO: 1, and partially hydrolyzed recombinant collagen fragments was prepared, dried, and visually examined. Example 46 was prepared using the method described in Example 45, except that Polymer 1 was replaced with polyvinylpyrrolidone (molecular weight of 40 kDa; Sigma). A 5 wt % 40 kDa PVP solution was prepared by dissolving 0.25 g of 40 kDa PVP in 4.75 g of water. A blend of recombinant collagen fragment and hydrolyzed recombinant collagen fragment solution was prepared using the method described in Example 45. The two solutions were mixed at a 1:1 (w/w) ratio in a 50 ml conical tube and mixed at 5 RPM for 2 hours. The conical tube was placed vertically on a rack overnight to assess the solution stability. The final solution was transparent. A film was prepared using the method set forth in Example 3, except using an aluminum weighing pan. The dried film was visually transparent indicating that the polymers were miscible in solution and solid.

### Example 47 (Biphasic HA and Blend of Recombinant Collagen Fragment and Hydrolyzed Recombinant Collagen Fragment—0.1 mg/mL Papain)

A solution of biphasic crosslinked hyaluronic acid (comprising crosslinked HA and uncrosslinked HA) and a blend of a recombinant collagen fragment with a molecular weight of 50 kDa having SEQ ID NO: 1, and partially hydrolyzed recombinant collagen fragments was prepared and visually examined. Example 47 was prepared using the method described in Example 45, except Polymer 1 was replaced with a 2.4% (w/v) biphasic crosslinked HA with a particle size of 0.10-0.25 mm (Shandong Runxin Biotechnology Co., Ltd. (Qufu City, Shandong Province, China). A solution containing a blend of recombinant collagen fragments with a molecular weight of 50 kDa having SEQ ID NO: 1 and hydrolyzed recombinant collagen fragments was prepared using the method as described in Example 45. The two solutions were mixed at a 1:1 (w/w) ratio in a 50 ml conical tube and vortexed for 3 minutes and mixed at 5 RPM for 3 hours. The conical tube was placed vertically on a rack overnight to assess the solution stability. The solution was visually slightly hazy due to visible crosslinked HA particle texture.

### Example 48 (Sodium CMC and Blend of Recombinant Collagen Fragment and Hydrolyzed Recombinant Collagen Fragment—0.1 mg/mL Papain)

A solution of sodium carboxymethylcellulose and blend of a recombinant collagen fragment with a molecular weight of 50 kDa having SEQ ID NO: 1, and partially hydrolyzed recombinant collagen fragments was prepared, dried, and visually examined. Example 48 was prepared using the method described in Example 45, except that Polymer 1 was replaced with sodium carboxymethylcellulose (molecular weight of ˜250 kDa and a degree of substitution of 1.22; Sigma). A 5 wt % sodium carboxymethylcellulose solution was prepared by dissolving 0.5 g of sodium carboxymethylcellulose in 9.5 g DI water and mixing in a conical tube for 16 hours at 5 RPM. A blend of recombinant collagen fragment and hydrolyzed recombinant collagen fragment solution was prepared using the method described in Example 45. The two solutions were mixed at a 1:1 (w/w) ratio in a 50 ml conical tube and mixed at 5 RPM for 2 hours. The conical tube was placed vertically on a rack overnight to assess the solution stability. The final solution was transparent indicating that the polymers were miscible in solution. A film was prepared using the method set forth in Example 3 except using an aluminum weighing pan. The dried film was visually hazy with uniformly distributed haziness throughout the film.

### Example 49 (Sodium Carboxymethylcellulose and SEQ ID NO: 1007)

A sodium carboxymethylcellulose and recombinant collagen polymer having the amino acid sequence set forth in SEQ ID NO: 1007 solution was prepared, dried, and visually examined. SEQ ID NO: 1007 is 98% identical to SEQ ID NO: 1. A 5 wt % solution comprising the recombinant collagen polymer having the amino acid sequence set forth in SEQ ID NO: 1007 was made by dissolving 0.5 g of the recombinant collagen fragment in 9.5 g of water. A 5 wt % sodium carboxymethylcellulose (molecular weight of ˜250 kDa and a degree of substitution of 1.22; Sigma) solution was prepared by dissolving 0.5 g of sodium carboxymethylcellulose in 9.5 g DI water and mixing in a conical tube for 16 hours at 5 RPM. The two solutions were mixed at a 1:1 (w/w) ratio in a 50 ml conical tube and mixed at 5 RPM for 2 hours. The conical tube was placed vertically on a rack overnight to assess the solution stability. The final solution was transparent indicating that the polymers were miscible in solution. A film was prepared using the method set forth in Example 3, except using a silicone mold. The dried film was visually hazy with uniformly distributed haziness throughout the film.

### Example 50 (PVP and SEQ ID NO: 1007)

A polyvinylpyrrolidone and recombinant collagen polymer having the amino acid sequence set forth in SEQ ID NO: 1007 solution was prepared, dried, and visually examined. SEQ ID NO: 1007 is 98% identical to SEQ ID NO: 1. A 5 wt % solution comprising the recombinant collagen fragment having amino acid sequence set forth in SEQ ID NO: 1007 was made by dissolving 0.5 g of the recombinant collagen fragment in 9.5 g of water. A 5 wt % 40 kDa PVP solution was prepared by dissolving 0.25 g of 40 kDa PVP in 4.75 g of water. The two solutions were mixed at a 1:1 (w/w) ratio in a 50 ml conical tube and mixed at 5 RPM for 2 hours. The conical tube was placed vertically on a rack overnight to assess the solution stability. The final solution was transparent. A film was prepared using the method set forth in Example 3, except using a silicone mold. The dried film was visually transparent indicating that the polymers were miscible in solution and in the solid state.

### Example 51 (Biphasic HA and SEQ ID NO: 1007)

A biphasic crosslinked hyaluronic acid (comprising crosslinked HA and uncrosslinked HA) and blend of recombinant collagen fragment having the amino acid sequence set forth in SEQ ID NO: 1007 and hydrolyzed recombinant collagen fragment solution was prepared and visually examined. SEQ ID NO: 1007 is 98% identical to SEQ ID NO: 1. A 2.4% (w/v) biphasic crosslinked HA with a particle size of 0.10-0.25 mm purchased from Shandong Runxin Biotechnology Co., Ltd. (Qufu City, Shandong Province, China) was used as Polymer 1. A 5 wt % solution comprising the recombinant collagen fragment having the amino acid sequence set forth in SEQ ID NO: 1007 was made by dissolving 0.5 g of the recombinant collagen fragment in 9.5 g of water. The two solutions were mixed at a 1:1 (w/w) ratio in a 50 ml conical tube and vortexed for 3 minutes and mixed at 5 RPM for 3 hours. The conical tube was placed vertically on a rack overnight to assess the solution stability. The solution was visually transparent. In addition, the same conical tube was placed vertically on a rack in a fridge at 4° C. overnight and the solution remained transparent indicating that the polymers were miscible in solution.

### Example 52 (HA:SEQ ID NO: 1 at Different Ratios)

A hyaluronic acid (HA) and collagen (recombinant collagen fragment with a molecular weight of 50 kDa having SEQ ID NO: 1) mixture was prepared and dried, and visually examined. A 5 wt % 50 kDa HA solution was prepared by dissolving 2.5 g HA in 47.5 g of DI water. A 5% (w/v) collagen solution including the recombinant collagen fragment of SEQ ID NO: 1 was made by dissolving 2.5 g of the recombinant collagen fragment in 47.5 g of water. The two solutions were mixed at a 1:9, 1:3, 2:3, 1:1, 3:2, 3:1, and 9:1 (w/w) ratio of HA:collagen with 10 g total solution weight in 50 ml conical tubes and mixed at 5 RPM for 3 hours. The conical tubes were placed vertically on a rack overnight to assess the solution stability. The final solutions were transparent indicating that all ratios are miscible in solution. Films were prepared using the same method as described in Example 3 except using a silicone mold. The dried film with HA:collagen at a ratio of 1:9 was visually transparent. The dried film with HA:collagen at a ratio of 1:3 was visually transparent to slightly hazy. The dried films of the remaining ratios were visually hazy with uniformly distributed haziness throughout the film indicating that miscibility in the solid state is composition dependent. In addition, aliquots of HA:collagen solution mixtures for those seven ratios were placed in a fridge at 4° C. overnight and the solutions remained transparent.

### Example 53 (HA:5% Hydroxylation)

A mixture of hyaluronic acid and a 5% hydroxylated recombinant collagen fragment with a molecular weight of 50 kDa having SEQ ID NO: 1 was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 53 was prepared using the method described in Example 21, except that Polymer 2 was replaced with a 5.41% hydroxylated collagen (molecular weight of 50 kDa). A 2 wt % 50 kDa HA solution (polymer 1) was prepared by dissolving 0.2 g of HA in 9.8 g of MQ water. A 5% hydroxylated collagen had a solid content of 12% and pH was about 6. The hydroxylated collagen solution was then diluted to 2 wt % with water and purified by centrifugation at 15,000 RPM for 10 min. The supernatants of the hydroxylated collagen solution and the HA solution were mixed at a 1:1 (w/w) ratio in a 15 ml Falcon tube and mixed at 10 RPM for 16 hours. The solution was transparent indicating that the polymers were miscible in solution. A film was prepared using the method set forth in Example 3, except in a silicon mold. The dried film was visually hazy.

### Example 54 (HA:12% Hydroxylation)

A mixture of hyaluronic acid and a 12% hydroxylated recombinant collagen fragment with a molecular weight of 50 kDa having SEQ ID NO: 1 was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 54 was prepared using the method described in Example 21, except using a 12.47% hydroxylated collagen with a molecular weight of 50 kDa as Polymer 2. A 2 wt % 50 kDa HA solution (Polymer 1) was prepared by dissolving 0.2 g of HA in 9.8 g of MQ water. A 12% hydroxylated collagen had a solid content of 3% and pH was about 6. The hydroxylated collagen solution was then diluted to 2 wt % with water and purified by centrifugation at 15,000 RPM for 10 min. The supernatants of the hydroxylated collagen solution and the HA solution were at a 1:1 (w/w) ratio in a 15 ml Falcon tube and mixed at 10 RPM for 16 hours. The solution was transparent indicating that the polymers were miscible in solution. A film was prepared using the method set forth in Example 3, except in a silicon mold. The dried film was visually hazy.

### Example 55 (HA:5% Hydroxylation in HCl)

A mixture of hyaluronic acid and a 5% hydroxylated recombinant collagen fragment with a molecular weight of 50 kDa having SEQ ID NO: 1 was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 55 was prepared using the method described in Example 21, except using a 5.41% hydroxylated collagen with a molecular weight of 50 kDa as Polymer 2. A 2 wt % 50 kDa HA solution (Polymer 1) was prepared by dissolving 0.2 g of HA in 9.8 g of MQ water. A 5% hydroxylated collagen had a solid content of 12% and pH was about 6. The hydroxylated collagen solution was then diluted to 2 wt % with water and pH adjusted to ˜2.2 using 12N HCl. The collagen solution was purified by centrifugation at 15,000 RPM for 10 minutes. The supernatants of the hydroxylated collagen solution and the HA solution were mixed at a 1:1 (w/w) ratio in a 15 ml Falcon tube and mixed at 10 RPM for 16 hours. The solution was turbid. The collagen and HA solution was neutralized using ION NaOH. The solution was transparent. A film was prepared using the method set forth in Example 3, except in a silicon mold. The dried film was visually hazy.

### Example 56 (HA:12% Hydroxylation in HCl)

A mixture of hyaluronic acid and a 12% hydroxylated recombinant collagen fragment with a molecular weight of 50 kDa having SEQ ID NO: 1 was prepared and visually examined. The recombinant collagen fragment was hydroxylated by the process described in International Application No. WO2021/163485, and the percent hydroxylation was measured by the process described in International Application No. WO2021/163485, the disclosure of which is incorporated herein in its entirety. Example 56 was prepared using the method described in Example 21, except using a 12.47% hydroxylated collagen with a molecular weight of 50 kDa as Polymer 2. A 2 wt % 50 kDa HA solution (Polymer 1) was prepared by dissolving 0.2 g of HA in 9.8 g of MQ water. A 12% hydroxylated collagen had a solid content of 3% and pH was about 6. The hydroxylated collagen solution was then diluted to 2 wt % with water and pH adjusted to ˜2.2 using 12N HCl. The collagen solution was purified by centrifugation at 15,000 RPM for 10 minutes. The supernatants of the hydroxylated collagen solution and the HA solution were mixed at a 1:1 (w/w) ratio in a 15 ml Falcon tube and mixed at 10 RPM for 16 hours. The solution was turbid. The collagen and HA solution was neutralized using 10N NaOH. The solution was transparent. A film was prepared using the method set forth in Example 3, except in a silicon mold. The dried film was visually hazy.

### Example 57 (SEQ ID NO: 1 Foam Preparation)

A stock solution of recombinant collagen (SEQ ID NO: 1) in water was prepared at a weight concentration of 16.7% (w/w). Baymedix FD103 and Baymedix® AD111 are each aqueous polyurethane dispersions. A stock solution of Baymedix (Bayer MaterialScience, Pittsburgh, PA) FD103:AD111 was prepared at a ratio of 70:30 based on the weight of polyurethane dispersion. A stock solution of Rheolate 208 (Elementis, London, UK) in water was prepared at a weight concentration of 10% (w/w). 9.16 g of recombinant collagen stock solution and 11.50 g of Baymedix FD103:AD111 stock solution were mixed in a 50 mL beaker with an overhead mixer. After that, 1.55 g of Rheolate 208 stock solution was added and mixed. Then 0.72 g of ChemTex (Cumberland, RI) 2216, 0.65 g of ChemTex 2317, and 0.03 g of ChemTex 2243 were added and the resulting mixture was mixed further. The resulting mixture was mechanically frothed by vigorously mixing with the overhead mixer. After frothing, the frothed mixture was coated on a glass plate at a gap setting of 600 μm. The coating was dried at 75° C. for 30 minutes, and a porous foam sheet was achieved after drying.

### Example 58 (Collagen Protein Release Kinetics)

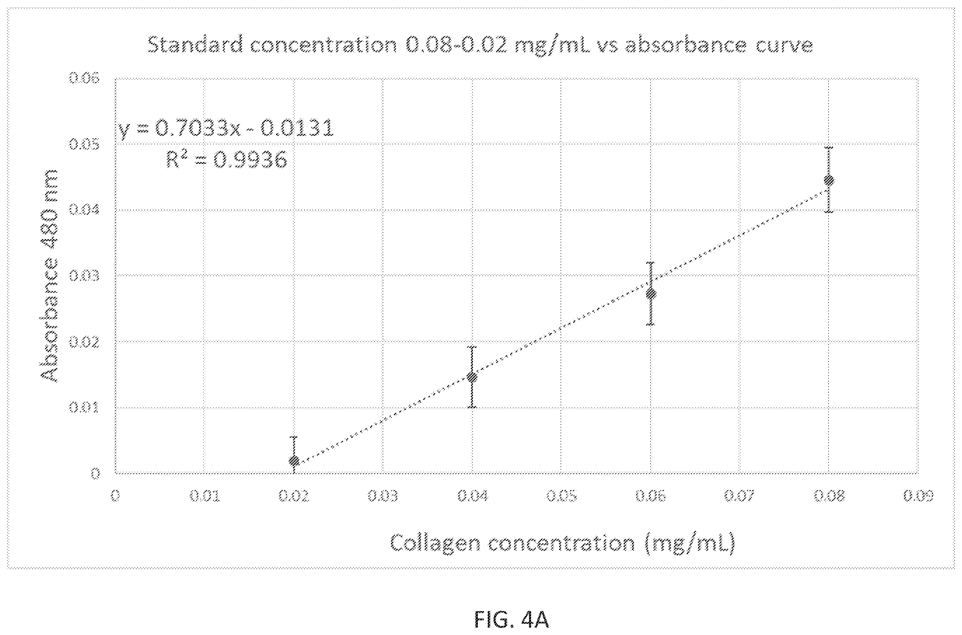

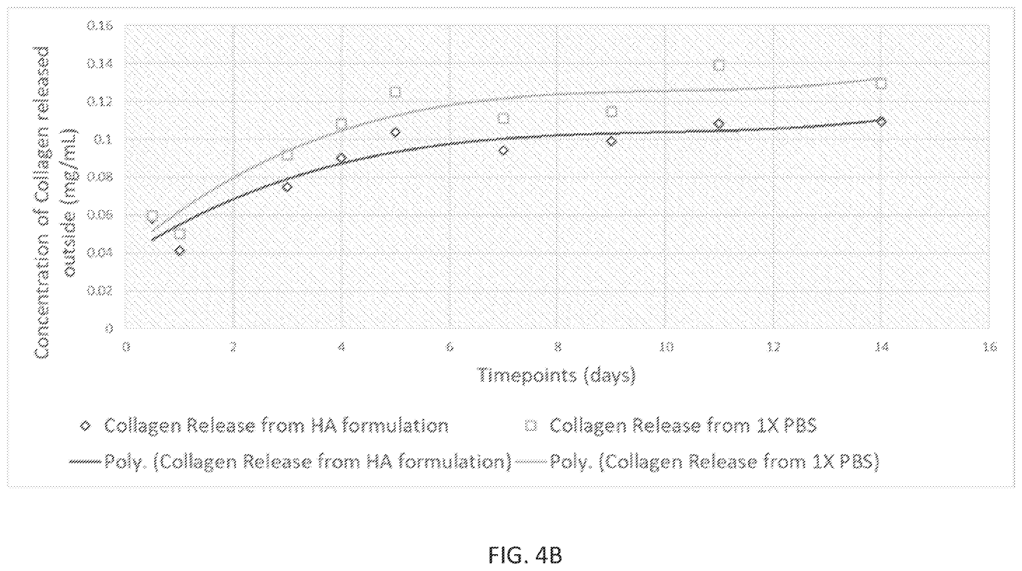

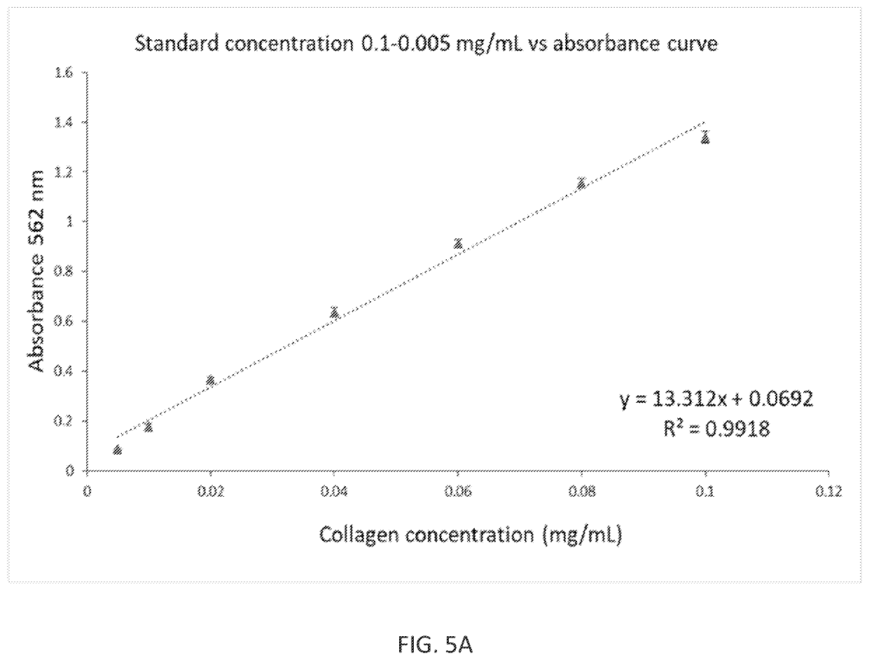

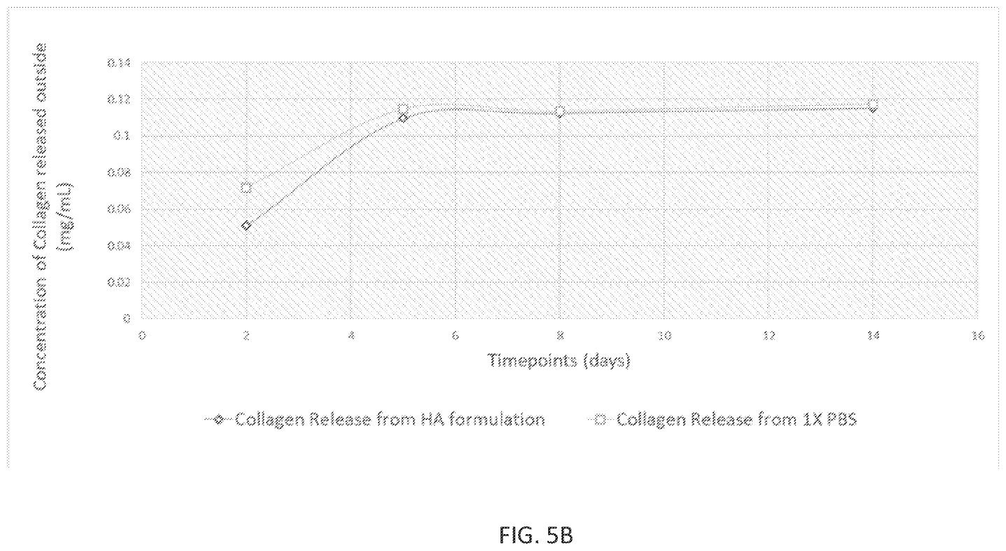

The release kinetics of a solution of (A) a recombinant collagen having amino acid sequence of SEQ ID NO: 1, and (B) a formulation of a recombinant collagen having amino acid sequence of SEQ ID NO: 1 and hyaluronic acid were investigated. The results are shown in FIGS. 4A, 4B, 5A, and 5B.

### Analysis

Standard curve: Triplicate absorbance values obtained of each sample and dilution were averaged and subtracted from the blank (1×PBS+0.5% sodium benzoate) value to obtain mean absorbance values (MAV) only from collagen in each sample/dilution. MAV of serial dilutions from 10 to 0.5 mg/mL were plotted against their respective concentrations. The same was done for dilutions from 0.08-0.02 mg/mL. A linear trendline was plotted for the 0.08-0.02 mg/mL range. The equation y=0.7033x-0.0131 was obtained with an R² value of 0.9936. This equation was used to calculate protein concentrations of the test samples of both control (A) and formulation (B) from their respective MAVs (FIG. 4A). Collagen mass per sample was calculated by multiplying the total solution volume present outside during the diffusion experiment (90 mL, 89.8 mL, 89.6 mL or 89.4 mL).

Concentrations were plotted against the timepoints to obtain a release trend. 2 such trends were obtained finally, one for Solution (A) and the other for Formulation (B) (FIG. 4B). As shown, in the figures, the protein is gradually released from both solution A and formulation B, which contains HA. The rate at which protein is released from formulation B appears to be slightly slower than from the solution not containing HA. This indicates a weak, but measurable, interaction between the HA and the collagen, which is likely because the solutions are miscible.

### Micro BCA Assay

The Micro BCA™ Protein Assay Kit protocol was referred to for analysis using the micro BCA Assay.

### Analysis

Triplicate absorbance values obtained from each sample and dilution were averaged and subtracted from the blank (1×PBS+0.5% sodium benzoate) value to obtain mean absorbance values (MAV) only from collagen in each sample/dilution. Standard deviation and error were calculated for each value.

MAV of serial dilutions from 10 to 0.5 mg/mL were plotted against their respective concentrations. The same was done for dilutions from 0.08-0.02 mg/mL. A linear trendline was plotted for the 0.08-0.02 mg/mL range. The equation y=13.312x+0.0692 was obtained with an R² value of 0.9918. This equation was used to calculate protein concentrations of the test samples of both control (A) and formulation (B) from their respective MAVs (FIG. 5A). Collagen mass per sample was calculated by multiplying the total solution volume present outside during the diffusion experiment (90 mL, 89.8 mL, 89.6 mL or 89.4 mL). Concentrations were plotted against the timepoints to obtain a trend of the amount of protein releasing out from a solution/formulation. Two such trends were obtained finally, one for Solution (A) and the other for Formulation (B) (FIG. 5B). As shown, protein gradually diffused from both Solution (A) and Formulation (B). Protein appears to diffuse more slowly from Formulation (B), indicating an attraction between the HA and the collagen.

### Example 59 (Fluorescent Labeling for Collagen Protein Release Kinetics)

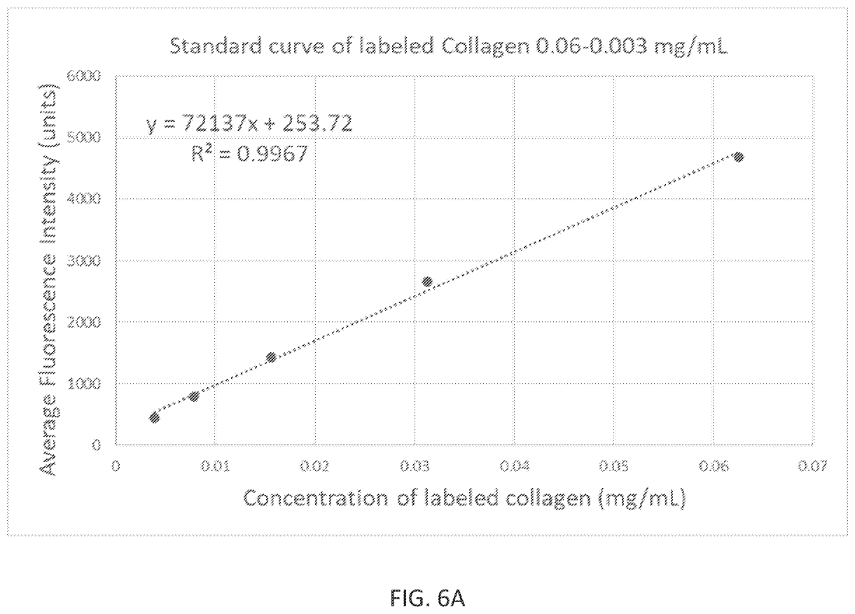

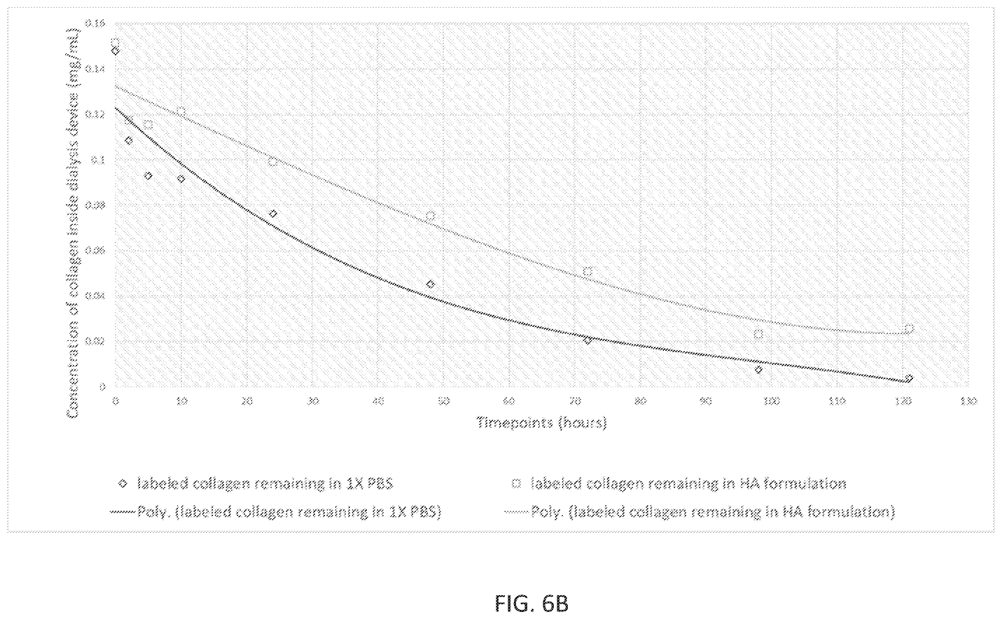

The release kinetics of Control solution (A) a recombinant collagen having amino acid sequence of SEQ ID NO: 1, and Formulation (B) a formulation of a recombinant collagen having amino acid sequence of SEQ ID NO: 1 and hyaluronic acid were fluorescently labeled for displaying release kinetics data. The results are shown in FIGS. 6A and 6B.

### Analysis

All dilutions for standard curve and timepoints from inside for (A) and (B) were pipetted on 96 well plates. 3 technical replicates of 100 uL each were pipetted. The fluorescence intensity was measured by setting excitation and emission wavelengths at 680 and 702 nm.

Standard curve: Triplicate intensity values obtained of each sample and dilution were averaged to obtain mean intensity values (MIV). MIV of serial dilutions from 0.0625, 0.03125, 0.015625, 0.0078125, 0.00390625 mg/mL were plotted against their respective concentrations. A linear trendline was plotted (FIG. 6A). The equation y=72137x+253.72 was obtained with an R2 value of 0.9967. This equation was used to calculate protein concentrations of the test samples of both solution (A) and formulation (B) from their respective MIVs. Unlabeled collagen concentration was calculated by multiplying labeled concentration values by 50. These concentrations were plotted against timepoints to give a trend of the amount of protein remaining inside the solution/formulation (FIG. 6B). As shown in FIG. 13B, the collagen diffuses from both Solution (A) and Formulation (B) at a gradual rate. The collagen appears to diffuse more slowly from the Formulation with HA (Formulation (B)), indicating an attraction between the HA and the collagen.

### Example 60 (Collagen Protein Release Kinetics/Crosslinked HA)

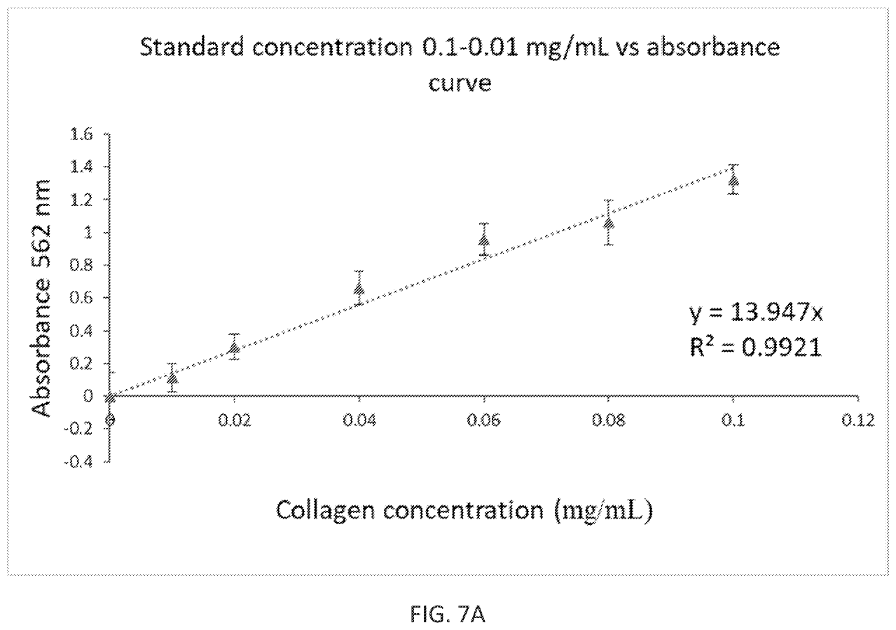

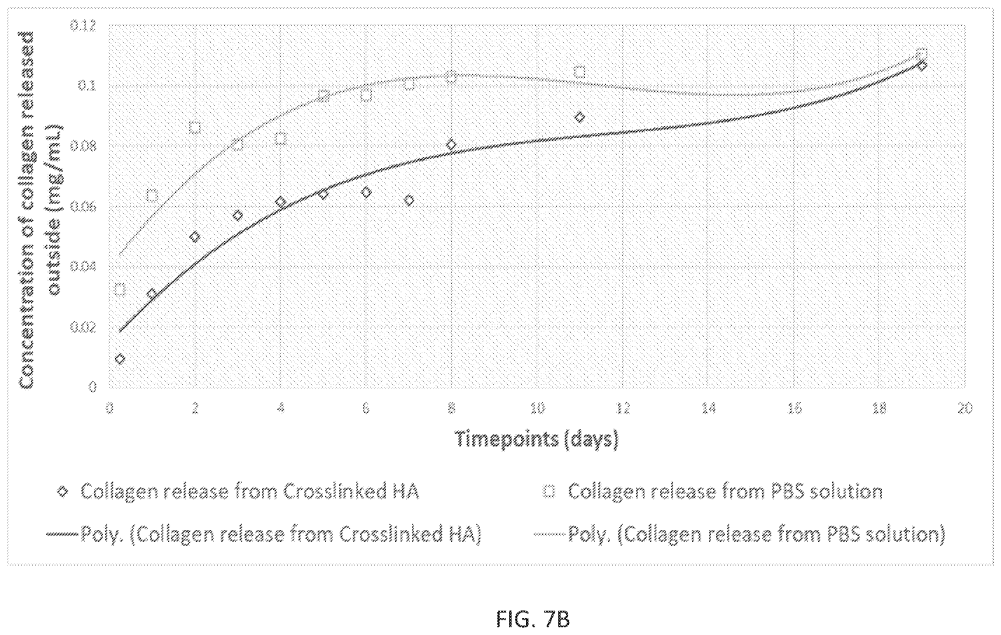

The release kinetics of a solution of (A) a 1% solution of recombinant collagen having amino acid sequence of SEQ ID NO: 1, and (B) a formulation of a recombinant collagen having amino acid sequence of SEQ ID NO: 1 and crosslinked hyaluronic acid were investigated. The results are shown in FIGS. 7A and 7B.

### Analysis

Triplicate absorbance values were obtained of each sample and dilutions were averaged and subtracted from the blank (1×PBS+value to obtain mean absorbance values (MAV) only from collagen in each sample/dilution. Standard deviation and error were calculated for each value.

MAV of serial dilutions from 0.2 to 0.01 mg/mL were plotted against their respective concentrations. A linear trendline was plotted for the 0.2-0.01 mg/mL range (FIG. 7A). The equation y=13.947x was obtained with an R2 value of 0.9921. The trendline is in good agreement with expectation and is suitable for use converting absorbance values to concentration for the diffusion experiment.

This equation was used to calculate collagen concentrations of the test samples of both Control (A) and Formulation (B) from their respective MAVs. Collagen mass per sample was calculated by multiplying the total solution volume present outside during the diffusion experiment (95 mL, 94 mL, 93 mL or 92 mL).

Concentrations were plotted against the timepoints to obtain a trend of the amount of collagen releasing out from a solution/formulation (FIG. 7B). Two such trends were obtained finally, one for collagen solution (A) and the other for collagen/HA formulation (B). As shown in FIG. 7B, the collagen release rate from the control solution (A) was similar to previous control examples, however, the collagen released much more slowly from the crosslinked HA/collagen formulation (B). For example, the amount of protein released from Formulation (B) at 8 days required only 3 days on average to diffuse from the control solution (A). After 19 days the collagen had fully released from the formulation (B).

### Example 61

A polyethylene glycol (PEG) and collagen mixture was prepared and dried, and visually examined. Example 61 was prepared using the method described in Example 1, except Polymer 1 was replaced with polyethylene glycol (molecular weight of 8 kDa; Sigma). The final solution was visually transparent, indicating that the two polymers were miscible in solution. Films were prepared using the method described in Example 3. The dried film separated into two phases where small, cracked pieces of a brittle and opaque material was surrounded by a ring of a brown and transparent material.

### Example 62

Hyaluronic acid and collagen mixture was prepared and visually examined. Example 28 was prepared using the method described in Example 15 except polymer 1 was replaced with HumaColl21® powder from Geltor. A 2 wt % 50 kDa HA solution was prepared using the method described in Example 15. The two solutions were mixed at a 1:1 (w/w) ratio in a 15 ml conical tube and mixed with on a stir plate at 10 RPM for 16 hours. The final solution was visually transparent. A film was prepared using the method as described in Example 3, except using an aluminum weighing pan. The dried film was transparent with a milky ring along the edge of the film, which was likely the preservative.

This example demonstrates that solutions including HumaColl21® powder and 50 kDa HA are miscible.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

1. A composition comprising (i) a recombinantly produced collagen and (ii) at least one member selected from the group consisting of hyaluronic acid, polyvinylpyrrolidone, polyacrylamide, poly(ethylene oxide), poly(2-oxazoline)s, polyethylenimine, sodium carboxymethylcellulose, chondroitin sulfate, acetylated hyaluronate, zinc hyaluronate, and alginic acid sodium salt, wherein the recombinant collagen is fully miscible with the at least one member selected from the group, and wherein the recombinant collagen has a sequence identity of at least about 85% to the amino acid sequence set forth in SEQ ID NO: 1 or at least about 85% to the amino acid sequence set forth in any one of SEQ ID NOs: 2-1002.

2.-8. (canceled)

9. The composition of claim 1, wherein the molecular weight of the recombinant collagen is from about 5 kDa to about 150 kDa.

10. The composition of claim 1, wherein the hyaluronic acid has a molecular weight of from about 3 kDa to about 10,000 kDa.

11.-60. (canceled)

61. A method of medical or cosmetic treatment comprising administering or applying an effective amount of the composition of claim 1 to a subject in need thereof.

62.-65. (canceled)

66. The method of claim 61, wherein the treatment comprises treating an inflammatory skin disease or mucous membrane disease.

67. The method of claim 61, wherein the treatment comprises treating lipoatrophy.

68.-82. (canceled)

83. A method of preparing a composition comprising collagen and hyaluronic acid, wherein the collagen is fully miscible with the hyaluronic acid, the method comprising:
a) dissolving hyaluronic acid in an NaOH solution;
b) adding collagen; and
c) adjusting the pH by adding HCl,
wherein the collagen is a recombinant collagen having an amino acid sequence identity of at least about 85% to SEQ ID NO: 1, and/or one or more collagen fragment sequence variants having an amino acid sequence identity of at least about 85% to the amino acid sequence set forth in in any one of SEQ ID NOs: 975-1002.

84. (canceled)

85. A method of preparing a composition comprising collagen and hyaluronic acid, wherein the collagen is fully miscible with the hyaluronic acid, the method comprising:
a) dissolving hyaluronic acid in water to form a hyaluronic acid solution;
b) dissolving collagen in water to form a collagen solution; and
c) mixing the hyaluronic acid and collagen solutions,
wherein the collagen is a recombinant collagen fragment having an amino acid sequence identity of at least about 85% to SEQ ID NO: 1, and/or one or more collagen fragment sequence variants having an amino acid sequence identity of at least about 85% to the amino acid sequence set forth in in any one of SEQ ID NOs: 975-1002.

86.-133. (canceled)

134. The composition of claim 1, wherein the polyvinylpyrrolidone has a molecular weight of from about 10 kDa to about 400 kDa.

135. The composition of claim 1, wherein the polyacrylamide has a molecular weight of from about 30 kDa to about 50 kDa, or from about 5,000 kDa to about 6,000 kDa.

136. The composition of claim 1, wherein the carboxymethylcellulose has a molecular weight of about 250 kDa and a degree of substitution of from about 0.70 to about 0.90 or from about 1.00 to about 1.25.

137. The composition of claim 1, wherein the chondroitin sulfate has a molecular weight of about 50 kDa.
